(19) United States
(12) Patent Application Publication
(10) Pub. No.: US 2016/0002358 A1
(43) Pub. Date: Jan. 7, 2016

(54) BIOMASS PROCESSING USING IONIC LIQUIDS

(71) Applicant: HYRAX ENERGY, INC., Mountain View, CA (US)

(72) Inventors: Rodrigo E. Teixeira, Palo Alto, CA (US); Kurtis G. Knapp, Menlo Park, CA (US)

(21) Appl. No.: 14/772,319

(22) PCT Filed: Mar. 4, 2014
(86) PCT No.: PCT/US14/20375

§ 371 Date: Sep. 2, 2015

Related U.S. Application Data
(60) Provisional application No. 61/772,538, filed on Mar. 5, 2013.
(60) Provisional application No. 61/772,535, filed on Mar. 5, 2013.
(60) Provisional application No. 61/780,676, filed on Mar. 13, 2013.
(60) Provisional application No. 61/832,797, filed on Jun. 8, 2013.
(60) Provisional application No. 61/862,420, filed on Aug. 5, 2013.

(51) Int. Cl. C08B 1/00; C12P 7/10; C13K 13/00

(52) U.S. Cl. CPC C08B 1/003; C13K 13/007; C12P 7/10; C12P 2201/00

(57) ABSTRACT

Without limitation, the disclosure provides processes for (a) dissolving biomass in ionic liquids, (b) deconstructing cellulose, hemicellulose and/or lignin into derivatives including fermentable sugars, (c) separating the biomass derivatives from the ionic liquid, and (d) converting the biomass derivatives to useful fuels or chemicals, either dissolved within or separated from the ionic liquid. It should be understood that processes described herein can be used in isolation or in combination with each other.

### CROSS-REFERENCE

This application claims the benefit of U.S. Provisional Application No. 61/772,535 filed on Mar. 5, 2013, U.S. Provisional Application No. 61/772,538 filed on Mar. 5, 2013, U.S. Provisional Application No. 61/780,676 filed on Mar. 13, 2013, U.S. Provisional Application No. 61/832,797 filed on Jun. 8, 2013 and U.S. Provisional Application No. 61/862,420 filed on Aug. 5, 2013, each of which is incorporated herein by reference in its entirety.

### BACKGROUND

The disclosure relates to industrial biotechnology and biomass processing using ionic liquids.

Plant material can be a feedstock for the production of renewable fuels and chemicals. However, realizing this objective is dependent on the development of a process for breaking ligno-cellulose (i.e., plant biomass) down into its components including lignin, lignin-derivatives, and fermentable sugars derived from cellulose and hemicellulose.

Ionic liquids (“IL” or “ILs”) are salts (e.g., comprising cations and anions) that are a liquid. Interest has grown regarding using ionic liquids in various chemical processes.

### SUMMARY

Some ionic liquids can dissolve plant biomass or components thereof (i.e., cellulose and/or lignin). However, an ionic liquid-based process that dissolves biomass, deconstructs it into its component parts (e.g., lignin, lignin derivatives and fermentable sugars), and performs separations to recover the ionic liquid and biomass components is needed. Without limitation, the disclosure provides processes for (a) dissolving biomass in ionic liquids, (b) deconstructing cellulose, hemicellulose and/or lignin into derivatives including fermentable sugars, (c) separating the biomass derivatives from the ionic liquid, and (d) converting the biomass derivatives to useful fuels or chemicals, either dissolved within or separated from the ionic liquid. It should be understood that processes described herein can be used in isolation or in combination with each other.

In some embodiments, the disclosure provides a method for extracting one or more biomass components comprising: contacting a composition comprising one or more biomass components in an ionic liquid with a supercritical or near-supercritical fluid.

In some embodiments, the method further comprises recovering the extracted one or more biomass components from the supercritical or near-supercritical fluid.

In some embodiments, the composition comprising one or more biomass components in an ionic liquid is obtained by dissolving a biomass in an ionic liquid and hydrolyzing the biomass in the ionic liquid.

In some embodiments, the one or more biomass components comprise sugars, furanic compounds, lipids, ash, fatty acids, resin acids, waxes, terpenes, acetates, acetic acids, alcohols, amino acids, sugar acids, phenols, aldehydes, ethers or combinations thereof.

In some embodiments, the one or more biomass components are recovered from the supercritical or near-supercritical fluid using supercritical chromatography.

In some embodiments, the one or more biomass components are recovered from the supercritical or near-supercritical fluid by lowering the pressure of the fluid. In some embodiments, the pressure is not lowered below the critical pressure of the supercritical or near-supercritical fluid.

In some embodiments, the one or more biomass components are recovered from the supercritical or near-supercritical fluid by lowering the temperature of the fluid. In some embodiments, the one or more biomass components are recovered from the supercritical or near-supercritical fluid by raising the temperature of the fluid.

In some embodiments, the one or more biomass components are sequentially extracted from the ionic liquid in a plurality of supercritical or near-supercritical fluids.

In some embodiments, the supercritical or near-supercritical fluid comprises a co-solvent. In some embodiments, the co-solvent is selected from water, alcohol, acetic acid, acetate, acetone, carboxylic acids, organic polar acids or any combination thereof. In some embodiments, the co-solvent is derived from the biomass.

In some embodiments, the supercritical or near-supercritical fluid is methane, ethane, propane, ethylene, propylene, nitrogen, hydrogen, helium, argon, oxygen, nitrous oxide, or any combination thereof. In some embodiments, the supercritical or near-supercritical fluid is carbon dioxide.

In some embodiments, the biomass components comprise carbohydrates, the molecular weight of the carbohydrates is reduced in the ionic liquid to form sugars, and the sugars are extracted from the ionic liquid.

In some embodiments, ionic liquid is rejected from the supercritical or near-supercritical fluid by increasing the pressure of the fluid following extraction and before recovery of the biomass components from the fluid. In some embodiments, water is extracted from the composition in the supercritical or near-supercritical fluid.

In some embodiments, the disclosure provides a method for extracting a biomass component from an ionic liquid mixture comprising: contacting an ionic liquid mixture containing a biomass component with a supercritical fluid to form a post-extraction supercritical fluid mixture and a post-extraction ionic liquid mixture, wherein the post-extraction ionic liquid mixture has less amount of the biomass component than the amount contained in the ionic liquid mixture, wherein the post-extraction supercritical fluid mixture has more amount of the biomass component than the amount contained in the supercritical fluid.

In some embodiments, the post-extraction supercritical fluid mixture has a pressure such that ionic liquid is rejected from the post-extraction supercritical fluid mixture. In some embodiments, water is extracted from the ionic liquid mixture into the post-extraction supercritical fluid mixture.

In some embodiments, the disclosure provides a method for extracting one or more biomass components comprising: contacting a solution comprising one or more biomass components in an ionic liquid with a fluid, wherein substantially none of the ionic liquid dissolves in the fluid, and wherein at least some of the biomass components dissolve in the fluid.

In some embodiments, the disclosure provides a method for extracting one or more biomass components comprising: contacting a solution comprising one or more biomass components in an ionic liquid with a fluid, wherein at least some of the biomass components dissolve in the fluid, and increasing the pressure so that substantially none of the ionic liquid dissolves in the fluid.

In some embodiments, the fluid is miscible in the ionic liquid. In some embodiments, the fluid is a supercritical or near-supercritical fluid.

In some embodiments, the disclosure provides a method for recovering biomass components from an ionic liquid comprising: contacting a composition comprising an ionic liquid, water and a hydrogen bonding solute with a fluid to form a first phase comprising an ionic liquid and a second phase comprising water and the hydrogen bonding solute.

In some embodiments, the method further comprises partitioning the second phase from the first phase. In some embodiments, contacting the composition with the fluid forms a third phase comprising the fluid.

In some embodiments, the hydrogen bonding solute is derived from biomass. In some embodiments, the hydrogen bonding solute has at least one hydroxyl group. In some embodiments, the hydrogen bonding solute comprises sugar, an aldose, a ketose, or any combination thereof. In some embodiments, the ionic liquid is hydrophilic.

In some embodiments, the fluid is a pressurized gas. In some embodiments, the fluid is a liquefied gas. In some embodiments, the fluid is a supercritical or near-supercritical fluid. In some embodiments, the fluid is non-polar. In some embodiments, the fluid comprises carbon dioxide.

In some embodiments, the composition is contacted with the fluid at a pressure greater than atmospheric pressure.

In some embodiments, contacting the composition with the fluid decreases the viscosity of the composition. In some embodiments, the viscosity of the first phase is less than the viscosity of the composition without contact with the fluid.

In some embodiments, the dielectric constant of the first phase is less than the dielectric constant of the ionic liquid.

In some embodiments, the concentration of the water in the hydrolysis reaction is such that the concentration of the hydrogen bonding solute in the second phase is near saturation.

In some embodiments, water is added to the hydrolysis reaction at a rate such that the concentration of ionic liquid in the second phase is less than 25% (w/w).

In some embodiments, the disclosure provides a method for recovering biomass components from an ionic liquid, the method comprising: forming a first phase and a second phase from a hydrolyzed biomass composition comprising an ionic liquid, water and one or more biomass components, wherein the first phase comprises an ionic liquid and the second phase comprises water and one or more biomass components.

In some embodiments, the hydrolyzed biomass composition is obtained by hydrolyzing the biomass and/or biomass component in the ionic liquid.

In some embodiments, the biomass component is a sugar. In some embodiments, the sugar comprises glucose. In some embodiments, the sugar at least partially stabilizes the second phase. In some embodiments, the concentration of the water in the hydrolysis reaction is such that the concentration of the sugar in the second phase is near saturation.

In some embodiments, water is added to the hydrolysis reaction at a rate such that the concentration of ionic liquid in the second phase is less than 25% (w/w).

In some embodiments, the composition is pressurized to form the first phase and the second phase. In some embodiments, the temperature of the composition is reduced to form the first phase and the second phase. In some embodiments, the composition is contacted with pressurized carbon dioxide to form the first phase and the second phase.

In some embodiments, the hydrolysis of biomass provides solutes that induce the formation of the first phase and the second phase. In some embodiments, the solutes comprise sugar, oil, methanol, or any combination thereof.

In some embodiments, the disclosure provides a method for separating water and a hydrogen bonding solute from a composition comprising an ionic liquid, water and hydrogen bonding solute, wherein the ratio of the mass of water to the mass of hydrogen bonding solute when separated is approximately equal to the ratio of the mass of water to the mass of hydrogen bonding solute in the composition.

In some embodiments, the ratio of the mass of water to the mass of hydrogen bonding solute when separated is within about 20% of the ratio of the mass of water to the mass of hydrogen bonding solute in the composition.

In some embodiments, the disclosure provides a method for separating hydrogen bonding solute from a composition comprising an ionic liquid and hydrogen bonding solute, wherein the concentration of the ionic liquid increases when the hydrogen bonding solute is separated from the composition.

In some embodiments, the ionic liquid is not diluted in the separation. In some embodiments, the separation does not comprise concentrating the ionic liquid by evaporating water.

In some embodiments, the disclosure provides a method for separating water and hydrogen bonding solute from a composition comprising an ionic liquid, water and hydrogen bonding solute, wherein the hydrogen bonding solute is separated from the ionic liquid at a concentration of at least 10% (w/w).

In some embodiments, the disclosure provides a method for separating a solute from an ionic liquid comprising reducing the dielectric constant of a composition comprising an ionic liquid and a solute by contacting the composition with a pressurized gas.

In some embodiments, the solute is precipitated from the ionic liquid. In some embodiments, the solute comprises sugar.

In some embodiments, the disclosure provides a method for producing fermentable sugar comprising hydrolyzing a polysaccharide in an ionic liquid to produce sugar and continuously removing the sugar from the ionic liquid.

In some embodiments, the disclosure provides a method for producing fermentable sugar comprising hydrolyzing a polysaccharide in an ionic liquid to produce sugar and continuously cooling the hydrolysate.

In some embodiments, the mass of furanic compounds produced is less than 1% of the mass of sugar produced in the ionic liquid.

In some embodiments, the sugar is removed from the ionic liquid at an optionally variable rate such that the mass of furanic compounds produced is less than 1% of the mass of sugar produced in the ionic liquid.

In some embodiments, the rate of sugar removal from the ionic liquid is approximately equal to the rate of sugar production. In some embodiments, the sugar is continuously removed by extraction in a supercritical or near-supercritical fluid.

In some embodiments, the sugar is fermentable when removed from the ionic liquid.

In some embodiments, the disclosure provides a composition comprising an ionic liquid, a pressurized gas, water and a biomass.

In some embodiments, the disclosure provides a multi-phasic system comprising: (a) a first phase comprising a pressurized gas, water and one or more biomass components; and (b) a second phase comprising an ionic liquid and one or more biomass components.

In some embodiments, the disclosure provides a multi-phasic system comprising: (a) a first phase comprising a pressurized gas, water and one or more biomass components; (b) a second phase comprising a pressurized gas, water, one or more biomass components and an ionic liquid; and (c) a third phase comprising an ionic liquid and one or more biomass components.

In some embodiments, the pressurized gas is a supercritical or near-supercritical fluid. In some embodiments, the first phase comprises less than about 0.5% ionic liquid.

In some embodiments, the disclosure provides a multi-phasic system comprising: (a) a first phase comprising an ionic liquid; (b) a second phase comprising water and one or more biomass components; and (c) optionally a third phase comprising a fluid.

In some embodiments, the fluid is a pressurized gas. In some embodiments, the fluid is a liquefied gas. In some embodiments, the second phase comprises less than about 25% ionic liquid.

In some embodiments, the disclosure provides a method for recovering a furanic compound from an ionic liquid comprising: contacting a composition comprising a furanic compound and an ionic liquid with a fluid.

In some embodiments, the composition comprising a furanic compound is produced by contacting an ionic liquid with a biomass, a polysaccharide, a sugar, or a combination thereof. In some embodiments, the ionic liquid further comprises a catalyst.

In some embodiments, the fluid is a pressurized gas, liquefied gas, or supercritical or near-supercritical fluid. In some embodiments, the furanic compound is extracted in the supercritical or near-supercritical fluid.

In some embodiments, the ionic liquid comprises water, contact with the fluid creates an aqueous phase, and the furanic compound is recovered in the aqueous phase. In some embodiments, the ionic liquid comprises water, contact with the fluid creates an organic phase, and the furanic compound is recovered in the organic phase.

In some embodiments, contacting the ionic liquid with a fluid forms a first phase comprising the ionic liquid and a second phase comprising the furanic compound and the furanic compound is recovered from the ionic liquid by partitioning the second phase from the first phase. In some embodiments, the furanic compound is hydroxymethylfurfural, 2,5-dimethylfuran, furfural, or a combination thereof.

In some embodiments, the disclosure provides a method for manufacturing or purifying an ionic liquid, comprising removing non-ionic components from the ionic liquid by contacting the ionic liquid with a pressurized gas.

In some embodiments, the method further comprises synthesizing the ionic liquid by mixing ionic components prior to removing non-ionic components from the ionic liquid. In some embodiments, the method further comprises synthesizing the ionic liquid by creating ionic components in a reaction prior to removing non-ionic components from the ionic liquid.

In some embodiments, the disclosure provides a method for separating a sugar from an ionic liquid comprising contacting a composition comprising an ionic liquid and a biomass component with a fluid, wherein less than 10 grams of ionic liquid is lost per kilogram of biomass component separated.

In some embodiments, less than 1 gram of ionic liquid is lost per kilogram of biomass component separated. In some embodiments, less than 0.1 gram of ionic liquid is lost per kilogram of biomass component separated.

In some embodiments, the disclosure provides a sugar composition comprising water, a sugar and carbon dioxide, wherein the sugar is derived from cellulose, hemicellulose, or a combination thereof.

In some embodiments, the disclosure provides a sugar composition as described herein, further comprising ionic liquid.

In some embodiments, the disclosure provides a sugar composition as described herein, wherein the concentration of ionic liquid is detectable and less than 1%.

In some embodiments, the disclosure provides a sugar composition comprising water, a sugar and an ionic liquid, wherein the sugar is derived from cellulose, hemicellulose, or a combination thereof.

In some embodiments, the disclosure provides a sugar composition as described herein, further comprising carbon dioxide.

In some embodiments, the disclosure provides a sugar composition as described herein, wherein the concentration of carbon dioxide is detectable and less than 1%.

In some embodiments, the disclosure provides a fermentable sugar comprising a sugar and an ionic liquid, wherein the mass of sugar is at least 20 times greater than the mass of the ionic liquid, and wherein the sugar is derived from cellulose, hemicellulose, or a combination thereof.

In some embodiments, the disclosure provides a fermentable sugar, wherein the sugar comprises at least one component selected from furanics, phenols, ethers, aldehydes, ash, lignin, and lignin derivatives.

In some embodiments, the disclosure provides a fermentable sugar, wherein the concentration of at least one of: furanics, phenols, ethers, aldehydes, ash, lignin, and lignin derivatives, or any combination thereof is less than 1% (w/w).

In some embodiments, the disclosure provides a method for recovering biomass components from an ionic liquid comprising: (a) contacting a composition comprising ionic liquid, water and a hydrogen bonding solute with a fluid to form a first phase comprising ionic liquid and a second phase comprising water and the hydrogen bonding solute; (b) recovering or concentrating at least some of the hydrogen bonding solute from the second phase; and (c) returning at least some of the hydrogen bonding solute from (b) to the mixture.

In some embodiments, the hydrogen bonding solute is recovered or concentrated by reverse osmosis.

In some embodiments, the disclosure provides a method for recovering biomass components from an ionic liquid comprising: (a) contacting a composition comprising ionic liquid, water and a hydrogen bonding solute with a fluid to form a first phase comprising ionic liquid and a second phase comprising ionic liquid, water and the hydrogen bonding solute; and (b) recovering or concentrating at least some of the ionic liquid from the second phase.

In some embodiments, In some embodiments, the ionic liquid is recovered by electrodialysis. In some embodiments, an aqueous biphasic system (ABS) is produced.

In some embodiments, the method further comprises lowering the temperature of the composition, first phase and/or second phase. In some embodiments, the method further comprises recovering the fluid from the first phase and/or the second phase. In some embodiments, the fluid is recovered using a flash tank or a heated tank.

In some embodiments, the method further comprises compressing and/or re-contacting the recovered fluid to the composition.

In some embodiments, the method further comprises partitioning the second phase from the first phase. In some embodiments, contacting the composition with the fluid forms a third phase comprising the fluid.

In some embodiments, the hydrogen bonding solute is derived from biomass. In some embodiments, the hydrogen bonding solute has at least one hydroxyl group. In some embodiments, the hydrogen bonding solute comprises sugar, an aldose, a ketose, or any combination thereof.

In some embodiments, the ionic liquid is hydrophilic. In some embodiments, the fluid is a pressurized gas. In some embodiments, the fluid is a liquefied gas. In some embodiments, the fluid is a supercritical or near-supercritical fluid. In some embodiments, the fluid is non-polar. In some embodiments, the fluid comprises carbon dioxide.

In some embodiments, the composition is contacted with the fluid at a pressure greater than atmospheric pressure. In some embodiments, contacting the composition with the fluid decreases the viscosity of the composition. In some embodiments, the viscosity of the first phase is less than the viscosity of the composition without contact with the fluid. In some embodiments, the dielectric constant of the first phase is less than the dielectric constant of the ionic liquid.

In some embodiments, the concentration of the water in the hydrolysis reaction is such that the concentration of the hydrogen bonding solute in the second phase is near saturation. In some embodiments, water is added to the hydrolysis reaction such that the concentration of ionic liquid in the second phase is less than about 25% (w/w).

In some embodiments, the composition comprising an ionic liquid, water and a hydrogen bonding solute is obtained by dissolving a biomass in an ionic liquid and hydrolyzing the biomass in the ionic liquid.

In some embodiments, the disclosure provides a method for recovering biomass components from an ionic liquid, the method comprising: (a) forming a first phase and a second phase from a hydrolyzed biomass composition comprising ionic liquid, water and one or more biomass components, wherein the first phase comprises ionic liquid and the second phase comprises water and one or more biomass components; (b) recovering or concentrating at least some of the biomass components from the second phase; and (c) returning at least some of the biomass components from (b) to the hydrolyzed biomass composition. In some embodiments, the biomass components are recovered or concentrated by reverse osmosis.

In some embodiments, the disclosure provides a method for recovering biomass components from an ionic liquid, the method comprising: (a) forming a first phase and a second phase from a hydrolyzed biomass composition comprising ionic liquid, water and one or more biomass components, wherein the first phase comprises ionic liquid and the second phase comprises ionic liquid, water and one or more biomass components; and (b) recovering or concentrating at least some of the ionic liquid from the second phase.

In some embodiments, the ionic liquid is recovered by electrodialysis. In some embodiments, an aqueous biphasic system (ABS) is produced. In some embodiments, the hydrolyzed biomass composition is obtained by hydrolyzing the biomass and/or biomass component in the ionic liquid. In some embodiments, the biomass component is a sugar. In some embodiments, the sugar comprises glucose.

In some embodiments, the sugar at least partially stabilizes the second phase. In some embodiments, the concentration of the water in the hydrolysis reaction is such that the concentration of the sugar in the second phase is near saturation. In some embodiments, water is added to the hydrolysis reaction at a rate such that the concentration of ionic liquid in the second phase is less than about 25% (w/w).

In some embodiments, the composition is pressurized to form the first phase and the second phase. In some embodiments, the temperature of the composition is reduced to form the first phase and the second phase. In some embodiments, the composition is contacted with pressurized carbon dioxide to form the first phase and the second phase.

In some embodiments, the hydrolysis of biomass provides solutes that induce the formation of the first phase and the second phase. In some embodiments, the solutes comprise sugar, oil, methanol, or any combination thereof.

In some embodiments, the method further comprises lowering the temperature of the composition, first phase and/or second phase.

In some embodiments, the disclosure provides a extractor capable of performing the methods of the disclosure.

In some embodiments, the disclosure provides an extractor comprising an inlet, a first outlet and a second outlet, wherein the inlet is configured to feed a composition comprising ionic liquid, water and a solute into the extractor, the extractor is capable of forming a first phase comprising ionic liquid and a second phase comprising water and the solute; and at least one of: (a) the first outlet is in fluid communication with a first unit capable of recovering or concentrating the solute in the second phase; and (b) the second outlet is in fluid communication with a second unit capable of recovering gases dissolved in the first phase.

In some embodiments, the first unit is a reverse osmosis unit. In some embodiments, the second unit is a flash tank or a heated tank. In some embodiments, the second unit is connected to a compressor capable of compressing the recovered gases and injecting the recovered gases into the extractor.

In some embodiments, the disclosure provides a method for recovering biomass components from an ionic liquid, the method comprising: pressurizing a hydrolyzed biomass composition comprising ionic liquid, water and one or more biomass components to form a first phase and a second phase, wherein the first phase comprises ionic liquid and the second phase comprises water and one or more biomass components.

In some embodiments, the composition is pressurized to greater than atmospheric pressure. In some embodiments, the composition is pressurized to less than atmospheric pressure. In some embodiments, the composition is pressurized by contacting with a pressurized gas. In some embodiments, the gas is carbon dioxide. In some embodiments, the gas is not carbon dioxide. In some embodiments, the gas is nitrogen or a noble gas.

In some embodiments, the disclosure provides a method for recovering biomass components from an ionic liquid comprising: contacting a composition comprising ionic liquid and water with a pressurized gas to form a first phase comprising ionic liquid and a second phase comprising water.

In some embodiments, the composition further comprises a hydrogen bonding solute. In some embodiments, the second phase comprises the hydrogen bonding solute. In some embodiments, the gas is carbon dioxide. In some embodiments, the gas is not carbon dioxide. In some embodiments, the gas is nitrogen or a noble gas.

In some embodiments, the disclosure provides a method for recovering biomass components from an ionic liquid, the method comprising: forming a first phase and a second phase from a hydrolyzed biomass composition comprising an ionic liquid, water and one or more biomass components, wherein the first phase comprises an ionic liquid and the second phase comprises water and one or more biomass components.

In some embodiments, the hydrolyzed biomass composition is obtained by hydrolyzing the biomass and/or biomass component in the ionic liquid.

In some embodiments, the biomass component is a sugar. In some embodiments, the sugar comprises glucose. In some embodiments, the sugar at least partially stabilizes the second phase. In some embodiments, the concentration of the water in the hydrolysis reaction is such that the concentration of the sugar in the second phase is near saturation.

In some embodiments, water is added to the hydrolysis reaction at a rate such that the concentration of ionic liquid in the second phase is less than 25% (w/w).

In some embodiments, the composition is pressurized to form the first phase and the second phase. In some embodiments, the temperature of the composition is reduced to form the first phase and the second phase. In some embodiments, the composition is contacted with pressurized carbon dioxide to form the first phase and the second phase.

In some embodiments, the hydrolysis of biomass provides solutes that induce the formation of the first phase and the second phase. In some embodiments, the solutes comprise sugar, oil, methanol, or any combination thereof.

In some embodiments, the ionic liquid is a lignin-dissolving ionic liquid. In some embodiments, the ionic liquid dissolves lignin. In some embodiments, the ionic liquid dissolves lignin, but not cellulose. In some embodiments, the biomass components comprise lignin or derivatives thereof.

In some embodiments, the hydrogen bonding solute is a kosmotropic salt. In some embodiments, the hydrogen bonding solute is an acid or a base.

In some embodiments, the method uses two ionic liquids. In some embodiments, the method uses a lignin-dissolving ionic liquid followed by a cellulose-dissolving ionic liquid.

In some embodiments, the disclosure provides a method for processing biomass, the method comprising: (a) contacting the biomass with a lignin-dissolving ionic liquid; (b) recovering cellulose from the lignin-dissolving ionic liquid; and (c) contacting cellulose with a cellulose-dissolving ionic liquid.

In some embodiments, the method further comprises hydrolyzing the cellulose in the cellulose-dissolving ionic liquid. In some embodiments, the method further comprises hydrolyzing the hemi-cellulose in the cellulose-dissolving ionic liquid, the lignin-dissolving ionic liquid, or a combination thereof. In some embodiments, the method further comprises fractionating and/or performing chemical reactions on the lignin in the lignin-dissolving ionic liquid. In some embodiments, the method further comprises recovering solutes from the lignin-dissolving ionic liquid by forming an aqueous biphasic system.

In some embodiments, the solutes comprise C5 sugars, lignin, lignin derivatives, or any combination thereof.

In some embodiments, the method further comprises recovering solutes from the cellulose-dissolving ionic liquid by forming an aqueous biphasic system. In some embodiments, the solutes comprise C6 sugars.

In some embodiments, lignin, cellulose, hemi-cellulose, ash, or any combination thereof are precipitated from the lignin-dissolving ionic liquid by contacting with a fluid. In some embodiments, the fluid is a pressurized gas.

In some embodiments, the disclosure provides a method for dissolving biomass and components thereof, the method comprising contacting the biomass or component thereof with an ionic liquid at a pressure greater than atmospheric pressure.

In some embodiments, the pressure is imposed directly into the liquid. In some embodiments, the pressure is imposed between the ionic liquid and a surface. In some embodiments, the pressure is imposed indirectly. In some embodiments, the pressure is imposed by first compressing a fluid other than the ionic liquid.

In some embodiments, the pressure is increased to more than 2 atmospheres, more than 5 atmospheres, or more than 20 atmospheres.

In some embodiments, the applied pressure is stationary or non-stationary. In some embodiments, the non-stationary pressure takes the form of vibration, acoustic waves, ultrasound, agitation, and the like. In some embodiments, the pressure is oscillated.

In some embodiments, the increased pressure increases the rate at which the biomass or components thereof dissolve in the ionic liquid by at least 1% relative to the rate at which the biomass dissolves in the ionic liquid at atmospheric pressure.

In some embodiments, the increased pressure increases the solubility of the biomass or components thereof in the ionic liquid by at least 1% relative to the solubility of the biomass in the ionic liquid at atmospheric pressure.

In some embodiments, the ionic liquid is contacted with a pressurized gas. In some embodiments, the pressurized gas is air. In some embodiments, the pressurized gas is carbon dioxide, methane, ethane, propane, butane, natural gas, methanol, ethanol, propanol, butanol, nitrous oxide, ammonia, water, or any combination thereof.

In some embodiments, the pressurized gas at least partially dissolves in the ionic liquid. In some embodiments, contacting the ionic liquid with the pressurized gas reduces the viscosity of the ionic liquid by at least 5%. In some embodiments, the biomass has a solubility of at least 3% in the ionic liquid.

In some embodiments, the rate at which the biomass dissolves in the ionic liquid is at least 20% of the maximum rate at pressures between atmospheric pressure and 100 atm. In some embodiments, the solubility of the biomass in the ionic liquid is at least 20% of the maximum solubility at pressures between atmospheric pressure and 100 atm.

In some embodiments, the method further comprises agitating the ionic liquid. In some embodiments, the method further comprises ultrasounding the ionic liquid.

In some embodiments, the biomass is contacted with the ionic liquid at a temperature, the ionic liquid is a liquid at the temperature when in contact with the pressurized gas, and the ionic liquid is a solid at the temperature when not in contact with the pressurized gas.

In some embodiments, the disclosure provides a method for dissolving biomass comprising contacting biomass with ionic liquid, wherein the ionic liquid is in contact with a pressurized gas.

In some embodiments, the rate at which the biomass dissolves in the ionic liquid is at least 5% greater than the rate at which the biomass dissolves in the ionic liquid when the ionic liquid is not in contact with the pressurized gas. In some embodiments, the solubility of the biomass in the ionic liquid is at least 1% greater than the solubility of the biomass in the ionic liquid when the ionic liquid is not in contact with the pressurized gas.

In some embodiments, contacting the ionic liquid with the pressurized gas reduces the viscosity of the ionic liquid by at least 5%. In some embodiments, the biomass has a solubility of at least 3% in the ionic liquid.

In some embodiments, the rate at which the biomass dissolves in the ionic liquid is at least 20% of the maximum rate at pressures between atmospheric pressure and 100 atm. In some embodiments, the solubility of the biomass in the ionic liquid is at least 20% of the maximum solubility at pressures between atmospheric pressure and 100 atm.

In some embodiments, the method further comprises agitating the ionic liquid. In some embodiments, the method further comprises ultrasounding the ionic liquid.

In some embodiments, the gas comprises carbon dioxide.

In some embodiments, the biomass is contacted with the ionic liquid at a temperature, the ionic liquid is a liquid at the temperature when in contact with the pressurized gas, and the ionic liquid is a solid at the temperature when not in contact with the pressurized gas.

In some embodiments, the disclosure provides a method for hydrolyzing biomass, the method comprising adding water to a reaction mixture comprising ionic liquid and biomass, wherein the water is added at a rate such that the ratio (by mass) of side-products (HMF, furan) to sugar is less than the ratio (by mass) of side-products (HMF, furan, etc) to sugar obtained by the identical reaction comprising a fixed amount of water added.

In some embodiments, the disclosure provides a method for hydrolyzing biomass, the method comprising adding water to a reaction mixture comprising ionic liquid and biomass, wherein the water is added at a rate such that biomass solubilization is not substantially inhibited and hydrolysis is not substantially inhibited.

In some embodiments, the disclosure provides a method for hydrolyzing biomass, the method comprising adding water to a reaction mixture comprising ionic liquid and biomass, wherein the water is added at a rate that is approximately equal to the rate at which water is consumed in the reaction.

In some embodiments, the disclosure provides a method for hydrolyzing biomass, the method comprising adding water to a reaction mixture comprising ionic liquid and biomass, wherein the water is added at a rate that maintains the water concentration in the reaction mixture below about 5% (w/w).

In some embodiments, the biomass is at least partially dissolved in the ionic liquid. In some embodiments, the reaction mixture comprises non-dissolved biomass. In some embodiments, the reaction mixture further comprises a pressurized gas. In some embodiments, the reaction mixture further comprises an acid. In some embodiments, the reaction mixture further comprises carbon dioxide and at least some of the carbon dioxide is present as carbonic acid.

In some embodiments, the disclosure provides a method for hydrolyzing biomass comprising adding water to a mixture, wherein the mixture comprises biomass, water and ionic liquid, and wherein the water is added at a rate that maintains the ratio of the concentration of water to the concentration of the biomass in the mixture.

In some embodiments, the ratio is maintained between 50% and 150% of the ratio before water is added. In some embodiments, the ratio is maintained between 80% and 120% of the ratio before water is added.

In some embodiments, the ratio is maintained until the biomass is at least 50% hydrolyzed. In some embodiments, the ratio is maintained until the biomass is at least 75% hydrolyzed.

In some embodiments, the method further comprises, when the biomass is at least 90% hydrolyzed, adding water to the mixture to increase the ratio to at least 200% of the ratio before water is added.

In some embodiments, the mixture further comprises an acid.

In some embodiments, the disclosure provides a method for hydrolyzing biomass comprising adding water to a mixture, wherein the mixture comprises biomass, water and ionic liquid, and wherein the water is added at a rate that maintains the ratio of the concentration of water to the concentration of the ionic liquid in the mixture.

In some embodiments, the ratio is maintained between 50% and 150% of the ratio before water is added. In some embodiments, the ratio is maintained between 80% and 120% of the ratio before water is added. In some embodiments, the ratio is maintained until the biomass is at least 50% hydrolyzed. In some embodiments, the ratio is maintained until the biomass is at least 75% hydrolyzed.

In some embodiments, the method further comprises, when the biomass is at least 90% hydrolyzed, adding water to the mixture to increase the ratio to at least 200% of the ratio before water is added. In some embodiments, the mixture further comprises an acid.

In some embodiments, the disclosure provides a method for hydrolyzing biomass, comprising adding water to a mixture, wherein the mixture comprises ionic liquid, water and biomass having glycosidic bonds, and wherein the water is added to the mixture at a rate that maintains a greater than stoichiometric concentration of water relative to the glycosidic bonds. In some embodiments, the mixture further comprises an acid.

In some embodiments, the ratio of the concentration of water to the concentration of glycosidic bonds is about 2. In some embodiments, the ratio of the concentration of water to the concentration of glycosidic bonds is at least 50 when the biomass is about 90% hydrolyzed.

In some embodiments, the disclosure provides a method for hydrolyzing biomass, wherein water is added to a mixture, wherein the mixture comprises ionic liquid, water and biomass, and wherein the water is added at a rate that reduces the electrical conductivity of the reaction mixture over time.

In some embodiments, the electrical conductivity is reduced in proportion to the extent to which the biomass is hydrolyzed. In some embodiments, the mixture further comprises an acid.

In some embodiments, the disclosure provides a method for hydrolyzing biomass, wherein a volume of water is added to a mixture comprising biomass and ionic liquid at least 2 times during the time period in which the biomass is being hydrolyzed. In some embodiments, a volume of water is added at least 4 times during the time period in which the biomass is being hydrolyzed. In some embodiments, a volume of water is added at least 6 times during the time period in which the biomass is being hydrolyzed. In some embodiments, the two volumes of water are different amounts of water.

In some embodiments, the disclosure provides a method for hydrolyzing biomass, the method comprising: (a) dissolving at least some biomass in an ionic liquid; (b) hydrolyzing at least some of the biomass; and (c) adding water to the biomass and ionic liquid.

In some embodiments, the water is added after at least some of the biomass is converted to water-soluble carbohydrates.

In some embodiments, the disclosure provides a method for hydrolyzing biomass, the method comprising: (a) contacting biomass with ionic liquid; and (b) adding water to the biomass and ionic liquid at least 5 minutes after the contacting. In some embodiments, water is added at least 20 minutes after the contacting. In some embodiments, the biomass and ionic liquid are contacted in the presence of an acid.

In some embodiments, the disclosure provides a method for hydrolyzing biomass, the method comprising (a) adding biomass comprising cellulose to ionic liquid such that the cellulose dissolves in the ionic liquid over a period of time; and (b) adding water to the ionic liquid when the degree of polymerization of the dissolved cellulose is less than 99% of the degree of polymerization of the cellulose in the biomass before being dissolved in the ionic liquid.

In some embodiments, water is added when the degree of polymerization is less than 80% of the degree of polymerization of the cellulose in the biomass. In some embodiments, water is added when the degree of polymerization is less than 50% of the degree of polymerization of the cellulose in the biomass.

In some embodiments, the disclosure provides a method for hydrolyzing biomass, the method comprising (a) adding biomass comprising hemicellulose to ionic liquid such that the hemicellulose dissolves in the ionic liquid over a period of time; and (b) adding water to the ionic liquid when the degree of polymerization of the dissolved hemicellulose is less than 99% of the degree of polymerization of the hemicellulose in the biomass before being dissolved in the ionic liquid. In some embodiments, water is added when the degree of polymerization is less than 80% of the degree of polymerization of the hemicellulose in the biomass. In some embodiments, water is added when the degree of polymerization is less than 50% of the degree of polymerization of the hemicellulose in the biomass.

In some embodiments, the disclosure provides a method for hydrolyzing biomass comprising adding water to a mixture of biomass and ionic liquid after the degree of polymerization of cellulose or hemicellulose dissolved in the ionic liquid has been reduced by at least 1% compared with the degree of polymerization of the cellulose or hemicellulose in the biomass before being dissolved in the ionic liquid.

In some embodiments, the concentration of water increases over time. In some embodiments, the concentration of ionic liquid decreases over time. In some embodiments, water is added at a faster rate than it is consumed in the hydrolysis reaction. In some embodiments, water is added to the biomass and ionic liquid at a continuous rate. In some embodiments, water is added to the biomass and ionic liquid in discrete intervals.

In some embodiments, the disclosure provides a method for hydrolyzing biomass, comprising adding water to a mixture of biomass and ionic liquid at a rate such that the concentration of furanic compounds in the mixture is less than 1% (w/w).

In some embodiments, the disclosure provides a method for hydrolyzing a biomass polysaccharide substrate comprising hydrolyzing a reaction mixture comprising the biomass polysaccharide substrate and an ionic liquid in which the biomass polysaccharide substrate is soluble and adding water to the reaction mixture, wherein water is added at a rate such that the polysaccharide of the biomass polysaccharide substrate is not precipitated from the reaction mixture and hydrolysis is not substantially inhibited, and following hydrolysis, lowering the temperature of the reaction mixture from the temperature at which hydrolysis is performed.

In some embodiments, the reaction mixture further comprises acid. In some embodiments, the biomass polysaccharide substrate is lignocellulosic biomass. In some embodiments, hydrolysis is continued until the monosaccharide yield is 50% or higher.

In some embodiments, the amount of acid ranges from about 5 weight % to 40 weight % relative to the amount of biomass polysaccharide substrate in the reaction. In some embodiments, the amount of acid ranges from about 10 weight % to 25 weight % relative to the amount of biomass polysaccharide substrate in the reaction.

In some embodiments, the reaction mixture is heated to a temperature of about 70 to 140° C. during hydrolysis. In some embodiments, the reaction mixture is cooled to a temperature of about 20 to 100° C. following hydrolysis.

In some embodiments, the ionic liquid comprises chloride, trifluoroacetate, trichloroacetate, tribromoacetate or thiocyanate. In some embodiments, the cation of the ionic liquid is an imidazolium or a pyridinium. In some embodiments, the ionic liquid is [EMIM]Cl, [BMIM]Cl, 1-ethyl-2,3-dimentylimidazolium chloride or 1-alkylpyridinium chloride.

In some embodiments, water is added such that the total amount of water in the reaction mixture is less than 20 weight %. In some embodiments: (a) a total water level of 20 weight % with respect to the total reaction mixture is added by 3-10 minutes after initiation of hydrolysis; (b) a total water level of 20 weight % with respect to the total reaction mixture is added by 10 minutes after initiation of hydrolysis; (c) a total water level of 20-35 weight % with respect to the total reaction mixture is added within 10-30 minutes after initiation of hydrolysis; (d) a total water level of 35-45 weight % with respect to the total reaction mixture is added within 30-60 minutes after initiation of hydrolysis; or (e) a total water level of 40-45 weight % with respect to the total reaction mixture is added within 60 minutes after initiation of hydrolysis.

In some embodiments, the temperature is lowered such that the yield of 5-hydroxymethylfurfural in the hydrolysis product is 10% or less.

In some embodiments, a co-solvent is added to the reaction mixture in an amount ranging from 1 to 25 weight % of the reaction mixture.

In some embodiments, the disclosure provides a hydrolysis product prepared by the methods of the disclosure.

In some embodiments, the disclosure provides a method for making a monosaccharide feedstock which comprises preparing a hydrolysis product and separating the hydrolysis product from ionic liquid.

In some embodiments, the disclosure provides a method for generating ethanol by fermentation which comprises employing the hydrolysis product of the methods described herein as a monosaccharide feedstock for fermentation by an ethanologenic microorganism.

In some embodiments, the disclosure provides a method for hydrolyzing a biomass polysaccharide substrate comprising hydrolyzing a reaction mixture comprising the biomass polysaccharide substrate and an ionic liquid in which the biomass polysaccharide substrate is soluble and adding water to the reaction mixture, wherein water is added at a rate such that the polysaccharide of the biomass polysaccharide substrate is not precipitated from the reaction mixture and hydrolysis is not substantially inhibited, wherein the pressure at which hydrolysis is performed is not atmospheric pressure.

In some embodiments, the pressure is greater than atmospheric pressure. In some embodiments, the pressure is less than atmospheric pressure. In some embodiments, the pressure is increased as the hydrolysis reaction proceeds. In some embodiments, the pressure is decreased as the hydrolysis reaction proceeds. In some embodiments, the reaction mixture further comprises acid. In some embodiments, the biomass polysaccharide substrate is lignocellulosic biomass.

In some embodiments, hydrolysis is continued until the monosaccharide yield is 50% or higher. In some embodiments, the amount of acid ranges from about 5 weight % to 40 weight % relative to the amount of biomass polysaccharide substrate in the reaction. In some embodiments, the amount of acid ranges from about 10 weight % to 25 weight % relative to the amount of biomass polysaccharide substrate in the reaction.

In some embodiments, the reaction mixture is heated to a temperature of about 70 to 140° C. during hydrolysis. In some embodiments, the reaction mixture is cooled to a temperature of about 20 to 100° C. following hydrolysis.

In some embodiments, the ionic liquid comprises chloride, trifluoroacetate, trichloroacetate, tribromoacetate or thiocyanate. In some embodiments, the cation of the ionic liquid is an imidazolium or a pyridinium. In some embodiments, the ionic liquid is [EMIM]Cl, [BMIM]Cl, 1-ethyl-2,3-dimentylimidazolium chloride or 1-alkylpyridinium chloride.

In some embodiments, water is added such that the total amount of water in the reaction mixture is less than 20 weight %. In some embodiments: (a) a total water level of 20 weight % with respect to the total reaction mixture is added by 3-10 minutes after initiation of hydrolysis; (b) a total water level of 20 weight % with respect to the total reaction mixture is added by 10 minutes after initiation of hydrolysis; (c) a total water level of 20-35 weight % with respect to the total reaction mixture is added within 10-30 minutes after initiation of hydrolysis; (d) a total water level of 35-45 weight % with respect to the total reaction mixture is added within 30-60 minutes after initiation of hydrolysis; or (e) a total water level of 40-45 weight % with respect to the total reaction mixture is added within 60 minutes after initiation of hydrolysis. In some embodiments, the pressure is such that the yield of 5-hydroxymethylfurfural in the hydrolysis product is 10% or less. In some embodiments, a co-solvent is added to the reaction mixture in an amount ranging from 1 to 25 weight % of the reaction mixture.

In some embodiments, the disclosure provides a hydrolysis product prepared by the methods described herein.

In some embodiments, the disclosure provides a method for making a monosaccharide feedstock which comprises preparing a hydrolysis product and separating the hydrolysis product from ionic liquid.

In some embodiments, the disclosure provides a method for generating ethanol by fermentation which comprises employing the hydrolysis product of the method as a monosaccharide feedstock for fermentation by an ethanologenic microorganism.

In some embodiments, the disclosure provides a method for hydrolyzing biomass, the method comprising applying a variable pressure to a mixture comprising biomass, water and ionic liquid.

In some embodiments, the pressure is decreased as the biomass is hydrolyzed. In some embodiments, the pressure is increased as the biomass is hydrolyzed. In some embodiments, the pressure is varied such that the solubility of the biomass in the ionic liquid is not substantially decreased and the rate of hydrolysis is not substantially decreased. In some embodiments, the mixture further comprises acid.

In some embodiments, the disclosure provides a method for hydrolyzing biomass, the method comprising contacting biomass with ionic liquid at a pressure greater than atmospheric pressure, wherein the biomass is hydrolyzed in the ionic liquid.

In some embodiments, the ionic liquid is in contact with a pressurized gas. In some embodiments, the pressure is greater than 5 atm. In some embodiments, the ionic liquid comprises acid. In some embodiments, the rate of hydrolysis is at least 20% greater than the rate of hydrolysis at atmospheric pressure.

In some embodiments, water is added to the biomass and ionic liquid at a rate that is approximately equal to the rate at which water is consumed in the hydrolysis reaction.

In some embodiments, the disclosure provides a method for hydrolyzing biomass comprising contacting biomass with ionic liquid, wherein the ionic liquid is in contact with a pressurized gas and the biomass is hydrolyzed in the ionic liquid.

In some embodiments, the rate of hydrolysis is at least 5% greater than the rate of hydrolysis when the ionic liquid is not in contact with the pressurized gas.

In some embodiments, the ionic liquid comprises acid. In some embodiments, the gas comprises carbon dioxide. In some embodiments, the ionic liquid comprises carbonic acid. In some embodiments, the gas is pressurized to at least 2 atm.

In some embodiments, water is added to the biomass and ionic liquid at a rate that is approximately equal to the rate at which water is consumed in the hydrolysis reaction.

In some embodiments, the biomass is contacted with the ionic liquid at a temperature, the ionic liquid is a liquid at the temperature when in contact with the pressurized gas, and the ionic liquid is a solid at the temperature when not in contact with the pressurized gas.

In some embodiments, contacting the ionic liquid with the pressurized gas reduces the viscosity of the hydrolysis reaction by at least 5%. In some embodiments, the pressure is adjusted as the hydrolysis reaction proceeds such that the rate of hydrolysis decreases by no more than about 50% during the course of the hydrolysis reaction.

In some embodiments, the pressure is increased as the hydrolysis reaction proceeds. In some embodiments, the pressure is decreased as the hydrolysis reaction proceeds.

In some embodiments, the method further comprises extracting hydrolysis products or derivatives thereof in the gas.

In some embodiments, the disclosure provides a method comprising: (a) adding biomass to a vessel comprising ionic liquid; and (b) adding a pressurized gas to the vessel, wherein the biomass is dissolved and hydrolyzed to sugar in the ionic liquid and at least one of (i) lignin is not dissolved in the ionic liquid, (ii) lignin is precipitated from the ionic liquid, (iii) the sugar is extracted in an aqueous phase, (iv) the sugar is extracted in the pressurized gas, (v) oils are removed by phase separation, and (vi) oils are extracted in the pressurized gas.

In some embodiments, the vessel is a column. In some embodiments, the vessel maintains a pressure gradient. In some embodiments, the ionic liquid comprises acid.

In some embodiments, the disclosure provides a method comprising (a) contacting biomass with a mixture comprising ionic liquid and gas, and (b) applying a varying pressure, wherein the contacting and varying pressure results in a first phase comprising ionic liquid and a second phase comprising sugar.

In some embodiments, the second phase comprises water. In some embodiments, the gas is carbon dioxide. In some embodiments, the method further comprises recovering lignin and/or oils from the ionic liquid.

In some embodiments, the disclosure provides a method comprising hydrolyzing biomass in ionic liquid in a vessel and separating the hydrolysate from the ionic liquid in the vessel.

In some embodiments, the vessel is a column. In some embodiments, the vessel maintains a pressure gradient. In some embodiments, the vessel comprises pressurized gas. In some embodiments, the gas comprises carbon dioxide.

In some embodiments, the water soluble sugars of the hydrolysate are separated from the ionic liquid in a water phase. In some embodiments, the water soluble sugars of the hydrolysate are extracted from the ionic liquid in the pressurized gas.

In some embodiments, the solids of the hydrolysate are separated from the ionic liquid with a filter. In some embodiments, the solids comprise lignin, ash, or any combination thereof. In some embodiments, the oils of the hydrolysate are separated from the ionic liquid in an oil phase.

In some embodiments, the disclosure provides a method for removing a biomass component from ionic liquid, the method comprising contacting a fluid with ionic liquid having a dissolved biomass component, wherein the biomass component precipitates from the ionic liquid.

In some embodiments, the fluid is miscible in the ionic liquid. In some embodiments, the fluid comprises carbon dioxide. In some embodiments, the fluid is a gas pressurized above atmospheric pressure. In some embodiments, the fluid is a supercritical or near-supercritical fluid. In some embodiments, the biomass component is derived from lignocellulose. In some embodiments, the biomass component is lignin, cellulose, hemicellulose, ash, protein, starch, or any combination thereof. In some embodiments, the method further comprises adding a co-solvent to the ionic liquid. In some embodiments, the co-solvent is water, ethanol, a ketone, or any combination thereof.

In some embodiments, the method further comprises partitioning the precipitated biomass component from the ionic liquid, optionally washing the partitioned biomass component, and optionally drying the washed biomass component. In some embodiments, the biomass component is partitioned by filtration or centrifugation. In some embodiments, the biomass component is washed with water, ethanol, or any combination thereof. In some embodiments, ionic liquid is recovered from the wash.

In some embodiments, the partitioned, washed and/or dried biomass component comprises less than 1% (w/w) ionic liquid. In some embodiments, the partitioned, washed and/or dried biomass component comprises less than 0.1% (w/w) ionic liquid.

In some embodiments, the method further comprises hydrolyzing the biomass component. In some embodiments, the biomass component comprises lignin and at least one of cellulose and hemicellulose, the cellulose and/or hemicellulose are hydrolyzed in the ionic liquid and the lignin is precipitated by contacting the fluid with the ionic liquid. In some embodiments, contacting the fluid with the ionic liquid decreases the dielectric constant of the ionic liquid.

In some embodiments, the disclosure provides a method for removing solids from an ionic liquid, the method comprising contacting a pressurized gas with ionic liquid having dissolved solids, wherein the solids precipitate from the ionic liquid.

In some embodiments, the solids comprise lignin, cellulose, hemicellulose, ash, or any combination thereof. In some embodiments, the gas comprises carbon dioxide. In some embodiments, the gas is pressurized to greater than atmospheric pressure.

In some embodiments, the disclosure provides a method comprising: (a) providing a composition comprising ionic liquid and dissolved solids; (b) providing an extractant above the boiling point temperature of the extractant; and (c) contacting said composition with said extractant, wherein said contacting precipitates the solids.

In some embodiments, the method further comprises recovering the precipitated solids from the ionic liquid. In some embodiments, the solids are recovered by filtration. In some embodiments, the dissolved solids comprise lignin, ash, cellulose, hemicellulose, protein, or any combination thereof. In some embodiments, the extractant comprises carbon dioxide.

In some embodiments, the disclosure provides a method comprising providing a composition comprising ionic liquid and solids dissolved therein and recovering the solids from the ionic liquid, wherein recovering the solids results in a loss of less than 1% (w/w) of the ionic liquid.

In some embodiments, the method results in a loss of less than 0.5 (w/w) of the ionic liquid. In some embodiments, the method results in a loss of less than 0.1 (w/w) of the ionic liquid. In some embodiments, the method results in a loss of less than 0.01 (w/w) of the ionic liquid. In some embodiments, the method results in a loss of less than 0.001 (w/w) of the ionic liquid.

In some embodiments, the solids comprise lignin, cellulose, hemicellulose, ash, or any combination thereof.

In some embodiments, the disclosure provides a method comprising providing a composition comprising ionic liquid and solids dissolved therein and recovering the solids from the ionic liquid, wherein the recovered solids comprise less than 1% (w/w) ionic liquid. In some embodiments, the recovered solids comprise less than 0.1% (w/w) ionic liquid. In some embodiments, the recovered solids comprise less than 0.01% (w/w) ionic liquid. In some embodiments, the recovered solids comprise less than 0.001% (w/w) ionic liquid.

In some embodiments, the solids comprise lignin, cellulose, hemicellulose, ash, or any combination thereof.

In some embodiments, the method further comprises rapidly de-pressurizing the lignin. In some embodiments, the lignin is de-pressurized such that the ionic liquid is more readily recovered from the lignin.

In some embodiments, the method further comprises dissolving the lignin. In some embodiments, the lignin is dissolved in a lignin-dissolving ionic liquid. In some embodiments, the disclosure provides the solids produced by any of the methods. In some embodiments, the disclosure provides the lignin produced by any of the methods.

In some embodiments, the disclosure provides an aromatic compound, concrete additive, antioxidant, asphalt additive, carbon fiber or other fiber, board binder, foam, plastic or other polymer, dust control product, paper product, chemical product, battery, fuel, heat, grease, dispersant, or fertilizer produced from the lignin described herein.

In some embodiments of the present disclosure, the methods described herein can include: (a) providing a biomass hydrolysate comprising ionic liquid, water and a sugar; (b) forming an aqueous biphasic system (ABS) that comprises a first phase comprising ionic liquid and a second phase comprising water and sugar; and (c) extracting sugar from the second phase using a boronic acid.

The organic phase can be any organic solvent that dissolves, but does not react with the boronic acid or the sugar-boronic acid complex. In some embodiments, the organic phase comprises an organic molecule that is immiscible with ionic liquid.

In some embodiments, the boronic acid has the formula: R-x-B(OH)₂ (I); wherein x is a bond or an alkyl or alkenyl chain of 1-10 carbons, R comprises at least 1 aromatic ring, wherein optionally at least one ring is substituted by one or more alkyl groups comprising 1-10 carbons. In some embodiments, s is a bond or an alkyl or alkenyl chain of 1-4 carbons. In some embodiments, x is a bond or an alkyl or alkenyl chain of 1-2 carbons. In some embodiments, x is a —C═C—.

In some embodiments, R comprises 1, 2, or 3 aromatic rings. In some embodiments, R is a benzene, optionally comprising 1 or 2 methyl groups. In some embodiments, R is a naphthalene.

In some embodiments, the boronic acid is phenylboronic acid, 3,5-dimethylphenylboronic acid, 4-tert-butylphenylboronic acid, trans-P-styreneboronic acid, or naphthalene-2-boronic acid.

In some embodiments, provided herein is a method of removing a sugar from a solution, comprising: (a) providing a solution comprising ionic liquid, water and sugar; (b) separating the solution into an ionic liquid phase and an aqueous phase; (c) providing an organic phase comprising a boronic acid; (d) contacting the aqueous phase with the boronic acid to form a sugar-boronic acid complex, (e) separating the organic phase and the aqueous phase, wherein the organic phase contains the sugar-boronic acid complex, and optionally (f) separating the sugar from the organic phase.

In some embodiments, (f) comprises adding stripping solution comprising a stripping agent to the organic solution, such that the sugar-boronic acid complex dissociates and the sugar moves into the stripping solution. In some cases, the stripping solution is aqueous and the stripping agent is an acid which decrease the pH of the organic phase.

In some embodiments, the organic solution further comprises an organic solvent which ensures the boronic acid is fully dissolved in the organic phase. In some embodiments, the organic solvent is n-hexane, 1-octanol, or a mixture thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings (also referred to as “Figures” or “FIGS.”) of which:

FIG. 1 shows an example of a multi-phasic system.

FIG. 2 shows an example of a multi-phasic system;

FIG. 3 shows an example of a method for extracting a biomass component from an ionic liquid mixture.

FIG. 4 shows an example of recovering one or more biomass components from a fluid.

FIG. 5 shows an example of extracting biomass components from an ionic liquid using two sequential fluid extractions.

FIG. 6 shows an example of pressurizing a fluid to reject ionic liquid from the fluid when extracting biomass components from an ionic liquid.

FIG. 7 shows an example of recovering biomass components from an ionic liquid by forming an aqueous phase.

FIG. 8 shows an example of recovering biomass components from an ionic liquid by contacting the ionic liquid with a fluid to form an aqueous phase.

FIG. 9 is a picture of a solution of ionic liquid, water and glucose after extraction with supercritical carbon dioxide.

FIG. 10 is a picture of a product collected from a supercritical extraction of glucose from ionic liquid using carbon dioxide and water co-solvent.

FIG. 11 is a picture of a collection vessel filling with vapor during a supercritical extraction of glucose from carbon dioxide with water co-solvent.

FIG. 12 is a picture of the fluid captured during a supercritical extraction of glucose from carbon dioxide with water co-solvent.

FIG. 13 is a picture of precipitate forming during drying of collected liquid extract during a supercritical extraction of glucose from carbon dioxide with water co-solvent.

FIG. 14 is a picture of an aqueous phase and an ionic liquid phase with a glucose solute.

FIG. 15 shows a graph of the recovery of glucose in an aqueous phase from an ionic liquid-glucose solution.

FIG. 16 shows a logarithmic graph of the recovery of glucose in an aqueous phase from an ionic liquid-glucose solution.

FIG. 17 shows the formation of an aqueous phase over time.

FIG. 18 is a picture of an aqueous phase and an ionic liquid phase.

FIG. 19 shows an exemplary glucose concentration in a water phase over time period of being in contact with an ionic liquid-water-glucose solution.

FIG. 20 shows a relationship between ionic liquid concentration and conductivity.

FIG. 21 shows the conductivity and ionic liquid concentration after 12 hours of a water phase added on top of an ionic liquid-water-glucose solution.

FIG. 22 shows various frames of a video showing an aqueous phase and an ionic liquid phase in the presence of a glucose solute.

FIG. 23 shows an illustration of sugar effecting auto-separation of a mixture of water and ionic liquid into two phases.

FIG. 24 shows an illustration of the behavior of ionic liquid and carbon dioxide at low and high pressure.

FIG. 25 shows an illustration of increasing the pressure above the lower critical endpoint pressure (LCEP).

FIG. 26 shows a plot of pressure vs. CO₂ mol fraction for [BMIM]PF₆.

FIG. 27 shows experimental data and theoretical curves for solubility of pressurized CO₂ in [BMIM]Cl at several temperatures.

FIG. 28 shows an example of aqueous biphasic system formed by an ionic liquid phase and a sugar phase.

FIG. 29 shows solubility curves plotted in semi-logarithmic scale.

FIG. 30 shows compositions of an aqueous biphasic system (ABS) formed by [AMIM]Cl, sucrose and water.

FIG. 31 shows a phase diagram for a [BMIM]BF₄ with sucrose aqueous biphasic system.

FIG. 32 shows phase diagrams for the ternary systems composed by [BMIM][CF₃SO₃]+carbohydrate+H₂O at 298 K.

FIG. 33 shows an illustration of the individual and combined effects of an ionic liquid/CO₂ phase excluding water, and a water/sugar phase excluding ionic liquid.

FIG. 34 shows an illustration of an extractor having a “sugar driver” (on top) and a “CO₂ driver” (on bottom).

FIG. 35 shows an illustration of a process that uses a salting-out agent (e.g., kosmotropic salt) to form an aqueous biphasic system and recover a solute (e.g., sugar).

FIG. 36 shows an illustration of a process that uses two ionic liquids to process ligno-cellulosic biomass.

FIG. 37 shows a schematic drawing of separation employing ionic liquid (IL), a kosmotropic salt and alcohol precipitation of the salts.

FIG. 38 shows a photograph of salts precipitated with methanol.

FIG. 39 shows a clear polyethylene glycol (PEG) layer on top of a clear phosphate buffer (PB) layer.

FIG. 40 shows a schematic drawing of separation employing ammonia (NH₃) and carbon dioxide (CO₂).

FIG. 41 shows a picture of a high-pressure apparatus.

FIG. 42 shows a binary phase diagram for a weak ABS and for a strong ABS.

FIG. 43 shows partition coefficients for ionic liquid and glucose plotted with respect to the total concentration of phosphate buffer and ionic liquid at the start of ABS formation.

FIG. 44 shows an example of ABS formation without the addition of salt.

FIG. 45 shows [BMIM]Cl (left vial) and [BMPYR]Cl (right vial) ABS at equilibrium at room temperature with a bottom phase of PB adjusted to pH=9.4.

FIG. 46 shows the kinetics of ABS formation with normalized concentration trajectories for the IL-rich phase composition.

FIG. 47 shows a schematic drawing of separation employing IL and CO₂.

FIG. 48 shows an example of a ternary system phase diagram represented in two dimensions.

FIG. 49 shows the design of a multi-stage liquid-liquid extraction.

FIG. 50 shows an example of a process configuration for filtering the precipitate.

FIG. 51 shows an example of a process configuration for filtering and washing the precipitate.

FIG. 52 shows an example of a process configuration for filtering and repeatedly washing the precipitate.

FIG. 53 shows an example of a process configuration for filtering, repeatedly washing and drying the precipitate.

FIG. 54 shows an example of a process for dissolving and optionally performing chemical conversion of lignin in a lignin solvent (e.g., a lignin-dissolving ionic liquid).

FIG. 55 shows a drawing of hydrolysis and extraction in a single pot.

FIG. 56 shows a drawing of a column capable of fractionating biomass hydrolysate from an ionic liquid.

FIG. 57 shows a drawing of the isolated solids as viewed under a microscope.

FIG. 58 shows an example of a process that recovers sugars with boronic acids.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the spirit and scope of the invention. It should be understood that various alternatives to the embodiments of the invention described herein may be employed.

### Ionic Liquids

An “ionic liquid” (“IL”) refers to salts (e.g., comprising cations and anions) that are liquid. In some cases, the ionic liquid is a liquid at the conditions (e.g., temperature, presence of materials mixed with the ionic liquid) used in the process. Ionic liquids can have a relatively low melting point (e.g., are liquid at temperatures below a certain low temperature). In some cases, the melting point is below about 300° C., below about 200° C., below about 150° C., below about 130° C., below about 100° C., below about 75° C., below about 50° C., and the like. In some embodiments, the ionic liquid is a liquid at ambient and/or room temperature. The melting point can refer to the melting point of the pure (e.g., at least 90% pure, at least 95% pure, at least 96% pure, at least 97% pure, at least 98% pure, at least 99% pure) ionic liquid, or can refer to the melting point of the ionic liquid when mixed with other components as used in the process (e.g., water). Mixtures of one or more ionic liquids can also be used. In some embodiments, a mixture of 1, 2, 3, 4, 5 or more ionic liquids can be used. Many salts exist that are ionic liquids, and their use is contemplated by the methods, apparatus, and processes described herein.

In some embodiments, the anion component of the ionic liquid includes for example and without limitation chloride, acetate, bromide, iodide, fluoride and nitrate.

In some embodiments, the ionic liquid comprises immidazolium-based, pyridinium-based and/or choline-based cations.

Herein, for clarity and without limitation, ionic liquids can include for example but are not limited to 1-propyl-3-methylimidazolium chloride and/or 1-butyl-3-methylimidazolium chloride. Some further exemplary ionic liquids include but are not limited to 1-allyl-3-methylimidazolium chloride, 1-butyl-3-methylimidazolium chloride, 1-ethyl-3-methylimidazolium chloride, 1-(2-hydroxylethyl)-3-methylimidazolium chloride, 1-butyl-1-methylpyrrolidinium decanoate. For clarity, additional ionic liquids may be known in the art and can be employed with the methods of the present invention. In some embodiments, the ionic liquid is selected from the group consisting of 1-butyl-3-methylimidazolium chloride, 1-allyl-3-methylimidazolium chloride, 1-propyl-3-methylimidazolium chloride, 1-ethyl-3-methylimidazolium chloride, 1-(2-hydroxylethyl)-3-methylimidazolium chloride, 1-butyl-1-methylpyrrolidinium decanoate and any combination thereof.

In some embodiments, 1-butyl-3-methylimidazolium chloride, which has an anion, a cation, and a melting point of about 65° C. is an ionic liquid. In some cases, the term “molten salt” is used interchangeably with ionic liquid. In some cases, a molten salt is not an ionic liquid (e.g., molten sodium chloride, which has a high melting point).

The invention also encompasses using mixtures of ionic liquids and/or adding any suitable enhancer, modifier, or the like. In some cases, the ionic liquid comprises a plurality of species of cation and/or anion. In some cases, the overall charge of an ionic liquid is optionally neutral.

The invention also encompasses using materials convertible to, and/or converted to an ionic liquid. For example, some ionizable compounds can become more dissociated into ions when mixed with an ionic liquid.

The ionic liquids can be hydrophilic, meaning that they are miscible in any proportion with water. In some cases, the ionic liquids are hydrophobic. Hydrophobic ionic liquids can contain some water. Hydrophobic ionic liquids are not miscible (i.e., immiscible) with water and at certain concentrations, for example, form a water phase and an ionic liquid phase.

In some embodiments, the ionic liquid is a biomass dissolving ionic liquid (e.g., an ionic liquid that is capable of dissolving biomass). The solubility of biomass in the ionic liquid can be any suitable value including about 1%, about 3%, about 5%, about 10%, about 15%, about 20%, about 25%, about 30%, about 35%, about 40%, about 50%, and the like by mass. In some embodiments, the solubility can be about 1% to about 50%, about 3% to about 40%, about 5% to about 35%, about 10% to about 30%, or about 15% to about 25% by mass. In some embodiments, the solubility of biomass in the ionic liquid is at least 1%, at least 3%, at least 5%, at least 10%, at least 15%, at least 20%, at least 25%, at least 30%, at least 35%, at least 40%, at least 50%, and the like by mass.

In some embodiments, the ionic liquid is insoluble in a fluid (e.g., supercritical or near-supercritical fluid). In various embodiments, a fluid dissolves about 5%, about 1%, about 0.5%, about 0.1%, about 0.05%, about 0.01%, about 0.005%, about 0.001%, about 0.0005%, and the like ionic liquid by mass in comparison to the mass of the fluid. In some embodiments, the fluid dissolves about 0.0005 to about 5%, about 0.001% to about 1%, 0.005% to about 0.5%, or about 0.01% to about 0.1% ionic liquid by mass in comparison to the mass of the fluid. In various embodiments, a fluid dissolves at most about 5%, at most about 1%, at most about 0.5%, at most about 0.1%, at most about 0.05%, at most about 0.01%, at most about 0.005%, at most about 0.001%, at most about 0.0005%, and the like ionic liquid by mass in comparison to the mass of the fluid. In some embodiments, the fluid dissolves at most about 0.0005 to about 5%, about 0.001% to about 1%, 0.005% to about 0.5%, or about 0.01% to about 0.1% ionic liquid by mass in comparison to the mass of the fluid.

In certain embodiments, the ionic liquid is non-toxic, biodegradable, non-flammable, or has other properties that result in a safe and environmentally friendly process.

### Use of High Melting Point Ionic Liquids

Provided herein are methods for using high melting point ionic liquids to dissolve and/or hydrolyze biomass. As shown here, the “liquid” range of ionic liquids can be expanded (e.g., to lower temperatures) by dissolving CO₂ in the ionic liquid. For instance, the melting point of pure [BMIM]CH₃SO₃ is 72° C., but can be lowered to 52° C. under 15 MPa of CO₂ pressure. Other ionic liquids, such as [NBu₄]BF₄ are solid at temperatures sometimes used in biomass dissolution and hydrolysis (Tₘ=156° C.), yet 15 MPa of CO₂ pressure lowers the melting point to about 36° C. The presence of water in the hydrolysis reaction also helps to expand the liquid range of the ionic liquid to lower temperatures.

The use of high melting point ionic liquids at temperatures lower than the melting point of the pure ionic liquid (e.g., by using CO₂ pressure) can expand the type of ionic liquids that are suitable for performing the methods of dissolution and hydrolysis explained here. For example, the ionic liquid depicted below has two charge centers, potentially resulting in a higher concentration of free chloride ion and a higher solubility and/or rate of solubilization of biomass than ionic liquids having only one charge center. However, the melting point of such a compound is relatively high (e.g., about Tₘ>200° C., a temperature at which degradation of hydrolysis products may proceed). Use of this ionic liquid at mild temperatures (e.g., <110° C.) may be possible when pressurized with CO₂.

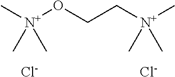

### Ionic Liquid Recovery

The methods described herein use ionic liquids efficiently. In some cases, very little of the ionic liquid is lost in the process. In some instances, the process includes recovering biomass components from the ionic liquid. Other examples of process where the ionic liquid can be used efficiently include for example without limitation manufacturing and/or purification of the ionic liquid, use of the ionic liquid in electrochemical devices such as batteries and capacitors, use of ionic liquids in chemical processes including fossil fuel processing.

In some embodiments, the method for separating a biomass component from an ionic liquid comprises losing less than 10 grams of ionic liquid per kilogram of biomass component separated. In some embodiments, less than 1 gram of ionic liquid is lost per kilogram of biomass component separated. In some instances, less than 0.1 gram of ionic liquid is lost per kilogram of biomass component separated. In some instances, less than 0.01 gram of ionic liquid is lost per kilogram of biomass component separated. In some instances, less than 0.001 gram of ionic liquid is lost per kilogram of biomass component separated. In some instances, the method comprises contacting a composition comprising an ionic liquid and a biomass component with a fluid. In some embodiments, less than about 10 gram to about 0.001 gram of ionic liquid is lost per kilogram of biomass component separated. In some embodiments, less than about 1 gram to about 0.001 gram of ionic liquid is lost per kilogram of biomass component separated. In some embodiments, less than about 1 gram to about 0.01 gram of ionic liquid is lost per kilogram of biomass component separated. In some embodiments, less than about 0.1 gram to about 0.001 gram of ionic liquid is lost per kilogram of biomass component separated. In some embodiments, less than about 0.1 gram to about 0.01 gram of ionic liquid is lost per kilogram of biomass component separated.

The ionic liquid can be recovered to any suitable level. In some instances, at least 95%, at least 96%, at least 97%, at least 98%, at least 99%, at least 99.5%, at least 99.9%, at least 99.99%, at least 99.999%, at least 99.9999%, or at least 99.99999% of the ionic liquid is recovered (e.g., per batch or per week of operation). In some embodiments, the ionic liquid is recovered in a range of at least 95% to at least 99.99999%, at least 96% to at least 99.999%, at least 97% to at least 99.99%, at least 98% to at least 99.9%, or at least 99% to at least 99.5%.

The purity of the ionic liquid following the process is any suitable level. In some instances, the ionic liquid is at least 95%, at least 96%, at least 97%, at least 98%, at least 99%, at least 99.5%, at least 99.9%, at least 99.99%, at least 99.999%, at least 99.9999%, or at least 99.99999% pure. In some embodiments, the ionic liquid has a purity in a range of at least 95% to at least 99.99999%, at least 96% to at least 99.999%, at least 97% to at least 99.99%, at least 98% to at least 99.9%, or at least 99% to at least 99.5%.

In some instances, the ionic liquid is re-used after the process (e.g., after recovering biomass components from the ionic liquid).

The process using the ionic liquid can be without limitation a batch process, a continuous process, a semi-batch process, or any combination thereof.

Suitable methods for determining the amount of ionic liquid lost from the process include, but are not limited to determining the mass of ionic liquid before and after the process, or operating the process for a period of time and observing a loss in ionic liquid over that time period.

### Biomass and Biomass Components

In some embodiments, the invention provides methods for separating biomass and/or biomass components from ionic liquids. The biomass can be any suitable material, including mixed material or materials that can change or are changed over time. In some embodiments, the present invention may be practiced in a feedstock-flexible biorefinery.

The biomass can include for example and without limitation plant matter, algae, seaweed, agricultural or forestry residue, industrial or municipal waste, or any other suitable material, as well as any combinations of these materials. As used herein, “biomass” includes any component of the biomass (e.g., lipids, proteins, cellulose, lignin) and/or derivatives of the plant material and/or derivatives of its components (e.g., cellulose hydrolyzed to sugars, sugars dehydrated to furanic compounds).

The biomass can be purposely grown for processing as described herein, or the biomass can grow and/or be grown for any purpose and be processed in whole or in part using the methods described herein. The biomass can be farmed (including both food crops and energy crops) or grow wild. The biomass can be for example genetically modified, wild type, and/or selectively bred in various embodiments.

In some instances the biomass is cellulosic, meaning that it comprises cellulose or derivatives thereof. Cellulose is a polymer of glucose monomers (e.g., beta 1-4 linked, a polysaccharide). In some instances, the cellulose is broken down and/or hydrolyzed (e.g., to sugars).

In some instances, the biomass is lignocellulosic, meaning that it comprises cellulose and lignin. Lignin is a complex chemical compound that forms part of some plants (e.g., cell walls). Lignin is generally heterogeneous and lacks of a defined primary structure. Lignin can comprise biopolymers of p-coumaryl alcohol, coniferyl alcohol and/or sinapyl alcohol. In some instances, the biomass has no lignin or a small amount of lignin (e.g., less than 5%, less than 3%, or less than 1%).

Cellulosic and/or lignocellulosic biomass may also comprise hemicellulose. A hemicellulose can comprise any of several heteropolymers, such as arabinoxylans, present along with cellulose in some plant cell walls. Hemicellulose can contain many different sugar building blocks. In contrast, cellulose generally contains only anhydrous glucose. For instance, besides glucose, sugar building blocks in hemicellulose can include xylose, mannose, galactose, rhamnose, and arabinose. Hemicelluloses can contain pentose (5 carbon) sugars. In some instances, xylose is the sugar monomer present in the largest amount, but mannuronic acid and galacturonic acid may also be present among others. In some instances, hemicellulose is broken down and/or hydrolyzed into sugars.

The biomass may be an energy plant and/or energy crop. Exemplary energy crops include without limitation farmed trees such as Pinus radiata, and fast growing plants such as Miscanthus giganteus and Panicum virgatum. Energy cane, sorghum, sweet sorghum are further examples of energy crops. Energy crops can comprise lignocellulose and sometimes require less water, fertilizer, and the like to grow rapidly compared with a food crop. In some cases, energy crops are grown on land unsuitable for growing food crops. The biomass may also be all or part of a plant that is more traditionally a food crop, such as corn (Zea mays) or sugar cane.

In some embodiments, the biomass is algae, which includes but is not limited to eukaryotic microalgae, cyanobacteria, diatoms, macroalgae, and the like as well as any combinations thereof. Algae are generally photosynthetic, but lack roots, leaves and other structures found in plants. Some algae live in aqueous rather than terrestrial environments. Algae are distinct from plants. Exemplary algae species include, but are not limited to Chlamydomonas moewusii, Chlamydomonas reinhardii, Neochloris pseudostigmata, Scenedesmus quadricauda, Chlorella vulgaris, Chlorococcum hypnosporum, Dunaliella salina and Chlorella pyrenoidosa.

In some embodiments, the algae may be processed using the methods described herein in a substantially aqueous form. That is, drying and/or dewatering the algae may be unnecessary, which may reduce the amount of energy needed to grow algae and isolate useful materials therefrom. In various embodiments, the algae may comprise at least 95% water, at least 90% water, at least 80% water, at least 70% water, at least 60% water, at least 50% water, at least 30% water and the like.

In some embodiments, the biomass is a mixture of algae and lignocellulose. In some embodiments, water is added to the ionic liquid. In some embodiments, the water can comprise algae biomass (or any other biomass) wherein algae and lignocellulose are co-processed.

In some instances, biomass components are removed from ionic liquids. The biomass can optionally be broken down into its components in the ionic liquid, or may be broken down by other means and added to an ionic liquid. In some instances, the biomass components are not only removed from the ionic liquid, but also fractionated. For instance, carbohydrates can be fractionated from lipids and/or proteins (e.g., biomass components are isolated or separated from each other). In some embodiments, various sugars may be isolated from each other, such as for example glucose from other sugars such as arabinose and xylose. Any of these operations and/or combinations of operations can result in a biomass mixture.

Exemplary biomass components in a biomass mixture include, but are not limited to nucleic acids, proteins, lipids, fatty acids, resin acids, waxes, terpenes, acetates (e.g., ethyl acetate, methyl acetate), carbohydrates, polysaccharides cellulose, hemicellulose, alcohols, sugars, sugar acids, glucose, fructose, xylose, galactose, arabinose, mannose, rhamnose, mannuronic acid, galacturonic acid, lignin, alcohols (e.g., methanol, ethanol), phenols, aldehydes, ethers, p-coumaryl alcohol, coniferyl alcohol, sinapyl alcohol, pectin, D-galacturonic acid, amino acids, acetic acid, ash, water, any derivative thereof (e.g., furanic compounds), or any combination thereof. Any suitable biomass component can be recovered from the biomass mixture as described herein. Any of these individual components and/or mixtures thereof can be separated from one another.

In some embodiments, the biomass components include carbohydrates. Carbohydrates have the chemical formula Cₘ(H₂O)ₙ, where m and n are integers. In some cases, the biomass component is a carbohydrate derivative (e.g., chloroglucose (C₆H₁₁O₅Cl)). Carbohydrates include water-soluble carbohydrates and water-insoluble carbohydrates.

Polysaccharides are also biomass components (e.g., cellulose, starch, or hemicellulose). In various embodiments, the biomass may comprise polysaccharides of any average degree of polymerization and/or profile or range of degrees of polymerization. In some instances, cellulose may have 7,000-15,000 glucose molecules per polymer and hemicellulose may have about 500-3,000 sugar units. In some examples, the degree of polymerization of the polysaccharide is reduced in the ionic liquid. In some embodiments, polysaccharides that have a degree of polymerization of at most about 20, at most about 5, at most 2, or at most one (i.e., monosaccharides) are recovered from the ionic liquid as described herein. In some embodiments, the polysaccharides recovered are water-soluble and/or fermentable. In some cases, the recovered polysaccharides comprise between 1 and about 2, about 3, about 4, about 5, about 6, about 7, about 8, about 9 or about 10 sugar units. In some embodiments, low molecular weight carbohydrates (e.g., polysaccharides) are continuously removed from the ionic liquid reaction as the polysaccharides are continuously broken down to lower molecular weight carbohydrates (e.g., sugars). As used herein, “continuously” can generally include being performed over repeatedly small time intervals such as about 1 second, 10 seconds, 30 seconds, 1 minute, 5 minutes or 10 minutes.

In some embodiments, the biomass components include sugars. Sugars include monosaccharides, disaccharides and oligosaccharides.

In some instances, the sugars are fermentable. Fermentable sugars are capable of nourishing and/or sustaining a culture of microbes (e.g., E. coli and/or yeast). Various microorganisms are capable of using various sugars, so while arabinose may be fermentable by one organism it may not be by another. For the purposes of clarity, a sugar is fermentable if there is at least one microorganism known to be capable of growing on the sugar and/or metabolizing the sugar. Exemplary fermentable sugars include but are not limited to glucose, fructose, xylose, or combinations thereof. Fermentable sugars need not be monosaccharides.

As used herein, biomass includes derivatives of biomass and/or derivatives of biomass components. Also, as used herein, biomass components include derivatives of biomass components. In some cases, at least some of the mass of the derivative (e.g., at least some atoms) are traceable back to biomass and/or biomass component (e.g., plant material and/or cellulose). For example, furanic compounds (e.g., hydroxymethylfurfural, 2,5-dimethylfuran) can be produced by the dehydration of sugars, so are an example of a biomass derivative. A method for producing furanic compounds from biomass is described for example in U.S. Patent Pub. No. 2010/0004437, which is herein incorporated by reference in its entirety. Those of ordinary skill in the art will be aware of many biomass derivatives including polyols, and the like.

### Biomass Hydrolysis

In some embodiments, the biomass is hydrolyzed. Hydrolysis includes cleavage of glycosidic bonds between sugar building blocks in a polysaccharide (e.g., cellulose, hemicellulose, starch). Hydrolysate is biomass that has at least partially undergone a hydrolysis reaction. In hydrolysate, the average degree of polymerization of the polysaccharides comprising the biomass can be reduced. The biomass need not be hydrolyzed to monomeric sugars. Biomass can be hydrolyzed to any suitable extent. In some embodiments, about 5%, about 10%, about 20%, about 30%, about 40%, about 50%, about 60%, about 70%, about 80%, about 90%, about 95%, about 96%, about 97%, about 98%, about 99%, about 99.5% or about 99.9% of the glycosidic bonds are hydrolyzed. In some embodiments, at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5% or at least 99.9% of the glycosidic bonds are hydrolyzed.

In some cases, the biomass is provided in hydrolyzed form. In some instances, the biomass is hydrolyzed in an ionic liquid and the hydrolysate or components thereof is recovered from the ionic liquid. Biomass can be hydrolyzed using any suitable method (e.g., by acids, by enzymes, in ionic liquids). In some cases, the biomass is hydrolyzed according to the methods described for example in U.S. Patent Pub. No. 2011/0065159, which is herein incorporated by reference in its entirety.

In some embodiments, the biomass is at least partially dissolved in the ionic liquid. In some cases, the reaction mixture comprises some non-dissolved biomass (including components of biomass such as lignin). In some instances, solubilization and hydrolysis of the biomass occurs simultaneously in the ionic liquid. The ionic liquid can contain a catalyst. The catalyst can catalyze hydrolysis in some cases (e.g., acid). In some embodiments, the catalyst increases the rate of production of furanic compounds.

Lignin may be dissolved, partially dissolved, or undissolved in the hydrolysate mixture. In some embodiments, the biomass mixture comprises some non-dissolved solids. In some embodiments, non-dissolved solids comprise lignin. In some embodiments, non-dissolved solids comprise ash. In some embodiments, non-dissolved solids comprise humin.

In some embodiments, the ionic liquid further comprises an acid (e.g., hydrochloric acid, sulfuric acid, carbonic acid, sulfuric acid, nitric acid, phosphoric acid, maleic acid). In some embodiments, the acid is immobilized (e.g., onto a surface such as silicon oxide particles). In some cases, the ionic liquid is acidic (e.g., the ionic liquid comprises an acidic functionality and/or is acid-functionalized). Examples of acidic ionic liquids include, but are not limited to 1-butyl-3-methylimidazolium bisulfate (C4mimHSO₄) and 1-(4-sulfobutyl)-3-methylimidazolium bisulfate (SbmimHSO₄).

In some cases, the rate and/or timing of water addition to a hydrolysis reaction is such that the yield and/or rate of hydrolysis is high (e.g., a yield of at least 40%, at least 50%, at least 60%, at least 70%, at least 80%, at least 90%, at least 95%, or at least 99%, wherein the yield is achieved in less than 5 minutes, less than 10 minutes, less than 30 minutes, less than 1 hour, less than 3 hours, less than 5 hours, or less than 9 hours). In some embodiments, the rate and/or timing of water addition provides water as a reactant in hydrolysis, while maintaining a high solubility of biomass in the ionic liquid (e.g., at least 50%, at least 60%, at least 70%, at least 80%, at least 90%, at least 95% or at least 99% of the solubility when no water is present).

In some embodiments, provided herein is a method for hydrolyzing biomass, the method comprising adding water to a reaction mixture comprising ionic liquid and biomass, wherein the water is added at a rate such that the solubility of the biomass is not substantially inhibited (e.g., at least 50%, at least 60%, at least 70%, at least 80%, at least 90%, at least 95% or at least 99% of the solubility when no water is present) and hydrolysis is not substantially inhibited (e.g., at least 50%, at least 60%, at least 70%, at least 80%, at least 90%, at least 95% or at least 99% of the maximum hydrolysis rate).

In another aspect, provided herein is a method for hydrolyzing biomass, the method comprising adding water to a reaction mixture comprising ionic liquid and biomass, wherein the water is added at a rate such that the rate of biomass solubilization is not substantially inhibited (e.g., at least 50%, at least 60%, at least 70%, at least 80%, at least 90%, at least 95% or at least 99% of the rate of solubilization when no water is present) and hydrolysis is not substantially inhibited (e.g., at least 50%, at least 60%, at least 70%, at least 80%, at least 90%, at least 95% or at least 99% of the maximum hydrolysis rate).

In another aspect, provided herein is a method for hydrolyzing biomass, the method comprising adding water to a reaction mixture comprising ionic liquid and biomass, wherein the water is added at a rate that is approximately equal to the rate at which water is consumed in the reaction (e.g., the water addition and consumption rates are no more than about 1%, about 3%, about 5%, about 10%, about 20%, or about 50% different).

In another aspect, provided herein is a method for hydrolyzing biomass, the method comprising adding water to a reaction mixture comprising ionic liquid and biomass, wherein the water is added at a rate that maintains the water concentration in the reaction mixture below about 45%, below about 35%, below about 25%, below about 15%, below about 10%, below about 5%, below about 3%, or below about 1% (w/w). In some embodiments, the water concentration in the reaction mixture is maintained between 0% and about 5%, between 0% and about 10%, 1% and about 5% or between 1% and about 10%.

In another aspect, provided herein is a method for hydrolyzing biomass, the method comprising adding water to a reaction mixture comprising ionic liquid and biomass, wherein the temperature of the mixture is such that the yield of sugars is at least 2 times, at least 5 times, at least 10 times, at least 20 times, at least 50 times, at least 100 times, or at least 1000 times greater than the yield of furanic compounds.

In some embodiments, a method for hydrolyzing biomass comprises adding water to a reaction mixture comprising ionic liquid and biomass, wherein the water is added at a rate such that the ratio (by mass) of side-products (HMF, furan) to sugar is less than the ratio (by mass) of side-products (HMF, furan, etc) to sugar obtained by the identical reaction comprising a fixed amount of water added.

In some embodiments, a method for hydrolyzing biomass comprises adding water to a reaction mixture comprising ionic liquid and biomass, wherein the water is added at a rate such that biomass solubilization is not substantially inhibited (e.g., at least 80%, at least 90%, or at least 95% of the maximal rate) and hydrolysis is not substantially inhibited (e.g., at least 80%, at least 90%, or at least 95% of the maximal rate).

In some embodiments, a method for hydrolyzing biomass comprises adding water to a reaction mixture comprising ionic liquid and biomass, wherein the water is added at a rate that is approximately equal to (e.g., within about 10%, about 5%, or about 1%) the rate at which water is consumed in the reaction.

In some embodiments, a method for hydrolyzing biomass comprises adding water to a reaction mixture comprising ionic liquid and biomass, wherein the water is added at a rate that maintains the water concentration in the reaction mixture below about 10%, 7%, 5%, 3%, 2% or 1% (w/w).

In some embodiments, the biomass is at least partially dissolved in the ionic liquid. In some embodiments, the reaction mixture comprises non-dissolved biomass. In some embodiments, the reaction mixture further comprises a pressurized gas. In some embodiments, the reaction mixture further comprises an acid.

In some embodiments, the reaction mixture further comprises carbon dioxide and at least some of the carbon dioxide is present as carbonic acid.

In some embodiments, a method for hydrolyzing biomass comprises adding water to a mixture, wherein the mixture comprises biomass, water and ionic liquid, and wherein the water is added at a rate that maintains the ratio of the concentration of water to the concentration of the biomass in the mixture.

In some embodiments, the ratio (of water to biomass) is maintained between 50% and 150% of the ratio before water is added. In some embodiments, the ratio is maintained between 80% and 120% of the ratio before water is added. In some embodiments, the ratio is maintained until the biomass is at least 50% hydrolyzed. In some embodiments, the ratio is maintained until the biomass is at least 75% hydrolyzed.

In some embodiments, the method further comprises, when the biomass is at least 90% hydrolyzed, adding water to the mixture to increase the ratio (of water to biomass) to at least 200% of the ratio before water is added. In some embodiments, the mixture further comprises an acid.

In some embodiments, a method for hydrolyzing biomass comprises adding water to a mixture, wherein the mixture comprises biomass, water and ionic liquid, and wherein the water is added at a rate that maintains the ratio of the concentration of water to the concentration of the ionic liquid in the mixture within a specified range.

In some embodiments, the ratio (of water to IL) is maintained between 50% and 150% of the ratio before water is added. In some embodiments, the ratio is maintained between 80% and 120% of the ratio before water is added.

In some embodiments, the ratio (of water to IL) is maintained until the biomass is at least 50% hydrolyzed. In some embodiments, the ratio is maintained until the biomass is at least 75% hydrolyzed.

In some embodiments, the method further comprises, when the biomass is at least 90% hydrolyzed, adding water to the mixture to increase the ratio (of water to IL) to at least 200% of the ratio before water is added. In some embodiments, the mixture further comprises an acid.

In some embodiments, a method for hydrolyzing biomass comprises adding water to a mixture, wherein the mixture comprises ionic liquid, water and biomass having glycosidic bonds, and wherein the water is added to the mixture at a rate that maintains a greater than stoichiometric concentration of water relative to the glycosidic bonds. In some embodiments, the mixture further comprises an acid.

In some embodiments, the ratio of the concentration of water to the concentration of glycosidic bonds is about 2. In some embodiments, the ratio of the concentration of water to the concentration of glycosidic bonds is at least 50 when the biomass is about 90% hydrolyzed.

In some embodiments, a method for hydrolyzing biomass comprises adding water to a mixture, wherein the mixture comprises ionic liquid, water and biomass, and wherein the water is added at a rate that reduces the electrical conductivity of the reaction mixture over time.

In some embodiments, the electrical conductivity is reduced in proportion to the extent to which the biomass is hydrolyzed. In some embodiments, the mixture further comprises an acid.

A method for hydrolyzing biomass, wherein a volume of water is added to a mixture comprising biomass and ionic liquid at least 1, 2, 3, 4, 5, 6, 7, 8, 9, 10 or more times during the time period in which the biomass is being hydrolyzed. In some embodiments, the volume of water is added to a mixture comprising biomass and ionic liquid at least 1 time during the time period in which the biomass is being hydrolyzed. In some embodiments, the volume of water is added to a mixture comprising biomass and ionic liquid at least 2 times during the time period in which the biomass is being hydrolyzed. In some embodiments, a method for hydrolyzing biomass comprises adding a volume of water at least 4 times during the time period in which the biomass is being hydrolyzed. In some embodiments, a volume of water is added at least 6 times during the time period in which the biomass is being hydrolyzed. In some embodiments, the two volumes of water are different amounts of water.

In some embodiments, a method for hydrolyzing biomass comprises: (a) dissolving at least some biomass in an ionic liquid; (b) hydrolyzing at least some of the biomass; and (c) adding water to the biomass and ionic liquid. In some embodiments, the water is added after at least some of the biomass is converted to water-soluble carbohydrates.

In some embodiments, a method for hydrolyzing biomass comprises: (a) contacting biomass with ionic liquid; and (b) adding water to the biomass and ionic liquid at least 5 minutes after the contacting. In some embodiments, the water is added at least 20 minutes after the contacting. In some embodiments, the biomass and ionic liquid are contacted in the presence of an acid.

In some embodiments, a method for hydrolyzing biomass comprises: (a) adding biomass comprising cellulose to ionic liquid such that the cellulose dissolves in the ionic liquid over a period of time; and (b) adding water to the ionic liquid when the degree of polymerization of the dissolved cellulose is less than 99% of the degree of polymerization of the cellulose in the biomass before being dissolved in the ionic liquid. In some embodiments, water is added when the degree of polymerization is less than 90% of the degree of polymerization of the cellulose in the biomass. In some embodiments, water is added when the degree of polymerization is less than 80% of the degree of polymerization of the cellulose in the biomass. In some embodiments, water is added when the degree of polymerization is less than 70% of the degree of polymerization of the cellulose in the biomass. In some embodiments, water is added when the degree of polymerization is less than 60% of the degree of polymerization of the cellulose in the biomass. In some embodiments, water is added when the degree of polymerization is less than 50% of the degree of polymerization of the cellulose in the biomass.

In some embodiments, a method for hydrolyzing biomass comprises: (a) adding biomass comprising hemicellulose to ionic liquid such that the hemicellulose dissolves in the ionic liquid over a period of time; and (b) adding water to the ionic liquid when the degree of polymerization of the dissolved hemicellulose is less than 99% of the degree of polymerization of the hemicellulose in the biomass before being dissolved in the ionic liquid. In some embodiments, water is added when the degree of polymerization is less than 90% of the degree of polymerization of the hemicellulose in the biomass. In some embodiments, water is added when the degree of polymerization is less than 80% of the degree of polymerization of the hemicellulose in the biomass. In some embodiments, water is added when the degree of polymerization is less than 70% of the degree of polymerization of the hemicellulose in the biomass. In some embodiments, water is added when the degree of polymerization is less than 60% of the degree of polymerization of the hemicellulose in the biomass. In some embodiments, water is added when the degree of polymerization is less than 50% of the degree of polymerization of the hemicellulose in the biomass.

In some embodiments, a method for hydrolyzing biomass comprises: adding water to a mixture of biomass and ionic liquid after the degree of polymerization of cellulose or hemicellulose dissolved in the ionic liquid has been reduced by at least about 1%, 3%, 5%, or 10% compared with the degree of polymerization of the cellulose or hemicellulose in the biomass before being dissolved in the ionic liquid.

In some embodiments, the concentration of water increases over time. In some embodiments, the concentration of ionic liquid decreases over time. In some embodiments, water is added at a faster rate than it is consumed in the hydrolysis reaction. In some embodiments, water is added to the biomass and ionic liquid at a continuous rate. In some embodiments, water is added to the biomass and ionic liquid in discrete intervals.

In some embodiments, a method for hydrolyzing biomass comprises adding water to a mixture of biomass and ionic liquid at a rate such that the concentration of furanic compounds in the mixture is less than about 0.1%, 0.5%, 1%, 3%, 5% or 10% (w/w).

### Lowering Temperature Following Biomass Hydrolysis

In some embodiments, a method for hydrolyzing a biomass polysaccharide substrate comprises hydrolyzing a reaction mixture comprising the biomass polysaccharide substrate and an ionic liquid in which the biomass polysaccharide substrate is soluble and adding water to the reaction mixture, wherein water is added at a rate such that the polysaccharide of the biomass polysaccharide substrate is not precipitated from the reaction mixture and hydrolysis is not substantially inhibited, and following hydrolysis, lowering the temperature of the reaction mixture from the temperature at which hydrolysis is performed.

In some embodiments, the reaction mixture further comprises acid. In some embodiments, the amount of acid ranges from about 5 weight % to 40 weight % relative to the amount of biomass polysaccharide substrate in the reaction. In some embodiments, the amount of acid ranges from about 10 weight % to 25 weight % relative to the amount of biomass polysaccharide substrate in the reaction.

In some embodiments, the biomass polysaccharide substrate is lignocellulosic biomass. In some embodiments, hydrolysis is continued until the monosaccharide yield is 50%, 60%, 70%, 80%, 90% or higher.

In some embodiments, the reaction mixture is heated to a temperature of about 70 to 140° C. during hydrolysis. In some embodiments, the reaction mixture is cooled to a temperature of about 20 to 100° C. following hydrolysis.

In some embodiments, the ionic liquid comprises chloride, trifluoroacetate, trichloroacetate, tribromoacetate or thiocyanate. In some embodiments, the cation of the ionic liquid is an imidazolium or a pyridinium. In some embodiments, the ionic liquid is [EMIM]Cl, [BMIM]Cl, 1-ethyl-2,3-dimentylimidazolium chloride or 1-alkylpyridinium chloride.

In some embodiments, water is added such that the total amount of water in the reaction mixture is less than 20 weight %.

In some embodiments, (a) a total water level of 20 weight % with respect to the total reaction mixture is added by 3-10 minutes after initiation of hydrolysis; (b) a total water level of 20 weight % with respect to the total reaction mixture is added by 10 minutes after initiation of hydrolysis; (c) a total water level of 20-35 weight % with respect to the total reaction mixture is added within 10-30 minutes after initiation of hydrolysis; (d) a total water level of 35-45 weight % with respect to the total reaction mixture is added within 30-60 minutes after initiation of hydrolysis; or (e) a total water level of 40-45 weight % with respect to the total reaction mixture is added within 60 minutes after initiation of hydrolysis.

In some embodiments, the temperature is lowered such that the yield of 5-hydroxymethylfurfural in the hydrolysis product is at most about 10%, 5%, 3%, 2%, 1%, 0.5%, 0.1%, or less.

In some embodiments, a co-solvent is added to the reaction mixture in an amount ranging from 1% to 25 weight % of the reaction mixture.

In some embodiments, the disclosure includes a hydrolysis product prepared by the methods described herein.

In some embodiments, a method for making a monosaccharide feedstock comprises preparing a hydrolysis product and separating the hydrolysis product from ionic liquid.

In some embodiments, a method for generating ethanol by fermentation comprises employing the hydrolysis product of the method as a monosaccharide feedstock for fermentation by an ethanologenic microorganism.

### Fluids Including Pressurized Gases and Supercritical Fluids

In some instances, the methods described herein use fluids. Exemplary fluids include but are not limited to gases, liquids, pressurized gases, liquefied gases, sub-critical fluids, volatile liquids, and/or supercritical or near-supercritical fluids. For example, in one embodiment, a composition comprising an ionic liquid, water and a hydrogen bonding solute is contacted with a gas to form a first phase comprising an ionic liquid and a second phase comprising water and a hydrogen bonding solute. In another embodiment, a composition comprising a furanic compound and an ionic liquid is contacted with a pressurized gas. In yet another embodiment, a composition comprising one or more biomass components in an ionic liquid is contacted with a supercritical or near-supercritical fluid.

In some instances, fluids and/or pressurized gases (e.g., greater than atmospheric pressure) are used in processes for dissolving and/or hydrolyzing biomass.

In some embodiments, the fluid is selected from the group consisting of CO₂, NO₂, NH₃, water, acetic acid, methanol, ethanol, n-butane, nitrogen, hydrogen, helium, argon, oxygen, methane, ethane, propane, ethylene, propylene, and combinations thereof. In some embodiments, the fluid is CO₂.

As used herein, “contacted with a gas” does not necessarily mean that the fluid is a gas when contacted with the ionic liquid and/or biomass mixture. In some cases, the gas can be pressurized such that it is a dense phase (e.g., liquefied gas or supercritical fluid) when contacted. As used herein, a gas is a material that is a vapor at International Union of Pure and Applied Chemistry (IUPAC) standard temperature and pressure (0° C. and 1 bar). A pressurized gas is any gas at a pressure greater than 1 bar.

In some embodiments, the biomass mixture and/or ionic liquid is contacted with a pressurized gas. In some instances, the gas is pressurized to an absolute pressure greater than atmospheric pressure. In some embodiments, the pressure is about 1 bar, about 2 bar, about 5 bar, about 10 bar, about 20 bar, about 30 bar, about 40 bar, about 50 bar, about 100 bar, about 200 bar, about 300 bar or about 400 bar. In some embodiments, the pressure is at least 1 bar, at least 2 bar, at least 5 bar, at least 10 bar, at least 20 bar, at least 30 bar, at least 40 bar, at least 50 bar, at least 100 bar, at least 200 bar, at least 300 bar or at least 400 bar.

In some embodiments, the biomass mixture and/or ionic liquid is contacted with a liquefied gas. Examples of gases that can be liquefied include propane, hydrogen, nitrogen, n-butane and carbon dioxide.

In some embodiments, the biomass mixture and/or ionic liquid is contacted with a volatile liquid (e.g., a liquid that turns into a vapor at a temperature of about 60° C., about 100° C., about 150° C., or about 200° C. at atmospheric pressure). Examples of liquids that are readily volatile include propanone, methanol and ethanol.

The critical temperature of a fluid is the temperature above which a distinct liquid phase does not exist (e.g., regardless of pressure). The vapor pressure of a fluid at its critical temperature is its critical pressure. At temperatures and pressures above its critical temperature and pressure (e.g., its critical point), a fluid is called a supercritical fluid. Many fluids can form supercritical fluids provided they do not degrade or decompose at temperatures below their critical temperature.

In some instances, the methods of the present invention can use any suitable supercritical or near-supercritical fluid. Information on supercritical fluids can be found in “Fundamentals of Supercritical Fluids” by Tony Clifford (ISBN: 978-0198501374), “Supercritical Carbon Dioxide: Separations and Processes” by Aravamudan S. Gopalan (ISBN: 978-0841238367), and “Supercritical Fluid Extraction” by Larry T. Taylor (ISBN: 978-0471119906), each of which is herein incorporated by reference in its entirety. Exemplary supercritical or near-supercritical fluid include but are not limited to CO₂, NO₂, NH₃, water, acetic acid, methanol, ethanol, n-butane, nitrogen, hydrogen, helium, argon, oxygen, methane, ethane, propane, ethylene, propylene, and any combinations thereof.

The fluid can be supercritical, in that both the temperature is at or above its critical temperature and the pressure is at or above its critical pressure. In some embodiments, the pressure is about 100%, about 120%, about 150%, about 200%, about 300%, about 500%, and the like of the fluid's critical pressure. In some embodiments, the pressure is at least about 100%, at least about 120%, at least about 150%, at least about 200%, at least about 300%, at least about 500%, and the like of the fluid's critical pressure. In some embodiments, the temperature is about 100%, about 120%, about 150%, about 200%, about 300%, about 500%, and the like of the fluid's critical temperature. In some embodiments, the temperature is at least about 100%, at least about 120%, at least about 150%, at least about 200%, at least about 300%, at least about 500%, and the like of the fluid's critical temperature. In some embodiments, the pressure is between about 80% and 400% of the fluid's critical pressure. In some embodiments, the temperature is between about 80% and 400% of the fluid's critical temperature.

The fluid can be sub-critical (e.g., near-supercritical), in that one or both of the temperature is below the fluid's critical temperature and the pressure is below its critical pressure. A near-supercritical fluid may have properties similar or near the properties of a supercritical fluid. In various embodiments, the pressure is about 99%, about 98%, about 95%, about 90%, about 85%, about 75%, about 50%, about 20%, and the like of the fluid's critical pressure. In various embodiments, the pressure is at least about 99%, at least about 98%, at least about 95%, at least about 90%, at least about 85%, at least about 75%, at least about 50%, at least about 20%, and the like of the fluid's critical pressure. In various embodiments, the temperature is about 99%, about 98%, about 95%, about 90%, about 85%, about 75%, about 50%, about 20%, and the like of the fluid's critical temperature. In various embodiments, the temperature is at least about 99%, at least about 98%, at least about 95%, at least about 90%, at least about 85%, at least about 75%, at least about 50%, at least about 20%, and the like of the fluid's critical temperature.

In some embodiments, fluids with low critical temperatures and/or pressures may be employed (e.g., to reduce the amount of energy that needs to be put into the process to heat and/or pressurize the fluid). In some embodiments, fluids with low temperatures are employed (e.g., to preserve heat labile reactants and/or products). In some embodiments, the temperature is sufficiently low to avoid decomposition of the biomass components (e.g., less than 200° C., less than 150° C., less than 100° C., less than 80° C., less than 60° C., less than 40° C., less than 30° C., less than 20° C., or less than 10° C.).

Supercritical fluids can have densities, viscosities, and other properties that are intermediate between those of the fluid in its gaseous and in its liquid state. Table 1 lists some supercritical properties of four compounds. These four fluids are examples of fluids that have relatively moderate critical temperatures (e.g., less than 200° C., less than 150° C., less than 100° C., less than 80° C., less than 60° C., less than 40° C., less than 30° C., less than 20° C., or less than 10° C.) and critical pressures (e.g., less than 200 atm, less than 150 atm, less than 120 atm, less than 110 atm, less than 100 atm, less than 90 atm, less than 80 atm, less than 70 atm, less than 60 atm, less than 50 atm, less than 40 atm, less than 30 atm, or less than 20 atm).

TABLE 1
Supercritical properties of exemplary fluids

| | Critical | Critical | Critical Point | Density |
|---|---|---|---|---|
| | Temperature, | Pressure, | Density, | at 400 |
| Fluid | °C. | atm | g/mL | atm, g/mL |
| CO₂ | 13.2 | 72.9 | 0.47 | 0.96 |
| N₂O | 36.5 | 71.7 | 0.45 | 0.94 |
| NH₃ | 132.5 | 112.5 | 0.24 | 0.40 |
| n-Butane | 152.0 | 37.5 | 0.23 | 0.50 |

In some cases, supercritical fluids dissolve solutes in proportion to the density of the fluid. In some embodiments, the supercritical or near-supercritical fluid has a density of about 0.05, about 0.1, about 0.15, about 0.2, about 0.25, about 0.3, about 0.35, about 0.4, about 0.5, about 0.6, about 0.7, about 0.8, about 0.9, or about 1.0 g/mL. In some embodiments, the supercritical or near-supercritical fluid has a density of at least 0.05, at least 0.1, at least 0.15, at least 0.2, at least 0.25, at least 0.3, at least 0.35, at least 0.4, at least 0.5, at least 0.6, at least 0.7, at least 0.8, at least 0.9, or at least 1.0 g/mL. In some embodiments, the supercritical or near-supercritical fluid has a density of between about 0.2 and 0.9 g/mL.

In various embodiments, the supercritical or near-supercritical fluid is capable of extracting solutes having a molecular weight of about 100, about 200, about 300, about 400, about 500, about 600, about 800, about 1000, about 1200, about 1500, about 2000, or about 3000 atomic mass units (amu). In some embodiments, the supercritical or near-supercritical fluid is capable of extracting solutes having a molecular weight of less than 100, less than 200, less than 300, less than 400, less than 500, less than 600, less than 800, less than 1000, less than 1200, less than 1500, less than 2000, or less than 3000 atomic mass units (amu). In some embodiments, the supercritical or near-supercritical fluid is capable of extracting solutes having a molecular weight of greater than 100, greater than 200, greater than 300, greater than 400, greater than 500, greater than 600, greater than 800, greater than 1000, greater than 1200, greater than 1500, greater than 2000, or greater than 3000 atomic mass units (amu). In some embodiments, the supercritical or near-supercritical fluid is capable of extracting solutes having a molecular weight of between about 10 and 600 amu. Solutes can include for example but are not limited to biomass components.

In some embodiments, the supercritical or near-supercritical fluid is selected from the group consisting of CO₂, NO₂, NH₃, water, acetic acid, methanol, ethanol, n-butane, nitrogen, hydrogen, helium, argon, oxygen, methane, ethane, propane, ethylene, propylene, and combinations thereof. In some embodiments, the supercritical or near-supercritical fluid is CO₂.

In some embodiments, the fluid is substantially pure (e.g., at least 80%, 90%, 95%, 99%, 99.5, or 99.9% pure). In some embodiments, the fluid is a mixture of more than one chemical species (e.g., compounds).

In one embodiment, the supercritical or near-supercritical fluid is CO₂ comprising any amount of water. In some instances, water increases the solubility of sugars in CO₂. Without being held to any particular theory, it is thought that polar and/or hydrogen bonding interactions between a polar co-solvent such as water and the sugar increases the solubility of sugars in the supercritical or near-supercritical CO₂ phase.

In some embodiments, the fluid is non-toxic, biodegradable, non-flammable, or has other properties that result in a safe and environmentally friendly process.

In some embodiments, the formation of precipitate may be enhanced by cooling, heating, vibrating, sounding (acoustic wave), or any combination thereof.

### Biomass Dissolution

Mixing polar organic solvents with ionic liquids can decrease viscosity and enhance dispersion of biomass in a liquid, therefore enhancing dissolution rates (sometimes dramatically relative to pure ionic liquids). In some instances, biomass is mixed with a polar organic such as N,N-dimethylformamide, N,N-dimethylacetamide, pyrrolidinone, valorolactam, sulfolane, acetylacetone, dimethylsulfoxide and any combinations thereof. This allows biomass to swell, potentially increasing mass transfer rates around cellulose fibers. In some embodiments, the addition of ionic liquids such as [BMIM]Cl or other biomass-dissolving ionic liquids completes dissolution. The potential advantages is to speed up dissolution and/or reduce the amount of ionic liquid required. Some combinations of polar organics with ionic liquids allow dissolution of cellulose to occur with less than 0.20 mole fraction of ionic liquid while increasing dissolution rates tremendously.

In some cases, the dissolution process is rate-limited by mass transfer. In some cases, a simple co-solvent such as the polar organic solvent can quickly penetrate coalesced cellulose fibers and act as a low-viscosity conduit for ionic liquid to interact with and dissolve the cellulose. In some embodiments, polar organic solvents can act as catalysts for dissolution.

Like polar organic solvents, CO₂ can penetrate cellulosic fibers. The CO₂ can lower the viscosity of ionic liquid, allowing much faster mass transfer. Unlike polar organic solvents, after dissolution the CO₂ can be removed by simple decompression (and accelerated by vibration, agitation, or some common degassing process). Polar organic solvents, on the other hand, require a different process such as distillation or liquid-liquid extraction for separation, which is usually more expensive in both capital and operation.

Some combinations of the polar organic solvents above allow dissolution of cellulose to occur with less than 0.20 mole fraction of ionic liquid while increasing dissolution rates tremendously.

In some cases, a pressurized gas (e.g., CO₂) is used to preferentially dissolve some biomass components over others (e.g., polysaccharide more than lignin relative to their solubilities and/or rates of solubilization without the pressurized gas). Some biomass dissolving ionic liquids may dissolve some lignin, but dissolve almost none when pressurized with CO₂.

In some embodiments, a method for dissolving biomass and components thereof comprises contacting the biomass or component thereof with an ionic liquid at a pressure greater than atmospheric pressure.

In some embodiments, the pressure is imposed directly into the liquid. In some embodiments, the pressure is imposed between the ionic liquid and a surface. In some embodiments, the pressure is imposed indirectly. In some embodiments, the pressure is imposed by first compressing a fluid other than the ionic liquid.

In some embodiments, the pressure is increased to more than 2 atmospheres, more than 5 atmospheres, more than 10 atmospheres or more than 20 atmospheres. In some embodiments, the applied pressure is stationary or non-stationary. In some embodiments, the non-stationary pressure takes the form of vibration, acoustic waves, ultrasound, agitation, and the like. In some embodiments, the pressure is oscillated.

In some embodiments, the increased pressure increases the rate at which the biomass or components thereof dissolve in the ionic liquid by at least 1% relative to the rate at which the biomass dissolves in the ionic liquid at atmospheric pressure. In some embodiments, the increased pressure increases the solubility of the biomass or components thereof in the ionic liquid by at least 1% relative to the solubility of the biomass in the ionic liquid at atmospheric pressure.

In some embodiments, the ionic liquid is contacted with a pressurized gas. In some embodiments, the pressurized gas is air. In some embodiments, pressurized gas is carbon dioxide, methane, ethane, propane, butane, natural gas, methanol, ethanol, propanol, butanol, nitrous oxide, ammonia, water, or any combination thereof. In some embodiments, the pressurized gas at least partially dissolves in the ionic liquid.

In some embodiments, contacting the ionic liquid with the pressurized gas reduces the viscosity of the ionic liquid by at least 5%. In some embodiments, the biomass has a solubility of at least 3% in the ionic liquid.

In some embodiments, the rate at which the biomass dissolves in the ionic liquid is at least 20% of the maximum rate at pressures between atmospheric pressure and 100 atm. In some embodiments, the solubility of the biomass in the ionic liquid is at least 20% of the maximum solubility at pressures between atmospheric pressure and 100 atm.

In some embodiments, the method further comprises agitating the ionic liquid. In some embodiments, the method further comprises ultrasounding the ionic liquid.

In some embodiments, the biomass is contacted with the ionic liquid at a temperature. The ionic liquid is a liquid at the temperature when in contact with the pressurized gas. The ionic liquid is a solid at the temperature when not in contact with the pressurized gas.

In some embodiments the present description provides a method for dissolving biomass comprises contacting biomass with ionic liquid, wherein the ionic liquid is in contact with a pressurized gas.

In some embodiments, the rate at which the biomass dissolves in the ionic liquid is at least 5% greater than the rate at which the biomass dissolves in the ionic liquid when the ionic liquid is not in contact with the pressurized gas. In some embodiments, the solubility of the biomass in the ionic liquid is at least 1% greater than the solubility of the biomass in the ionic liquid when the ionic liquid is not in contact with the pressurized gas.

In some embodiments, contacting the ionic liquid with the pressurized gas reduces the viscosity of the ionic liquid by at least 5%, 10%, 15% or 20%. In some embodiments, the biomass has a solubility of at least 3% in the ionic liquid.

In some embodiments, the rate at which the biomass dissolves in the ionic liquid is at least 20% of the maximum rate at pressures between atmospheric pressure and 100 atm. In some embodiments, the solubility of the biomass in the ionic liquid is at least 20% of the maximum solubility at pressures between atmospheric pressure and 100 atm.

In some embodiments, the method further comprises agitating the ionic liquid. In some embodiments, the method further comprises ultrasounding the ionic liquid.

In some embodiments, the gas comprises carbon dioxide.

In some embodiments, the biomass is contacted with the ionic liquid at a temperature, the ionic liquid is a liquid at the temperature when in contact with the pressurized gas, and the ionic liquid is a solid at the temperature when not in contact with the pressurized gas.

### Use of Pressure for Biomass Hydrolysis

Several aspects of hydrolyzing biomass can be enhanced by the application of pressure. In one aspect, the method for hydrolyzing biomass comprises contacting biomass with ionic liquid at a pressure greater than atmospheric pressure, wherein the biomass is hydrolyzed in the ionic liquid.

An increase in pressure on a solution of biomass in ionic liquid and water can increase the rate of hydrolysis above the rate of an identical hydrolysis reaction performed at atmospheric pressure. The dissociation constant of pure water (i.e., water to hydronium and hydroxyde ions) increases significantly at elevated pressure (e.g., by a few orders of magnitude). This effect can be similar to transforming water into an acid catalyst for hydrolysis.

For the case of a dilute solution of water in ionic liquid, the ionic strength of the ionic liquid can increase the dissociation of water, also creating a higher effective acidity. In some cases, the effective acidity of dilute water in ionic solutions is not a low enough pH to catalyze hydrolysis. However, increasing the pressure (to any suitably high pressure) in a dilute (e.g., 1%, 3%, 5%, 10%) solution of water in ionic liquid can catalyze hydrolysis (e.g., by further lowering the effective acidity) in some instances. In some cases, the combination of pressure and a dilute solution of water in ionic liquid is inadequate to catalyze hydrolysis at a suitable rate, but does decrease the amount of acid that is needed to be added to the hydrolysis reaction and/or makes weaker acids suitable (e.g., carbonic acid).

There are several ways to increase pressure, which can be categorized into using a gas, or using a surface. In the case of a gas, one applies pressure to a gas, and the pressure is transmitted to the ionic liquid solution. One may or may not get observe dissolution along with the pressure (e.g., depending on the gas species). In the second case, the pressure is applied by the surface. This could oscillatory pressure (e.g., vibration, acoustic wave, ultrasound, etc), or non-oscillatory (e.g., a piston compressor).

In some embodiments, a method for hydrolyzing a biomass polysaccharide substrate comprises hydrolyzing a reaction mixture comprising the biomass polysaccharide substrate and an ionic liquid in which the biomass polysaccharide substrate is soluble and adding water to the reaction mixture, wherein water is added at a rate such that the polysaccharide of the biomass polysaccharide substrate is not precipitated from the reaction mixture and hydrolysis is not substantially inhibited, wherein the pressure at which hydrolysis is performed is not atmospheric pressure.

In some embodiments, the pressure is greater than atmospheric pressure. In some embodiments, the pressure is less than atmospheric pressure. In some embodiments, the pressure is increased as the hydrolysis reaction proceeds. In some embodiments, the pressure is decreased as the hydrolysis reaction proceeds.

In some embodiments, the reaction mixture further comprises acid. In some embodiments, hydrolysis is continued until the monosaccharide yield is 50% or higher. In some embodiments, the amount of acid ranges from about 5 weight % to 40 weight % relative to the amount of biomass polysaccharide substrate in the reaction. In some embodiments, the amount of acid ranges from about 10 weight % to 25 weight % relative to the amount of biomass polysaccharide substrate in the reaction.

In some embodiments, the biomass polysaccharide substrate is lignocellulosic biomass.

In some embodiments, the reaction mixture is heated to a temperature of about 70 to 140° C. during hydrolysis. In some embodiments, the reaction mixture is cooled to a temperature of about 20 to 100° C. following hydrolysis.

In some embodiments, the ionic liquid comprises chloride, trifluoroacetate, trichloroacetate, tribromoacetate or thiocyanate. In some embodiments, the cation of the ionic liquid is an imidazolium or a pyridinium. In some embodiments, the ionic liquid is [EMIM]Cl, [BMIM]Cl, 1-ethyl-2,3-dimentylimidazolium chloride or 1-alkylpyridinium chloride.

In some embodiments, water is added such that the total amount of water in the reaction mixture is less than 20 weight %, less than 10% weight, less than 5% weight or less than 1% weight.

In some embodiments, (a) a total water level of 20 weight % with respect to the total reaction mixture is added by 3-10 minutes after initiation of hydrolysis; (b) a total water level of 20 weight % with respect to the total reaction mixture is added by 10 minutes after initiation of hydrolysis; (c) a total water level of 20-35 weight % with respect to the total reaction mixture is added within 10-30 minutes after initiation of hydrolysis; (d) a total water level of 35-45 weight % with respect to the total reaction mixture is added within 30-60 minutes after initiation of hydrolysis; or (e) a total water level of 40-45 weight % with respect to the total reaction mixture is added within 60 minutes after initiation of hydrolysis.

In some embodiments, the pressure is such that the yield of 5-hydroxymethylfurfural in the hydrolysis product is 10% or less.

In some embodiments, a co-solvent is added to the reaction mixture in an amount ranging from 1 to 25 weight % of the reaction mixture.

An aspect of the disclosure provides a hydrolysis product prepared by the methods described herein.

Another aspect of the disclosure provides a method for making a monosaccharide feedstock that comprises preparing a hydrolysis product and separating the hydrolysis product from ionic liquid.

Another aspect of the disclosure provides a method for generating ethanol by fermentation comprising employing the hydrolysis product described herein as a monosaccharide feedstock for fermentation by an ethanologenic microorganism.

Another aspect of the disclosure provides a method for hydrolyzing biomass comprising applying a variable pressure to a mixture comprising biomass, water and ionic liquid. In some embodiments, the pressure is decreased as the biomass is hydrolyzed. In some embodiments, the pressure is increased as the biomass is hydrolyzed. In some embodiments, pressure is varied such that the solubility of the biomass in the ionic liquid is not substantially decreased and/or the rate of hydrolysis is not substantially decreased (i.e., the solubility and/or rate are at least 70%, at least 80%, or at least 90% of maximum).

In some embodiments, the mixture further comprises acid. In some embodiments, the ionic liquid comprises acid.

Another aspect of the present disclosure provides a method for hydrolyzing biomass comprising contacting biomass with ionic liquid at a pressure greater than atmospheric pressure, wherein the biomass is hydrolyzed in the ionic liquid. In some embodiments, the ionic liquid is in contact with a pressurized gas. In some embodiments, the pressure is greater than 1, 2, 3, 4, 5, 6, 7, 8, 9, 10, 15, 20, or 30 atm. In some embodiments, the rate of hydrolysis is at least 10%, 20%, 30%, 40% or 50% greater than the rate of hydrolysis at atmospheric pressure. In some embodiments, water is added to the biomass and ionic liquid at a rate that is approximately equal to the rate at which water is consumed in the hydrolysis reaction.

Biomass Hydrolysis with Pressurized Eases

Gases (e.g., CO₂) can be used under pressure to improve dissolution and hydrolysis of biomass. In some cases, the CO₂ enhances dissolution rate and carrying capacity of the ionic liquid. In some instances, the CO₂ enhances hydrolysis rate. In some embodiments, the CO₂ creates a biphasic or multiphasic system where reaction and extraction can occur simultaneously as described herein.

The use of CO₂ to create the pressure onto a hydrolysis mixture also dissolves some CO₂ in the hydrolysis reaction solution. The CO₂ can lower the viscosity of the ionic liquid-rich phase. In the presence of water, CO₂ can also lower the pH by reacting with water and forming carbonic acid. The combined effect of lower viscosity and lower pH can enhance hydrolysis.

To avoid precipitation of solids while still enhancing hydrolysis, one can time the pressure changes (e.g., pressure scheduling). In some cases, a hydrolysis reaction is started and the pressure is gradually built up once the biomass-ionic liquid solution viscosity has dropped. A drop in viscosity can be indicative of a major reduction in the degree of polymerization of cellulose and hemicellulose, and therefore an increase of water-soluble fragments, thus avoiding precipitation of longer chain polysaccharides.

Some ionic liquids that are solids at room temperature not only become liquids with dissolved carbon dioxide, but also retain the ability to dissolve cellulose. Here, the pressure of CO₂ can modulate the viscosity and solubility to better control dissolution and hydrolysis reactions. In some cases, the same vessel can be used to separate the hydrolysis fractions produced. Many configurations would be possible, such as a column with internals to maintain a pressure gradient, effecting continuous reaction and separation.

In some embodiments, a method for hydrolyzing biomass comprises contacting biomass with ionic liquid, wherein the ionic liquid is in contact with a pressurized gas and the biomass is hydrolyzed in the ionic liquid.

In some embodiments, the rate of hydrolysis is at least 5% greater than the rate of hydrolysis when the ionic liquid is not in contact with the pressurized gas.

In some embodiments, the ionic liquid comprises acid (e.g., hydrochloric acid or carbonic acid). In some embodiments, the gas comprises carbon dioxide.

In some embodiments, the ionic liquid comprises carbonic acid. In some embodiments, the gas is pressurized to at least 1, 2, 3, 4, 5, 6, 7, 8, 9, 10, 15, 20 or 30 atm.

In some embodiments, water is added to the biomass and ionic liquid at a rate that is approximately equal to the rate at which water is consumed in the hydrolysis reaction (e.g., within about 99%, 95%, 90%, 80% or 70% of each other).

In some embodiments, the biomass is contacted with the ionic liquid at a temperature, the ionic liquid is a liquid at the temperature when in contact with the pressurized gas, and the ionic liquid is a solid at the temperature when not in contact with the pressurized gas.

In some embodiments, contacting the ionic liquid with the pressurized gas reduces the viscosity of the hydrolysis reaction by at least 3%, 4%, 5%, 10%, 15% or 20%.

In some embodiments, the pressure is adjusted as the hydrolysis reaction proceeds such that the rate of hydrolysis decreases by no more than about 10%, 20%, 30%, 50%, 60%, 70% or 80% during the course of the hydrolysis reaction.

In some embodiments, the pressure is increased as the hydrolysis reaction proceeds. In some embodiments, the pressure is decreased as the hydrolysis reaction proceeds.

In some embodiments, the method further comprises extracting hydrolysis products or derivatives thereof in the gas.

### Fluid Extraction

In some embodiments, described herein is a method for extracting one or more biomass components comprising contacting a solution comprising one or more biomass components in an ionic liquid with a fluid, wherein at least some of the biomass components dissolve in the fluid and/or become un-dissolved in the ionic liquid solution.

In some embodiments, the fluid is miscible in the ionic liquid (e.g., to any small or large extent). In some embodiments, substantially none of the ionic liquid dissolves in the fluid. For example, in some cases, the concentration of the ionic liquid in the fluid is about 1%, about 0.5%, about 0.1%, about 0.05%, about 0.01%, about 0.005%, about 0.001%, about 0.0005%, or about 0.0001% by mass (w/w). In some cases, the concentration of the ionic liquid in the fluid is less than 1%, less than 0.5%, less than 0.1%, less than 0.05%, less than 0.01%, less than 0.005%, less than 0.001%, less than 0.0005%, or less than 0.0001% by mass (w/w).

In some embodiments, some ionic liquid dissolves in the fluid at the conditions at which the ionic liquid and the fluid are contacted. When an appreciable and/or unsuitably high amount (e.g., greater than 1%, or greater than 0.1%) of ionic liquid dissolves in the fluid, the method further comprises adjusting the pressure and/or temperature so that substantially none of the ionic liquid dissolves in the fluid. In some embodiments, the pressure and/or temperature of the fluid is adjusted such that the concentration of ionic liquid in the fluid is about 1%, about 0.5%, about 0.1%, about 0.05%, about 0.01%, about 0.005%, about 0.001%, about 0.0005%, or about 0.0001% by mass (w/w). In some embodiments, the pressure and/or temperature of the fluid is adjusted such that the concentration of ionic liquid in the fluid is less than 1%, less than 0.5%, less than 0.1%, less than 0.05%, less than 0.01%, less than 0.005%, less than 0.001%, less than 0.0005%, or less than 0.0001% by mass (w/w). In some cases, the pressure is increased above the critical point of the fluid. In some cases, the temperature is increased above the critical point of the fluid.

In some embodiments, the fluid is a supercritical or near-supercritical fluid. The fluid may comprise carbon dioxide, optionally with a co-solvent such as water. The biomass components can include, but are not limited to carbohydrates (e.g., sugars), proteins, lipids, lignin, biomolecules or derivatives thereof. In some embodiments, the concentration of the sugar in the ionic liquid is at least 5%, at least 1%, at least 0.5%, or at least 0.1% by mass (w/w). In some embodiments, the concentration of the sugar in the ionic liquid is between 2% and about 15%.

### Fluid Phases

In some embodiments, described herein is a composition comprising an ionic liquid, a pressurized gas, water and a biomass. In some methods, the biomass (and/or components and/or derivatives thereof) is extracted from the composition. The composition can be separated into two or more phases in some instances.

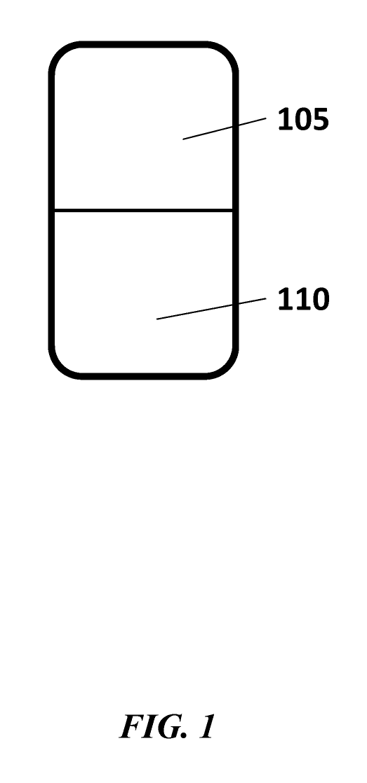

In some embodiments as described herein provide multi-phasic system. With reference to FIG. 1, a first phase 105 comprises a pressurized gas, water and one or more biomass components and a second phase 110 comprises an ionic liquid and one or more biomass components. In some cases, the lignin is in the second phase. In some cases, the pressurized gas is a supercritical or near-supercritical fluid. In some instances, the first phase comprises less than 0.5%, less than 0.1%, less than 0.05%, less than 0.01%, or less than 0.005% ionic liquid by mass. The second phase may comprise at least 50% ionic liquid.

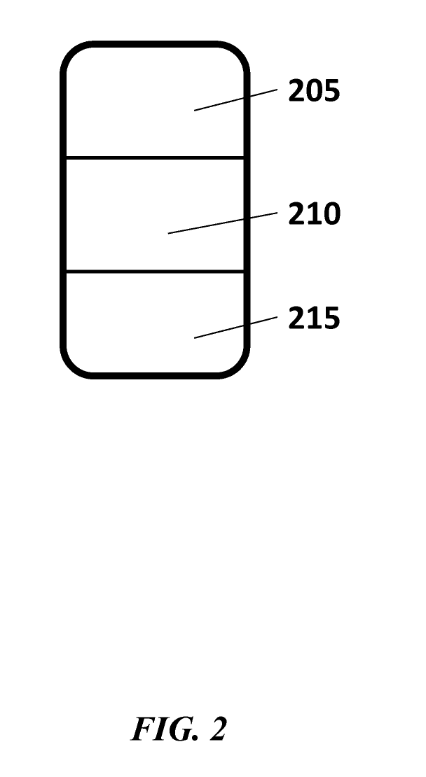

In some embodiments, a multi-phasic system comprises at least three phases. With reference to FIG. 2, a first phase 205 comprises a pressurized gas, water and one or more biomass components, a second phase 210 comprises a pressurized gas, water, one or more biomass components and an ionic liquid, and a third phase 215 comprises an ionic liquid and one or more biomass components. In some cases, the lignin is in the third phase. In some instances, the first phase comprises less than 0.5%, less than 0.1%, less than 0.05%, less than 0.01%, or less than 0.005% ionic liquid by mass. The second phase may comprise at least 50% water. The third phase may comprise at least 50% ionic liquid.

In some embodiments, described herein is a method for extracting one or more biomass components comprising contacting a composition comprising one or more biomass components in an ionic liquid with a supercritical or near-supercritical fluid.

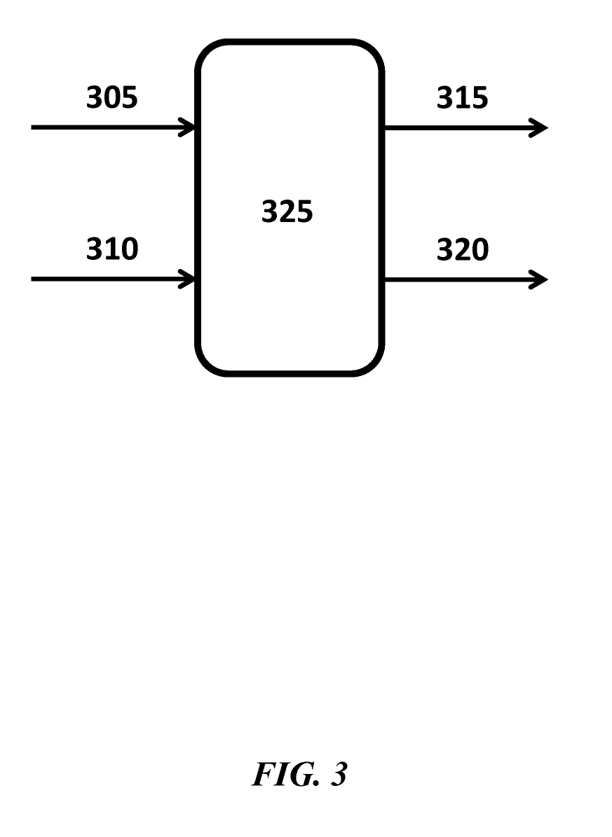

With reference to FIG. 3, a method for extracting a biomass component from an ionic liquid mixture can comprise contacting an ionic liquid mixture containing a biomass component 305 with a pressurized gas, supercritical or near-supercritical fluid (e.g., examples of “fluids”) 310 to form a post-extraction fluid mixture 315 and a post-extraction ionic liquid mixture 320. In some cases, the lignin is precipitated from the ionic liquid mixture 320. In some embodiments, the post-extraction ionic liquid mixture has less amount of the biomass component than the amount contained in the ionic liquid mixture and the post-extraction pressurized gas, supercritical or near-supercritical fluid mixture has more amount of the biomass component than the amount contained in the pressurized gas, supercritical or near-supercritical fluid. The extraction can be performed in any suitable vessel 325. In some cases, the vessel is appropriately shaped and sized to allow for adequate contacting of the ionic liquid mixture and the pressurized gas, supercritical or near-supercritical fluid and/or to allow for adequate partitioning of the post-extraction pressurized gas, supercritical or near-supercritical fluid mixture from the post-extraction ionic liquid mixture.

In some embodiments, the post-extraction pressurized gas, supercritical or near-supercritical fluid mixture has a pressure such that ionic liquid is rejected from the post-extraction pressurized gas, supercritical or near-supercritical fluid mixture (e.g., has less than 0.5%, less than 0.1%, less than 0.05%, less than 0.01%, or less than 0.005% ionic liquid by mass).

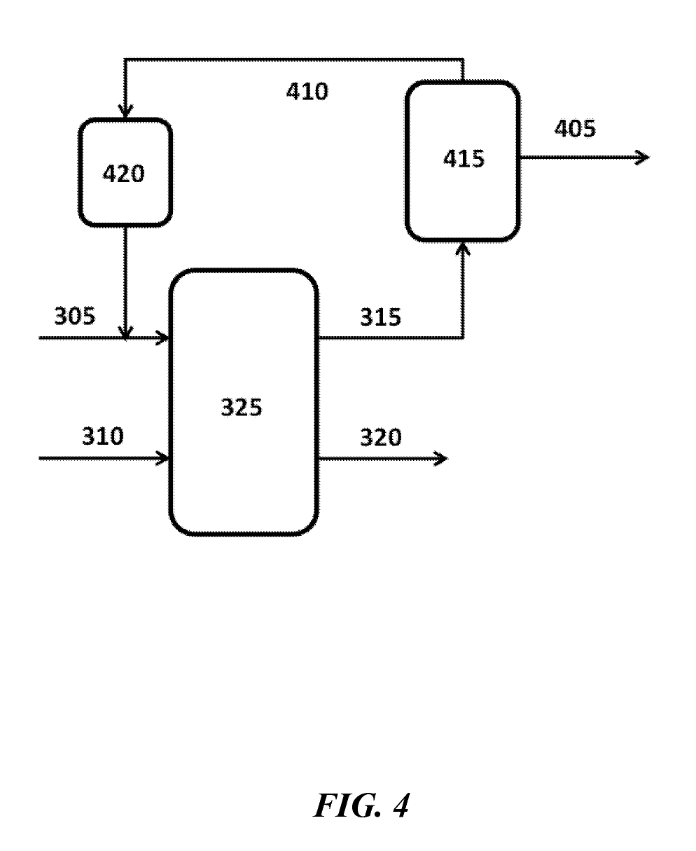

In some instances, the method further comprises recovering the extracted one or more biomass components from the pressurized gas, supercritical or near-supercritical fluid. In some cases, the lignin is recovered from the ionic liquid mixture 320. With reference to FIG. 4 where like numerals indicate like elements, the one or more biomass components 405 are recovered from the pressurized gas, supercritical or near-supercritical fluid (post-extraction pressurized gas, supercritical or near-supercritical fluid mixture) 315. In some embodiments, following recovery of the one or more biomass components, the fluid is recycled and/or re-used 410. In some cases, the lignin is recovered from the ionic liquid mixture 320.

The one or more biomass components can be recovered in any suitable way and/or in any suitable vessel 415. In some embodiments, the one or more biomass components are recovered from the pressurized gas, supercritical or near-supercritical fluid by lowering the pressure of the fluid.

The pressure can be lowered to any level (e.g., to a level such that the biomass components are recovered from the fluid). Following recovery of the biomass components, the fluid can be re-pressurized and used again 410. The fluid can be re-pressurized in any suitable apparatus 420 including a compressor, a pump, or any combination thereof. In some cases, pressurization using a pump consumes less energy than pressurization using a compressor. The pressure of fluids above their critical point can be increased with a pump. In some cases, the pressure of the fluid is not lowered below the critical pressure of the supercritical or near-supercritical fluid. In some embodiments, the pressure is not lowered more than 5%, more than 10%, or more than 20% below the critical pressure of the supercritical or near-supercritical fluid.

The pressure of the fluid can be lowered at any rate. In some embodiments, the pressure of the fluid is lowered in stages where various biomass components are recovered from the fluid at various pressure stages. For example, larger molecules can be fractionated from smaller molecules by lowering the pressure in stages. In some cases, various biomass components can be fractionated from each other. Groups of molecules can be fractionated from each other such as 5 carbon sugars from 6 carbon sugars or oils from sugars. In some cases, molecular species are fractionated from each other such as glucose from xylose. Biomass components can be fractionated based on the conditions at which they are recovered from the fluid. In some cases, biomass components are fractionated based on miscibility (e.g., oil from an aqueous solution comprising sugars).

In some cases, the one or more biomass components are recovered from the fluid using supercritical chromatography. In some embodiments, the vessel 415 is a supercritical chromatograph. Decreasing the pressure in the supercritical chromatograph 415 may cause various dissolved biomass components to become insoluble in the fluid at various pressures, resulting in separation of the biomass components. Separation of the biomass components can also be achieved by decreasing the temperature below the critical temperature. Separation of the biomass components can also be achieved by differential strength of interaction with a chromatography resin packed into the supercritical chromatography unit 415. In various embodiments, separation can be achieved through any combination of changes in pressure of the fluid, changes in temperature of the fluid, and interactions between the biomass components and a chromatography resin. One or more fractions comprising various biomass components may be recovered from the supercritical chromatograph. In some embodiments, the fractions are recovered in water. In some embodiments, the fractions are sufficiently pure and/or concentrated to be used directly, such as in a fermentation process.

In some cases, the one or more biomass components are recovered from the fluid by changing the temperature of the fluid (either raising or lowering the temperature). The temperature can be changed at any suitable rate (e.g., in stages, to fractionate biomass components) or to any suitable extent (e.g., so that biomass components are recovered). In some cases, the fluid is re-heated or re-cooled and used again 410. Recovery of biomass components by changing the temperature may be preferable to recovery of biomass components by pressure changes because thermal energy is more easily recovered (e.g., using a heat exchanger) than mechanical energy in some instances.

The supercritical or near-supercritical fluid can have any suitable polarity. In some instances, the fluid is non-polar (e.g., carbon dioxide). In some embodiments, the fluid is polar (e.g., ammonia). Various fluids can be mixed (in any ratio), for example to achieve a certain polarity.

In some embodiments, the supercritical or near-supercritical fluid comprises a co-solvent. The co-solvent can be used at any suitable concentration (e.g., about 0.1%, about 0.5%, about 1%, about 5%, or about 10% of the mass of the supercritical or near-supercritical fluid).

The co-solvent can be polar or non-polar. In some embodiments, the co-solvent is polar when the supercritical or near-supercritical fluid is non-polar.

The co-solvent can be derived from the biomass and/or present in the hydrolysate. In some embodiments, the co-solvent is selected from water, alcohol, acetic acid, acetate, acetone, carboxylic acids, organic polar acids or any combination thereof.

In some embodiments, one or more biomass components are sequentially extracted from the ionic liquid in a plurality of supercritical or near-supercritical fluids (optionally comprising co-solvents). In some cases, polar biomass components are extracted in a polar supercritical or near-supercritical fluid and non-polar biomass components are extracted in a non-polar supercritical or near-supercritical fluid.

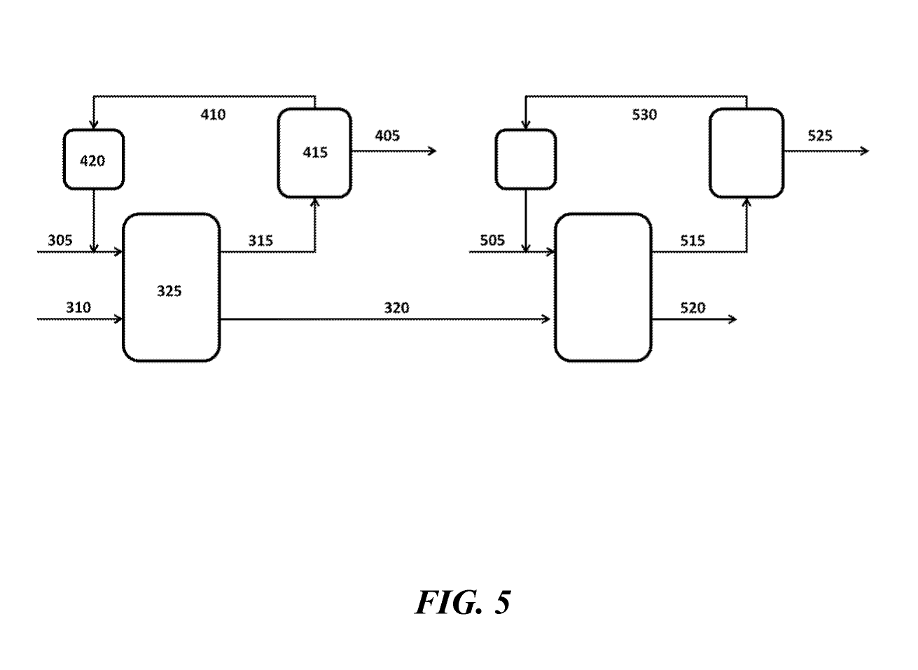

With reference to FIG. 5, where like numbers represent like elements, the ionic liquid mixture 310 is contacted with a first fluid 310 to form a first post-extraction fluid mixture 315 and a first post-extraction ionic liquid mixture 320. The first post-extraction ionic liquid mixture is contacted with a second fluid 505 to form a second post-extraction fluid mixture 515 and a second post-extraction ionic liquid mixture 520. Biomass components 525 can be recovered from the second post-extraction fluid mixture and the second fluid can be re-used 530. In some cases, the lignin is recovered from the first post-extraction ionic liquid mixture 320 and/or the second post-extraction ionic liquid mixture 520.

The method can achieve a high recovery of ionic liquid. In some embodiments, ionic liquid is rejected from the fluid by increasing the pressure of the fluid. In some embodiments, ionic liquid is rejected from the fluid by increasing the pressure of the fluid following extraction and before recovery of the biomass components from the fluid (e.g., increasing the pressure such that the recovered biomass components have less than 0.5%, less than 0.1%, less than 0.05%, less than 0.01%, or less than 0.005% ionic liquid by mass).

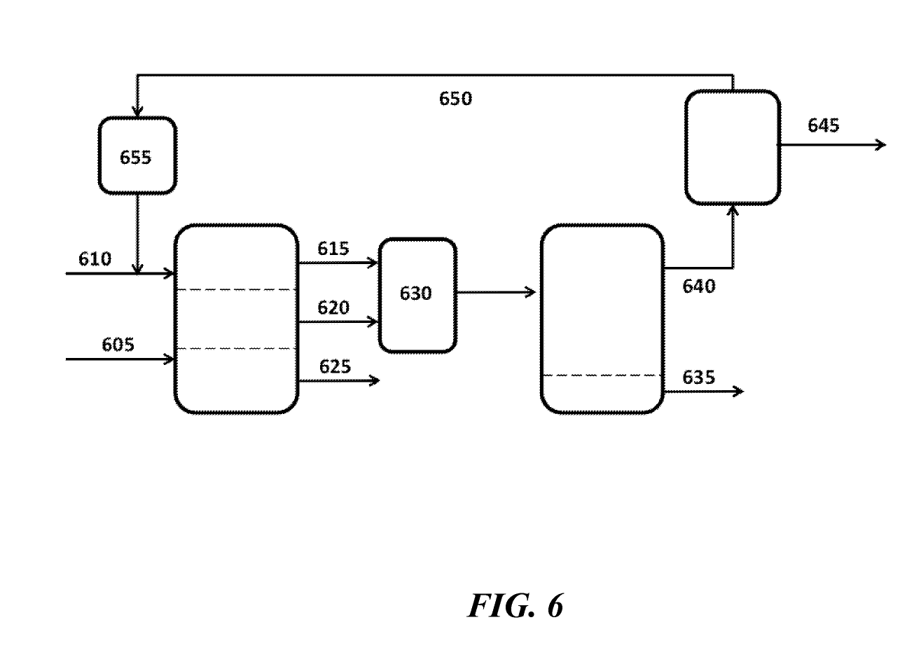

Turning attention to FIG. 6, an ionic liquid mixture comprising biomass components 605 is contacted with a fluid 610 to form a post-extraction fluid mixture 615, a post-extraction ionic liquid mixture 625, and optionally an aqueous phase 620. While three phases are shown (represented by dashed phase partitions), in some cases, an aqueous phase 620 is not formed. The ionic liquid mixture and fluid are contacted at a first pressure and temperature. In some cases, the first pressure and temperature does not reject a sufficiently high proportion of the ionic liquid from the post-extraction fluid mixture (and/or aqueous phase).

In some cases, the post-extraction fluid mixture 615 (and optionally an aqueous phase 620) is further compressed 630 to a second pressure. The second pressure further rejects ionic liquid 635 from the fluid. Biomass components can be recovered from the fluid 645 and the fluid can be recompressed 655 and re-used 650. In some cases, the lignin is recovered from the first post-extraction ionic liquid mixture 625 and/or the second post-extraction ionic liquid mixture 635.

The composition (comprising one or more biomass components in an ionic liquid) can be obtained in any suitable way, including by dissolving a biomass in an ionic liquid and hydrolyzing the biomass in the ionic liquid as described above. In some instances, the ionic liquid comprises a catalyst (e.g., an acid). In some instances, the ionic liquid comprises acid (e.g., hydrochloric acid). In some cases, the fluid is carbon dioxide and the ionic liquid comprises carbonic acid.

In some instances, the ionic liquid is re-used to dissolve and/or hydrolyze biomass following extraction of biomass components from the ionic liquid (e.g., in a closed-loop process). In some cases, biomass dissolution and/or hydrolysis is more efficient when the concentration of water in the ionic liquid is (e.g., initially) low (e.g., less than 10%, less than 5%, less than 3%, or less than 1%). In some embodiments, water is extracted from the composition in the fluid (e.g., to less than 10%, less than 5%, less than 3%, or less than 1%). In some embodiments, following extraction, the concentration of water in the ionic liquid is between 0% and about 10%, between 0% and about 5%, between 0% and about 3%, or between 0% and about 1%.

In some embodiments, the biomass components comprise carbohydrates, the molecular weight of the carbohydrates is reduced in the ionic liquid to form sugars, and the sugars are extracted from the ionic liquid.

### Recovery of Biomass Components in an Aqueous Phase

In another aspect, biomass components are recovered from ionic liquids in an aqueous phase. An aqueous phase is any composition in which water is the major solvent. An aqueous phase may have less than 50% water (e.g., a solution of 51% glucose in 49% water). In contrast, an ionic liquid-rich phase is any composition in which ionic liquid is the major solvent. In some embodiments, the biomass components are not extracted in a supercritical or near-supercritical phase. Lignin can be recovered from an aqueous phase and/or an ionic liquid phase.

Described herein is a multi-phasic system and a method for formation thereof. The multi-phasic system comprises a first phase comprising an ionic liquid; a second phase comprising water and one or more biomass components; and optionally a third phase comprising a fluid.

In some embodiments, the first phase comprises an ionic liquid, as described herein. The first phase (e.g., ionic liquid-rich phase) may contain the majority of the ionic liquid originally in the composition (e.g., at least 80%, at least 90%, at least 95%, or at least 99%). The first phase can comprise at least 50%, at least 60%, at least 70%, at least 80%, at least 90%, at least 95%, or at least 99% ionic liquid by mass in some instances.

In some embodiments, the second phase comprises water, as described herein. The second phase (e.g., aqueous or water-rich phase) may contain the majority of the water originally in the composition (e.g., at least 80%, at least 90%, at least 95%, or at least 99%). In some embodiments, the second phase comprises less than 25%, less than 20%, less than 15%, less than 10%, less than 5%, less than 1%, less than 0.5%, or less than 0.1% ionic liquid by mass (w/w). In some cases, the second phase comprises a detectable amount of ionic liquid (e.g., at least 0.00001% in some instances). The biomass components can be recovered in the second phase (e.g., aqueous phase).

In some embodiments, the third phase comprises a fluid, as described herein. Not all embodiments have a third phase. In some embodiments, there are more than three phases. The fluid can be without limitation a pressurized gas, a liquefied gas, a near-supercritical fluid, or a supercritical fluid. In some embodiments, the fluid is pressurized such that the first phase and second phase form.

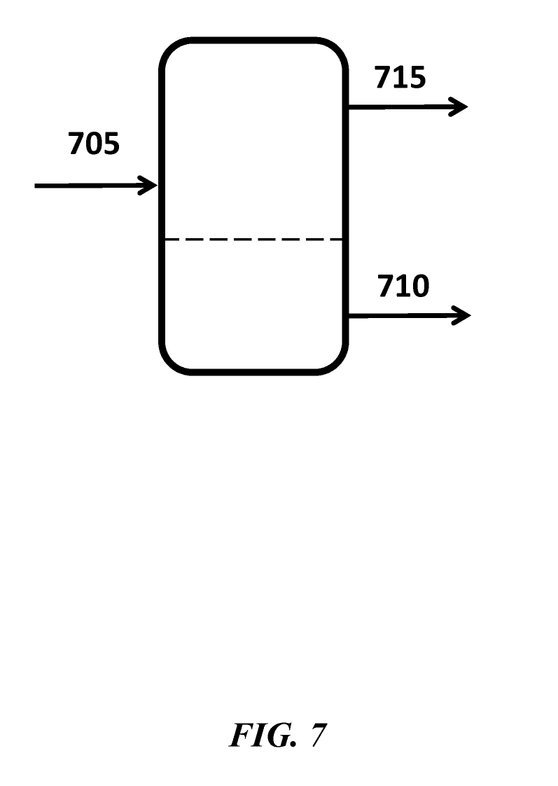

In some embodiments, provided herein with reference to FIG. 7 is a method for recovering biomass components from an ionic liquid. The method comprises forming a first phase and a second phase from a hydrolyzed biomass composition 705 comprising an ionic liquid, water and one or more biomass components, wherein the first phase 710 comprises an ionic liquid and the second phase 715 comprises water and one or more biomass components. In some instances, the second phase is portioned from the first phase to recover biomass components. The second phase is not necessarily less dense than the first phase. In some instances, the first phase floats on top of the second phase. In some instances, the second phase floats on top of the first phase. Lignin can be recovered from the first phase 710 and/or the second phase 715.

The hydrolyzed biomass composition can be obtained by hydrolyzing the biomass and/or biomass component in the ionic liquid. In some cases, the biomass component is a sugar (e.g., glucose). The sugar can be recovered in the second (e.g., aqueous) phase. As used herein, compounds (e.g., biomass components, sugars) are recovered when they are removed from the ionic liquid. In some embodiments, recovered biomass components can be used in further methods (e.g., recovered sugars can be fermented). In some cases, recovered, partitioned, separated, purified, isolated are used interchangeably. These terms are not absolute (e.g., the methods do not require complete separation, absolute purity, and the like).

The formation and/or stability of separate ionic liquid-rich and aqueous phases can be affected by the presence of a solute. The solute can be dissolved in the composition comprising ionic liquid and water, can be dissolved in the aqueous phase, can be dissolved in the ionic liquid-rich phase, or any combination thereof. The solute can be added to the composition and/or phases. In some instances, at least some of the solute is derived from the biomass. Examples of solutes (optionally derived from biomass) include, but are not limited to sugar, oil, methanol, or any combination thereof.

In some embodiments, the hydrolysis of biomass provides solute(s) that induce the formation of the first phase and the second phase. Induction of phase formation means that two or more separate phases (e.g., aqueous phase and ionic liquid-rich phase) do not form at a given set of conditions (e.g., temperature and pressure) without the presence of the solute. Induction of phase formation can also mean that two or more separate phases (e.g., aqueous phase and ionic liquid-rich phase) form under a given set of conditions (e.g., temperature and pressure) with the presence of the solute. In some instances, phases and/or separate phases are compositions that are immiscible or partially miscible with each other. In some cases, phases and/or separate phases have different densities from each other. In some cases, phases and/or separate phases have different major components (e.g., solvents such as water or ionic liquid) from each other.

In some embodiments, the hydrolysis of biomass provides solute(s) that at least partially stabilize the second (aqueous) phase. Stabilization of phases means that two or more separate phases (e.g., aqueous phase and ionic liquid-rich phase) remain distinct for a longer period of time at a given set of conditions (e.g., temperature and pressure) with the solute present when compared to without the presence of the solute.

In some embodiments, the solutes are hydrogen bonding solutes. A hydrogen bonding solute is any molecule capable of forming one or more hydrogen bonds. In some cases, the hydrogen bonding solute is capable of forming one or more hydrogen bonds with an ionic liquid and/or water. The hydrogen bonding solute can have at least one hydroxyl group. In various embodiments, the hydrogen bonding solute can be a carbohydrate, a sugar, an aldose, a ketose, or any combination thereof. In some cases, the hydrogen bonding solute is derived from biomass. Glucose is an example of a hydrogen bonding solute.

The concentration of the solute (e.g., hydrogen bonding solute, optionally derived from biomass and/or a biomass component) in the composition comprising ionic liquid and water can be any suitable concentration (e.g., for the formation and/or stability of phases). In some embodiments, the concentration of the solute in the composition is about 0.01%, about 0.05%, about 0.1%, about 0.5%, about 1%, about 2%, about 4%, about 6%, about 8%, about 10%, about 15%, about 20%, or about 25%. In some embodiments, the concentration of the solute in the composition is at least 0.01%, at least 0.05%, at least 0.1%, at least 0.5%, at least 1%, at least 2%, at least 4%, at least 6%, at least 8%, at least 10%, at least 15%, at least 20%, or at least 25%. In some embodiments, the concentration of the solute in the composition is between 1% and 25%. In some cases, the concentration of the solute in the composition is at least high enough to induce the formation of an aqueous phase. In some cases, the concentration of the solute in the composition is at least high enough to stabilize an aqueous phase.

Following the formation of an aqueous phase, the concentration of the solute in the aqueous phase can be any suitable concentration (e.g., for the stability of phases). In some instances, the concentration of the solute is higher in the aqueous phase than the concentration of the solute in the ionic liquid phase and/or in the composition before the formation of phases. In some embodiments, the concentration of the solute is at least 10%, at least 20%, at least 50%, at least 100%, or at least 200% higher in the aqueous phase than the concentration of the solute in the ionic liquid phase and/or in the composition before the formation of phases.

In some embodiments, the concentration of the solute in the aqueous phase is about 0.1%, about 0.5%, about 1%, about 2%, about 4%, about 6%, about 8%, about 10%, about 15%, about 20%, about 25%, about 30%, about 40%, about 50%, about 60%, or about 70%. In some embodiments, the concentration of the solute in the aqueous phase is at least 0.1%, at least 0.5%, at least 1%, at least 2%, at least 4%, at least 6%, at least 8%, at least 10%, at least 15%, at least 20%, at least 25%, at least 30%, at least 40%, at least 50%, at least 60%, or at least 70%. In some cases, the concentration of the solute in the aqueous phase is at least high enough to induce the formation of an aqueous phase. In some cases, the concentration of the solute in the aqueous phase is at least high enough to stabilize an aqueous phase.

In some embodiments, the hydrolyzed biomass composition and/or solute can be obtained by hydrolyzing the biomass in the ionic liquid. As described above, hydrolysis can involve the addition of water to a hydrolysis reaction. The amount and/or rate of water addition can be used to control the concentration of the solute in the composition and/or aqueous phase. In some embodiments, the concentration of the water in the hydrolysis reaction is such that the concentration of the solute in the second phase is near saturation (e.g., at least 50%, at least 70%, at least 90%, at least 95%, or at least 99% of saturation). In some cases, the solute is a sugar or mixture of sugars. In some instances, the solubility of a sugar or mixture of sugars in the aqueous phase is between about 3% and 78% by mass at 25° C. In some instances, the solubility of a sugar or mixture of sugars in the aqueous phase is between about 55% and 67% by mass.

In some embodiments, water is added to the hydrolysis reaction at a rate such that the concentration of ionic liquid in the aqueous phase (e.g., second phase) is low (e.g., less than 25% by mass, less than 15%, less than 10%, less than 5%, less than 1%, less than 0.5%, or less than 0.1% ionic liquid by mass).

The temperature of the first phase, second phase and/or composition can be any of a variety of suitable temperatures (e.g., for the formation or stability of an aqueous phase). In some instances, the temperature of the composition is reduced to form the first phase and the second phase (e.g., reduced from the temperature at which hydrolysis is performed). In some embodiments, the temperature is about 50° C., about 45° C., about 40° C., about 35° C., about 30° C., about 25° C., about 20° C., about 15° C., about 10° C., or about 5° C. In some embodiments, the temperature is less than 50° C., less than 45° C., less than 40° C., less than 35° C., less than 30° C., less than 25° C., less than 20° C., less than 15° C., less than 10° C., less than 5° C., or less than 0° C. In some embodiments, the temperature is less than ambient temperature (e.g., room temperature, the temperature of the outdoor weather and/or building in which the process is housed).

In some embodiments, the composition and/or first phase and second phase are pressurized. The pressure can be any of a variety of suitable pressures (e.g., a pressure that provides for the formation or stability of an aqueous phase). In some instances, the pressure of the composition and/or first phase and second phase is greater than atmospheric pressure. In some embodiments, the pressure is about 1 bar, about 2 bar, about 5 bar, about 10 bar, about 20 bar, about 30 bar, about 40 bar, about 50 bar, about 100 bar, or about 200 bar. In some embodiments, the pressure is at least 1 bar, at least 2 bar, at least 5 bar, at least 10 bar, at least 20 bar, at least 30 bar, at least 40 bar, at least 50 bar, at least 100 bar, or at least 200 bar.

In some cases, the fluid is non-polar. In some embodiments, the fluid comprises carbon dioxide. In some embodiments, the composition and/or first phase and second phase are in contact with a pressurized gas. In some cases, the composition is contacted with pressurized carbon dioxide to form the first phase and the second phase.

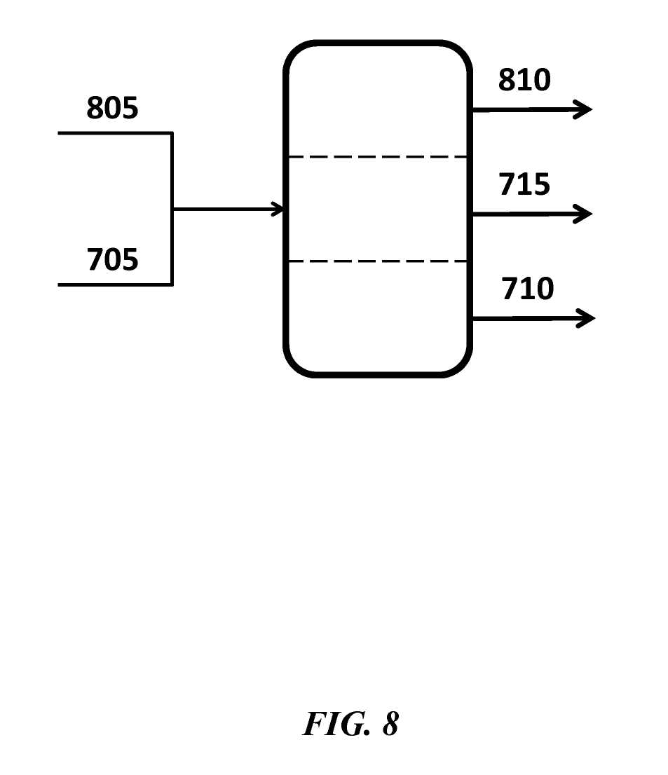

In some embodiments, provided herein is a method for recovering biomass components from an ionic liquid. With reference to FIG. 8, where like numerals indicate like elements, the method comprises contacting a composition 705 comprising an ionic liquid, water and a hydrogen bonding solute with a fluid 805 to form a first phase 710 comprising an ionic liquid and a second phase 715 comprising water and the hydrogen bonding solute. Contacting the composition with the fluid may form a third phase 810 comprising the fluid. The relative positions of the phases in FIG. 8 do not necessarily imply their relative densities. Lignin can be recovered from the first phase 710, the second phase 715 and/or the third phase 810.

In some embodiments, the method further comprises partitioning the second phase from the first phase. The phases can be partitioned in any suitable way. In some cases, the phases are piped (e.g., by a pump) from different regions of a vessel. In some cases, the phases have different densities and a less dense phase is drawn from an upper portion of a vessel and/or a more dense phase is drawn from a lower portion of a vessel. In some instances, centrifugation, filtration, decantation, or any suitable method can be used to partition the phases. In some instances, phases are not in contact with each other when they are partitioned from each other.

In some embodiments, the fluid is a pressurized gas. The gas can be pressurized to any suitable pressure (e.g., for the formation of the phases). In some embodiments, the gas is pressurized to about 1 bar, about 2 bar, about 5 bar, about 10 bar, about 20 bar, about 30 bar, about 40 bar, about 50 bar, about 100 bar, or about 200 bar. In some embodiments, the gas is pressurized to at least 1 bar, at least 2 bar, at least 5 bar, at least 10 bar, at least 20 bar, at least 30 bar, at least 40 bar, at least 50 bar, at least 100 bar, or at least 200 bar. In some embodiments, the fluid is a liquefied gas. In some instances, the composition is contacted with the fluid at a pressure greater than atmospheric pressure.

In some embodiments, the fluid is a supercritical or near-supercritical fluid. The fluid can be pressurized to about 1%, about 5%, about 10%, about 25%, about 50%, about 75%, about 90%, about 95%, or about 99% of the critical pressure of the fluid. In some embodiments, the fluid is pressurized to at least 1%, at least 5%, at least 10%, at least 25%, at least 50%, at least 75%, at least 90%, at least 95%, or at least 99% of the critical pressure of the fluid.

In some cases, contacting the composition with the fluid increases the rate at which the aqueous phase is formed (e.g., increases the rate by at least 5 times, at least 10 times, at least 20 times, at least 30 times, at least 50 times, at least 500 times, or at least 5000 times). In some cases, the aqueous phase is formed in less than 1 minute, less than 5 minutes, less than 10 minutes, less than 30 minutes, or less than 2 hours.

Contacting the composition with the fluid may decrease the viscosity of the composition. In some instances, decreasing the viscosity of the composition increases the rate at which the aqueous phase forms. In some embodiments, the viscosity of the first phase is less than the viscosity of the composition without contact with the fluid. Viscosity generally refers to dynamic viscosity and can be measured in units of pascal-second. In some embodiments, the viscosity of the composition is decreased by at least 1%, at least 5%, at least 10%, at least 25%, at least 50% less, or at least 75%. In some embodiments, the viscosity of the first phase is at least 1%, at least 5%, at least 10%, at least 25%, at least 50% less, or at least 75% less than the viscosity of the composition without contact with the fluid.

The ionic liquid can be any ionic liquid. In some cases, the ionic liquid is a biomass dissolving ionic liquid. In some embodiments, the ionic liquid is hydrophilic. In some instances, the ionic liquid comprises a chloride anion. The ionic liquid is not 1-butyl-3-methylimidazolium tetrafluoroborate (i.e., [C4mim][BF4]) or 1-butyl-3-methylimidazolium trifluoromethanesulfonate (i.e., [C4mim][CF3 SO3]) in some embodiments. In some embodiments, the ionic liquid has a hydrogen bond basicity β greater than 0.57. In some embodiments, the dielectric constant of the first phase (e.g., ionic liquid-phase) is less than the dielectric constant of the ionic liquid.

### Hydrolysate Composition and Sugar Recovery in Water

Provided herein are methods for the recovery of sugars from ionic liquids with high efficiency and low cost. Sugar hydroxyl groups can form hydrogen bonds to ionic liquid. In some cases, an ionic liquid that is a good biomass solvent can also be a good sugar solvent. Even though sugar can interact strongly with ionic liquid, it generally interacts even more strongly with water. For instance, glucose is about 100-fold more soluble in water than in an ionic liquid that dissolves biomass at ambient temperatures. Since water is already present in the hydrolysate, sugar extraction can be achieved by extracting water from the hydrolysate (with sugars following the water). To accomplish this, the ionic liquid is turned hydrophobic (e.g., using CO₂).

In some cases, roughly ˜97% of the hydrolysate is comprises ionic liquid, sugar and water (see, Table 2 and Table 3).

TABLE 2
Major components of hydrolysate composition starting from loblolly pine.

| major components | % (w/w) | observation |
|---|---|---|
| water | 8 | co-solvent |
| ionic liquid | 82 | solvent |
| C6/C5 sugars | 7 | solute |
| lignin | 2.5 | particulates |

TABLE 3
Minor components of hydrolysate composition starting from loblolly pine.

| minor components | % (w/w) | observation |
|---|---|---|
| alcohols and acetate | 0.04 | solute |
| tall oils | 0.14 | emulsion |
| residual celluloses | <0.01 | particulates |
| proteins and humins | 0.32 | particulates |
| ash | 0.04 | particulates |

### IL-Water Phase Behavior Induced by CO₂

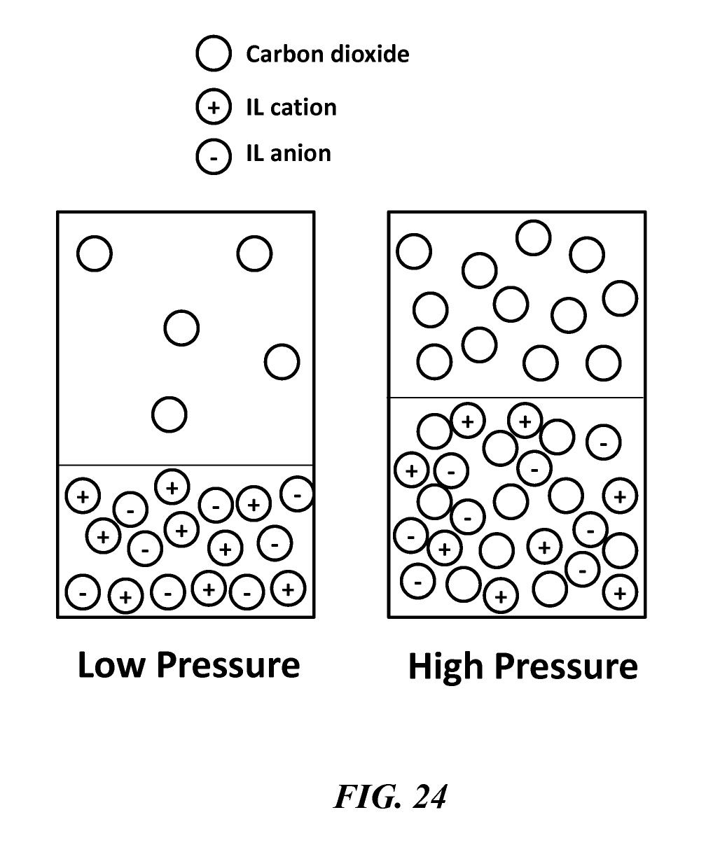

Several ionic liquids can form interesting systems when combined with CO₂. As seen in FIG. 24, when pressure is applied, CO₂ dissolves into the ionic liquid, whereas none or almost none of the ionic liquid dissolves into the pressurized CO₂, even at high pressure. The phase behavior is generally reversible and different from the behavior of mixtures of CO₂ with organic liquids or water. For increasing pressure, the molar volume (volume per mole of mixture) of most pure organics remain relatively unchanged, indicating that the total volume expands to accommodate the additional molecules. On the other hand, the molar volume of pure ionic liquids can be reduced by pressure, indicating little volume expansion as more CO₂ molecules are dissolved. The molar volume of water generally remains constant since very little CO₂ dissolves and form carbonic acid.

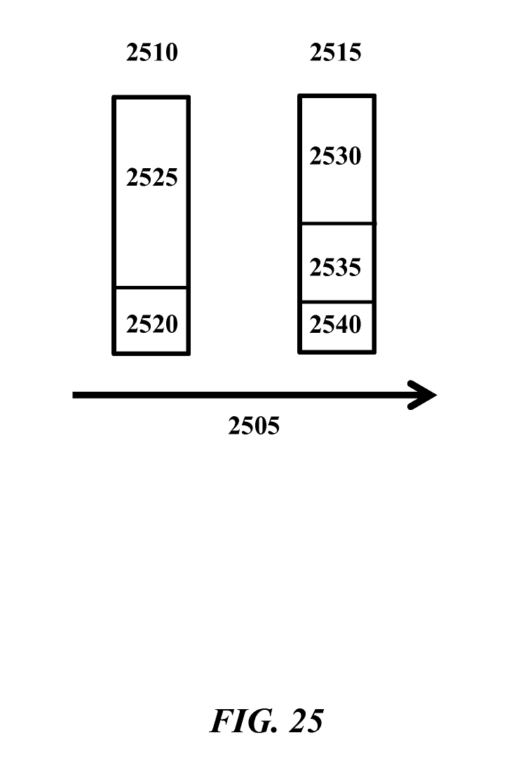

The differences in molar volume responses to pressure between ionic liquids, organics and water can underlie the ability of CO₂ to create and control an aqueous phase. As seen in FIG. 25, for a mixture of ionic liquid, water and CO₂, increasing the pressure above a lower critical end point pressure (LCEP) (determined by the composition of the system) can create separate ionic liquid-rich and water-rich phases. The dissolved CO₂ can act as an ionic liquid anti-solvent, excluding water from sub-critical pressures.

FIG. 25 shows CO₂-induced aqueous phase formation. The observed behavior is at ambient temperature as CO₂ pressure is increased 2505 from below 2510 to above a lower critical endpoint pressure (LCEP) 2515. Below the LCEP, a liquid phase (L) 2520 comprises water and ionic liquid and is in equilibrium with a vapor phase (V) 2525 comprising CO₂. Above the LCEP, a vapor phase (V) 2530 comprises CO₂ and water, a first liquid phase (L₁) 2535 comprises water, CO₂ and IL, and a second liquid phase (L2) 2540 comprises IL, CO₂ and water. In some cases L₂=L₁+V.

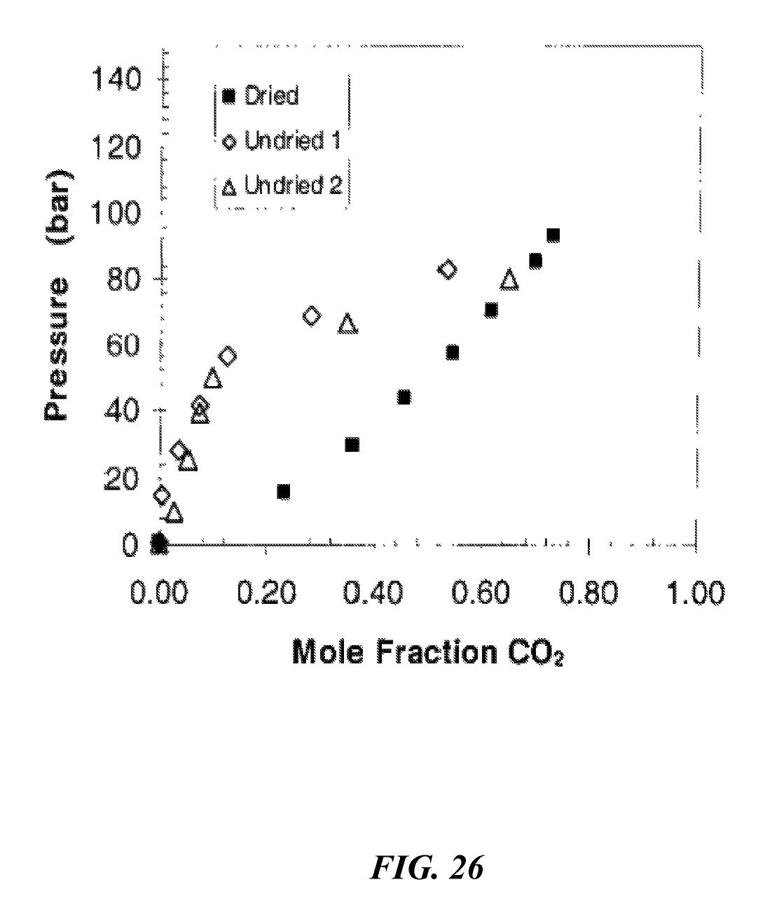

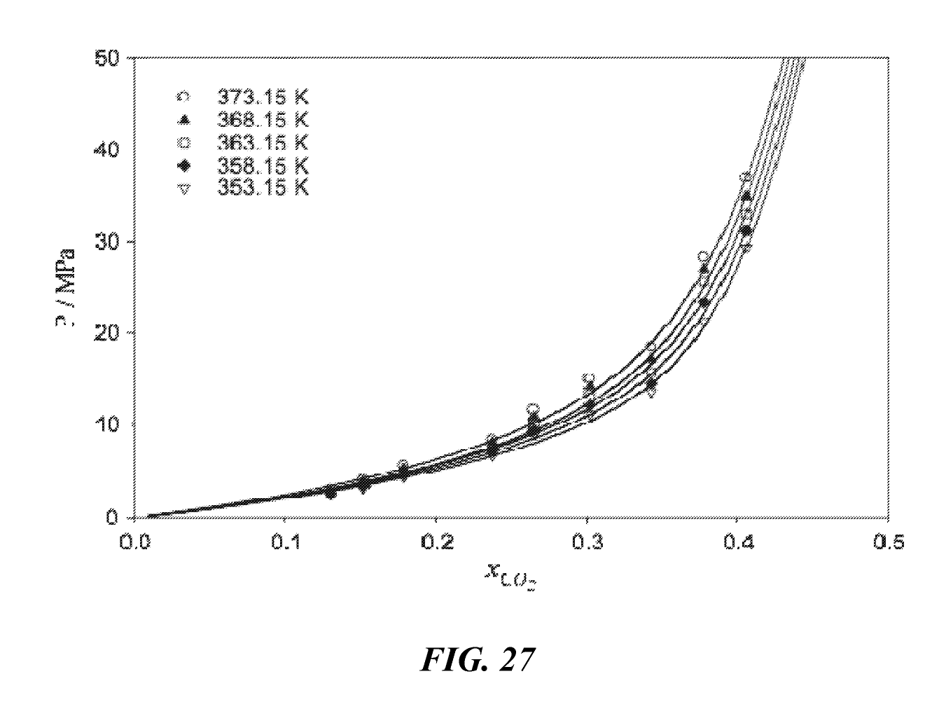

The ionic liquid [BMIM]Cl can be a suitable biomass solvent and hydrolysis medium. This ionic liquid is hydrophilic, which can make it more difficult to separate from water than hydrophobic ionic liquids. In one embodiment, starting with [BMIM]Cl at 25° C. and a starting ionic liquid concentration of 9.3 mol %, which corresponds to a solution of roughly 50% (w/w) ionic liquid, was successful in forming an aqueous phase in [BMIM]BF₄ (LCEP=5.1 MPa at 25° C.) and [BMIM]CF₃SO₃ (LCEP=4.9 MPa at 16° C.). In some cases, more CO₂ can become dissolved in both dried and undried ionic liquid as the pressure is raised and the temperature is lowered. As seen in FIG. 26, the pressure vs. CO₂ mol fraction curve (e.g., for undried [BMIM]PF₆) can be concave down, as opposed to concave up for dried ionic liquid. FIG. 27 shows the pressure vs. CO₂ mol fraction curve using dry [BMIM]Cl.

FIG. 26 and FIG. 27 show the solubility of pressurized CO₂ in ionic liquid. FIG. 26 shows the solubility of CO₂ in dried and wet [BMIM]PF₆ at 40° C. FIG. 27 shows experimental data and theoretical curves for solubility in [BMIM]Cl at several temperatures.

The ratio of the pressure vs. CO₂ solubility curves for both [BMIM]Cl and [BMIM]PF₆ at the same temperature (80° C.) is relatively constant at about 1.75. In some cases, undried data for [BMIM]PF₆ can be extrapolated to [BMIM]Cl. In some instances, an inflection point around 0.1 mol fraction and 60 bar (for [BMIM]PF₆) is associated with a disruption of water-ionic liquid interactions and microstructure. The water-ionic liquid disruption can occur in the hydrophilic [BMIM]Cl around 105 bar.

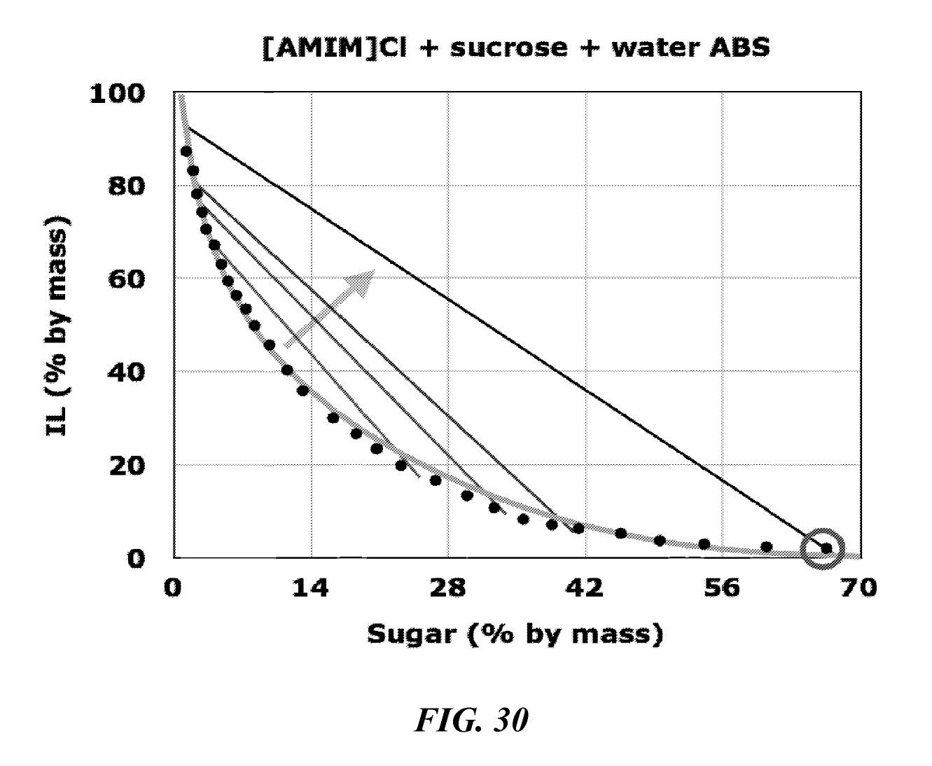

Dependence on the ionic liquid species is not limited to the anion. Either the anion or cation, or both can have an effect on dissolution and/or separation. In some cases, chloride is a strong hydrogen bond acceptor and used for biomass dissolution and hydrolysis. The choice of the cation may allow greater ease for separating water. For example, [AMIM]Cl, is also an suitable biomass solvent, and can separate spontaneously into ionic liquid and aqueous phases at atmospheric pressure upon the addition of sugars (see FIG. 30).

In some cases, the hydrolysates contain more components than water and sugars. Lignin and oils can be major components but in some cases are only slightly soluble in the ionic liquid (see Table 2). Alcohols, acetates, proteins, humins and ash can be minor components (see Table 3). In some instances, the solubilization of CO₂ in ionic liquid alters its solvation properties with regard to the other solutes (besides water). In some cases, salts (e.g., ammonium salts and zinc acetate) can be precipitated from several ionic liquid/organic mixtures. In some cases, CO₂ acts as a broad anti-solvent, essentially releasing solutes to nucleation and precipitation (or solubilization in other phases such as water). In some embodiments, a broad drop in solvating power coupled to a drop in viscosity allows for the recovery of various solutes from ionic liquids.

### IL-Water Phase Behavior Induced by Sugars

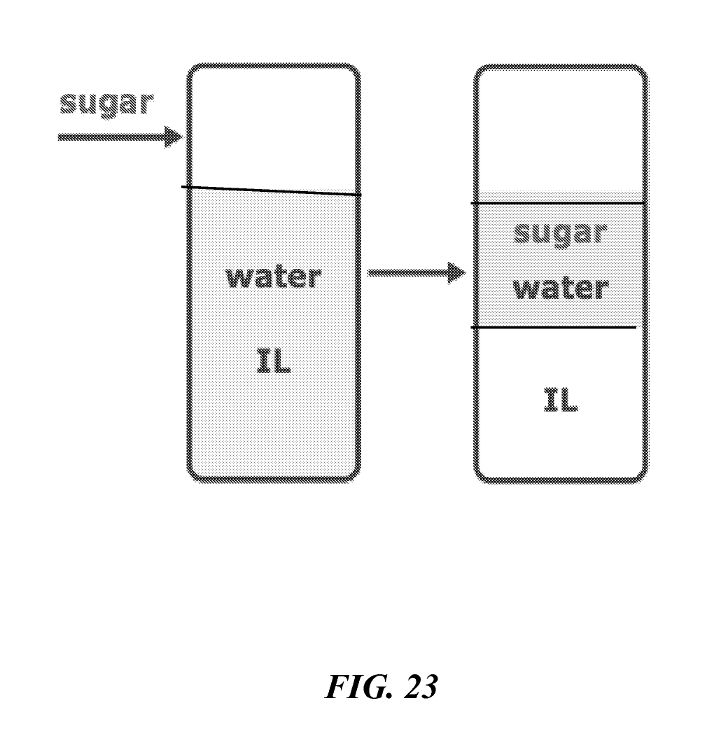

Water can be infinitely miscible in ionic liquids that are good biomass solvents (e.g., [BMIM]Cl and [AMIM]Cl). Also, sugars can dissolve in the same ionic liquids up to a few percent or higher depending on temperature. However, a concentrated solution of sugar in water may not dissolve significantly in the same ionic liquids. Similarly, starting from a solution of water in ionic liquid, the addition of sugar can eventually cause auto-separation into an ionic liquid phase and a “sugar phase” (see FIG. 23).

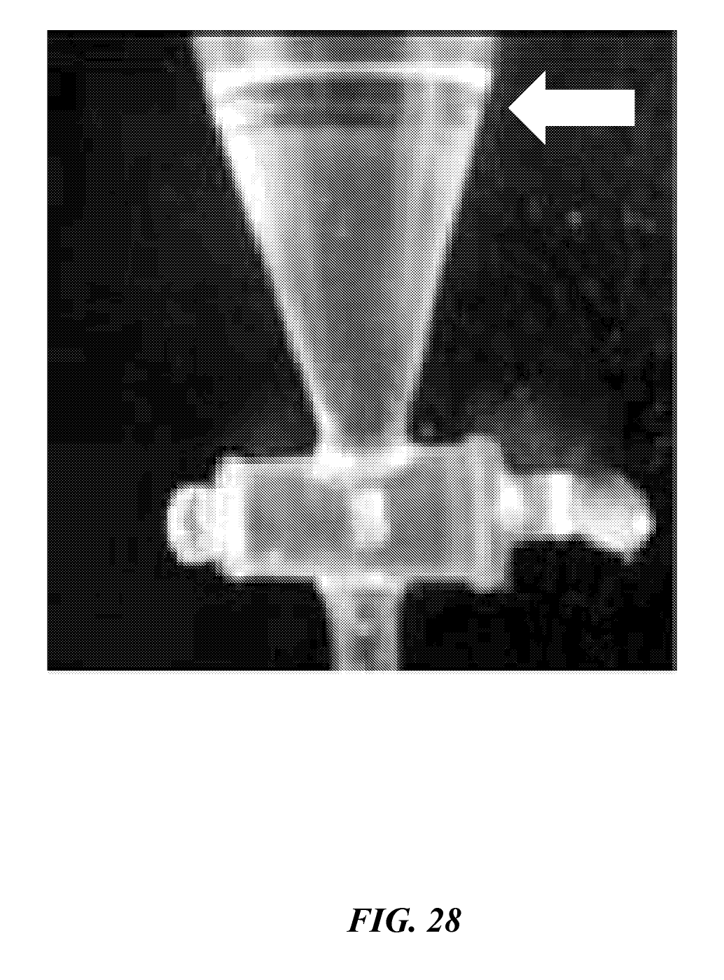

FIG. 28 is an example of aqueous biphasic system formed by an ionic liquid phase and a sugar phase. The ionic liquid, water and sucrose biphasic system is shown where sucrose solutions are generally denser than ionic liquids and separate towards the bottom.

Auto-separation of water from ionic liquid may be driven by the interaction between sugar and water, which can be much stronger than between sugar and ionic liquid. In some instances, the sugar sequesters the water molecules, creating molecular order and/or preventing those water molecules from solvating ions (i.e., kosmotropes).

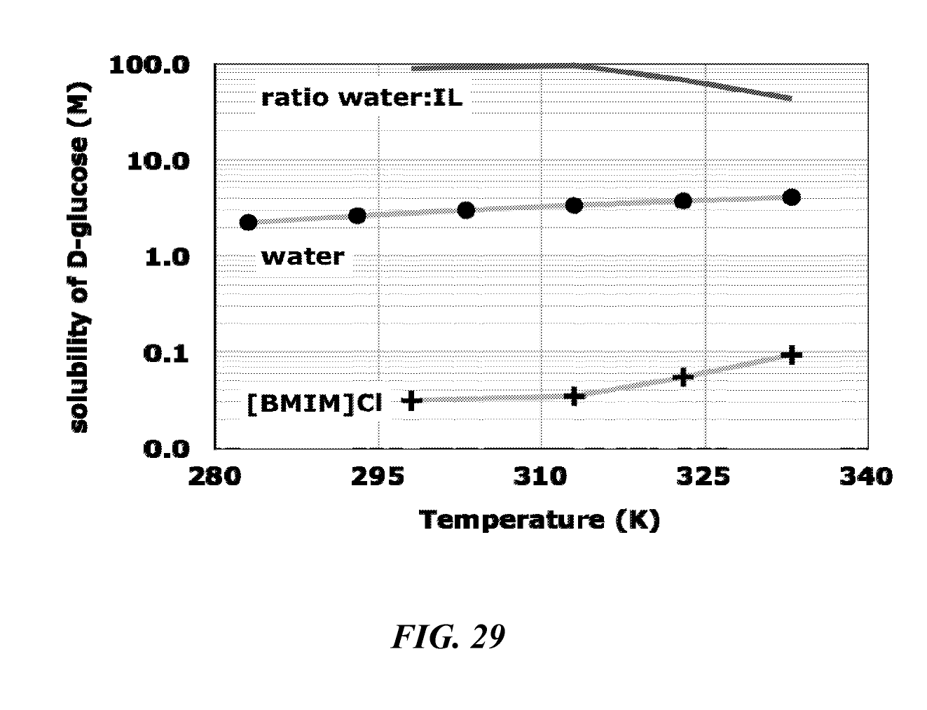

FIG. 29 shows solubility curves plotted in semi-logarithmic scale. At ambient temperatures, glucose is roughly 100-fold more soluble in water than in [BMIM]Cl. In some cases, the solubility ratio can be even higher than shown here for [BMIM]Cl. This relatively high ratio may be because of the larger number of hydrogen bonds between glucose and water than between glucose and ionic liquid or between ionic liquid and water. Between 40 and 60° C., glucose solubility in the ionic liquid increases faster than in water and the ratio of solubilities falls to about 40-fold. In some cases, ionic liquid-water biphasic systems are unstable above about 40° C. In some cases, the hydrolysate and/or other solutions are cooled in the methods described herein (e.g., to about 40° C., about 30° C., about 20° C., about 10° C., about 0° C., about −10° C., or less).

FIG. 30 shows an aqueous biphasic system (ABS) formed by [AMIM]Cl, sucrose and water. The presence of an ABS forming is evidenced by the visual contrast marking the interface. Samples from both phases are extracted with a syringe and analyzed by UV/Vis, showing a top ionic liquid-rich phase and a bottom sucrose-rich phase. Repeat measurements for varying relative amounts of ionic liquid, water and sugar are used to construct a phase diagram representing the ABS at the specified temperature.

For each starting composition, the mass fractions of ionic liquid and sugar in both phases (i.e., a total of four independent measurements (two data points)) are obtained after equilibrium is reached (black dots). This data was well-fitted with the Merchuk equation to give a binodal curve (curved line). In addition, tie-lines were measured, showing the correspondence between the compositions of the two phases at the point where the straight lines intersect the binodal curve. The gray arrow indicates the direction of increasingly better separations. The longest tie line corresponds to a separation where the sugar-rich stream reaches a sugar concentration of 68%, with about 98.4% sugar recovery and about 5 grams of ionic liquid per kg of sugar (circle).

The binodal curve for this phase behavior can be fitted with the Merchuk equation:

w₁=a exp(bw₂^{0.5}−cw₂³)

where w is the mass fraction; a, b and c are fitting constants, and the subscripts ‘1’ and ‘2’ denote the ionic liquid and sugar species, respectively. The error of the fit is on average 1%, which is generally within experimental noise. The tie lines are determined by fitting the following empirical equation:

$$\frac{1 - {w}_{1}^{i}}{{w}_{1}^{i}} = {{k}_{1} ( \frac{1 - {w}_{2}^{s}}{{w}_{2}^{s}} )}^{n}$$

where k₁ and n are fitting constants, and the superscripts ‘i’ and ‘s’ denote the ionic liquid-rich and sugar-rich phases, respectively. For example, w₂^{s} denotes the mass fraction of sugar in the sugar-rich phase. The error of the tie line fit is similar to the Merchuk fit. The two points formed by the intersection between the tie line and the binodal curve can indicate the composition of the phases.

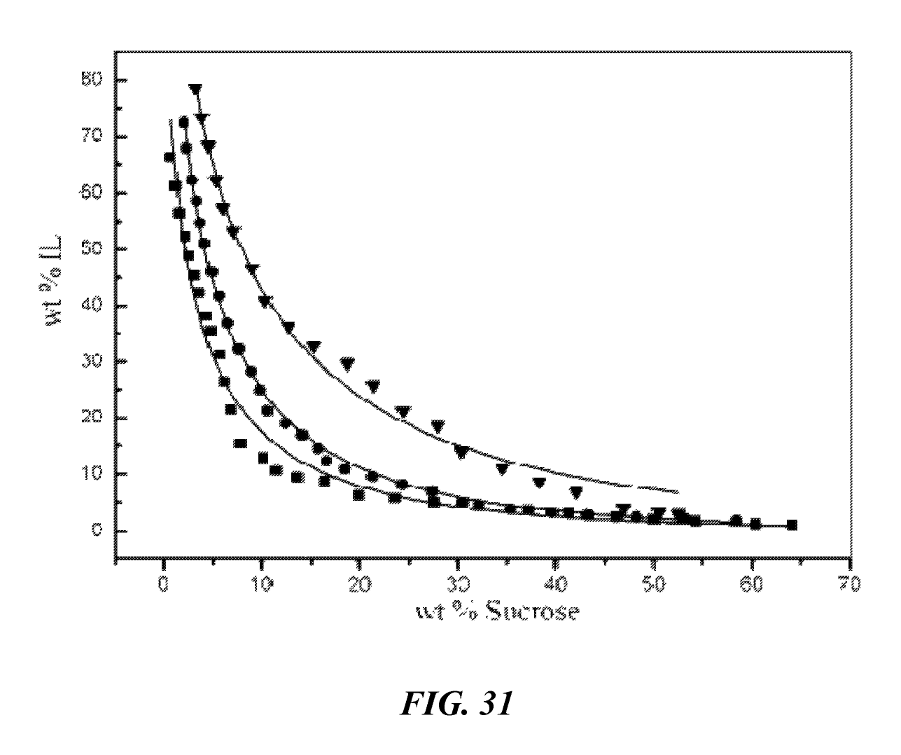

In some cases, ABS formation is influenced by temperature. Some ABS can be weakened at temperatures above about 35° C. (e.g., see FIG. 31). In some embodiments, cooling (e.g., towards the freezing point of water) creates a stronger phase split and better separation of components. In some cases, refrigeration is used to exploit this dependence on temperature.

FIG. 31 shows a phase diagram for a [Bmim]BF₄ with sucrose aqueous biphasic system. The various curves are for temperatures of: T=278 K (squares); T=298 K (circles); T=308 K (triangles). The lines shown are Merchuck model-fitted lines.

The methods described herein can be used to form an ABS with any biomass component (e.g., fermentable sugars). In some cases, the relative strengths of the ABS are different when formed with different sugars. In some instances, ABS strength is related to the curvature of the binodal curve.

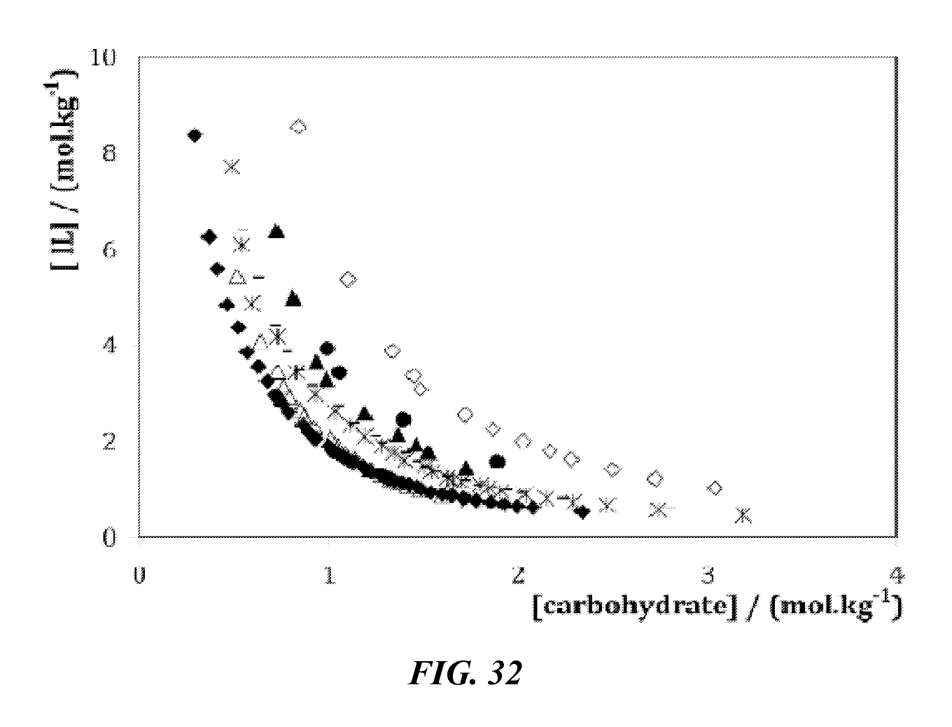

FIG. 32 shows phase diagrams for the ternary systems composed by [BMIM][CF₃SO₃]+carbohydrate+H₂O at 298 K. The various carbohydrates are depicted as follows: (black diamonds) D-(+)-glucose; (while triangles) D-(+)-galactose; (stars) D-(−)-fructose; (dashes) D-(+)-mannose; (black triangles) D-(−)-arabinose; (black circles) L-(+)-arabinose; (white diamonds) D-(+)-xylose. In some cases, the general strength of the ABS follow the rank: disaccharides>hexoses>pentoses.

### Combined CO₂-Induced and Sugar-Induced Phase Separation

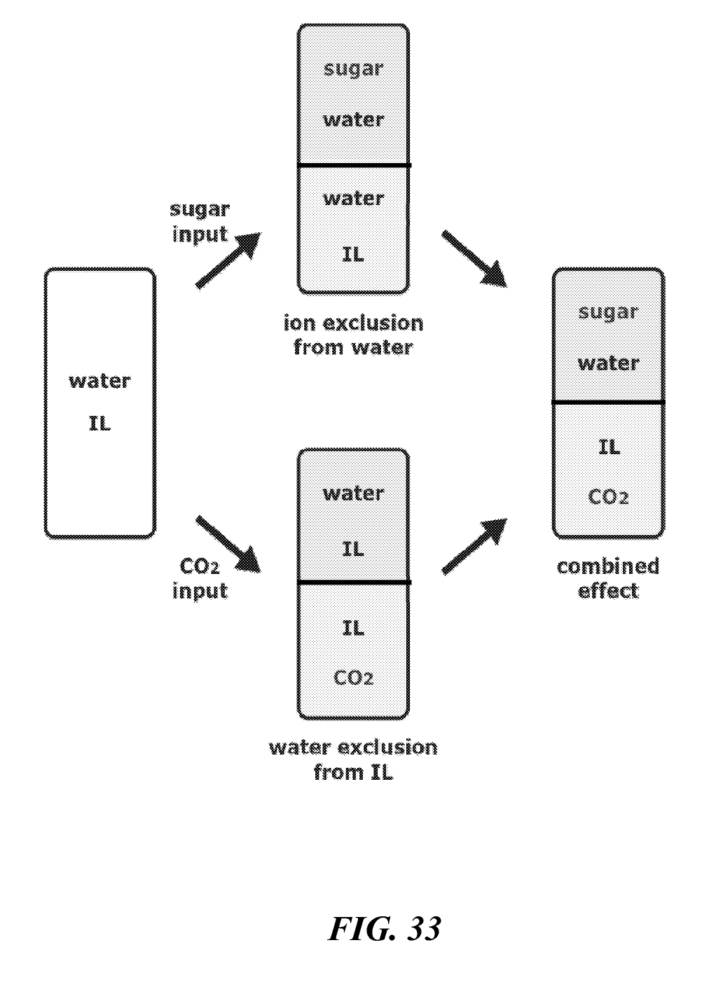

The methods described herein involving CO₂-induced phase separation and sugar-induced phase separation are complimentary and can be combined. As shown in FIG. 33, the combination of an ionic liquid/CO₂ phase excluding water, and a water/sugar phase excluding ionic liquid, can result in a fast and strong phase separation between water and biomass-dissolving ionic liquids (e.g., with a suitably high percentage of the ionic liquid in the ionic liquid phase and a suitably high percentage of the sugar in the water phase, such as in either case at least 90%, 95%, 99%, 99.5%, 99.9%, 99.99%, or 99.999%).

The sugars can induce the formation of two phases, potentially due to water solvation forces that exclude ions as it solvates sugars. In some cases, sugar recoveries are high (>98%). In some cases, any water remaining in the ionic liquid can solubilize sugars and reduce recovery. CO₂ inclusion into the ionic liquid can expel water to a separate phase. In some cases, water carbonation reduces the amount of ionic liquid that is dissolved in the aqueous phase. The combination of both effects, in essence, “cancel” each other's shortcomings, creating a stronger (cleaner) split. In some cases, the CO₂ pressure required for phase separation (LCEP) is reduced due to the presence of sugars. Sugars in a water/ionic liquid solution can be preferentially solvated by water, in which case water-ionic liquid interactions can be weaker, therefore requiring less effort for separation. In a synergistic manner, both CO₂-induced and sugar-induced phase separations can be more effective with decreasing temperature.

### Extractor and Operation Thereof

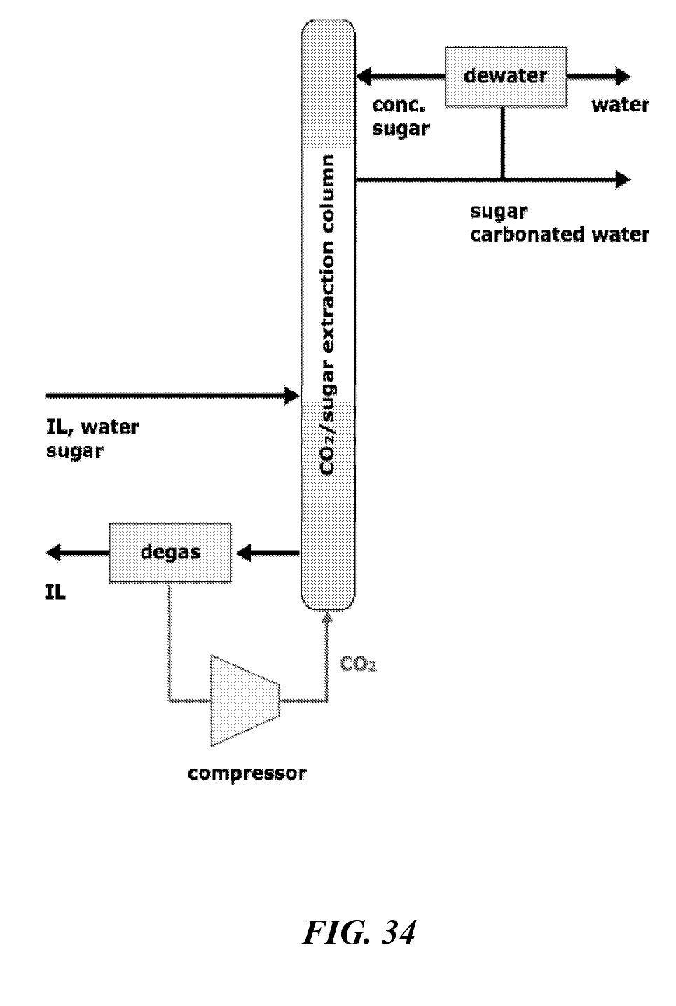

FIG. 34 shows an extractor having a “sugar driver” (on top) and a “CO₂ driver” (on bottom). 1. The extraction column can operate at sub-critical CO₂ pressures. The three phases depicted inside the column illustrate the CO₂-rich phase (top), water-rich phase (middle) and ionic liquid-rich phase (bottom).

The extraction column can be operated to effect separation when the composition of the hydrolysate entering the column is not optimal for formation of an ABS. In some cases, a composition that forms a strong ABS has an ionic liquid:water:sugar ratio of 1:1:1, whereas in some cases the ratio for the hydrolysate is about 8:1:1. In some embodiments, the ionic liquid concentration is controlled near the optimal by varying the top outlet flowrate relative to the bottom outlet. The sugar concentration in water can be pushed towards the saturation point to exclude ions. This can done by dewatering the sugar stream.

There are several possible ways to design and/or operate the extractor. One is to add a temperature gradient to the column, decreasing the temperature towards the top. For example, by having the top of the column refrigerated below ambient temperature while the bottom remains at ambient temperature. The objective in this instance is to get the sugar phase closer to the saturation point without hindering mass transport elsewhere.

In some cases, at least part of the aqueous stream drawn from the column is dewatered and a more concentrated sugar solution is returned to the column. Returning a more concentrated sugar stream (or solid sugar) can encourage the formation of an ABS.

In some instances, the aqueous stream drawn from the column is deionized. The ionic liquid ions are harvested and recycled from the sugar stream. Deionization can be used instead of, or in combination with concentrating sugars to push ionic liquid out. In some cases, the ionic liquid concentration is in the low parts-per-thousand range or less at the top (e.g., less than 0.001%, 0.01%, 0.1%, 0.3%, 0.5%, and the like). Deionization could be done in a number of ways, such as by using electrodialysis. Dewatering can also be done in a number of ways, such as by reverse osmosis.

At the bottom of the extractor, the ionic liquid can be degassed of CO₂. Degassing can be done in an agitated flash tank or an agitated heated tank. For the flash tank option, a compressor may be used. In some cases, a heated tank is used for degassing if the ionic liquid needs to be warmed and recycled back in the process to a hydrolysis and/or dissolution step. In this case, the CO₂ can be expanded and/or cooled before re-entering the column.

The extractor and methods of operation of the extractor can be used for hydrolysates and other solutions having components other than ionic liquid, water and sugar. Some hydrolysates have oils and solids. For the recovery of oils, an oil phase can form. The oil phase can be drawn off the column. In some cases, a method finds and tracks the oil layer, draws at the oil/water interface and decants. In some cases, some of the oil goes into the CO₂ phase (top), which can occur at sub-critical pressures. Solids (such as lignin) can be recovered by filtration at any point (e.g., before, after or during degassing).

### Pressure-Induced Phase Separation

In another aspect of the present disclosure, applying pressure to a mixture of water and ionic liquid can induce a phase separation into an aqueous phase and an ionic liquid phase. The pressure can be applied directly to the mixture (e.g., imposed by a surface) or indirectly to the mixture (e.g., by pressurizing a fluid such as a gas that is in contact with the mixture). In cases where the mixture is pressurized by contact with a gas, the gas can be any suitable gas (e.g., helium, neon, argon, krypton, xenon, hydrogen (H₂), nitrogen (N₂), oxygen (O₂), methane, ethane, and the like). The mixture can further comprise a hydrogen bonding solute such as a sugar. In some cases, pressurization is used in combination with decreasing the temperature of the mixture, introducing a hydrogen bonding solute such as sugar to the mixture, contacting with pressurized CO₂, or any combination thereof.

In some cases, the pressure is increased relative to ambient pressure. The pressure can be any suitable pressure. In some cases, the pressure is about 1 atmosphere (atm), about 2 atm, about 5 atm, about 10 atm, about 50 atm, about 100 atm, or about 500 atm. In some instances, the pressure is at least about 1 atm, at least about 2 atm, at least about 5 atm, at least about 10 atm, at least about 50 atm, at least about 100 atm, or at least about 500 atm. In some instances, the pressure is at most about 1 atm, at most about 2 atm, at most about 5 atm, at most about 10 atm, at most about 50 atm, at most about 100 atm, or at most about 500 atm.

In some cases, the pressure is decreased relative to ambient pressure. The pressure can be any suitable pressure. In some cases, the pressure is about 1 atmosphere (atm), about 0.5 atm, about 0.1 atm, about 0.05 atm, or about 0.01 atm. In some instances, the pressure is at most about 1 atm, at most about 0.5 atm, at most about 0.1 atm, at most about 0.05 atm, or at most about 0.01 atm. In some instances, the pressure is at least about 1 atm, at least about 0.5 atm, at least about 0.1 atm, at least about 0.05 atm, or at least about 0.01 atm.

### Kosmotrope-Induced Phase Separation

Provided herein are methods for recovering biomass components from an ionic liquid. The method can comprise forming a first phase and a second phase from a hydrolyzed biomass composition comprising an ionic liquid, water and one or more biomass components, where the first phase comprises an ionic liquid and the second phase comprises water and one or more biomass components.

While any suitable method can be used to form the first phase and the second phase, in some cases, the phases are formed by adding a kosmotrope (a solute that contributes to the stability and structure of water-water interactions, in some cases a salting-out agent) to the hydrolyzed biomass composition. Examples of kosmotropes and/or salting-out agents include hydrogen-bonding solutes such as glucose, as disclosed herein. Further examples include, but are not limited to kosmotropic salts (e.g., sulfate, phosphate, magnesium²⁺, lithium¹⁺, zinc²⁺ and aluminum³⁺), amino-acids (e.g., proline), polymers, bases (e.g., KOH, NaOH), acids (e.g., HCl, H₂SO₄), carbohydrates (e.g., glucose, trehalose), tert-butanol and polyols. In general, the more negative the free energy of hydration for a solute, the more kosmotropic the solute.

Upon addition of a kosmotrope, the hydrolysate can separate into two phases. The first phase can be composed primarily of water and the kosmotrope, and the second phase composed primarily of ionic liquid. The water-rich phase can accumulate the biomass components (e.g., sugars) produced by the hydrolysis reaction and therefore can be used to extract those sugars from the ionic liquid. Furthermore, the water-rich phase can reject the ionic liquid, which reduces or prevents loss of ionic liquid to the sugar stream.

Kosmotropes can be used in combination with each other and/or any other method for inducing an aqueous biphasic system (e.g., carbon dioxide, whether pressurized or not). In some cases, the kosmotrope is activated and/or created from a material that is not otherwise a kosmotrope. One example of the use of carbon dioxide at atmospheric pressure is the reaction between an amine and CO₂ in water to form a salt that acts like a kosmotropic salt in structuring water and promoting separation. Other gases or liquids may be used to form salting-out agents that effect separation.

In some cases, the kosmotrope is removed from the water-rich phase and/or solutes such as sugar. The kosmotrope can be removed following formation of the two phases in any suitable way.

In some cases, it becomes cost-effective to employ more expensive recovery methods (e.g., chromatography or electrodialysis) once the concentration of ionic liquid falls to a few percent (e.g., less than about 8%, less than about 5%, less than about 3% or less than about 1%). In some cases, the selectivity imparted by SMB is greatest when starting from an ionic liquid concentration of 10% or less.

One method is to use Simulated Moving Bed chromatography (SMB). SMB uses differences in solute retention on a solid support (column resin) in order to move solutes to different streams. Here, SMB can be used to recover kosmotropic salts, amino-acids, polymers or other salting-out agents from the water-rich phase according to the resin employed in the SMB. The kosmotrope could then be recycled back to the sugar extraction step.

Other methods for removing and/or recovering the kosmotrope include, but are not limited to Electrodialysis, Electrodialysis Reversal, Electrodeionization (EDI), Reverse Osmosis (RO), Nanofiltration (NF), Ultrafiltration (UF), or other liquid-liquid, liquid-solid or liquid-fluid extraction strategies. These methods can also accomplish the effect of extracting the salting-out agent from product or waste streams.

For electrodialysis, the recovery of [BMIM]Cl from water can recover about 3 kg of IL per kWh of electricity consumed and achieve satisfactory concentration differences at the raffinate and extract ends. Electrodialysis is vulnerable to fouling from some ash species such as silica, ionic calcium and suspended solids, so those should be removed upstream as much as possible to minimize downtime at the electrodialysis stage.

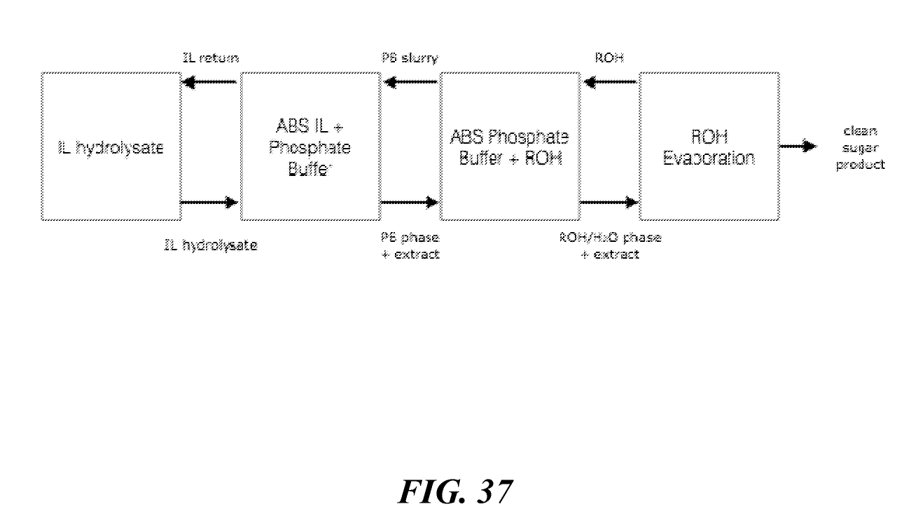

The kosmotropic salt can also be recovered using an anti-solvent or co-solvent such as an alcohol or ketone. In some cases, the kosmotropic salt is precipitated upon addition of the anti-solvent or co-solvent (i.e., is “salted out”). The strategy of employing a liquid-liquid (L-L) unit operation with IL and kosmotropic salt phases, followed by precipitation of salts is depicted in FIG. 37. Evaporation of the anti-solvent or co-solvent recovers sugar product and salts. In some cases, a ketone (e.g., acetone) is easier to recover from the aqueous solution than an alcohol (e.g., methanol) (e.g., because the ketone does not form an azeotrope with water, but the alcohol does form an azeotrope with water).

Salting out can change the solvent properties. Salt can dissolve to a large extend in pure water. But, if a significant amount of a co-solvent (e.g., say methanol or ethanol) is also dissolved in water, then the solubility of the salt drops. For example, addition of even 1 mL of ethanol to a saturated solution of potassium phosphate can cause some of the salt to precipitate.

In the methods of salting out described herein, a co-solvent can be added that precipitates the salt (e.g., an anti-solvent). The co-solvent can without limitation, (i) push the salt out of solution, (ii) forms no azeotrope, and (iii) have a low boiling point (for separation in a single flash). In some cases salting out leaves only a solution of sugar in a water/alcohol mixture or a water/ketone mixture. In some cases a water/ketone mixture is preferred over a water/alcohol because the water/ketone mixture does not form an azeotrope. An azeotrope is the point in the distillation curve where the vapor pressure of both components are the same, so one cannot enrich the mixture any longer. Furthermore, one can recover substantially all the acetone in a “single flash”, which means, a distillation column with no trays.

In some cases, the sugar is precipitated from solution (e.g., by addition of a co-solvent or anti-solvent as described herein or the application of a compressed gas such as CO₂ as described herein).

In an example, ionic liquid hydrolysate can be cooled and contacted with concentrated phosphate buffer (PB) in a continuous counter-current arrangement. This process can be done at ambient temperature and pressure. It can also be fast (on the order of 1 to 20 minutes). In some cases the selectivity (5) is about 120 and can be improved by optimizing pH and other conditions. By transporting sugars from IL to PB, a $10/kg chemical (i.e., IL) can be substituted for a $1/kg one (i.e., PB), which can be easier to remove due to its trivalent charge (at higher pH). Sugars contaminated with IL in the PB phase can flow into a vessel and be contacted with an alcohol such as methanol. As methanol mixes with water, the salts can precipitate. Then, evaporating the methanol produces an aqueous stream of sugars

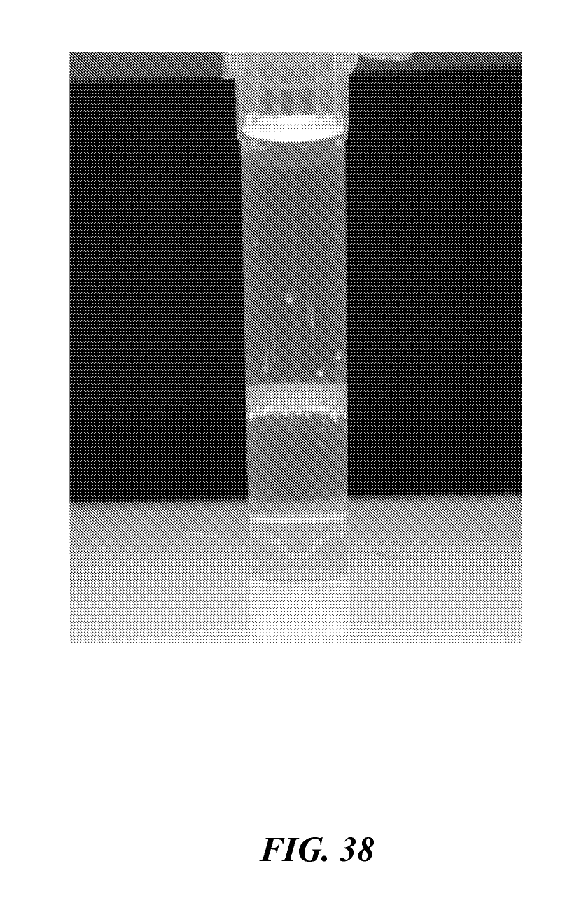

FIG. 38 shows precipitated salts. In this example, a methanol layer is pipetted over a potassium phosphate solution. The interface, where intermixing occurred, contains precipitated salt as a cloudy white layer.

The alcohol species and/or ratio of the PB phase and alcohol can be optimized to give the best selectivity. In some cases, a ketone is used instead of an alcohol species (e.g., acetone). Methanol has the lowest boiling point of any linear alcohol, as well as the highest solubility for glucose. However, it can also be poorly effective for precipitating salts, requiring a large amount of methanol. The tradeoff between selectivity for sugars and separation from water can determine the optimal point for this strategy. In some cases, an increase in selectivity can be achieved when the ratio of the PB phase to methanol is about 1:1.

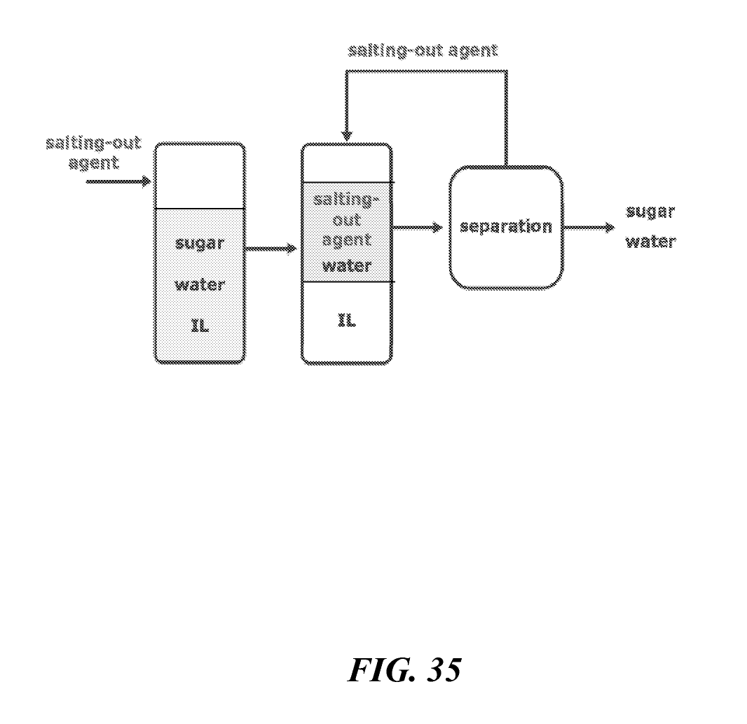

FIG. 35 shows an example of a process for separating solutes (e.g., hydrolysate sugar) from a mixture of ionic liquid and water. The salting-out agent (i.e., kosmotrope) can induce the formation of an aqueous phase and an ionic liquid phase. The aqueous phase can be drawn off. In some cases, the salting-out agent is recovered and optionally recycled to the phase formation stage of the process.

In some cases, the kosmotrope is not removed (e.g., remains in the aqueous sugar stream). In some cases, kosmotropic salts can be useful and/or tolerated by a fermentation process. Potentially useful and/or tolerable kosmotropic salts include potassium salts, phosphates and nitrates. For example, the product stream feeding a fermentation step could contain both macronutrients (e.g., sugars) and micronutrients (e.g., K₃PO₄).

ABS Formation with Polymers or Volatile Salts

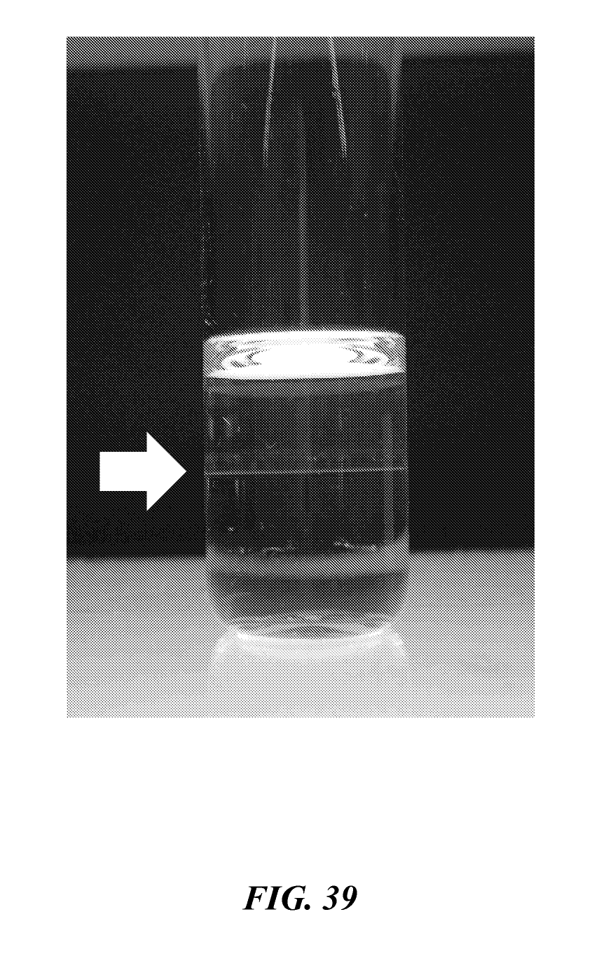

A polymer can be used to induce the formation of an aqueous biphasic system (ABS) for ionic liquid/water mixtures (e.g., biomass hydrolysate). In some cases, the polymer is used as a “back extraction” (i.e., as a way to recover the phosphate salts and deliver clean sugars) following ABS formation with kosmotropic salts. In some cases, the polymer is polyethylene glycol (PEG) having any degree of polymerization (e.g., about 2000 monomers). FIG. 39 shows a clear PEG layer on top of a clear PB layer. The liquid interface (arrow) can been seen even though both phases are clear. In some cases, a PEG phase (e.g., formed directly against an IL phase) can be used to extract hydrophobic solutes produced during hydrolysis, such as oily and extractive substances.

Volatile salts can also be used to form ABS. For example, CO₂ can be used in conjunction with NH₃. Both of these gases react with water according to the equations

NH₃+H₂O [symbol] NH₄⁺+HO⁻ (K_{b}=1.8×10⁻⁵)

CO₂+H₂O [symbol] H₂CO₃ (Kₕ=1.78×10⁻³)

thus forming aqueous ammonium carbonate, (NH₄)₂CO₃ (as well as some bicarbonate and carbamate). Both ammonium and carbonate ions can structure water fairly strongly as seen in Table 4, which shows free energies of hydration (Δ_{hyd}G expressed in units of kJ/mol). In order to obtain a more concentrated solution, cooling below room temperature can be performed. The solubility of this salt increases to about 35% at 0° C. Once sugars are extracted, the salt can be removed as neutral gases simply by heating to a mild temperature (e.g., 50-60° C.) or sparging with an inert gas.

TABLE 4
Free energies of hydration for selection ions.

| Cation | Δ_{hyd}G (kJ/mol) | Anion | Δ_{hyd}G (kJ/mol) |
|---|---|---|---|
| Cs⁺ | −259 | ClO₄⁻ | −213 |
| Rb⁺ | −280 | TcO₄⁻ | −251 |
| NH₄⁺ | −285 | NO₃⁻ | −306 |
| K⁺ | −305 | I⁻ | −309 |
| Na⁺ | −385 | Br⁻ | −324 |
| Li⁺ | −510 | Cl⁻ | −338 |
| H⁺ | −1056 | OH⁻ | −345 |
| UO²⁺ | −1329 | [CH₃CO₂]⁻ | −365 |
| Ca²⁺ | −1525 | HSO₄⁻ | −365 |
| Mn⁺ | −1740 | F⁻ | −460 |
| mg²⁺ | −1828 | CrO₄²⁻ | −958 |
| Fe²⁺ | −1866 | SO₄²⁻ | −1145 |
| Zn⁺ | −1880 | CO₃²⁻ | −1300 |
| CU²⁺ | −2017 | SO₃²⁻ | −1301 |
| Al³⁺ | −4537 | [C₆H₅O₇]³⁻ | −2765 |
| Th⁴⁺ | −5823 | PO₄³⁻ | −2835 |

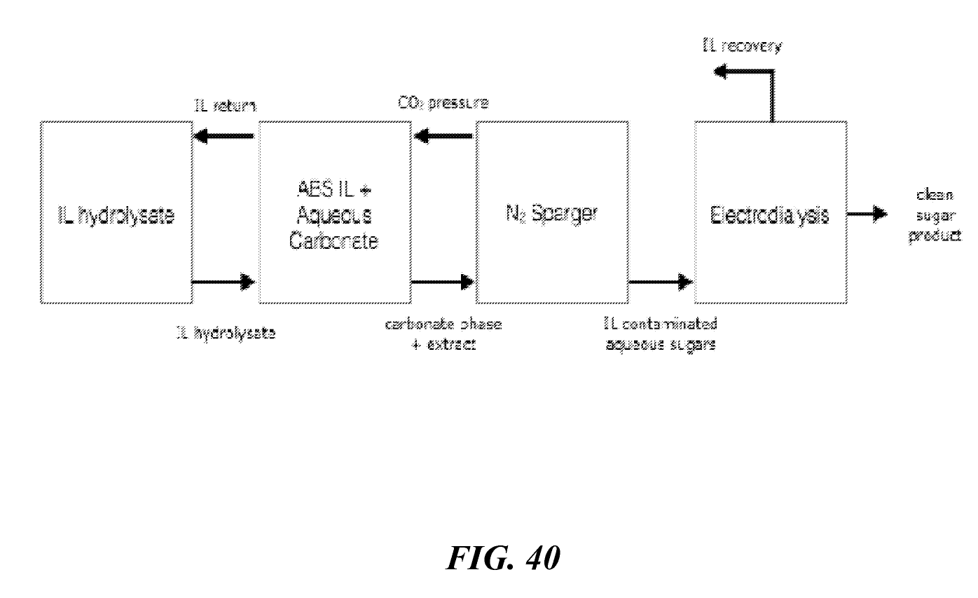

FIG. 40 shows a schematic drawing of separation employing ammonia (NH₃) and carbon dioxide (CO₂). In some cases, employing volatile salts can require lower pressures than ABS driven by only CO₂ (e.g., since both reactions neutralize each other, driving both equilibria to the right). In some instances, employing volatile salts can lower the salt recovery requirement downstream, as only a minor amount of IL would remain. Electrodialysis or ion-exchange can be used cost-effectively once the concentration of IL is dilute.

### Boronic Acids and Secondary Recovery

In some cases, a secondary recovery method is used to recover solutes and/or IL following formation of an ABS (regardless of the method for forming the ABS). Examples of secondary recovery methods include chromatography (e.g., simulated moving bed chromatography (SMB)), electrodialysis or extraction using boronic acids. In some instances, the use of these secondary recovery methods would be un-economical if used as a primary means for separation (i.e., without first forming an ABS). The secondary recovery methods can become economical when the concentration of the IL is reduced to about 10%, about 5%, about 3%, about 2%, about 1%, about 0.5%, about 0.1% or about 0.05% in the phase comprising the solutes.

A method for using boronic acids to separate sugars from ionic liquids is described in PCT Patent Publication No. WO2011/041455, which is incorporated herein by reference in its entirety. The method described therein extracts sugars directly from ionic liquid hydrolysate of biomass and can suffer from several deficiencies. In some cases, the method is most effective when the ionic liquid is dilute (e.g., 15% ionic liquid or less). Boronic acid extraction of sugars is effective from basic solutions, and often not from acidic solutions. Since ionic liquid hydrolysate is acidic, the method describe in the WO2011/041455 publication requires the addition of large amounts of a strong base, such as sodium hydroxide to bring the starting solution to a basic pH. This addition of base can be impractical and/or not cost effective. Another drawback of the WO2011/041455 procedure is that the resulting sugar solution is dilute (e.g., about 0.2M in some cases), which can result in additional expense or difficulty relating to concentration of the sugars. Yet another drawback of the WO2011/041455 procedure is that the recovered sugar can be contaminated with acid (e.g., HCl), which can result in additional expense or difficulty relating to neutralization and/or removal of the acid.

The methods of the present disclosure can avoid one or more of the aforementioned drawbacks of the procedure described in WO2011/041455. The methods described herein can include: (a) providing a biomass hydrolysate comprising ionic liquid, water and a sugar; (b) forming an aqueous biphasic system (ABS) that comprises a first phase comprising ionic liquid and a second phase comprising water and sugar; and (c) extracting sugar from the second phase using a boronic acid. The boronic acid can be dissolved in an organic phase (e.g., that does not dissolve ionic liquid (e.g., less than 1%, 0.1%, 0.01% or 0.001% ionic liquid)). In some cases, the ABS is formed using a phosphate salt (e.g., phosphate buffer). In some cases, the second phase comprises a phosphate salt (e.g., phosphate buffer). Operation (c) is a reactive extraction in that the sugars react with the boronic acid under basic conditions to form a covalent bond that is reversable under acidic conditions.

In contrast to the method described in WO2011/041455, the present method concentrates the ionic liquid rather than diluting it (when forming the ABS). The present method does not require the addition of a strong base in some embodiments because the sugars are transported from an acidic solution (the hydrolysate) to a basic solution (the phosphate buffer). The methods described herein can be used with any ionic liquid for which an ABS can be formed (by phosphate buffer or other methods).

The formation of an ABS can occur within a few minutes and at room temperature and pressure. Phosphate salt is cheap, safe and routinely used in industry, including the food industry. The pH of the phosphate buffer (PB) can be adjusted to any pH such that the ABS forms. The pH of the second phase can be about 8, about 9, about 10, about 11, about 12 or about 13. In some embodiments, the pH of the second phase is at least about 8, at least about 9, at least about 10, at least about 11, at least about 12 or at least about 13. In the pH range between 11 and 12, the pH is high enough to form a strong ABS, allowing fast and selective phase partition, the pH is not high enough to degrade sugars, and the pH is suitable for ionizing boronic acids and initiating sugar extraction.

The sugars can be recovered from the boronic acid. In some embodiments, the methods of the present disclosure further comprise contacting the boronic acid with an acid (to liberate the sugars from the boronic acid into an aqueous solution). The acid can be a strong acid such as HCl. In some embodiments, the acid is a weak acid such as carbonic acid (H₂CO₃). The use of carbonic acid to liberate sugars can avoid contaminating the resulting sugar with acid because H₂CO₃ can react with water and evolve from solution as CO₂. The CO₂ can be reused.

The organic phase can be any organic solvent that dissolves, but does not react with the boronic acid or the sugar-boronic acid complex. In some embodiments, the organic phase comprises an organic molecule that is immiscible with ionic liquid.

The “boronic acid” can be any molecule that reacts reversably with sugar, whether it comprises the element boron or not. In some embodiments, the boronic acid has the formula: R-x-B(OH)₂ (I); wherein x is a bond or an alkyl or alkenyl chain of 1-10 carbons, R comprises at least 1 aromatic ring, wherein optionally at least one ring is substituted by one or more alkyl groups comprising 1-10 carbons. In some embodiments, s is a bond or an alkyl or alkenyl chain of 1-4 carbons. In some embodiments, x is a bond or an alkyl or alkenyl chain of 1-2 carbons. In some embodiments, x is a —C═C—.

In some embodiments, R comprises 1, 2, or 3 aromatic rings. In some embodiments, R is a benzene, optionally comprising 1 or 2 methyl groups. In some embodiments, R is a naphthalene.

In some embodiments, the boronic acid is phenylboronic acid, 3,5-dimethylphenylboronic acid, 4-tert-butylphenylboronic acid, trans-P-styreneboronic acid, or naphthalene-2-boronic acid.

In some embodiments, provided herein is a method of removing a sugar from a solution, comprising: (a) providing a solution comprising ionic liquid, water and sugar; (b) separating the solution into an ionic liquid phase and an aqueous phase; (c) providing an organic phase comprising a boronic acid; (d) contacting the aqueous phase with the boronic acid to form a sugar-boronic acid complex, (e) separating the organic phase and the aqueous phase, wherein the organic phase contains the sugar-boronic acid complex, and optionally (f) separating the sugar from the organic phase.

In some embodiments, (f) comprises adding stripping solution comprising a stripping agent to the organic solution, such that the sugar-boronic acid complex dissociates and the sugar moves into the stripping solution. In some cases, the stripping solution is aqueous and the stripping agent is an acid which decrease the pH of the organic phase.

In some embodiments, the organic solution further comprises an organic solvent which ensures the boronic acid is fully dissolved in the organic phase. In some embodiments, the organic solvent is n-hexane, 1-octanol, or a mixture thereof.

A liquid-liquid extraction can be performed between an aqueous ionic liquid (IL) phase and a phosphate buffer (PB) phase. More generally, any other salting out agent or phase inducing agent may be used in lieu of the phosphate buffer. This operation can remove some of the water from the IL phase, making the IL more concentrated. This can happen because the PB phase is more hydrophilic than the IL phase. When removing water, sugars and other hydrophilic substances can also be removed. In addition, this operation also transfers sugars from an acidic to an alkaline environment. This can be carried out at ambient temperature and pressure. This operation can have a selectivity of about 10, about 100 or about 100 for sugars and against IL.

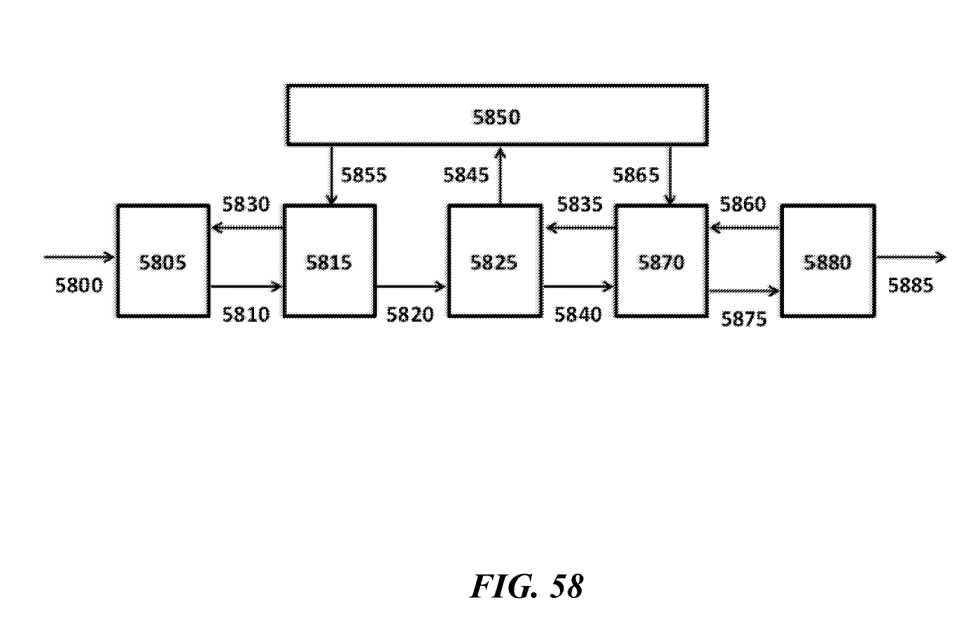

FIG. 58 shows an example of a separation process using formation of an ABS and reactive extraction of sugars with boronic acids. Biomass 5800 enters an ionic liquid hydrolysis reactor 5805. Ionic liquid hydrolysate 5810 exits the hydrolysis reactor to be separated into an ABS 5815 comprising an aqueous phase 5820 that moved to a liquid-liquid extraction vessel 5825 an ionic liquid phase 5850 that is returned to the hydrolysis reactor.

A liquid-liquid extraction can be performed between the PB phase and an organic phase. The organic phase can contain boronic acids. One example is an organic phase comprising 50-70 mM naphthalene-2-boronic acid. The organic phase can have a 85:15 (v/v) solution of n-hexane and 1-octanol. The organic phase can also contain 150 mM of a phase transfer catalyst (e.g., Aliquat 336). This operation can be carried out at ambient temperatures and pressures, and can take from several minutes to a couple hours. In some cases, the selectivity for this operation is about 1,000 to about 10,000 for sugars and against all ionic species (e.g., PB and IL).

Returning to FIG. 58, the phosphate buffer can be contacted with an organic phase comprising a boronic acid 5835. The sugars react with the boronic acid and are extracted 5840. The remaining phosphate buffer 5845 can be transferred to a dehydration and/or desalination unit 5850 that concentrates the phosphate buffer and returns it to the process 5855.

A liquid-liquid extraction can be performed between the organic phase and an aqueous acid phase. The aqueous acid can be carbonic acid formed by compressed CO₂ in water. In this operation, the boronic sugars carried in the organic phase are stripped to the aqueous phase. That is, when contacted with acid, the sugar-boronate bond is broken, liberating sugar to the aqueous phase. The organic phase is left with intact boronic acid. This operation can be carried at ambient temperature and elevated pressure (any pressure greater than atmospheric pressure).

Returning to FIG. 58, the sugars can be recovered by contact with an acid. In this example, carbon dioxide 5860 and water 5865 (producing carbonic acid) are contacted with the organic phase 5870 to lower the pH such that sugars are liberated into the water and removed from the organic phase 5875. A degasser 5880 can remove the carbonic acid as carbon dioxide to produce a stream of sugars 5885. The sugars can be clean and concentrated.

The degasser 5880 can be a heater or sparger with N₂ or some other inert gas.

The desalination operation 5850 can correct any water imbalance in the separation process. In some cases, the water that is removed from the IL hydrolyzate to the PB can be recycled. At the same time, the stripping step S865 can use fresh water. The desalination step can include evaporation and condensation of water and/or reverse osmosis.

### Lignin and Solids

Interest has grown regarding using lignin in various chemical processes on account of certain useful properties. In some applications, lignin can be used to produce aromatic compounds. In some applications, lignin can be used to produce carbon fiber. Realization of the potential benefits of chemical processes that use lignin is often limited by the quality of the starting lignin material.

Ionic liquids are salts (e.g., comprising cations and anions) that are a liquid. Interest has grown regarding using ionic liquids in various chemical processes. In some applications, ionic liquids can be used to dissolve material (e.g., cellulosic biomass). In some applications, ionic liquids can be used as a catalyst. Realization of the potential benefits of chemical processes based on ionic liquids has been limited by the high cost of ionic liquids.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Lignin is the second most abundant naturally-occurring substance and a major component of terrestrial plant biomass. Lignin is a complex organic polymer of mostly aromatic units. Lignin generally lacks a predefined polymeric structure and forms covalent bonds to hemicellulose in plants and trees (biomass), which imparts structural rigidity and can help conduct water.

Lignin can be extracted from biomass in a biorefinery such as a wet or dry corn mill, a paper pulping mill, and the like, resulting in a material with various degrees of quality depending on the process. For example, the sulfite pulping process can result in lignosulfonates, which is a derivatized lignin and has a high level of sulfonate impurity. Other processes extract lignin that contain cellulosic fibers, ash, humins, and the like.

Processes that transform lignin into other compounds can depend on the quality of the starting lignin. The quality of lignin can relate generally to the impurity level and the fragmentation level. Impurity level refers to the concentration of the various impurities present in lignin. Some impurities can be covalently bound to lignin. Some impurities can remain unbound by lignin. In some applications, a biorefinery produces derivatized lignin, a form of impure lignin. In some applications, a biorefinery produces lignin with a high concentration of ash. The impurity level of the produced lignin can be influenced by factors such as the starting biomass material and the process used in extracting lignin.

The level of fragmentation can also affect the quality of lignin. Fragmentation refers to the breaking of lignin molecules into smaller molecules. In some applications, a low level of fragmentation is desired. In some applications, substantially unfragmented lignin can be processed to yield a higher quality product. In most applications, substantially fragmented lignin is processed to a lower quality product. In some applications, the quality of carbon fiber can be improved by using lignin starting material with a lesser degree of fragmentation. The fragmentation level of the produced lignin can be influenced by factors such as the starting biomass material and the process used in extracting lignin.

In some aspects, the quality of the lignin starting material may limit the yield of chemical, material and/or fuel product. Yield can be the main driver for the economic success of a chemical process. Therefore, it may be highly advantageous to start with high quality lignin.

High quality lignin can be transformed into a wide range of chemical, material and/or fuel products. In some aspects, lignin can be transformed into benzene, toluene and xylene. In some aspects, lignin can be transformed into concrete. In some aspects, lignin can be transformed into antioxidant. In some aspects, lignin can be transformed into asphalt. In some aspects, lignin can be transformed into carbon fiber and related fibers. In some aspects, lignin can be transformed into board binders. In some aspects, lignin can be transformed into foams, plastics and other polymers. In some aspects, lignin can be transformed into dust control agents. In some aspects, lignin can be transformed into paper.

In some aspects, lignin can be transformed into various chemicals, such as cresols, catechols, resorsinols, quinones, vanillin, guaiacols, and the like. In some aspects, lignin can be transformed into various aromatic compounds. In some aspects, lignin can be transformed into fuels, such as gasoline replacements, diesel replacements, blendstocks, and the like. In some aspects, lignin can be transformed into heat. In some aspects, lignin can be transformed into grease. In some aspects, lignin can be transformed into dispersants.

In some aspects, lignin can be transformed into various agricultural chemicals, urea compositions, fertilizer compositions, dispersant compositions, emulsifier compositions, heavy metal sequestrate compositions, additive compositions, soil water retention agent compositions, and the like.

Recovery and/or Precipitation of Biomass Components

One or more biomass components can be recovered from the biomass mixture. In some embodiments, the biomass component forms a precipitate in the biomass mixture. A precipitate is any one or more biomass components that are partially dissolved or undissolved in the ionic liquid. In some embodiments, additional methods, described herein, are performed to cause a precipitate to form.

In some instances, one or more biomass components do not precipitate or precipitate only partially from the biomass and ionic liquid mixture. In these instances, it may be beneficial to induce or enhance precipitation by contacting the biomass and ionic liquid mixture with a suitable fluid. Exemplary fluids include but are not limited to gases, liquids, pressurized gases, liquefied gases, sub-critical fluids, volatile liquids, and/or supercritical or near-supercritical fluids. In some cases, the fluid is an anti-solvent. Water is an example of an anti-solvent and/or fluid. An “anti-solvent”, as used herein generally refers to a chemical species that decreases the solubility of a solute.

For example, in one embodiment, the biomass and ionic liquid mixture is contacted with a gas to form a solid precipitate. In another embodiment, the biomass and ionic liquid mixture is contacted with a pressurized gas. In yet another embodiment, the biomass and ionic liquid mixture comprising one or more biomass components in an ionic liquid is contacted with a supercritical or near-supercritical fluid.

In some embodiments, the fluid is selected from the group consisting of CO₂, NO₂, NH₃, water, acetic acid, methanol, ethanol, n-butane, nitrogen, hydrogen, helium, argon, oxygen, methane, ethane, propane, ethylene, propylene, and combinations thereof. In some embodiments, the fluid is CO₂.

For clarity, “contacted with a gas” does not necessarily mean that the fluid is a gas when contacted with the biomass mixture. In some cases, the gas can be pressurized such that it is a dense phase (e.g., liquefied gas or supercritical fluid) when contacted. As used herein, a gas is a material that is a vapor at International Union of Pure and Applied Chemistry (IUPAC) standard temperature and pressure (0° C. and 1 bar). A pressurized gas is any gas at a pressure greater than 1 bar.

In some embodiments, the biomass mixture and ionic liquid is contacted with a pressurized gas. In some instances, the gas is pressurized to an absolute pressure greater than atmospheric pressure. In some embodiments, the pressure is about 1 bar, about 2 bar, about 5 bar, about 10 bar, about 20 bar, about 30 bar, about 40 bar, about 50 bar, about 100 bar, about 200 bar, about 300 bar or about 400 bar. In some embodiments, the pressure is at least 1 bar, at least 2 bar, at least 5 bar, at least 10 bar, at least 20 bar, at least 30 bar, at least 40 bar, at least 50 bar, at least 100 bar, at least 200 bar, at least 300 bar or at least 400 bar.

In some embodiments, the biomass mixture is contacted with a liquefied gas. Examples of gases that can be liquefied include propane, hydrogen, nitrogen, n-butane and carbon dioxide.

In some embodiments, the biomass mixture is contacted with a volatile liquid. Examples of liquids that are readily volatile include propanone, methanol and ethanol.

The critical temperature of a fluid is the temperature above which a distinct liquid phase does not exist (e.g., regardless of pressure). The vapor pressure of a fluid at its critical temperature is its critical pressure. At temperatures and pressures above its critical temperature and pressure (e.g., its critical point), a fluid is called a supercritical fluid. Many fluids can form supercritical fluids provided they do not degrade or decompose at temperatures below their critical temperature.

In some instances, the methods of the present invention can use any suitable supercritical or near-supercritical fluid. Information on supercritical fluids can be found in “Fundamentals of Supercritical Fluids” by Tony Clifford (ISBN: 978-0198501374), “Supercritical Carbon Dioxide: Separations and Processes” by Aravamudan S. Gopalan (ISBN: 978-0841238367), and “Supercritical Fluid Extraction” by Larry T. Taylor (ISBN: 978-0471119906), each of which is herein incorporated by reference in its entirety.

The fluid can be supercritical, in that both the temperature is at or above its critical temperature and the pressure is at or above its critical pressure. In some embodiments, the pressure is about 100%, about 120%, about 150%, about 200%, about 300%, about 500%, and the like of the fluid's critical pressure. In some embodiments, the pressure is at least about 100%, at least about 120%, at least about 150%, at least about 200%, at least about 300%, at least about 500%, and the like of the fluid's critical pressure. In some embodiments, the temperature is about 100%, about 120%, about 150%, about 200%, about 300%, about 500%, and the like of the fluid's critical temperature. In some embodiments, the temperature is at least about 100%, at least about 120%, at least about 150%, at least about 200%, at least about 300%, at least about 500%, and the like of the fluid's critical temperature. In some embodiments, the pressure is between about 80% and 400% of the fluid's critical pressure. In some embodiments, the temperature is between about 80% and 400% of the fluid's critical temperature.

The fluid can be sub-critical (e.g., near-supercritical), in that one or both of the temperature is below the fluid's critical temperature and the pressure is below its critical pressure. A near-supercritical fluid may have properties similar or near the properties of a supercritical fluid. In various embodiments, the pressure is about 99%, about 98%, about 95%, about 90%, about 85%, about 75%, about 50%, about 20%, and the like of the fluid's critical pressure. In various embodiments, the pressure is at least about 99%, at least about 98%, at least about 95%, at least about 90%, at least about 85%, at least about 75%, at least about 50%, at least about 20%, and the like of the fluid's critical pressure. In various embodiments, the temperature is about 99%, about 98%, about 95%, about 90%, about 85%, about 75%, about 50%, about 20%, and the like of the fluid's critical temperature. In various embodiments, the temperature is at least about 99%, at least about 98%, at least about 95%, at least about 90%, at least about 85%, at least about 75%, at least about 50%, at least about 20%, and the like of the fluid's critical temperature.

In some embodiments, fluids with low critical temperatures and/or pressures may be employed (e.g., to reduce the amount of energy that needs to be put into the process to heat and/or pressurize the fluid). In some embodiments, fluids with low temperatures are employed (e.g., to preserve heat labile reactants and/or products). In some embodiments, the temperature is sufficiently low to avoid decomposition of the biomass components (e.g., less than 200° C., less than 150° C., less than 100° C., less than 80° C., less than 60° C., less than 40° C., less than 30° C., less than 20° C., or less than 10° C.).

Supercritical fluids can have densities, viscosities, and other properties that are intermediate between those of the fluid in its gaseous and in its liquid state. Table 1 lists some supercritical properties of four compounds. These four fluids are examples of fluids that have relatively moderate critical temperatures (e.g., less than 200° C., less than 150° C., less than 100° C., less than 80° C., less than 60° C., less than 40° C., less than 30° C., less than 20° C., or less than 10° C.) and critical pressures (e.g., less than 200 atm, less than 150 atm, less than 120 atm, less than 110 atm, less than 100 atm, less than 90 atm, less than 80 atm, less than 70 atm, less than 60 atm, less than 50 atm, less than 40 atm, less than 30 atm, or less than 20 atm).

In some cases, supercritical fluids dissolve solutes in proportion to the density of the fluid. In some embodiments, the supercritical or near-supercritical fluid has a density of about 0.05, about 0.1, about 0.15, about 0.2, about 0.25, about 0.3, about 0.35, about 0.4, about 0.5, about 0.6, about 0.7, about 0.8, about 0.9, or about 1.0 g/mL. In some embodiments, the supercritical or near-supercritical fluid has a density of at least 0.05, at least 0.1, at least 0.15, at least 0.2, at least 0.25, at least 0.3, at least 0.35, at least 0.4, at least 0.5, at least 0.6, at least 0.7, at least 0.8, at least 0.9, or at least 1.0 g/mL. In some embodiments, the supercritical or near-supercritical fluid has a density of between about 0.2 and 0.9 g/mL.

In some embodiments, the supercritical or near-supercritical fluid is selected from the group consisting of CO₂, NO₂, NH₃, water, acetic acid, methanol, ethanol, n-butane, nitrogen, hydrogen, helium, argon, oxygen, methane, ethane, propane, ethylene, propylene, and combinations thereof. In some embodiments, the supercritical or near-supercritical fluid is CO₂.

In some embodiments, the fluid is substantially pure (e.g., at least 80%, 90%, 95%, 99%, 99.5, or 99.9% pure). In some embodiments, the fluid is a mixture. For example, a mixture of water and sugar may be used to precipitate a biomass component.

In some embodiments, the fluid is non-toxic, biodegradable, non-flammable, or has other properties that result in a safe and environmentally friendly process.

In certain embodiments, the formation of precipitate may be enhanced by cooling, heating, vibrating, sounding (acoustic wave), or any combination thereof.

### Separation of Precipitate

In some embodiments, the precipitate is removed from the biomass and ionic liquid mixture. In some embodiments, the biomass and ionic liquid mixture is filtered to separate precipitate. In some embodiments, a drum filter is used. In some embodiments, a horizontal belt filter is used. In some embodiments, a horizontal table filter is used. In some embodiments, a tilting pan filter is used. In some embodiments, a disk filter is used. In some embodiments, a combination of two or more filters is used.

In some embodiments, the biomass mixture is centrifuged to separate precipitate. In some embodiments, a tubular centrifuge is used. In some embodiments, a disk centrifuge is used. In some embodiments, a nozzle discharge centrifuge is used. In some embodiments, a helical conveyor centrifuge is used. In some embodiments, a knife discharge centrifuge is used. In some embodiments, a combination of two or more centrifuges is used. In some embodiments, a combination of a centrifuge and filter are used.

### Recovery of Ionic Liquid and Removal of Impurities

In some embodiments, separated precipitate contains ionic liquid and other impurities. It is generally desirable to remove these components and obtain a clean product. In some embodiments, separated precipitate is washed with a fluid one or multiple times. In some embodiments, the fluid is substantially miscible in the biomass mixture. In some embodiments, the fluid comprises an alcohol, a ketone, an aldehyde, or any combination thereof. In some embodiments, the fluid comprises and acid or a base. In some embodiments, the fluid comprises water.

Biomass components can generally be cleaned to any purity level. In some embodiments, the total amount of impurity in product is less than 50%, less than 25%, less than 5%, less than 2.5%, less than 0.5%, less than 0.25%, less than 0.05%, less than 0.025%, less than 0.005%, less than 0.0025%, less than 0.0005%, less than 0.00025 or less than 0.000005% by mass.

In some embodiments, less than about 10 gram to about 0.001 gram of ionic liquid is lost per kilogram of biomass component separated. In some embodiments, less than about 1 gram to about 0.001 gram of ionic liquid is lost per kilogram of biomass component separated. In some embodiments, less than about 1 gram to about 0.01 gram of ionic liquid is lost per kilogram of biomass component separated. In some embodiments, less than about 0.1 gram to about 0.001 gram of ionic liquid is lost per kilogram of biomass component separated. In some embodiments, less than about 0.1 gram to about 0.01 gram of ionic liquid is lost per kilogram of biomass component separated.

In some embodiments, a clean lignin product is desired. Lignin can generally be cleaned to any suitable purity level. In some embodiments, the total amount of non-lignin in lignin product is less than 50%, less than 25%, less than 5%, less than 2.5%, less than 0.5%, less than 0.25%, less than 0.05%, less than 0.025%, less than 0.005%, less than 0.0025%, less than 0.0005%, less than 0.00025 or less than 0.000005% by mass.

The recovery of ionic liquid from solids is an aspect of the invention. In some embodiments, the method for lignin recovery from an ionic liquid comprises losing less than 10 grams of ionic liquid per kilogram of lignin component separated. In some embodiments, less than 1 gram of ionic liquid is lost per kilogram of lignin component separated. In some instances, less than 0.1 gram of ionic liquid is lost per kilogram of lignin component separated. In some instances, less than 0.01 gram of ionic liquid is lost per kilogram of lignin component separated. In some instances, less than 0.001 gram of ionic liquid is lost per kilogram of lignin component separated.

The ionic liquid can be recovered to any suitable level. In some instances, at least 95%, at least 96%, at least 97%, at least 98%, at least 99%, at least 99.5%, at least 99.9%, at least 99.99%, at least 99.999%, at least 99.9999%, or at least 99.99999% of the ionic liquid is recovered (e.g., per batch or per week of operation). In some embodiments, the ionic liquid is recovered in a range of at least 95% to at least 99.99999%, at least 96% to at least 99.999%, at least 97% to at least 99.99%, at least 98% to at least 99.9%, or at least 99% to at least 99.5%.

The purity of the ionic liquid following the process is any suitable level. In some instances, the ionic liquid is at least 95%, at least 96%, at least 97%, at least 98%, at least 99%, at least 99.5%, at least 99.9%, at least 99.99%, at least 99.999%, at least 99.9999%, or at least 99.99999% pure. In some embodiments, the ionic liquid has a purity in a range of at least 95% to at least 99.99999%, at least 96% to at least 99.999%, at least 97% to at least 99.99%, at least 98% to at least 99.9%, or at least 99% to at least 99.5%.

High quality lignin comprises lignin of any suitable level of fragmentation. In some embodiments, the total mass of lignin that is fragmented with respect to intact lignin in relation to intact lignin is less than 99.99999%, less than 99.9%, less than 75%, less than 50%, less than 25%, less than 5%, less than 1%, less than 0.01%, less than 0.001%, less than 0.0001%, less than 0.00001%, or less than 0.000001%.

In some instances, the ionic liquid is re-used after the process (e.g., after recovering biomass components from the ionic liquid).

The process separating lignin from hydrolysate mixture can include, for example and without limitation, a batch process, a continuous process, a semi-batch process, or combination thereof.

The process recovering ionic liquid from lignin can include, for example and without limitation, a batch process, a continuous process, a semi-batch process, or combination thereof.

Suitable methods for determining the amount of ionic liquid lost from the process include, but are not limited to determining the mass of ionic liquid before and after the process, or operating the process for a period of time and observing a loss in ionic liquid over that time period.

### Depressurization

Some gases, such as CO₂, can penetrate fibrous materials left over by biomass hydrolysis (e.g., lignin, residual cellulose, humins, etc). Some industrial gases (e.g., small molecules) diffuse quickly and/or possess an affinity to fibrous material. In some cases, gases penetrate the fiber structure. The use of gases as described herein can flush out molecules attached to the fibers, such as ionic liquid that can be recovered.

After contact with pressurized gas, the pressure can be quickly released (e.g., in order to break up the fiber structure at the microscopic level, prevent ionic liquids from being retained in the fibrous material, prevent wash fluids from being inaccessible to portions of the fibrous material). The pressure can be released over any suitable time period (e.g., less than 1 second, less than 10 seconds, less than 1 minute) and to any suitable extent (e.g., by at least 10%, at least 20%, at least 40%, at least 60%, at least 80%, and/or released down to atmospheric pressure). The pressure prior to depressurization can be obtained from other unit operations in the separation process, such as aqueous biphasic system formation. Depressurization can be performed before or during a filtration step. In some cases, depressurization may be used before or during other solid-liquid separation steps.

### Process Configurations for Recovery of Solid Precipitates (e.g., Lignin)

The lignin can be filtered at a pH at which the lignin does not form a gel. The lignin can form a gel at high pH (e.g., above the pKa for a phenol of about 10) that is difficult to filter (e.g., reduces the filtration rate by about 20%, 30%, 40%, 50%, 60%, 70%, 80%, or more relative to the rate when the lignin is not a gel). In some cases, the lignin forms a gel that is difficult to filter at a pH of about 8, about 9, about 10, about 11, about 12, about 13 or about 14. Thus the filtration can be performed at a pH of less than about 8, less than about 9, less than about 10, less than about 11, less than about 12, less than about 13 or less than about 14.

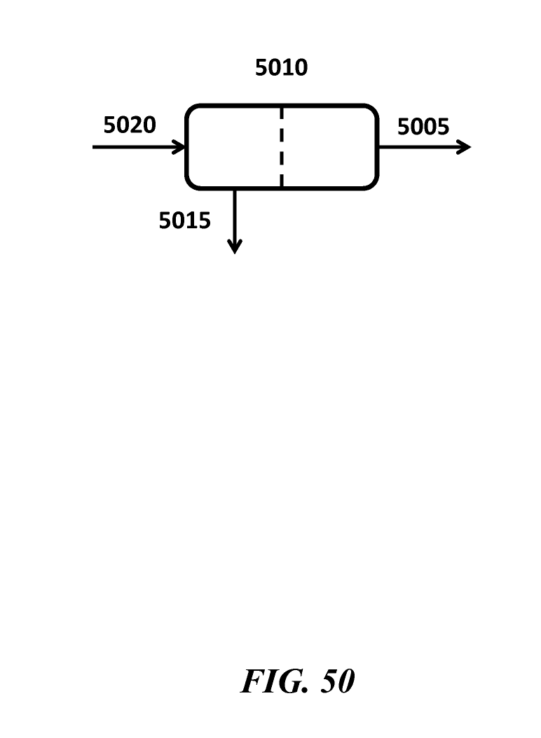

With reference to FIG. 50, in some embodiments, precipitate coming from elsewhere in the process (e.g., 5020) enters a filter 5010. Filtrate is created 5005 as the mixture passes through the membrane in 5010. Retentate is also created 5015, which is composed of mixture components that do not pass through the membrane.

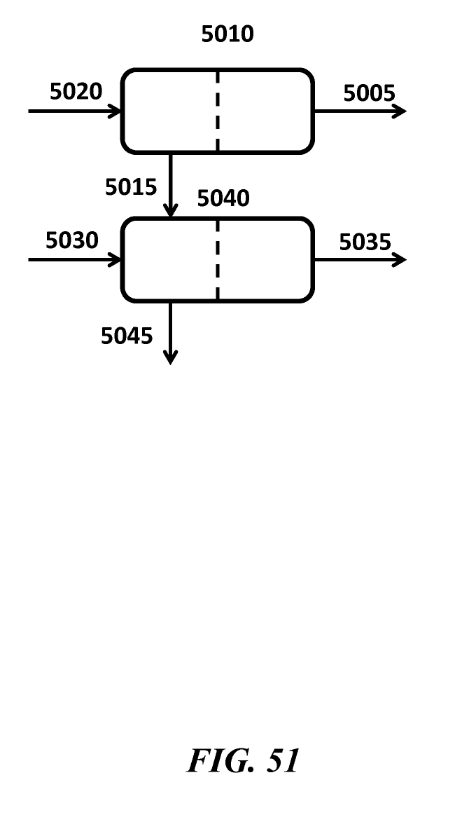

With reference to FIG. 51, in some embodiments, biomass mixture (e.g., comprising ionic liquid and lignin precipitate) coming from elsewhere in the process (e.g., 5020) enters a filter 5010. Filtrate is created 5005 when some of the mixture passes through the membrane in 5010. Retentate is also created 5015, which does not pass through the membrane. The retentate enters a washer 5040. A wash fluid is introduced to the washer 5030. The wash fluid can be any suitable fluid (e.g., ethanol or water) used in any quantity at any temperature or pressure. A membrane 5040 impedes the passage of a least some of the washed solids. Washed solids exit the process 5045. The remainder passes through the membrane 5035.

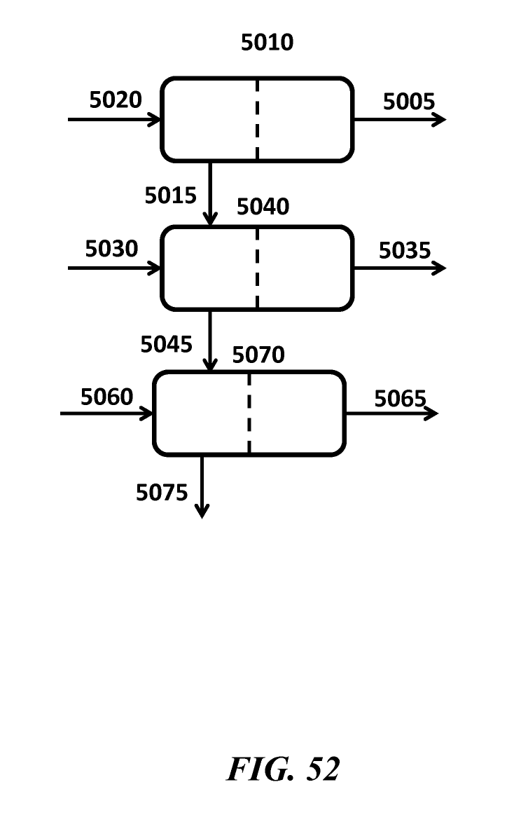

With reference to FIG. 52, in some embodiments, biomass mixture (e.g., comprising ionic liquid and lignin precipitate) coming from elsewhere in the process (e.g., 5020) enters a filter 5010. Filtrate is created 5005 when some of the mixture passes through the membrane in 5010. Retentate is also created 5015, which does not pass through the membrane. The retentate enters a washer 5040. A wash fluid is introduced to the washer 5030. A membrane 5040 impedes the passage of a least some of the washed solids. Washed solids exit the process 5045. The remainder passes through the membrane 5035. Washed solids 5045 enter another washer 5070, where the process is repeated in order to reduce the concentration of solid impurities further, or recover impurities. The first and/or second wash fluid can be any suitable fluid (e.g., ethanol or water) used in any quantity at any temperature or pressure.

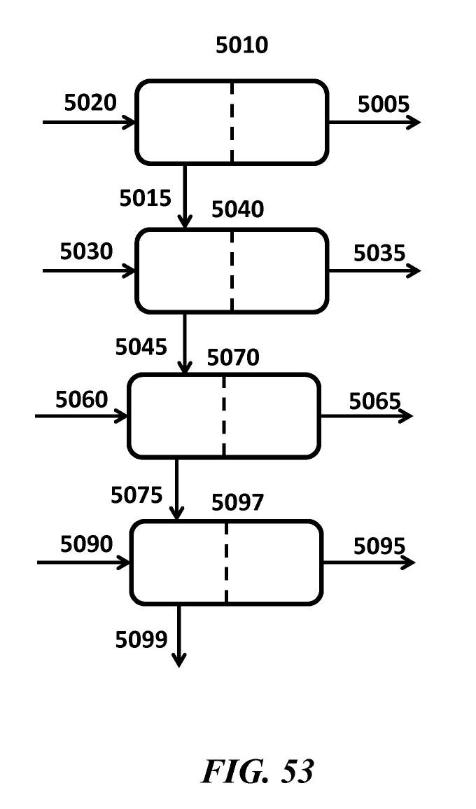

With reference to FIG. 53, in some embodiments, biomass mixture (e.g., comprising ionic liquid and lignin precipitate) coming from elsewhere in the process (e.g., 5020) enters a filter 5010. Filtrate is created 5005 when some of the mixture passes through the membrane in 5010. Retentate is also created 5015, which does not pass through the membrane. The retentate enters a washer 5040. A wash fluid is introduced to the washer 5030. A membrane 5040 impedes the passage of a least some of the washed solids. Washed solids exit the process 5045. The remainder passes through the membrane 5035. Washed solids 5045 enter another washer 5070, where the process is repeated in order to reduce the concentration of solid impurities further, or recover impurities. Washed solids 5075 enter a dryer 5097. In the drier, heat or energy 5090 enters the dryer, resulting in the creation of vapor 5099, and the drying of solids 5095. The first and/or second wash fluid can be any suitable fluid (e.g., ethanol or water) used in any quantity at any temperature or pressure. The drying can be performed at any suitable temperature for any suitable amount of time.

The performance of lignin separation can be improved by the addition of other species. For instance, the dissolution of CO₂ can increase the amount of lignin precipitate in the mixture prior to filtering, therefore increasing the total amount of recoverable solids.

Anti-solvents can impart a similar effect. For instance, anti-solvents such as alcohols and ketones can help in removing lignin and/or other solids. In some cases, other chemicals can prevent the formation of tight clumps that may become difficult to wash and may retain a quantity of ionic liquid residue. The use of kosmotropic salts (e.g., NaOH, K₃PO₄, Na₃PO₄) can improve the yield of lignin after separation. An adjustment of pH can also increase ionic liquid recovery yields. For instance, increasing pH can precipitate lignin and/or decrease its propensity for clumping together. This can, therefore, not only increase the yield of solids after separation, but also improve the performance of the separation process.

It can be beneficial to allow the mixture to settle (e.g., depending on the vessel configuration and design). Settling is an example of a method for passively pre-concentrating solids that are to be filtered or otherwise removed. Some particulates may float to the top, instead of settling to the bottom. In this case, a scraper or skimmer can be used in order to remove those solids. High shearing flows and/or acoustic waves can also be used to improve filtration by homogenizing the mixture and breaking up clumps.

Filtering aids can be added to improve performance. Filtering aids (e.g., diatomaceous earth), may be added to the hydrolysate mixture just before passing it through a screen. Filtering aids can help to disperse flow channels that reduce filtration performance (e.g., without impeding flow).

Lignin and other solids can be removed from the hydrolysate mixture by methods other than filtering. For example, centrifugation can be used in order to concentrate particulate (precipitate) matter without the need of a screen or membrane. There are many kinds of centrifuges, including batch, semi-batch and continuous operating units. Alternatively, a centrifuge can be used in conjunction with a membrane in order to obtain a more concentrated cake. In cases where particulates settle relatively quickly, a hydrocyclone can be used. Hydrocyclones can be cost-effective as they concentrate solids quickly and continuously. The solids output from the hydrocyclone can feed a filtration/wash process of reduced size and expense (in comparison to the process without the hydrocyclone).

The separation process can involve the use of pressurized gases. This can take place in one or several unit operations. At any pressure above atmospheric, the positive pressure can be used to improve separation of lignin and other solids. A positive pressure can be produced by either a gas or by reducing the volume of the compartment.

If a gas is used, the gas may dissolve in the hydrolysate. A dissolved gas may reduce the viscosity of the mixture. A reduced viscosity can cause the fluid to behave more predictably and can result in faster separation. Faster separations can be performed using smaller equipment, which can reduce cost.

In some cases, the separation may be performed at the same rate as it was without gas dissolution. In this case, separation can be less energy-intensive, and therefore also cheaper.

### Lignin Product Dissolution

The methods can further comprise dissolving the lignin (e.g., following recovery of the lignin from the ionic liquid). The dissolved lignin can be converted into various fuels, materials or chemicals. In some cases, the conversion of lignin into these components can benefit from the lignin being relatively clean as described herein.

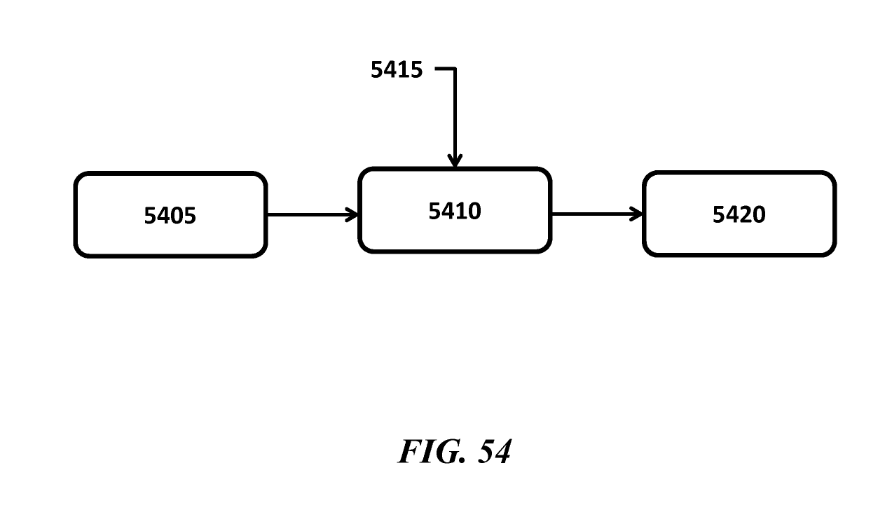

As shown in FIG. 54, lignin can be recovered using the methods described herein 1305. The lignin can then be dissolved 5410 with a lignin solvent 5415. Any suitable lignin solvent can be used, including ionic liquids, alcohols, ketones, and mixtures thereof. In some cases, one or several lignin solvents can be present during the solid-liquid separation process described herein (e.g., in order to limit the amount of materials, improve separation efficiency, improve separation rate, etc). In some cases, dissolving lignin also bypasses the need for drying the solids or removing residual compounds. Dissolved lignin is flowable, easily mixed and can be tied into any suitable downstream conversion process 5420.

The methods described herein can be used with any ionic liquid that dissolves any biomass or biomass component (e.g., cellulose or lignin). In some cases, ionic liquids are used that dissolve lignin. In some cases, the ionic liquids do not dissolve cellulose. Examples of lignin-dissolving ionic liquids are described in PCT Patent Publication Number WO2012/080702, which is hereby incorporated by reference in its entirety.

The lignin-dissolving ionic liquid can be formed from the reaction of an amine (e.g., tri-ethylamine) with sulfuric acid. In some cases, the ionic liquid comprises a cation (e.g., a protic cation) and an anion selected from C₁₋₂₀ alkyl sulfate [AlkylSO₄]⁻, C₁₋₂₀alkylsulfonate [AlkylSO₃]⁻, hydrogen sulfate [HSO₄]⁻, hydrogen sulphite [HSO₃]⁻, dihydrogen phosphate [H₂PO₄]⁻, hydrogen phosphate [HPO4]²⁻ and acetate, [CH₃CO₂]⁻. The lignin-dissolving ionic liquid can contain water (e.g., 10-40% v/v water when the anion is acetate).

Any biomass component, including but not limited to lignin, lignin fragments, lignin derivatives, hemicellulose, hemicellulose hydrolysate (e.g., xylose), cellulose, cellulose hydrolysate (e.g., glucose), acetate, and ash can be removed from the lignin-dissolving ionic liquids using the methods described herein. Non-limiting methods including the formation of an aqueous biphasic system and removal of the biomass components in the aqueous phase. Aqueous bi-phasic systems can be formed by contact with fluids (e.g., pressurized CO₂), addition of kosmotropes, or change of pH for example.

Methods described herein (e.g., formation of ABS) may be used for the extraction of sugars (and precipitation of lignin and other solutes) from the lignin-dissolving ionic liquid. Formation of a two-phase system rich in water and ionic liquid can separate water-soluble lignin fragments and other components such as acetate, which enrich in the water layer, from larger lignin fragments, which enrich in the ionic liquid layer. These methods can also remove water from ionic liquid, which can be an important step for keeping a suitable water concentration during the various steps of the process.

In-situ hydrolysis of cellulose and/or hemi-cellulose (e.g., by gradual addition of water) can be performed in the lignin-dissolving ionic liquid. Furthermore, if the lignin-dissolving ionic liquid is a protic ionic liquid, the amount of acid required for in situ hydrolysis can be much decreased relative to aprotic ionic liquids.

Methods described herein for the removal of lignin and recovery of ionic liquid can be used with lignin-dissolving ionic liquids. For example, lignin can be precipitated by applying a pressure of a gas with good solubility in the lignin-dissolving ionic liquid (e.g., CO₂).

### Single-Pot Hydrolysis and Recovery

In some cases, a single vessel can be used for hydrolysis and extraction (e.g., one-pot). Whereas biomass-dissolving ionic liquids are generally, but not exclusively hydrophilic, ionic liquids considered to be “hydrophobic” can also dissolve minor amounts of water. In hydrolysis, the stoichiometric ratio between water and carbohydrate is generally low (about 0.11 by mass in some instances). In an embodiment, if 10% of the solution is biomass, only ˜0.5% water is required. Hydrophobic ionic liquids can generally dissolve water to 0.5%. In some cases, hydrophobic ionic liquids are used that dissolve biomass to at least 1%(w/w), at least 2% (w/w), at least 3% (w/w), at least 4% (w/w), at least 5% (w/w), at least 6% (w/w), at least 7% (w/w), at least 8% (w/w), at least 9% (w/w), or at least 10% (w/w).

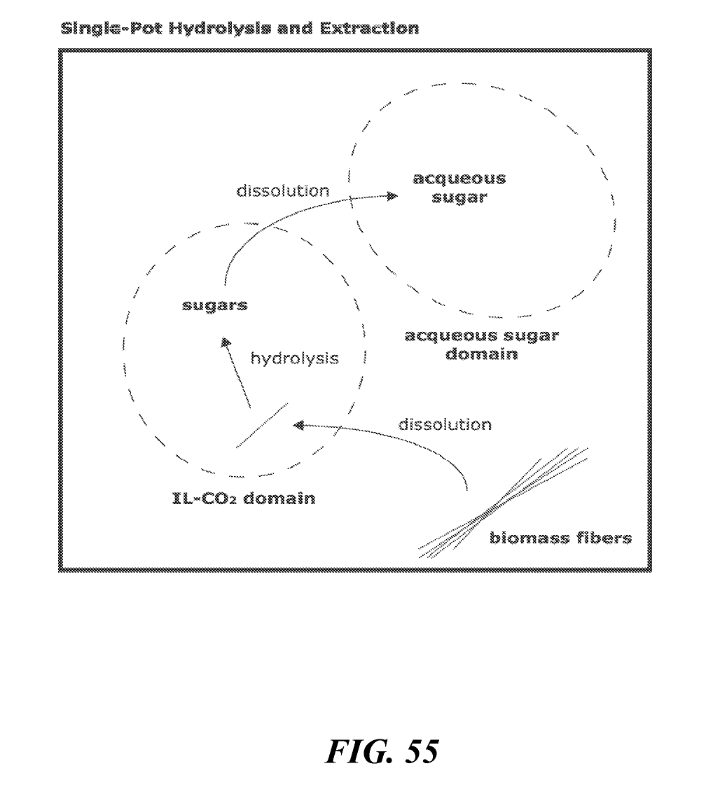

In some embodiments, a single-pot method uses an ionic liquid-CO₂ phase created by pressurized CO₂ in a biomass dissolving ionic liquid (e.g., [BMIM]Br). FIG. 55 shows an embodiment in which biomass fibers (e.g., cellulose) are dissolved in an ionic liquid/CO₂ domain where hydrolysis to sugars occurs. The sugars are then dissolved in an aqueous domain where they can be recovered from the ionic liquid. There can be two or more phases in a single vessel. The first phase is an ionic liquid-rich, ionic liquid-CO₂ phase that dissolves and hydrolyzes biomass (optionally aided by acid). The second phase is an aqueous sugar phase. In some cases, the CO₂ in ionic liquid-CO₂ results in the partial exclusion of water. In some instances, (e.g., if the sugar phase is near saturation with respect to sugar) the aqueous phase excludes ionic liquid (e.g., to less than 5%, 3%, 2%, or 1%).

In some embodiments, the reactor-extractor vessel inputs biomass and water, and outputs aqueous sugars. The aqueous fraction coming out can be processed to recover any ions from ionic liquid carried into the aqueous phase. The ions can be returned to the vessel. Separation processes for oils and lignin are also performed (e.g., phase separation for oils and filtration for lignin).

In some instances, the pressure is scheduled (e.g., varied according to a determined program) instead of using a single pressure “sweet spot”. In some cases, a lower pressure favors dissolution and hydrolysis, while a high pressure favors extraction. In some embodiments, the pressure is alternated between low and high pressures, with dwell times at each pressure according to the characteristic timescale of processes (e.g., dissolution, hydrolysis and extraction) in each of the phases.

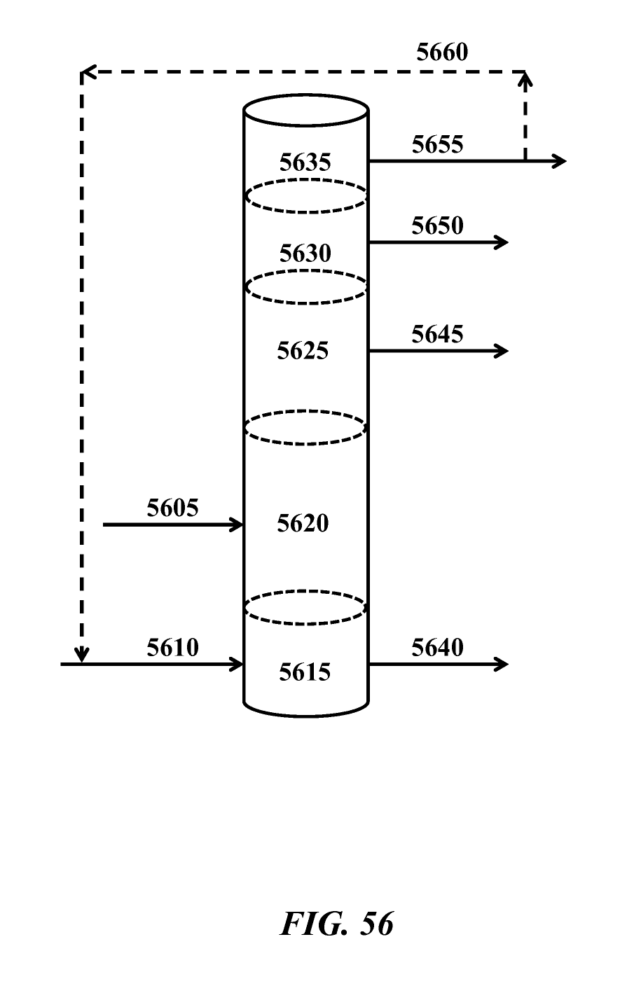

Turning attention now to FIG. 56, fractionation of biomass hydrolysate (e.g., into aqueous sugars, solids, and oils) can be performed in a single vessel. In some instances, the biomass is not hydrolyzed in the vessel 5600. The hydrolyzed biomass (in ionic liquid) 5605 can be pumped into a column. A pressurized gas such as CO2 is also introduced into the column 5610, optionally below the level at which the hydrolysate is introduced. The hydrolysate can separate into multiple phases as depicted by the dashed lines in FIG. 56. The phases might not form a distinct interface, and might blend together. In some cases, a first phase 5615 can comprise precipitated solids such as lignin and/or ash. The solids can be drawn off from the column 5640 and recovered from the ionic liquid as described herein, with the ionic liquid being returned to the column and/or to a hydrolysis reactor. A second phase can comprise ionic liquid hydrolysate 5620. An aqueous (third) phase 5625 can form that has water soluble sugars. The third phase can be drawn off from the column 5645 fed into a fermentor or to operations that recover residual ionic liquid. An oily (fourth) phase 5630 can contain for example tall oils and be drawn off the column 5650. A gaseous (fifth) phase (e.g., comprising CO₂) 5635 can be drawn off the column 5655 and optionally returned to the column 5660. In some cases, the gaseous phase is pressurized before being returned to the column.

In some embodiments, a method comprises: (a) adding biomass to a vessel comprising ionic liquid; and (b) adding a pressurized gas to the vessel, wherein the biomass is dissolved and hydrolyzed to sugar in the ionic liquid and at least one of (i) lignin is not dissolved in the ionic liquid, (ii) lignin is precipitated from the ionic liquid, (iii) the sugar is extracted in an aqueous phase, (iv) the sugar is extracted in the pressurized gas, (v) oils are removed by phase separation, and (vi) oils are extracted in the pressurized gas.

In some embodiments, the vessel is a column. In some embodiments, the vessel maintains a pressure gradient. In some embodiments, the ionic liquid comprises acid.

In some embodiments, a method comprises (a) contacting biomass with a mixture comprising ionic liquid and gas, and (b) applying a varying pressure, wherein the contacting and varying pressure results in a first phase comprising ionic liquid and a second phase comprising sugar.

In some embodiments, the second phase comprises water. In some embodiments, the gas is carbon dioxide. In some embodiments, the method further comprises recovering lignin and/or oils from the ionic liquid.

In some embodiments, a method comprises hydrolyzing biomass in ionic liquid in a vessel and separating the hydrolysate from the ionic liquid in the vessel. In some embodiments, the vessel is a column. In some embodiments, the vessel maintains a pressure gradient. In some embodiments, the vessel comprises pressurized gas. In some embodiments, the gas comprises carbon dioxide.

In some embodiments, the water soluble sugars of the hydrolysate are separated from the ionic liquid in a water phase. In some embodiments, the water soluble sugars of the hydrolysate are extracted from the ionic liquid in the pressurized gas. In some embodiments, the solids of the hydrolysate are separated from the ionic liquid with a filter. In some embodiments, solids comprise lignin, ash, or any combination thereof. In some embodiments, the oils of the hydrolysate are separated from the ionic liquid in an oil phase.

### Reduction of Dielectric Constant

Ionic liquids can have a high ionic strength and/or high dielectric constant. The dielectric constant (also referred to as static relative permittivity) is the ratio of the amount of electrical energy stored in a material by an applied voltage, relative to that stored in a vacuum. Dielectric constant is generally represented by the Greek letter epsilon and has no units (i.e., is a dimensionless number).

Some solutes dissolve in ionic liquids having a certain dielectric constant. In some embodiments, described herein is a method for separating a solute from an ionic liquid comprising reducing the dielectric constant of a composition comprising an ionic liquid and a solute. In some cases, the dielectric constant is reduced by contacting the composition with a pressurized gas. In some embodiments, the composition is not mixed with a liquid (e.g., water). In some embodiments, the dielectric constant of the ionic liquid is increased following separation of the solute by de-pressurizing the gas and/or separating the gas from the composition. The ionic liquid can be recycled and/or re-used.

The dielectric constant can be reduced by any suitable amount. In some embodiments, the dielectric constant is reduced by about 0.1%, about 0.5%, about 1%, about 2%, about 3%, about 5%, about 10%, about 15%, about 20%, about 25%, about 30%, about 40%, about 50%, about 60%, about 70%, about 80%, about 90%, about 95%, or about 99%. In some embodiments, the dielectric constant is reduced by at least 0.1%, at least 0.5%, at least 1%, at least 2%, at least 3%, at least 5%, at least 10%, at least 15%, at least 20%, at least 25%, at least 30%, at least 40%, at least 50%, at least 60%, at least 70%, at least 80%, at least 90%, at least 95%, or at least 99%. In some embodiments, the dielectric constant is reduced by at least about 0.1% to about 99%, about 0.5% to about 95%, about 1% to about 90%, about 2% to about 80%, about 3% to about 70%, about 5% to about 60%, or about 10% to about 70%.

The dielectric constant can be reduced to any suitable value. In some embodiments, the dielectric constant is reduced to an epsilon of about 2, about 4, about 6, about 8, about 10, about 12, about 14, about 16, about 18, about 20, about 25, about 30, about 40, about 50, or about 100. In some embodiments, the dielectric constant is reduced to an epsilon of less than 2, less than 4, less than 6, less than 8, less than 10, less than 12, less than 14, less than 16, less than 18, less than 20, less than 25, less than 30, less than 40, less than 50, or less than 100.

In some embodiments, the solute is precipitated from the ionic liquid. In some cases, the solute comprises sugar and/or a furanic compound. In some cases, the solute comprises lignin, ash and/or protein.

### Continuous Recovery

Methods for hydrolyzing biomass and methods for recovering biomass components from ionic liquids are described herein. Performing hydrolysis and recovering continuously can have certain advantages including recovery of high quality biomass components (e.g., at a high concentration and/or with few breakdown products).

In some embodiments, provided herein is a method for producing fermentable sugar. The method comprises hydrolyzing a polysaccharide in an ionic liquid to produce sugar and continuously removing the sugar from the ionic liquid. In some embodiments, the rate of sugar removal from the ionic liquid is approximately equal to the rate of sugar production. The sugar may be continuously removed by extraction in a supercritical or near-supercritical fluid for example.

The concentration of furanic compounds can be any concentration. In some embodiments, the sugars contain little furanic compounds. In some cases, the sugar is fermentable when removed from the ionic liquid. In some embodiments, the mass of furanic compounds in the sugar is about 30%, about 20%, about 10%, about 5%, about 3%, about 1%, about 0.5%, or about 0.1% of the mass of sugar produced in the ionic liquid. In some embodiments, the mass of furanic compounds in the sugar is at most 30%, at most 20%, at most 10%, at most 5%, at most 3%, at most 1%, at most 0.5%, or at most 0.1% of the mass of sugar produced in the ionic liquid.

In some embodiments, the sugar is removed from the ionic liquid at an optionally variable rate such that the mass of furanic compounds produced is about 30%, about 20%, about 10%, about 5%, about 3%, about 1%, about 0.5%, or about 0.1% of the mass of sugar produced in the ionic liquid. In some embodiments, the sugar is removed from the ionic liquid at an optionally variable rate such that the mass of furanic compounds produced is less than 30%, less than 20%, less than 10%, less than 5%, less than 3%, less than 1%, less than 0.5%, or less than 0.1% of the mass of sugar produced in the ionic liquid.

The hydrolysis reaction can break glycosidic bonds and/or decrease the degree of polymerization of the polysaccharide. Supercritical and near-supercritical fluids can extract smaller molecules from ionic liquids more efficiently than larger molecules in some instances. Coupling hydrolysis with sugar recovery by fluid extraction (e.g., supercritical and near-supercritical fluids) separates sugars (e.g., monosaccharides, disaccharides, small oligosaccharides up to about 3, 4, 5, or 6 sugar units) from larger polysaccharides in some instances. The polysaccharides can remain in the hydrolysis reaction and/or be returned to the hydrolysis reaction until the degree of polymerization is reduced to such an extent that the hydrolysate (e.g., sugars) become extractable in the fluid. In some embodiments, the product is continuously separated from the reactant (e.g., sugars from polysaccharides).

In some embodiments, the hydrolysis reaction is cooled. Provided herein is a method for producing fermentable sugar comprising hydrolyzing a polysaccharide in an ionic liquid to produce sugar and continuously cooling and/or lowering the temperature of the hydrolysate. The hydrolysate can be cooled such that a low concentration of furanic compounds are formed for example.

### Furanic Compounds

In some cases, production of furanic compounds is desired. Furanic compounds are considered to be biomass components and biomass derivatives. The composition comprising a furanic compound can be produced by contacting an ionic liquid with a biomass, a polysaccharide, a sugar, or a combination thereof. A method for producing furanic compounds from biomass is described in U.S. Patent Pub. No. 2010/0004437, which is herein incorporated by reference in its entirety. In some embodiments, the ionic liquid further comprises a catalyst. In some embodiments, the catalyst dehydrates the sugar (e.g., to a furanic compound). In some cases, the catalyst is CrCl₃. The furanic compound can be, but is not limited to hydroxymethylfurfural, 2,5-dimethylfuran, furfural, or a combination thereof.

In some embodiments, provided herein is a method for recovering a furanic compound from an ionic liquid comprising contacting a composition comprising a furanic compound and an ionic liquid with a fluid. In various embodiments, the fluid is a pressurized gas, liquefied gas, or supercritical or near-supercritical fluid. In some instances, the furanic compound is extracted in the supercritical or near-supercritical fluid.

In some embodiments, contacting the ionic liquid with a fluid forms a first phase comprising the ionic liquid and a second phase comprising the furanic compound and the furanic compound is recovered from the ionic liquid by partitioning the second phase from the first phase. In some cases, at least 90% of the ionic liquid is in the first phase and at least 90% of the furanic compound is in the second phase.

In some embodiments, the ionic liquid comprises water, contact with the fluid creates an aqueous or organic phase, and the furanic compound is recovered in the aqueous or organic phase.

### Ionic Liquid Manufacture and Purification

The methods described herein are not limited to processing of biomass and/or recovery of biomass components from ionic liquids. The methods can be used to remove any solute from an ionic liquid (e.g., increase the purity of the ionic liquid). In some embodiments, the methods are used in the manufacture and/or purification of ionic liquids.

In some embodiments, provided herein is a method for manufacturing or purifying an ionic liquid, comprising removing non-ionic components from the ionic liquid by contacting the ionic liquid with a pressurized gas. In some embodiments, the pressurized gas is carbon dioxide. In some embodiments, the pressurized gas is a supercritical or near-supercritical fluid. The non-ionic component can be any compound that is not charged. The non-ionic component can be polar. Water is an example of a non-ionic component that can be removed using the methods described herein.

The ionic liquid can be manufactured in any suitable way. In some embodiments, the ionic liquid is synthesized by mixing ionic components (optionally comprising non-ionic impurities) prior to removing non-ionic components from the ionic liquid. In some embodiments, the ionic liquid is synthesized by creating ionic components in a reaction prior to removing non-ionic components from the ionic liquid. The reaction can generate non-ionic by-products, non-ionic components may be impurities in the reactants, non-reacted reactants can be non-ionic components, and the like.

The ionic liquid can be synthesized in any suitable reactor, optionally in a microreactor. The ionic liquid can be synthesized from any suitable starting materials. In one example, a base is reacted with an alkylating agent in a quaternization reaction, which is then reacted with a molecule that serves as an anion source in a metathesis reaction.

### In Situ Synthesis of Ionic Liquid

In some cases, the ionic liquid is synthesized from precursors in the process. In some instances, the synthesis does not require a dedicated ionic liquid synthesis reactor. The synthesis can utilize a supercritical or near-critical CO₂ phase to synthesize ionic liquid in a near atom-efficient reaction. In general, any stream that consists of mostly ionic liquid (e.g., at least 90% by mass), but is mostly devoid of biomass components or products (e.g., less than 5% by mass) can be suitable for performing ionic liquid synthesis. One such stream can be the recycle return of ionic liquid to the hydrolysis reactor. Here, a minor amount of ionic liquid feedstock (e.g., 1-methyl imidazole and 1-chlorobutane) can be introduced into the main stream and allowed to react for an adequate amount of time. The heat evolved during the reaction can be absorbed by the media and used to heat the ionic liquid in preparation for dissolution and hydrolysis. In this way, ionic liquid can be replaced with negligible investment in either capital or operating costs besides the cost of feedstocks.

### Recovered Biomass Components

The biomass components recovered from the ionic liquids are relatively clean, pure and/or concentrated in some embodiments. The invention includes the biomass components produced by any of the methods described herein.

Sugars are one example of a biomass component. The sugar can include, but is not limited to glucose, xylose, mannose, or a combination thereof. In some cases, the sugars are recovered from the ionic liquid as a solution (e.g., dissolved in a solvent such as water). In some embodiments, described herein is a sugar composition comprising water and a sugar, wherein the sugar is derived from cellulose, hemicellulose, or a combination thereof. The sugar can further comprise carbon dioxide and/or ionic liquid.

The sugar composition can comprise any concentration of carbon dioxide (e.g., at any detectable concentration). In some instances, the concentration of carbon dioxide is about 0.0001%, about 0.0005%, about 0.001%, about 0.005%, about 0.01%, about 0.05%, about 0.1%, or about 0.5% by mass. In some instances, the concentration of ionic liquid is less than 0.0001%, less than 0.0005%, less than 0.001%, less than 0.005%, less than 0.01%, less than 0.05%, less than 0.1%, less than 0.5% by mass.

In some embodiments, the sugar composition comprises ionic liquid (e.g., at any detectable concentration). In some instances, the concentration of ionic liquid is about 0.001%, about 0.005%, about 0.01%, about 0.05%, about 0.1%, about 0.5%, about 1%, or about 5% by mass. In some instances, the concentration of ionic liquid is less than 0.001%, less than 0.005%, less than 0.01%, less than 0.05%, less than 0.1%, less than 0.5%, less than 1%, or less than 5% by mass.

In some embodiments, provided herein is a fermentable sugar comprising a sugar and an ionic liquid, wherein the sugar is derived from cellulose, hemicellulose, or a combination thereof. In some embodiments, the ionic liquid is detectable and the mass of sugar is at least 5 times, at least 10 times, at least 20 times, at least 50 times, at least 100 times, at least 1000 times, at least 10000 times, or at least 100000 times greater than the mass of the ionic liquid. In some cases, the sugar is fermentable.

In some embodiments, the sugar comprises at least one component selected from furanics, phenols, ethers, aldehydes, ash, lignin, and lignin derivatives. In some embodiments, the concentration of the furanics, phenols, ethers, aldehydes, ash, lignin, and lignin derivatives, or any combination thereof is less than 10%, less than 5%, less than 1%, less than 0.5%, less than 0.1%, less than 0.05%, or less than 0.01% by mass (w/w).

Oils are one example of a biomass component. The oils can include, but are not limited to terpenes, tall oils, lipids, triglycerides, or any combination thereof. In some cases, the oils are recovered from the ionic liquid. In some embodiments, described herein is an oil comprising carbon dioxide and/or ionic liquid, wherein the oil is derived from biomass.

The oil can comprise any concentration of carbon dioxide (e.g., at any detectable concentration). In some instances, the concentration of carbon dioxide is about 0.0001%, about 0.0005%, about 0.001%, about 0.005%, about 0.01%, about 0.05%, about 0.1%, or about 0.5% by mass. In some instances, the concentration of ionic liquid is less than 0.0001%, less than 0.0005%, less than 0.001%, less than 0.005%, less than 0.01%, less than 0.05%, less than 0.1%, less than 0.5% by mass.

In some embodiments, the oil comprises ionic liquid (e.g., at any detectable concentration). In some instances, the concentration of ionic liquid is about 0.001%, about 0.005%, about 0.01%, about 0.05%, about 0.1%, about 0.5%, about 1%, or about 5% by mass. In some instances, the concentration of ionic liquid is less than 0.001%, less than 0.005%, less than 0.01%, less than 0.05%, less than 0.1%, less than 0.5%, less than 1%, or less than 5% by mass.

Also encompassed within the invention are the nucleic acids, proteins, lipids, fatty acids, resin acids, waxes, terpenes, acetates (e.g., ethyl acetate, methyl acetate), carbohydrates, cellulose, hemicellulose, alcohols, sugars, sugar acids, glucose, fructose, xylose, galactose, arabinose, mannose, rhamnose, mannuronic acid, galacturonic acid, lignin, alcohols (e.g., methanol, ethanol), phenols, aldehydes, ethers, p-coumaryl alcohol, coniferyl alcohol, sinapyl alcohol, pectin, D-galacturonic acid, amino acids, acetic acid, ash, any derivative thereof (e.g., furanic compounds), or any combination thereof produced by the methods described herein.

### Recovery of Concentrated Components

In some embodiments, recovery of solutes from ionic liquids using chromatography results in an ionic liquid that is diluted (e.g., in water). In contrast, in some embodiments, the process described herein does not dilute the ionic liquid. That is, the process (e.g., separation of biomass components from an ionic liquid) does not comprise a step of concentrating the ionic liquid (e.g., by evaporating water from the ionic liquid).

In some embodiments, a method is described for separating a hydrogen bonding solute from a composition comprising an ionic liquid and a hydrogen bonding solute, wherein the concentration of the ionic liquid decreases by less than 100%, less than 50%, less than 20%, less than 10%, or less than 5% when the hydrogen bonding solute is separated from the composition. In some embodiments, the concentration of the ionic liquid decrease less than 5% to less than 100%, less than 10% to less than 50%, or less than 20% to less than 50% when the hydrogen bonding solute is separated from the composition. In some embodiments, the concentration of the ionic liquid in the composition is decreased by the addition of a solvent (e.g., water, ethanol) to the composition when the hydrogen bonding solute is recovered from the ionic liquid. The amount of solvent added to the composition is low in some cases.

In some embodiments, a method is described for separating a hydrogen bonding solute from a composition comprising an ionic liquid and a hydrogen bonding solute, wherein the concentration of the ionic liquid increases when the hydrogen bonding solute is separated from the composition. In some cases, water is separated from the composition along with the hydrogen bonding solute. The concentration of the ionic liquid can increase by any suitable percentage. In some instances, the concentration of the ionic liquid is increased by at least 1%, at least 3%, at least 5%, at least 10%, at least 20%, at least 30%, at least 50%, or at least 70%.

In some embodiments, chromatography dilutes the solute when recovered from the ionic liquid (i.e., the concentration of the solute in the ionic liquid is greater than the concentration of the solute when recovered). In contrast, in some embodiments, the process described herein results in a concentrated solute.

In some embodiments, a method is described for separating water and a hydrogen bonding solute from a composition comprising an ionic liquid, water and hydrogen bonding solute, wherein the ratio of the mass of water to the mass of hydrogen bonding solute when separated is approximately equal to the ratio of the mass of water to the mass of hydrogen bonding solute in the composition.

For example, the composition can contain 80% ionic liquid, 10% water and 10% hydrogen bonding solute. Here, the ratio of the mass of water to the mass of hydrogen bonding solute in the composition is 1.0. If, for example, a separated composition comprising 50% water and 50% hydrogen bonding solute is separated from the ionic liquid, the ratio of 1.0 is preserved. In this example, the ratio (of 1.0) is equal.

In some embodiments, the ratio of the mass of water to the mass of hydrogen bonding solute when separated is within about 5%, within about 10%, within about 20%, within about 30%, or within about 50% of the ratio of the mass of water to the mass of hydrogen bonding solute in the composition. In some embodiments, the mass of hydrogen bonding solute when separated is within about 5% to about 50%, about 10% to about 50%. about 20% to about 50%, or about 20% to about 30% of the ratio of the mass of water to the mass of hydrogen bonding solute in the composition.

In some embodiments, the hydrogen bonding solute is recovered from the ionic liquid in a concentrated solution. They recovered hydrogen bonding solute does not require any concentration steps (e.g., evaporation or distillation) in some instances.

In some embodiments, a method is described for separating water and hydrogen bonding solute from a composition comprising an ionic liquid, water and hydrogen bonding solute, wherein the hydrogen bonding solute is separated from the ionic liquid at a concentration of at least 5%, at least 10%, at least 20%, at least 30%, at least 40%, at least 50%, at least 60%, at least 70%, or at least 80% (w/w). In some embodiments, the hydrogen bonding solute is separated from the ionic liquid at a concentration of at least 5% to at least 80%, at least 10% to at least 70%, at least 20% to at least 60%, at least 30% to at least 50%, or least 40% to at least 50%.

In some embodiments, the hydrogen bonding solute is derived from biomass. A hydrogen bonding solute is any molecule capable of forming one or more hydrogen bonds. In some cases, the hydrogen bonding solute is capable of forming one or more hydrogen bonds with an ionic liquid and/or water. The hydrogen bonding solute can have at least one hydroxyl group. In various embodiments, the hydrogen bonding solute can be a carbohydrate, a sugar, an aldose, a ketose, or any combination thereof. In some cases, the hydrogen bonding solute is derived from biomass. In some cases, the hydrogen bonding solute is a carbohydrate (e.g., glucose, xylose, mannose, or galactose). In some cases, the hydrogen bonding solute is an alcohol (e.g., ethanol or methanol).

In some instances, the composition comprises the ionic liquid and a furanic compound and the furanic compound is separated from the ionic liquid at a concentration of at least 10% (w/w).

### Certain Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

The term “invention” or “present invention” as used herein is not meant to be limiting to any one specific embodiment of the invention but applies generally to any and all embodiments of the invention as described in the claims and specification.

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure.

### EXAMPLES

### Example 1

### Recovery of Glucose with Carbon Dioxide

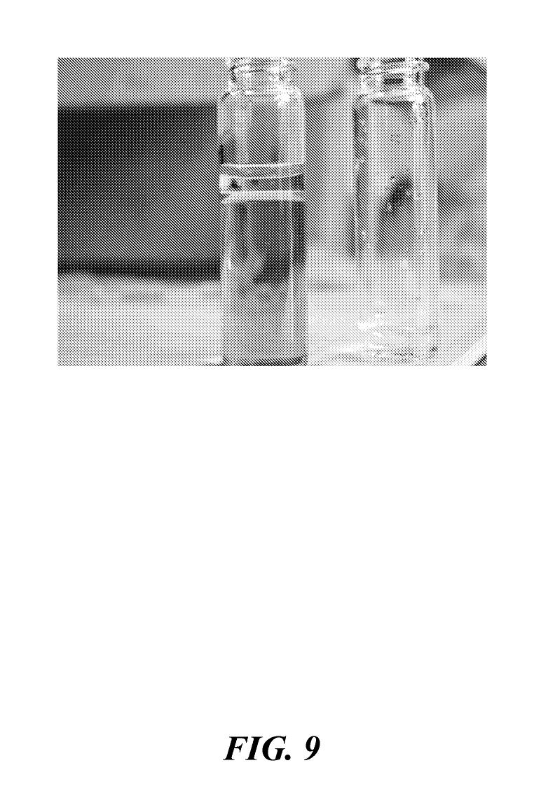

A 10 ml sample containing 85% ionic liquid 1-Butyl-3-methylimidazolium chloride, 10% water, and 5% glucose by mass was prepared. The solution was placed in a pressure vessel and pressurized using supercritical carbon dioxide at 2000 psi and 40° C. and left under these conditions for 15 minutes. Carbon dioxide was then flowed through the pressure vessel at 5 standard liters per minute for 10 minutes. Water was added to the carbon dioxide stream as a co-solvent at a rate of 2 ml/min. The carbon dioxide leaving the vessel was depressurized and vented while a minimal amount of extract from the ionic liquid-water-glucose solution was captured in a collection vial whereupon the carbon dioxide was shut-off and the pressure vessel was depressurized. FIG. 9 shows the sample with a clear phase on top shortly after removal from the pressure vessel.

A Bayer Breeze 2 Glucose Meter measured 529 mg/dL glucose in the clear phase immediately after extraction. The liquid captured from the depressurized extract stream was also measured immediately with no detectible levels of glucose found. This sample was then dried in an oven over several hours leaving observable deposits and particulates. Then, 0.03 mL of DI water was added to the dried sample and tested for glucose giving a reading of 49 mg/dL.

### Example 2

### Recovery of Glucose with Carbon Dioxide

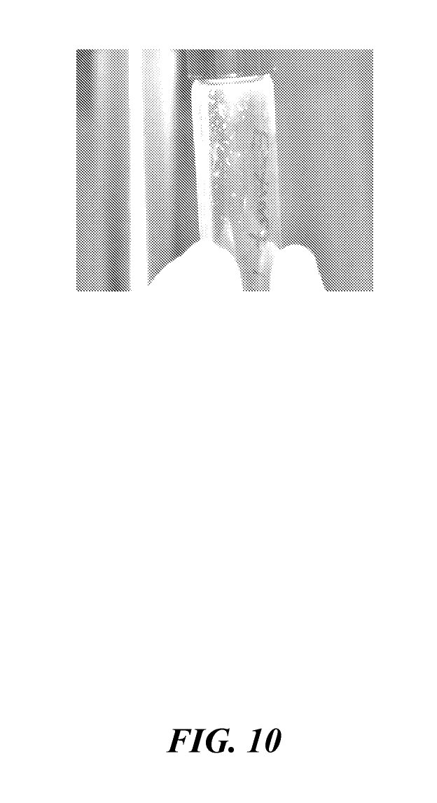

A 13 ml sample containing 80% ionic liquid 1-Butyl-3-methylimidazolium chloride, 10% water, and 10% glucose by mass was prepared. The solution was placed in a pressure vessel and pressurized using supercritical carbon dioxide at 3000 psi and 40° C. and left under these conditions for 17 minutes. Carbon dioxide was then flowed through the pressure vessel at 3.5 standard liters per minute for 39 minutes. Water was added to the carbon dioxide stream as a co-solvent at a rate of 0.75 ml/min. The carbon dioxide leaving the vessel was depressurized and vented while extract from the ionic liquid-water-glucose solution was captured in a collection vial. The collected extract was whitish in color. The collected Extract is shown in FIG. 10.

### Example 3

### Recovery of Glucose with Carbon Dioxide

A 13 ml sample containing 80% ionic liquid 1-Butyl-3-methylimidazolium chloride, 10% water, and 10% glucose by mass was prepared. The solution was placed in a pressure vessel and pressurized using supercritical carbon dioxide at 3000 psi and 40° C. Carbon dioxide was then flowed through the pressure vessel at 3.5 standard liters per minute. Water was added to the carbon dioxide stream as a co-solvent at a rate of 2.0 ml/min. The carbon dioxide leaving the vessel was depressurized and vented while extract from the ionic liquid-water-glucose solution was captured in a collection vial.

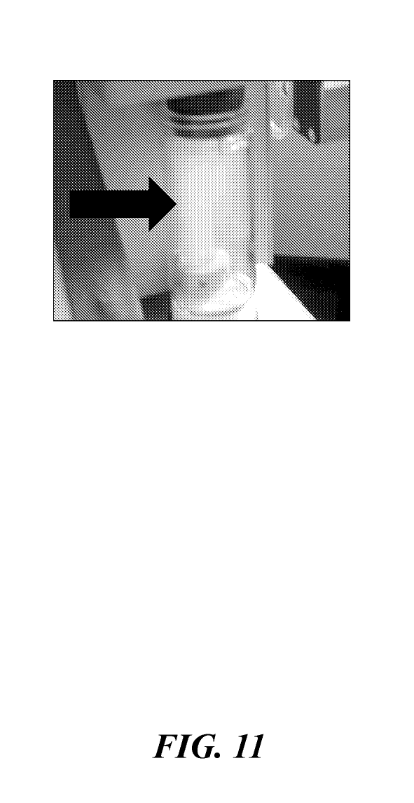

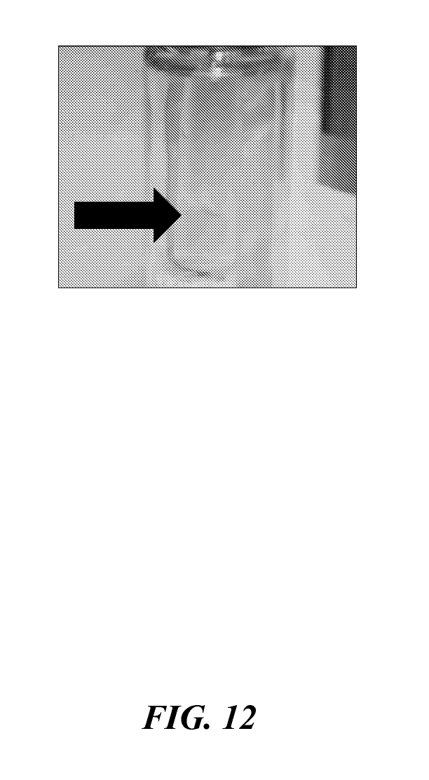

After 25 minutes of extraction, the collection vessel began to fog up with vapor. FIG. 11 shows an image of the collection vessel filling with vapor. Shortly after the collection vessel filled with vapor, liquid extract was captured. FIG. 12 shows collected liquid extract during the experiment.

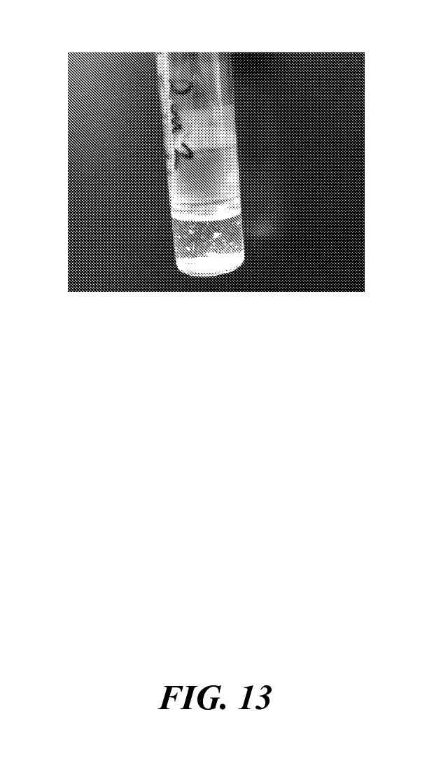

The collected liquid extract was concentrated by vacuum drying. While being concentrated under vacuum, small amounts of a very fine precipitate formed. FIG. 13 shows these particulates. The collected extract continued to concentrate under vacuum until visibly dry.

After drying, approximately 0.05 mL of water was added to the dried extract. The sample was tested for glucose using a Bayer Breeze 2 Glucose Meter which detected glucose giving a reading of “High”. An additional half milliliter of water was added to the sample and retested giving a reading of 173 mg/dL.

### Example 4

### Recovery of Glucose from an Ionic Liquid in an Aqueous Phase

An 11.5 mL solution of 90% ionic liquid 1-Butyl-3-methylimidazolium chloride and 10% glucose by weight was prepared. The sample was warmed and shaken until the glucose was visibly dissolved.

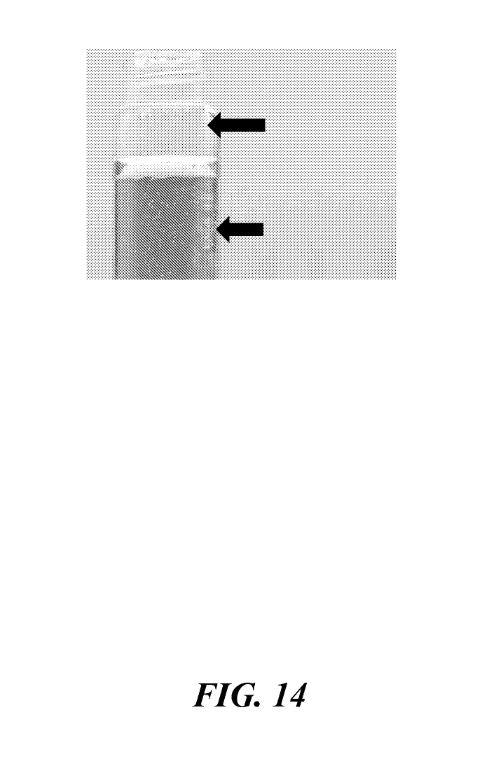

3.5 ml of water was added to the top of the sample with care taken to maintain an ionic liquid-water-glucose phase on the bottom and a visually immiscible water phase on top. FIG. 14 shows a representative illustration of the phase behavior where the immiscible top phase of water and bottom phase of ionic liquid-glucose solution are present.

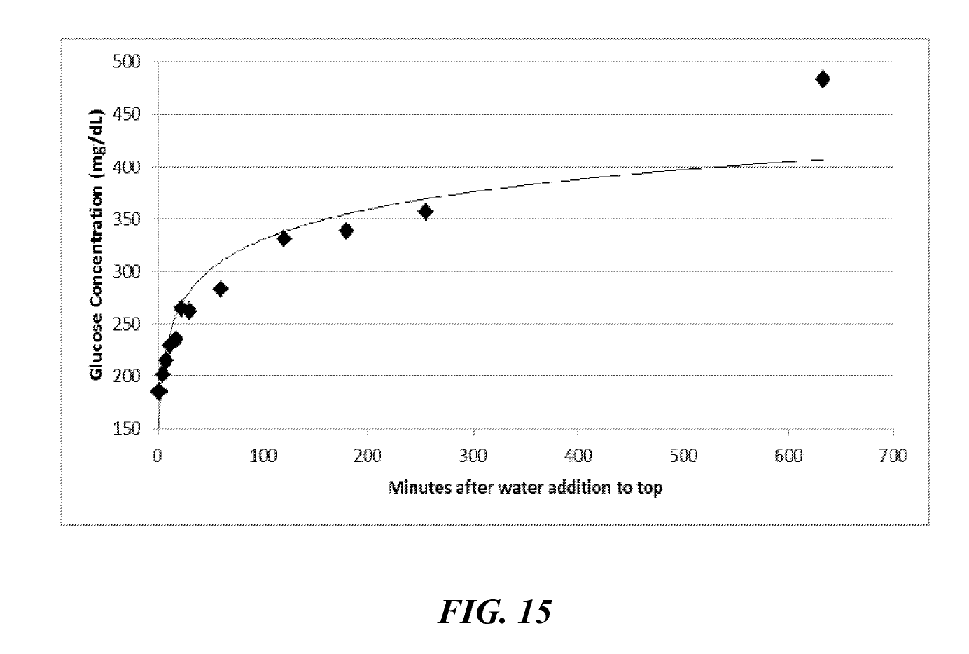

Using a Bayer Breeze 2 Glucose Meter the top of the water phase was immediately measured for glucose. The initial reading was 185 mg/dL glucose. Additional readings were taken every few minutes for an hour and then every few hours for eleven hours. After an initial and rapid increase in glucose concentration the glucose concentration in the water phase began to stabilize at approximately 300 minutes. FIG. 15 shows the glucose concentration in the water phase over time when added on top of an ionic liquid-glucose solution.

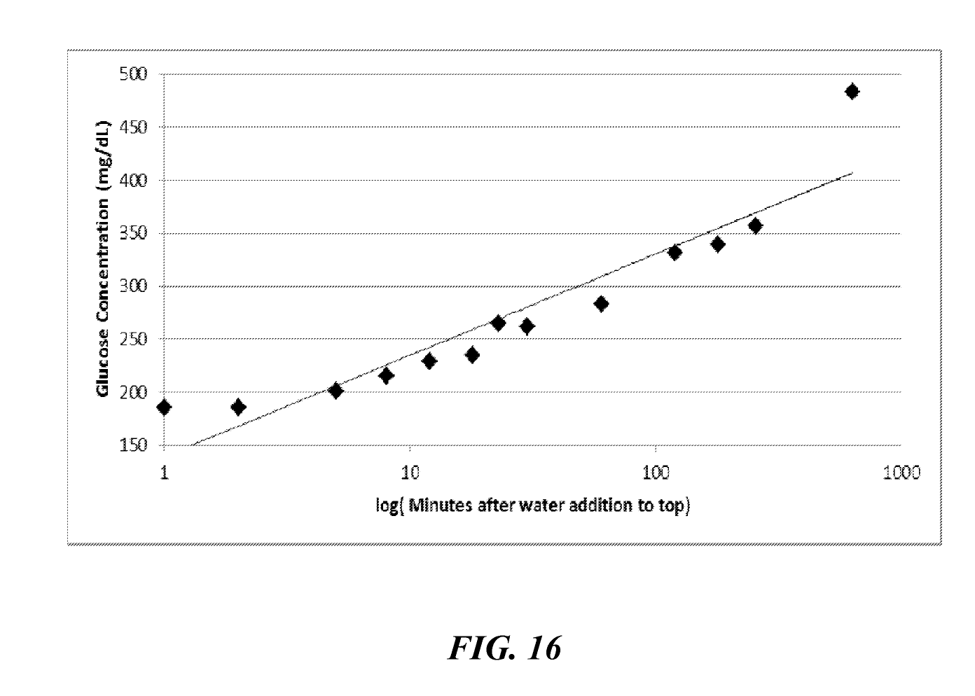

These same data were plotted on a log scale for time and show a very good semi-log fit between 3 and 300 minutes. FIG. 16 shows the glucose concentration in the water phase over the logarithmic time from when water was added on top of the liquid-glucose solution.

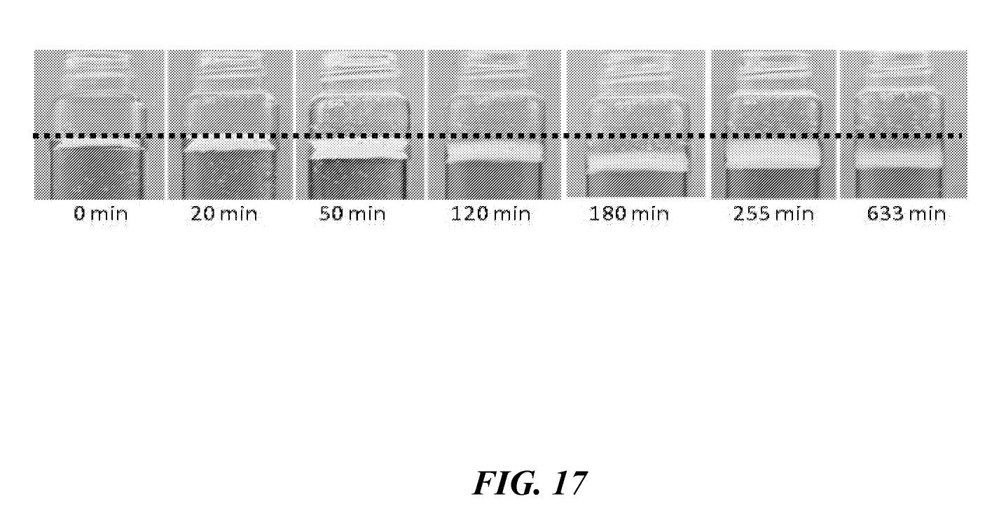

Throughout the experiment it was also observed that the distinct meniscus separating the ionic liquid-glucose bottom phase and the added water top phase gradually became blurred; however the water phase appeared generally unchanged on the top, and the ionic liquid-glucose region blurred by water generally appeared to remain at the same level of the original meniscus. FIG. 17 shows this phenomenon over time.

### Example 5

### Recovery of Glucose from an Ionic Liquid in an Aqueous Phase

Four solutions of 90% ionic liquid 1-Butyl-3-methylimidazolium chloride and 10% water by weight were prepared. Increasing amounts of glucose were added to each sample such that the four samples contained 0%, 3.3%, 6.6%, and 10% glucose. The samples were warmed and shaken until the glucose was visibly dissolved.

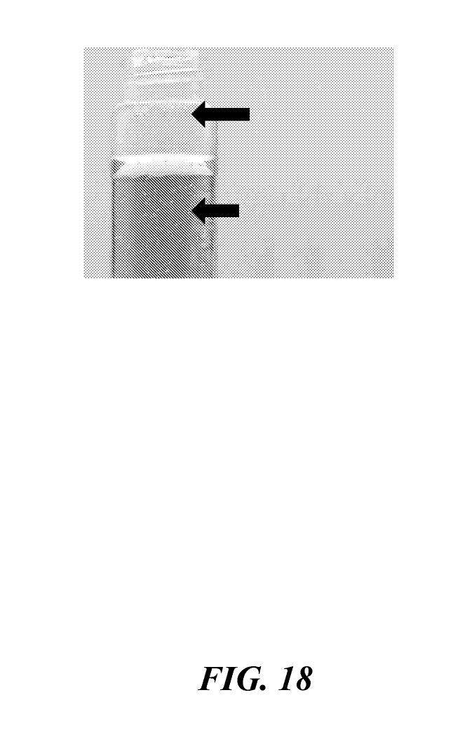

5 ml of water was added to the top of each sample with care taken to maintain an ionic liquid-water-glucose phase on the bottom and a visually immiscible water phase on top. FIG. 18 shows a representative illustration of the phase behavior where the immiscible top phase of water and bottom phase of ionic liquid-water-glucose solution are present.

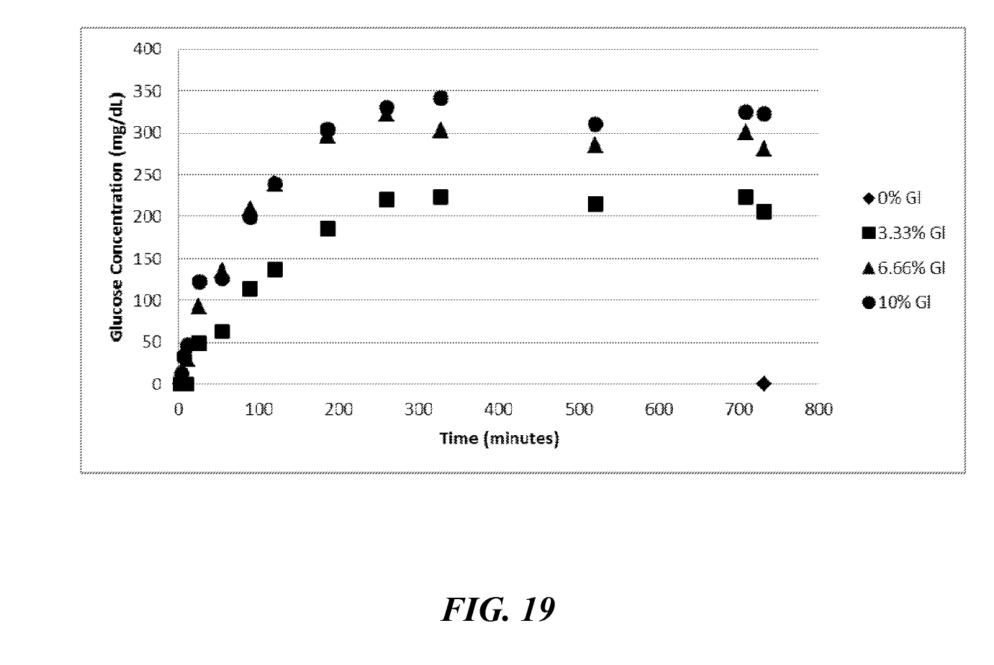

Using a Bayer Breeze 2 Glucose Meter the top of the water phase was measured for glucose. Glucose measurements were taken in this manner during a period of 12 hours. FIG. 19 shows the glucose concentration in the water phase for the four samples prepared containing varying amounts of glucose. After approximately 300 minutes, the increasing glucose migrating into the water phase tapered off.

After 12 hours, 2 ml of the added water phase on top of the ionic liquid-water-glucose solution was removed, diluted, and measured for conductivity using an Extech Instruments ExStik II Conductivity/TDS/Salinity Meter.

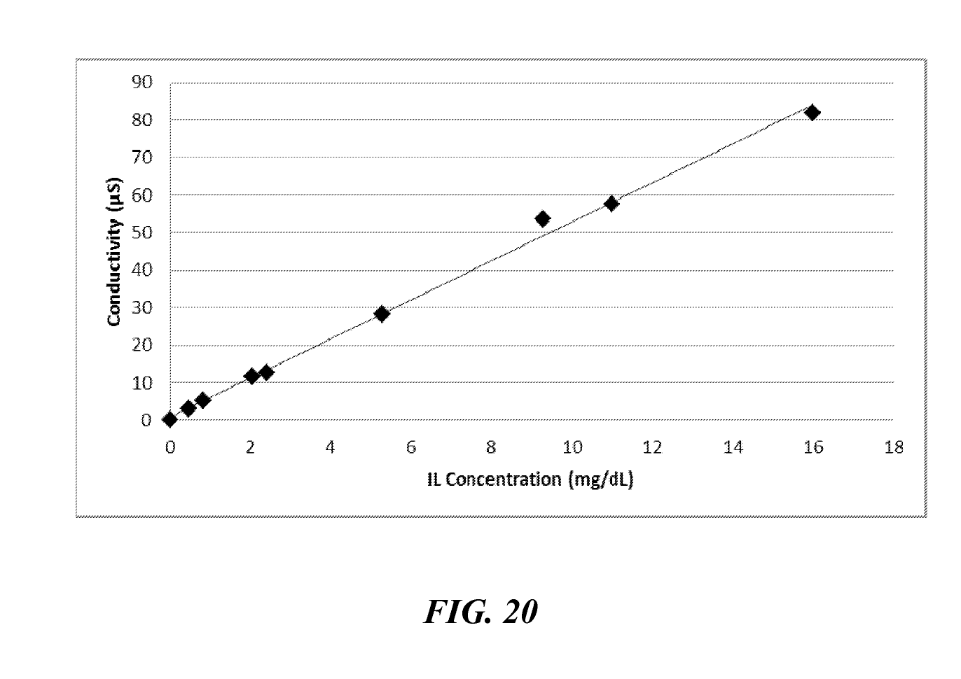

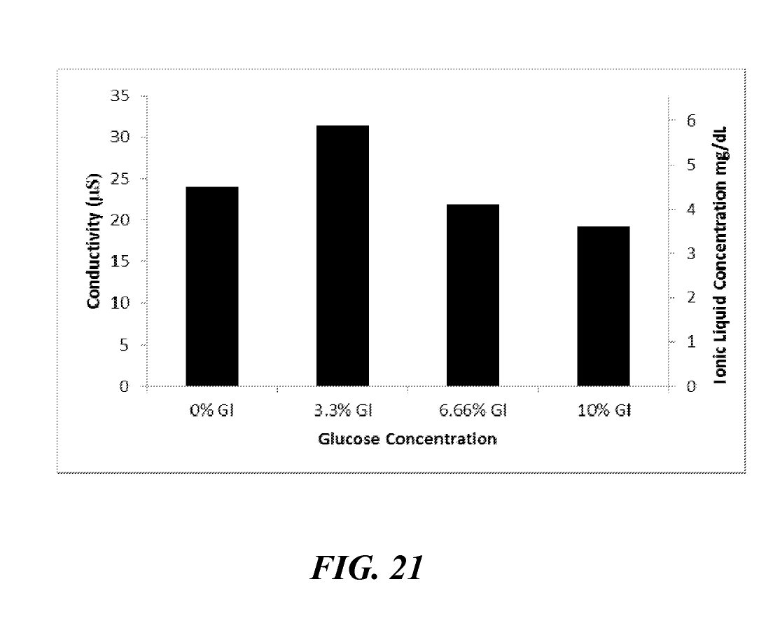

Conductivity was correlated to an ionic liquid concentration from experimental data measuring the conductivity of an ionic liquid solution prepared at various concentrations in deionized water. FIG. 20 shows the relationship of ionic liquid concentration to conductivity. FIG. 21 shows the conductivity and corresponding ionic liquid concentration of the added water phase after 12 hours.

The ionic liquid found in the water phase was approximately 5 mg/dL. For 5 mg/dL of ionic liquid from the ionic liquid-water-glucose solution to dissolve into the added water phase, less than 0.4 mg/dL glucose could also be present. The measured glucose was found to be far greater at approximately 300 mg/dL glucose. The 300 mg/dL glucose indicates that glucose migrated from the ionic liquid-water-glucose phase into the water phase at a rate much greater than expected 0.4 mg/dL glucose simply due from the ionic liquid-water-glucose solution dissolving directly into the water phase.

### Example 6

### Stabilization of an Aqueous Phase with Glucose

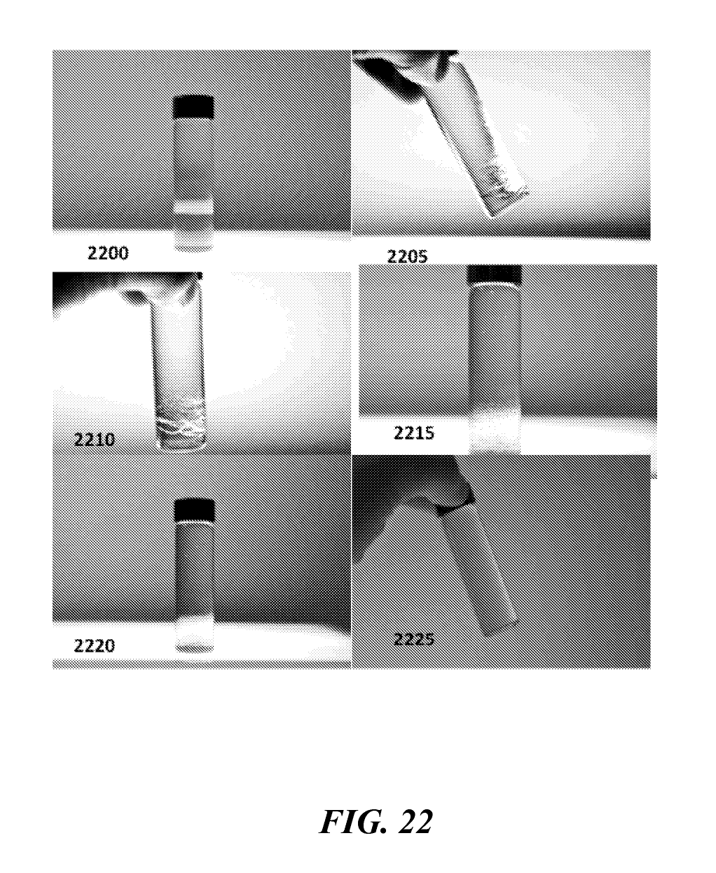

An 8 ml solution of 90% ionic liquid 1-Butyl-3-methylimidazolium chloride, 5% glucose and 10% water by weight was prepared. Eight (8) ml of water was added to the top of the sample with care taken to maintain an ionic liquid-water-glucose phase on the bottom and a visually immiscible water phase on top. The two phase sample was left to sit for several days. Although the distinct meniscus separating the two phases gradually blurred, the top portion of the sample vial remained clear with a darkening yellow gradient toward the bottom portion of the vial. FIG. 22 shows the undisturbed sample after a few days where the meniscus is beginning to blur 2200.

The sample vial was then gently rotated about a horizontal axis allowing the clear top and yellow bottom to mix. During this rotation and mixing, the clear phase and yellow phase did not appear to readily blend to a uniform material. In-homogeneous clear and yellow vanes were visibly apparent. FIG. 22 depicts this inhomogeneous mixing (2205 and 2210). Upon returning to its original orientation the top portion of the vial generally remained clear and the bottom portion of the vial generally remained yellow. The vial was continually rotated about a horizontal axis for several minutes and this same phenomenon was observed.

The sample was then rapidly shaken for approximately 10 seconds. A similar phenomenon of in-homogenous mixing with vanes of different colors was observed although the vanes were generally finer and shorter. FIG. 22 shows these finer and shorter vanes (2215 and 2220).

The sample was then rapidly and violently shaken several times over several hours whereupon the sample vial eventually took on a uniform homogenous appearance with no color gradients or vanes of apparently different composition. FIG. 22 shows the vial after this vigorous mixing over time with a visually uniform composition 2225.

### Example 7

### Use of Lignin-Dissolving Ionic Liquids

The methods described herein can be used with any ionic liquid that dissolves any biomass or biomass component (e.g., cellulose or lignin). In some cases, ionic liquids are used that dissolve lignin. In some cases, the ionic liquids do not dissolve cellulose. Examples of lignin-dissolving ionic liquids are described in PCT Patent Publication Number WO2012/080702, which is hereby incorporated by reference in its entirety.

The lignin-dissolving ionic liquid can be formed from the reaction of an amine (e.g., tri-ethylamine) with sulfuric acid. In some cases, the ionic liquid comprises a cation (e.g., a protic cation) and an anion selected from C₁₋₂₀ alkyl sulfate [AlkylSO₄]⁻, C₁₋₂₀alkylsulfonate [AlkylSO₃]⁻, hydrogen sulfate [HSO₄]⁻, hydrogen sulphite [HSO₃]⁻, dihydrogen phosphate [H₂PO₄]⁻, hydrogen phosphate [HPO4]²⁻ and acetate, [CH₃CO₂]⁻. The lignin-dissolving ionic liquid can contain water (e.g., 10-40% v/v water when the anion is acetate).

Any biomass component, including but not limited to lignin, lignin fragments, lignin derivatives, hemicellulose, hemicellulose hydrolysate (e.g., xylose), cellulose, cellulose hydrolysate (e.g., glucose), acetate, and ash can be removed from the lignin-dissolving ionic liquids using the methods described herein. Non-limiting methods including the formation of an aqueous biphasic system and removal of the biomass components in the aqueous phase. Aqueous bi-phasic systems can be formed by contact with fluids (e.g., pressurized CO₂), addition of kosmotropes, or change of pH for example.

Methods described herein (e.g., formation of ABS) may be used for the extraction of sugars (and precipitation of lignin and other solutes) from the lignin-dissolving ionic liquid. Formation of a two-phase system rich in water and ionic liquid can separate water-soluble lignin fragments and other components such as acetate, which enrich in the water layer, from larger lignin fragments, which enrich in the ionic liquid layer. These methods can also remove water from ionic liquid, which can be an important step for keeping a suitable water concentration during the various steps of the process.

In-situ hydrolysis of cellulose and/or hemi-cellulose (e.g., by gradual addition of water) can be performed in the lignin-dissolving ionic liquid. Furthermore, if the lignin-dissolving ionic liquid is a protic ionic liquid, the amount of acid required for in situ hydrolysis can be much decreased relative to aprotic ionic liquids.

Methods described herein for the removal of lignin and recovery of ionic liquid can be used with lignin-dissolving ionic liquids. For example, lignin can be precipitated by applying a pressure of a gas with good solubility in the lignin-dissolving ionic liquid (e.g., CO₂).

### Example 8

### Use of Two Ionic Liquids

The methods can be used with more than one ionic liquid, either as a mixture of two or more ionic liquids, or with different ionic liquids used in different portions of the process.

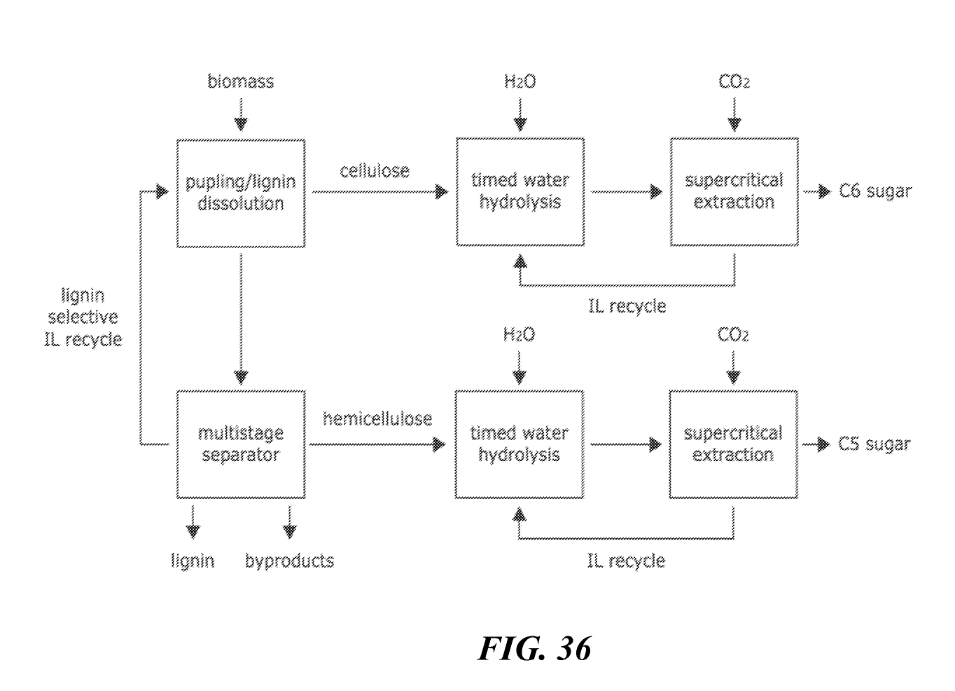

FIG. 36 shows an example of a process where the biomass is initially contacted with a lignin-dissolving (lignin selective) ionic liquid. The cellulose can be separated from the lignin-dissolving ionic liquid. In some cases, the cellulose is hydrolyzed with cellulase enzymes. As shown in FIG. 36, the cellulose can be hydrolyzed in an ionic liquid that dissolves the cellulose. Water can be added to the cellulose hydrolysis reaction as described herein (i.e., timed water hydrolysis). Sugars from the hydrolysis (e.g., C6 sugar) of cellulose can be recovered by aqueous biphasic system formation or supercritical fluid extraction. The cellulose-dissolving ionic liquid (e.g., [BMIM]Cl) can be recycled.

Returning attention to the lignin-dissolving ionic liquid of FIG. 36, lignin, lignin fragments, lignin derivatives, C5 sugars (e.g., xylose), or any other solute or non-dissolved material can be recovered from the lignin-dissolving ionic liquid. In some cases, this is by aqueous biphasic system formation as described herein.

Hemicellulose and its derivatives (e.g., C5 sugars) can become dissolved in the lignin-dissolving ionic liquid and/or the cellulose-dissolving ionic liquid. In some cases, the cellulose is hydrolyzed in the lignin-dissolving ionic liquid as shown in FIG. 36 (with or without timed water addition). The lignin-dissolving ionic liquid can be recycled to contact with the biomass.

In some cases, the lignin-dissolving ionic liquid is significantly cheaper than the cellulose-dissolving ionic liquid (e.g., 5-fold or 10-fold less expensive). In such an instance, the process shown in FIG. 36 that uses a cheaper lignin-dissolving ionic liquid initially, followed by hydrolysis of the cellulose in a separate ionic liquid can be more economical than using only a cellulose-dissolving ionic liquid. Introducing cellulose rather than ligno-cellulose into the cellulose-dissolving ionic liquid can result in higher reaction rates, higher sugar yields, require fewer process steps (e.g., eliminate the requirement of solids removal), and result in a cleaner ionic liquid recycle and less ionic liquid loss in the process loop.

When using a lignin-dissolving ionic liquid followed by a cellulose-dissolving ionic liquid, the cellulose hydrolysis reactor can require a shorter dissolution step, as the lack of lignin can improve dissolution rates. Hydrolysis may also require fewer steps (e.g., one, instead of two). The extraction of sugars from the cellulose-dissolving ionic liquid loop can be simplified due to the relative-absence of solutes other than sugars. In some cases, all steps related to the removal of lignin and other solids are eliminated.

Solids and ash may still need to be recovered from an ionic liquid when using two ionic liquids, but doing so from a less costly lignin-dissolving ionic liquid may be advantageous. In some cases, two ionic liquids can be used to result in separate C5 and C6 sugar streams, rather than a mixture of C5 and C6 sugars. Hydrolyzing the cellulose and hemi-cellulose separately as shown in FIG. 36, can allow uncoupled optimizations where the C5 hydrolysis has different operating conditions than the C6 hydrolysis.

When using two ionic liquids separately as shown in FIG. 36, the cellulose can be washed of the lignin-dissolving ionic liquid such that the cellulose-dissolving ionic liquid does not become contaminated with the lignin-dissolving ionic liquid.

### Example 9

### Use of Algicide as an Example of an Ionic Liquid

Benzalkonium chloride (structure shown below) is an algicide that has been on the consumer market for quite some time, many years before ionic liquids became a prominent research topic in chemistry. It is marketed as an algicide and antiseptic and sold diluted in water. However, the pure form is in fact an ionic liquid.

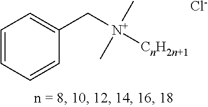

In some cases, benzalkonium chloride is available in bulk for lower prices than compounds marketed and sold as ionic liquids. The methods described herein can use benzalkonium chloride or another algicide in some cases.

### Example 10

### Separation Using a Potassium Phosphate Buffer

The hydrolyzate from gradual water addition contained approximately 60% IL and 4-7% sugars. In order for the loss rate of IL relative to sugars in the final sugar product stream to not exceed about 1%, the overall selectivity for sugars in the separation process should reach about 1800 (S=K_{IL}/K_{sugar}=1800). Here, K is the partition coefficient (e.g., the ratio of initial to final concentrations of IL or sugar).

The initial selection of sugars against IL was obtained by mixing a potassium phosphate buffer (PB) at pH=11. This mixture auto-separated rather quickly and coalesced into a top IL-rich layer and a bottom PB-rich layer. This occurred at ambient temperature and pressure. PB is a non-toxic and bulk industrial salt (e.g., available at about $ 1/kg). Separation was gentle and the integrity of both C₆ and C₅ sugar products were preserved. Therefore, this method could substitute IL with a cheap salt that is trivalent (negative 3 charge) and could therefore be cheaper to recover.

### Example 11

### High Pressure Apparatus

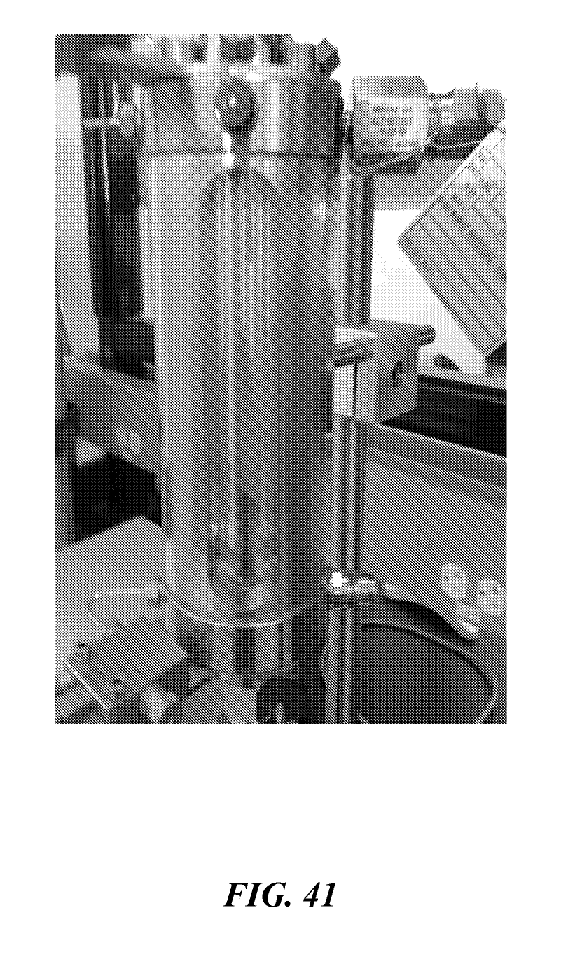

A high-pressure setup is assembled as shown in FIG. 41. The pressure cell is a long sapphire column of 15 mL to minimize the amount of IL required. The cell has an 11 mm inner diameter and 150 mm length. It can probe pressures up to 35 MPa (rated for 70 MPa) at 150° C., while allowing visual inspection of the entire column without a boroscope. Three sampling loops placed a different positions along the length can sample all phases simultaneously and without disturbing the system. The entire cell is enclosed in a jacket where various fluids can flow to maintain temperatures from below freezing up to the rated temperature (150° C.) by linking an external recirculating cooler/heater. Digital transducers placed inside the cell monitor both temperature and pressure. An impeller placed at the seat is driven by a controlled electrical motor for stirring. Alternatively, the electrical load on the motor can be measured against rotational speed in order to access fluid viscosity. A safety rupture disk is plumbed with thick stainless steel piping projected towards the lab ceiling to protect personnel from catastrophic failure of the sapphire wall. A camera equipped with a macro lens is used for monitoring and measuring the rate of phase interface formation. The pressure cell apparatus can be custom-made by Separex (Metz, France).

Externally, a canister with industrial-grade CO₂ and a pressure regulator (for safety) is plumbed with stainless steel tubing to a Waters 515 pump (Milford, Mass.). This pump injects compressed CO₂ in precisely metered amounts up to the rated pressure of the cell. Samples are analyzed by various methods, but mainly HPLC, gravimetric, ion conductivity, pH, UV-Vis, and Karl-Fisher titration. An Agilent Technologies (Santa Clara, Calif.) 1200 Series HPLC equipped with refractive index and photodiode array detectors is used for determining phase composition. Elution is driven by an isocratic pump and an Aminex HPX-87H column (300 mm by 7.8 mm) from Bio-Rad (Hercules, Calif.) using a 5 mM H₂SO₄ mobile phase at a flow rate of 0.6 mL/min at 65° C. Hydrolysate sugars, ionic liquids, inorganic salts and several other species can be resolved and detected with this method. Integration of chromatographic peaks results in areas that can be related to species amounts via calibration curves. This analysis is done in Agilent ChemStation software. Gravimetric measurements are done by a Mettler Toledo analytical balance (Columbus, Ohio) with ±0.5 mg precision. The moisture content of ionic liquids and concentrated inorganic salts is determined by a Mettler Toledo Karl-Fisher titrator (Columbus, Ohio) when necessary. Conductivity, pH and UV-Vis absorbance are determined by routine lab equipment.

### Example 12

### Use of Inorganic Salts

Inorganic salts were used to generate phase separations at ambient pressure. Some inorganic salts possess low free energies of hydration (Δ_{hyd}G) and could form stable hydration shells around the anion, leading to auto-separation from some ionic liquids. The carbonate anion (CO₃²⁻) created from the reaction of CO₂ with water also had a low hydration free energy (Δ_{hyd}G=−1300 kJ/mol). Inorganic salts could be used to form an ABS. The inorganic salt could be removed from the process downstream.

Whereas sugar was about 100-fold more soluble in water than pure [BMIM]Cl at room temperature, both phases in the ABS could have significant amounts of water, which could modulate phase density and polarity.

Potassium phosphate tribasic was selected as the inorganic salt. It is cheap, has a low toxicity, and has a very low free energy of hydration (K⁺ Δ_{hyd}G=−305 kJ/mol and PO₄³⁻ Δ_{hyd}G=−2835 kJ/mol). Note that Cl⁻ has Δ_{hyd}G=−338 kJ/mol. ABS formation was induced starting from mixtures designed to approximate the major components of an ionic liquid hydrolysate (e.g., IL, H₂O and glucose). A large starting concentration of glucose was prepared to evaluate if glucose itself could drive ABS formation with the help of a minority concentration of K₃PO₄. Table 5 shows ABS formation starting from a high glucose concentration. Note that percentages ignore water.

TABLE 5
Formation of ABS with phosphate buffer

| | Initial Mixture | Top, IL-Rich | Bottom, K₃PO₄-Rich | Performance | |
|---|---|---|---|---|---|
| glucose | 46.59% | 35.5% | 54.9% | K_{glu} = [glu]ₜₒₚ/[glu]_{bot} | 0.5 |
| K₃PO₄ | 3.84% | 4.6% | 28.1% | K_{IL} = [IL]ₜₒₚ/[IL]_{bot} | 4.8 |
| [BMIM]Cl | 49.57% | 57.3% | 9.9% | S = K_{IL}/K_{glu} | 8.9 |
| side-product | | 2.6% | 7.1% | | |

A combination of glucose with a small amount of K₃PO₄ induced a phase split and partitions glucose relative to IL with a selectivity of about 9 (i.e., the concentration of glucose was enriched ˜9-fold relative to IL). Partition coefficients (K) and selectivities (S) are thermodynamic quantities defined by:

$${K}_{glu} = \frac{{[ glu ]}_{rop}}{{[ glu ]}_{bottom}} , \\ {K}_{IL} = \frac{{[ IL ]}_{rop}}{{[ IL ]}_{bottom}} , \\ {S}_{glu / IL} = \frac{{K}_{IL}}{{K}_{glu}}$$

Glucose could be useful in driving ABS formation, and glucose partitions preferentially towards the bottom, salt-rich phase. Using glucose itself as the majority solute to self-extract can be attractive because it reduces the concentration of inorganic salt that could need to be recovered downstream. If the glucose concentration was higher than what was encountered in the hydrolysate, some of the sugar product could be returned to the process to maintain or establish the required ABS strength.

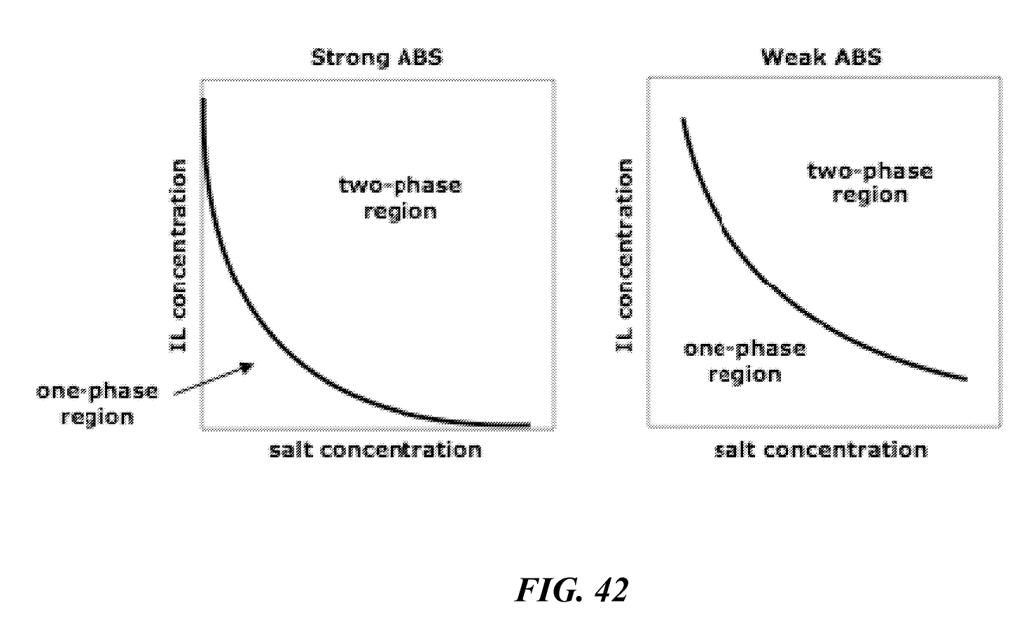

FIG. 42 shows a binary phase diagram for a weak ABS and for a strong ABS. These two binary phase diagrams illustrate a strong and a weak ABS formed by IL, salt and water (water is implicit). Any mixture with a composition inside the upper-right region of the phase diagram would split into two phases along its tie-line (not shown). Strong systems have a larger two-phase region (the curve approaches the axis origin more closely). Even though nearly a 10-fold glucose enrichment versus IL was obtained with only a few percent salt and in one step, IL could still constitute about 10% of the bottom phase.

### Example 13

### Sugar Stability

The stability of sugars in an alkaline environment experienced during ABS formation was tested. Concentrated xylose and glucose solutions were mixed in concentrated potassium phosphate solutions buffered to various pH from 6.5 to 14.1. The mixtures were left at room temperature for a few days. The un-buffered mixtures of xylose and glucose turned pale yellow after two or three days, whereas all buffered mixtures remained clear. Analysis by liquid chromatography confirmed that no degradation took place at pH 11, but at pH 14.1 about 30-40% of the initial xylose or glucose had decomposed (see Table 6).

TABLE 6
Stability of glucose and xylose in alkaline environments

| | | By-Product(s) | [glu] initial | [glu] final | [PB] initial |
|---|---|---|---|---|---|
| pH | Time Elapsed | (LC retention time in min) | (g/L) | (g/L) | (g/L) |
| 14.1 | ~3 days | 0.44 | 158 | | 208 |
| 9.4 | 48 h | none | 253 | 234 | 393 |
| 7.8 | 72 h | none | 253 | 232 | 160 |
| 6.5 | 147 h | none | 253 | 233 | 150 |
| | | | [xylose] initial | [xylose] final | |
| 14.1 | 48 h | 9.82, 10.17, 10.51, 11.79 (minor), 13.34 (minor) | 265 | 151 | 387 |
| 11.0 | 16 h | none | 265 | 253 | 500 |
| 9.4 | 48 h | none | 265 | 261 | 393 |
| 7.8 | 48 h | none | 265 | 249 | 160 |
| 6.5 | 48 h | none | 265 | 249 | 150 |

### Example 14

### Phosphate Buffer and pH

ABS strength was increased, while preserving sugar product. For this, a concentrated potassium phosphate buffer (PB) was prepared by mixing K₂HPO₄ and KH₂PO₄ in proportions adjusted so that pH=6.5. A composition was used that is representative of an ionic liquid hydrolysate (e.g., 65% IL, 30% H₂O and 5% glucose by mass). Known amounts of hydrolysate simulants were mixed in transparent 2-mL, 6-mL or 15-mL vials with buffered potassium phosphate solutions of known concentrations at ambient temperature and pressure (unless otherwise noted). The initial compositions and properties were measured and the mixtures were placed at rest for a time sufficient for the ABS to form and reach equilibrium. Both top and bottom phases were carefully sampled to avoid cross-contamination, diluted 10 or 20-fold in deionized water, and loaded into an HPLC for compositional analysis.

The simulated hydrolysate solutions were mixed with PB so that the final concentration of PB was about 20%. At this concentration and pH=6.5, ABS formed rather quickly. The selectivity of glucose over IL was 7.4, and IL concentration in the bottom (extract) phase reached 15%. Another attempt was made with a new PB buffer of 30% concentration (by mass) and pH adjusted to 8. The initial concentration of glucose was 7% and the combined PB and [BMIM]Cl concentration was 57%. Selectivity increased to 27.

In some cases, pH had an influence on the strength of the ABS split. The phosphate anion could form stable structures with water. However, phosphate was also a fairly strong base:

HPO₄²⁻+H₂O [symbol] PO₄³⁻+H₃O⁺ (pKₐ=12.37)

H₂PO₄⁻+H₂O [symbol] HPO₄²⁻+H₃O⁺ (pKₐ=7.20)

H₃PO₄+H₂O [symbol] H₂PO₄⁻+H₃O⁺ (pKₐ=2.15)

Hence, as pH was lowered, phosphates can become bound to protons. As the capacity to structure water can be dominated by the ion's valency, this resulted in weaker ABS. That is, for a fixed ion species concentration, the strength of ABS decreased in the order: PO₄³⁻>HPO₄²⁻>H₂PO₄⁻. In some cases, the objective was to increase the pH (and ABS strength) while preserving the integrity of sugars.

A phosphate buffer solution was prepared with pH=9.4 and a concentration of 56%. This buffer was prepared by dissolving K₂HPO₄ and adjusting the pH down with H₃PO₄. To probe the influence of glucose more quantitatively, the mass of glucose was gradually increased relative to PB. A total of 5 mixtures were created, ranging from 41% PB and 0% glucose (control), to 4.5% PB and 27% glucose. When starting from 6.6% glucose, 32% PB, and 26% IL, a selectivity for glucose of 74 was reached. In some cases, even though biomass-hydrolyzing ILs were hydrophilic, PB was able to absorb water more strongly, making the PB-rich phase an environment with a higher solubility for glucose than the IL-rich phase.

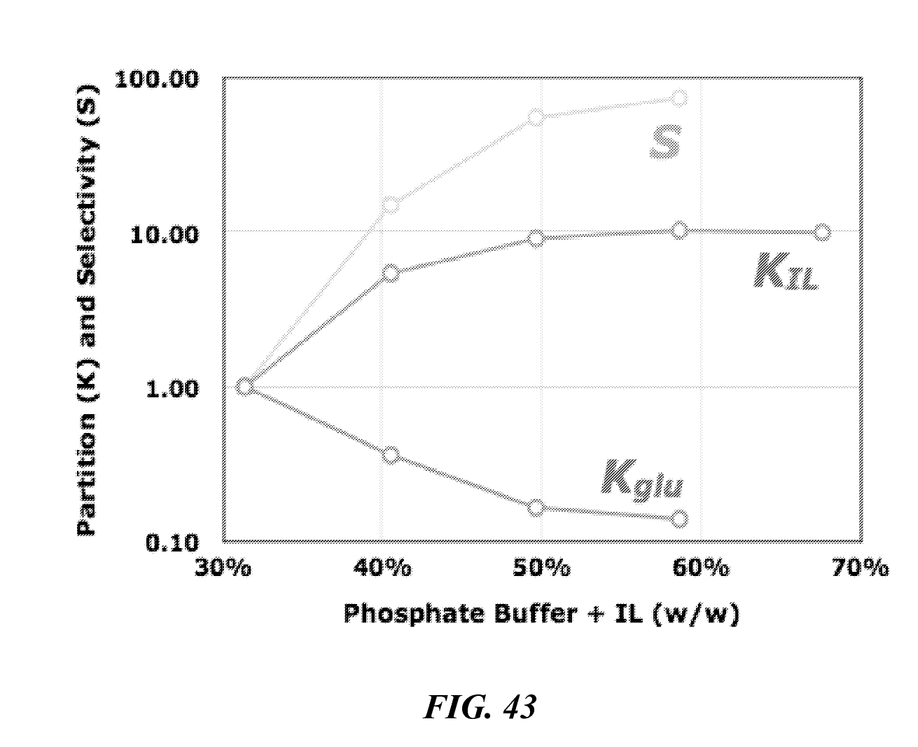

Several plots were created with respect to a composition axis. FIG. 43 shows partition coefficients for ionic liquid and glucose plotted with respect to the total concentration of phosphate buffer and ionic liquid at the start of ABS formation. Even though the concentration of water did not change as significantly, selectivity increases steeply from the 32% mark, where no ABS (or partition) was observed. At this pH (9.4) K_{IL} seems to plateau at about 10, but K_{glu} was still decreasing, suggesting an improving partition away from the IL-rich phase.

Increasing the combined concentrations of PB and IL, or concomitantly lowering the water content, could enhance the strength of the ABS. Even visually, mixtures prepared with less water, even though more viscous, could undergo faster auto-separation into distinct phases. ABS strength could also underlie more extreme partition coefficients and higher selectivities. One penalty for preparing higher concentration mixtures can be the cost of removing water from organic and inorganic salts.

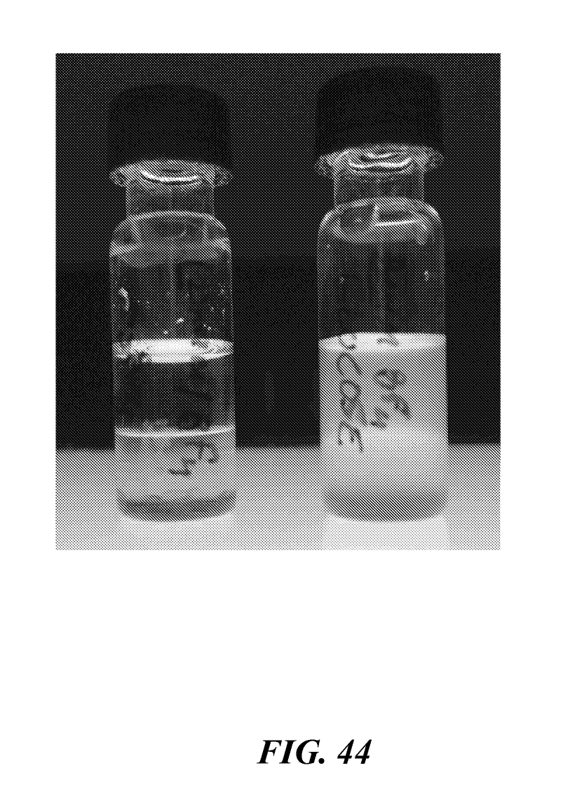

FIG. 44 shows ABS formation without the addition of salt. Less hydrophilic ILs such as [BMIM]BF₄ could auto-separate from glucose solutions (right vial) or with K₃PO₄. (left vial). A single glucose molecule could form 5 hydrogen bonds to water. In some cases, a mixture of glucose with less hydrophilic ILs could result in ABS formation. For example, [BMIM]BF₄ forms ABS when mixed with concentrated glucose solutions, as shown in FIG. 44. Even though [BMIM]BF₄ was miscible with water to all proportions and could dissolve a small amount of cellulose, it was unstable in water (via HF release) and not suitable for biomass processing. However, it did illustrate how the IL could be tailored to achieve both high sugar yields and efficient separation. In this regard, the gradual water addition hydrolysis reaction could use chloride as the anion, and variation could be tolerated at the cation (e.g., as had been shown by achieving hydrolysis to both [EMIM]Cl and [BMIM]Cl).

### Example 15

### Use of Various Ionic Liquids

The present example used the ionic liquid 1-butyl-4-methylpyridinium chloride (or [BMPYR]Cl). This IL produced greater sugar yields than [EMIM]Cl in some cases and could be readily synthesized from methylpyridine and chlorobutane, both bulk industrial chemicals. The larger aromatic structure at the cation (relative to BMIM) preserved solubility towards lignocellulose, but imparted greater hydrophobicity, facilitating separations from water and sugar.

The same pH=9.4 buffer was used with both [BMIM]Cl and [BMPYR]Cl. Each room temperature experiment was repeated at 1° C. (using a refrigerator) for a total of 4 vials. All samples were left to rest overnight to reduce the possibility that cold samples had not reached equilibrium. Initial compositions were adjusted to match the major components of IL hydrolysates and had about 24% water. ABS formation proceeded quickly, forming an IL-rich top phase with a density of 1.04-1.11 g/mL, and a PB-rich bottom phase of 1.70-1.73 g/mL.

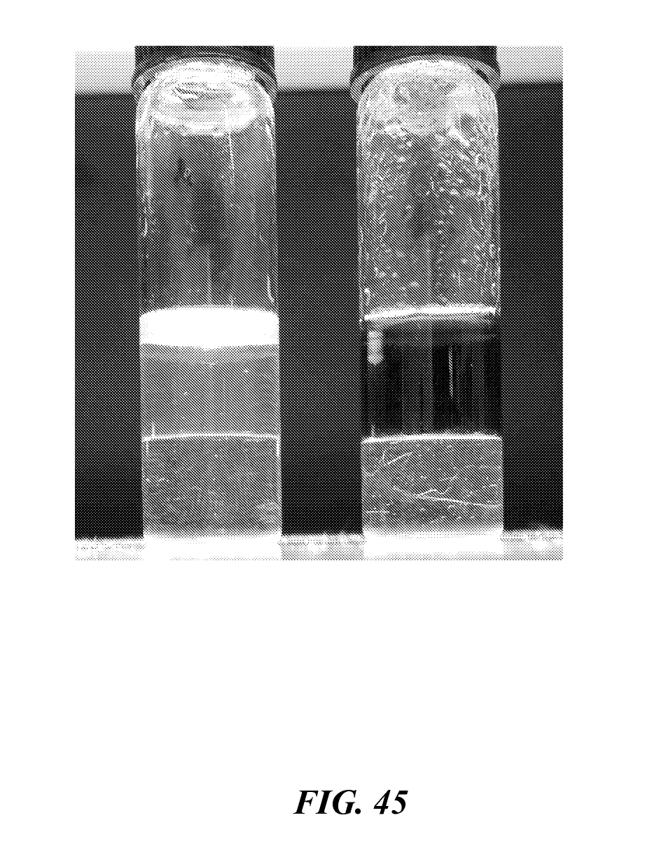

FIG. 45 shows [BMIM]Cl (left vial) and [BMPYR]Cl (right vial) ABS at equilibrium at room temperature with a bottom phase of PB adjusted to pH=9.4. [BMIM]Cl is a pale yellow solid that acquires a deeper hue when hydrated. The same happens with [BMPYR]Cl, which is orange when solid and dark red when hydrated. The bottom phase is clear, PB-rich and contains most of the glucose. In all cases, the ABS forms quickly and seems to be stable indefinitely. The same ABS at 1° C. had a similar appearance, but with a more corrugated interface.

In all four cases, the amount of water at the bottom phase was remarkably similar (i.e., 57-58%). However, the top phases possessed significantly more water in [BMIM] than [BMPYR] ILs (i.e., 30-32% and 18-19%, respectively). This suggested that the pyridinium structure conferred less hydrophilicity and was easier to dehydrate. Dehydration enabled both better glucose partition (and selectivity) and lowered the energy for drying the IL (so it could re-dissolve biomass).

As was summarized in Table 7, selectivities could reach 90 or more. Yet, the pH of PB could be raised further, as degradation of sugars could occur beyond a pH of 11. In a single step, the concentration of glucose became 10-fold greater in the extract phase (PB-rich phase), and the concentration of IL was reduced 10-fold.

TABLE 7
Partition coefficients and selectivities for two ILs and temperatures.

| | T = 21° C. | T = 1° C. |
|---|---|---|
| [BMIM]Cl | K_{glu} = 0.11 | K_{glu} = 0.11 |
| | K_{IL}= 10.3 | K_{IL} = 10.5 |
| | S = 90 | S = 96 |
| [BMPYR]Cl | K_{glu} = 0.12 | K_{glu} = 0.10 |
| | K_{IL} = 10.3 | K_{IL} = 10.6 |
| | S = 83 | S = 103 |

### Example 16

### Kinetics

Using [BMIM]Cl as the IL, the pH of the phosphate buffer (PB) was set to 11 with the initial water content at 26%. The mixture also included xylose in equal proportion to glucose. Xylose is one of several five-carbon (C5) sugars found in biomass, and a good representation for the different properties of C5 versus six-carbon (C6) sugars in separations.

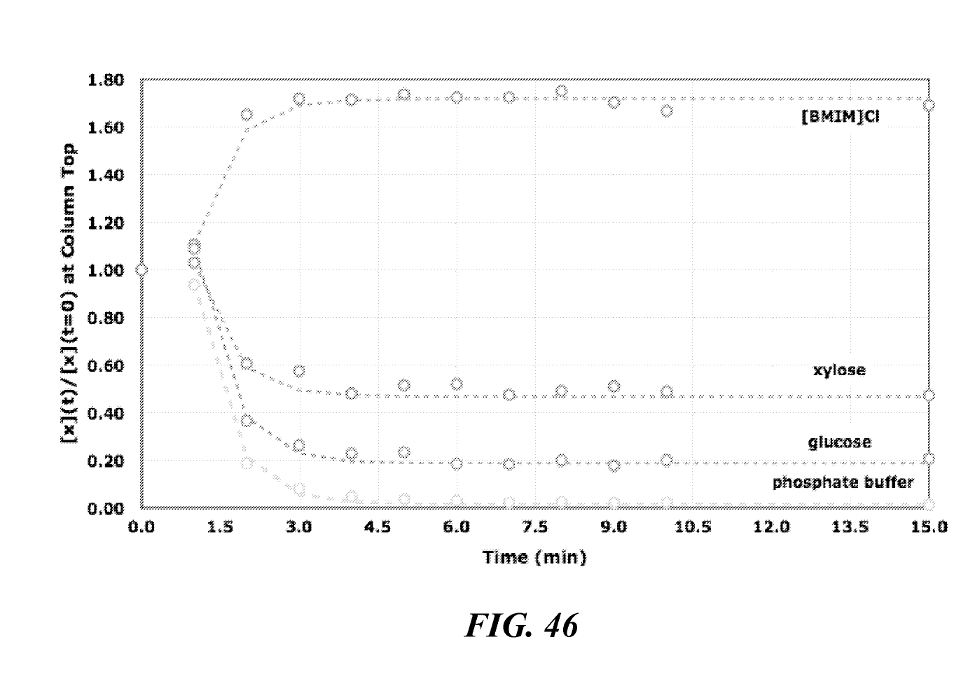

The kinetics of ABS formation was measured. Starting from a well-stirred mixture, samples were taken at a depth of 1-2 mm from the top of the IL-rich phase at pre-determined time intervals. Each sample was diluted 20-fold in deionized water and loaded onto the HPLC for analysis. The results are shown in FIG. 46. These ABSs form in a few minutes. In this experiment, 16.53 g of a mixture containing xylose, glucose, PB, IL and water was prepared and placed in a 20-mL vial. Starting from the moment vortexing of the mixture was stopped, there was a lag of less than about 1 min before concentrations started to evolve.

All concentrations of a given species were normalized with respect to its initial concentration in order to measure the characteristic timescale for ABS formation. As such, every species started from 1 and evolved towards its equilibrium (normalized) concentration value. The equation:

$$\frac{c ( t )}{{c}_{o}} = a + b {e}^{- t / τ}$$

was fitted to the concentration values obtained for each species via HPLC. Here, c(t) was the concentration of a given species at time t, cₒ is its initial concentration, and a, b, and τ are free parameters. The characteristic timescales (τ) for all 4 species were approximately equal. The baseline value a was set to the average value at long times, that is, the equilibrium concentration. Even though data is shown for only the first 15 minutes, data was recorded for several hours with no changes. The coefficient b was adjusted so that the fit coincided with the concentration value at t=1 min. The fitted equations are depicted by the dashed lines in FIG. 46. Notice that t=0 was disregarded.

FIG. 46 shows the kinetics of ABS formation with normalized concentration trajectories for the IL-rich phase composition. This analysis resulted in a characteristic time scale for ABS formation of τ=0.65 min. If equilibrium was taken as ˜5 τ, where concentration would be a mere e⁻⁵=0.7% from equilibrium (within error), then equilibrium was reached within 5τ+1=4.25 min. This mass transport occurred over the height of the vial (0.040 m).

Table 8 shows the compositions and densities of the initial, and at equilibrium top and bottom phases. At equilibrium, the sugar selectivity was S=K_{glu+xyl}/K_{IL}=119. The increase in selectivity from 90 to 119 was mostly due to the increase in pH of PB from 9.4 to 11. In some cases, a higher pH contributes to the speciation of the phosphate anion, increasing the concentration of bare PO₄³⁻. This trivalent species structured water more strongly than protonated forms (but left enough “free” water to solvate sugars). Besides pH, the relative starting concentrations of IL and PB could be optimized in order to maximize the volume of the bottom phase (at equilibrium), providing a more complete extraction of glucose in a single step.

TABLE 8
ABS compositions at start and at equilibrium.
[BMIM] Cl and PF pH =11 at 21° C.

| | mass (mg) | % (w/ w) | [ ] g/L | Stock Vol (uL) |
|---|---|---|---|---|
| glucose | 134 | 0.81% | 11.4 | 500 |
| xylose | 131 | 0.79% | 11.1 | |
| PB pH 11 | 5996 | 36.3% | 511 | 6000 |
| [BMIM]Cl | 5970 | 36.2% | 509 | |
| H₂O | 4293 | 26.0% | 366 | |
| TOTAL | 16531 | 100% | 1408 | |
| Density | 1.408 | g/mL | | |

Top Phase of [BMIM]Cl and PF pH =11 at 21° C.

| | % (w/ w) | molality | HPLC [ ] g/L |
|---|---|---|---|
| glucose | 0.16% | 0.04 | 1.79 |
| xylose | 0.44% | 0.13 | 4.76 |
| PB pH 11 | 0.26% | 0.06 | 2.86 |
| [BMIM]Cl | 75.9% | 18.71 | 827 |
| H₂O | 23.2% | | 253 |
| TOTAL | 100% | 18.94 | 1089 |
| Density | 1.089 | g/mL | |

Bottom Phase of [BMIM]C1 and PF pH =11 at 21° C.

| | % (w/ w) | molality | HPLC [ ] g/L |
|---|---|---|---|
| glucose | 1.76% | 0.27 | 32.3 |
| xylose | 1.26% | 0.23 | 23.2 |
| PB pH 11 | 57.0% | 8.91 | 1048 |
| [BMIM]Cl | 3.21% | 0.50 | 59.0 |
| H₂O | 36.8% | | 676 |
| TOTAL | 100% | 9.90 | 1838 |
| Density | 1.838 | g/mL | |

Partition coefficients and selectivities.

| | Value |
|---|---|
| K_{glu} = [glu]ₜₒₚ/[glu]_{bot} | 0.055 |
| K_{xyl} = [xyl]ₜₒₚ/[xyl]_{bot} | 0.20 |
| K_{sugars} = [glu + xyl]ₜₒₚ/[glu + xyl]_{bot} | 0.12 |
| K_{IL} = [IL]ₜₒₚ/[IL]_{bot} | 14 |
| S = K_{glu} + _{xyl}/K_{IL} | 119 |

Besides selectivity, other aspects of the partition were improved by the higher pH of the PB. Now, the top (raffinate) phase contained less water (19%) and retained less sugars (0.6% in total). Also, the bottom (extract) phase contained less IL, as its partition coefficient was now larger (about 14). The difference in density was also larger, 1.089 g/mL at the top and 1.838 g/mL at the bottom. These improvements were obtained even though xylose was present in about the same amount as glucose. Xylose does not partition to the extract as well as glucose in some cases, as shown by the lower partition coefficient of glucose versus xylose, 0.055 and 0.20, respectively. If glucose was the only product, the selectivity would have been about 250.

### Example 17

### CO₂ Pressure

CO₂ can be complimentary to ABS created by inorganic salts (e.g., potassium phosphate buffers). In water, CO₂ reacts forming H₂CO₃, which is an acid and lowers the pH. As the pH drops, the solubility for more of the gas also drops, requiring ever increasing pressures. On the other hand, the phosphate buffers that can form ABS and partition glucose favorably are bases. When combined with CO₂, they can help maintain a more neutral pH and allow a greater concentration of CO₃²⁻ species, which in turn can create stronger ABSs with higher selectivities. Also, the salt loading in the hydrolyzate is lowered so that the size of the downstream unit operation for recovering the salt can be reduced. CO₂ can be recovered by heating or sparging with an inert gas.

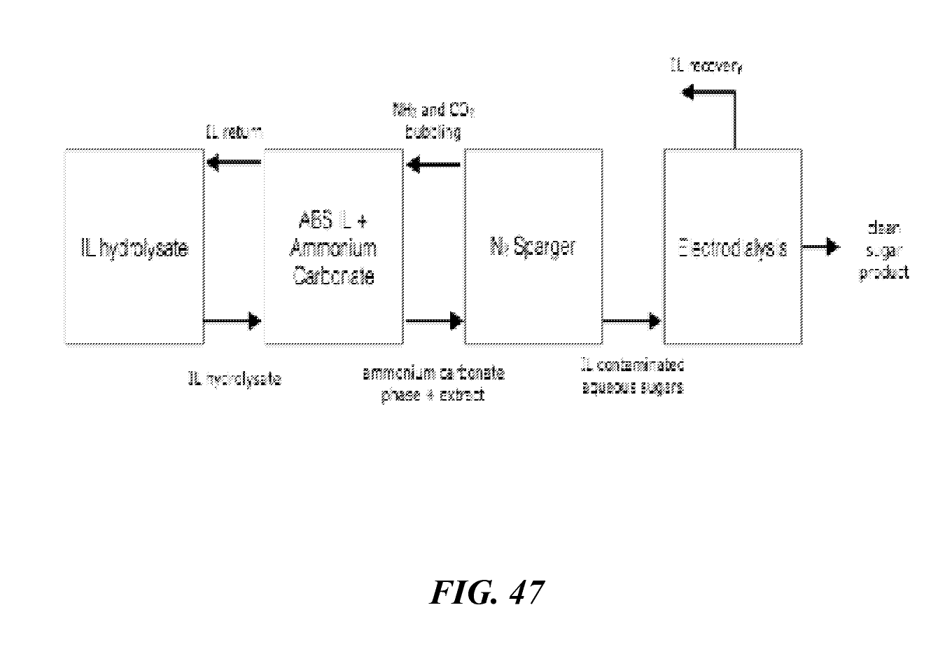

FIG. 47 shows a schematic drawing of separation employing IL and CO₂. Ion-exchange or electrodialysis can be used cost-effectively once the concentration of IL is dilute.

### Example 18

### Design of Liquid-Liquid Separations

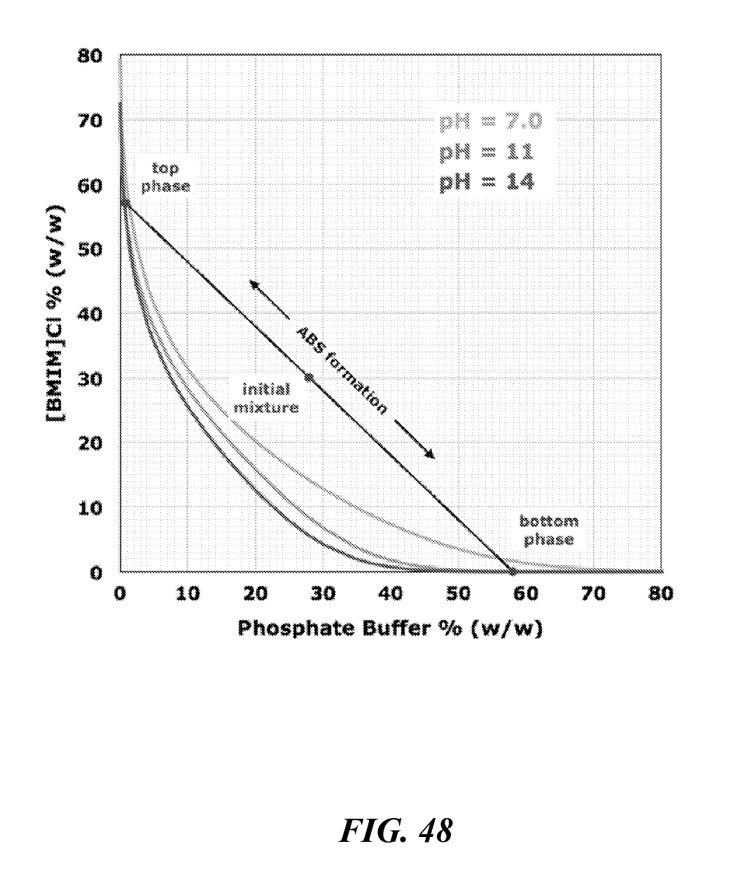

Industrial liquid-liquid (L-L) extraction can take place in a continuous and counter-current flow arrangement, and multiple stages. The L-L extraction design can be represented by a phase diagram, as shown in FIG. 48. Here, the Merchuk equation is fit to phase composition data. The Merchuk empirical model reads:

y(x)=a+exp(bx^{0.5}−cx^{y})

where y(x) is the % mass fraction of the y-axis (IL), x is the % mass fraction of the x-axis (inorganic salt), and a, b, and c are fitting parameters. Potassium phosphate buffers adjusted to three pH values are shown. The auto-separation of the mixture into top and bottom phases occurs along tie lines. As illustrated in FIG. 48, the initial mixture (circle at center) falls on the two-phase region (above the curve) and so it separates into top and bottom phases. The compositions of both resulting phases are given by the intercepts of the phase curve with the tie line positioned through the initial mixture composition. Higher pH can result in stronger ABS (less intermixing of IL and PB) and higher selectivities for sugars. In this phase diagram, this is shown by the closer proximity of the curve (and intercepts) to the axes at higher pH. All phase diagrams represent room temperature (298 K) experiments.

FIG. 48 shows an example of a ternary system phase diagram represented in two dimensions. Ionic liquid and phosphate buffer are plotted in perpendicular axes. The water concentration can be calculated as 100%-IL %-PB %. Sugars and other solutes are ignored. Three pH values for the PB are plotted, showing the differences in ABS strength (curves closer to the origin, and axes, are stronger.) Phase diagrams are Merchuk equation fits to ABS compositions at equilibrium at 298 K. Tie line is a schematic representation only for illustrating ABS formation.

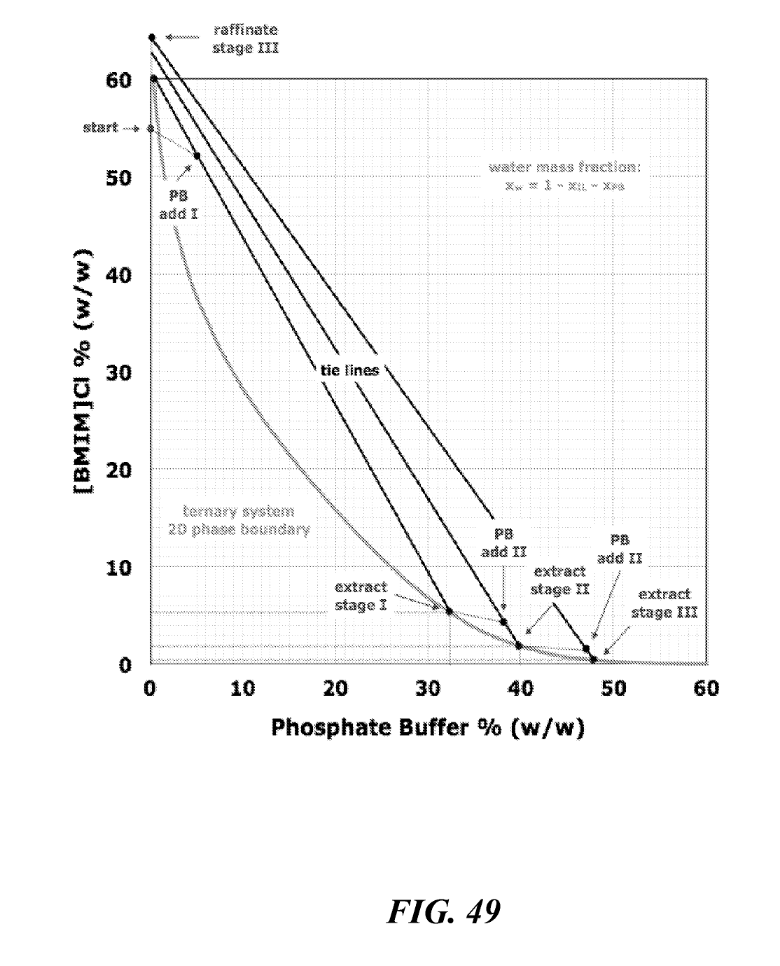

An illustration for a multistage design is shown in FIG. 49. The initial hydrolysate composition is depicted (“start”) as ˜60% IL and no PB. From here, PB is added, resulting in an increase in the PB fraction and concomitant decrease of the IL fraction (and water: x_{w}=1−x_{IL}−x_{PB}). This is represented by dotted black lines. By the end of PB addition, a new mixture composition exists inside the two-phase region. This mixture splits along its tie line, resulting in an IL-rich composition (raffinate) and a PB-rich composition (extract) indicated by the two intercepts between the tie line and phase curve. In the second stage, more fresh PB is added, resulting in a second split that produces both raffinate and extract phases of higher purities. A third stage is shown, resulting in a final IL concentration of <0.5% in the extract.

FIG. 49 shows the design of a multi-stage liquid-liquid extraction. This illustration shows the extraction of IL hydrolysate with PB at pH=11. Starting from 60% IL in the feed hydrolysate, PB is added to create the first split. Extract from the first stage (extract stage I) is shown, with an IL composition of ˜5%. Two additional stages are illustrated, showing compositional changes during PB addition (dotted black lines) and phase splits along tie lines (solid black lines). While the extract phase becomes enriched in PB (and sugars), it is depleted of IL. The raffinate phase becomes depleted of PB (and sugars) and is returned to hydrolysis. The phase diagram is a Merchuk fit to data but tie lines are drawn schematically for illustration purposes only.

### Example 19

### Dissolution of Loblolly Pine

About 0.5 L of [BMIM]Cl is loaded into a glass reactor. The temperature is set to 110° C. and the liquid is continuously agitated. To the heated liquid, 50 g of ground up loblolly pine sawdust added. The reactor is sealed and pressurized with 3 MPa with carbon dioxide. Dissolution is monitored visually. Complete disappearance of solid particulates, indicating full dissolution of the biomass, is observed at a first period of time. The method is repeated without carbon dioxide pressure (at atmospheric pressure). Complete disappearance of solid particulates is observed at a longer time than the first period of time.

### Example 20

### Hydrolysis of Rice Hulls

About 1 L of [EMIM]Cl is loaded into a glass reactor. The temperature is set to 110° C. and the liquid is continuously agitated. After 5 minutes of agitation, 100 mg of ground up rice hull is added and the reactor is closed and sealed. This mixture is stirred for 60 minutes to ensure all the hull has dissolved, forming a viscous mixture. The pressure inside the reactor is 0.1 MPa, (atmospheric pressure), and about 3 g of concentrated HCl is added under agitation. The viscosity subsequently drops and water is added in small (100 mL) aliquots after 10 min, 15 min, 20 min, and 30 min. After a total reaction time of about 75 min, soluble material is diluted and analyzed for glucose yield in an HPLC.

The procedure is repeated except that the pressure inside the reactor is raised to 10 MPa. Water is added in small (100 mL) aliquots after 10 min, 15 min, 20 min and at 30 min. After a total reaction time of 75 min, soluble material is diluted and analyzed for glucose yield in an HPLC. It is found that the yield of glucose is higher when the pressure is 10 MPa than when the pressure is atmospheric.

### Example 21

### Loblolly Pine Dissolution

About 10 grams of ground up and dry pine wood is mixed with 100 milliliters of 1-butyl-3-methylimidazolium chloride ionic liquid at 105° C. The mixture is agitated vigorously for 4 hours. Cellulose, hemicellulose and other polysaccharide components of the biomass dissolve completely. Lignin, ash and protein components of the biomass dissolve incompletely.

### Example 22

### Formation of Hydrolysate

Starting from the result of Example 21, Hydrogen chloride acid is added to the mixture to a concentration of about 1%. The temperature is kept at 105° C. and the mixture is agitated vigorously for another 2 hours. During this time, water is added in controlled amounts at 5 minute intervals to hydrolyze the majority of the cellulose, hemicellulose, and starch. By the end of the reaction the mixture is allowed to cool to room temperature. The result of the reaction is a hydrolysate mixture containing mostly low molecular weight sugars (about 8% by mass), water (about 13% by mass), precipitated solids (about 10% by mass), and ionic liquid solvent. Precipitated solids comprise lignin.

### Example 23

### Lignin Filtration

Starting from the result of Example 22, the hydrolysate is filtered by a screen or mesh. The filtrate passing through the screen becomes enriched in ionic liquid and ionic liquid soluble components. The retentate, which does not pass through the screen, becomes enriched in lignin. The retentate forms a cake against the filter screen. The filtrate portion and the cake adhered to the screen are stored for later use.

TABLE 9
Lignin Filtration.

| Filtration: Cake | | CAKE | FILTRATE |
|---|---|---|---|
| Formation | IN | OUT | OUT |
| Water | 62.41 | 1.45 | 60.96 |
| IL/HCl | 10694.91 | 247.83 | 10447.08 |
| Lignins | 327.61 | 327.61 | 0.00 |
| Sugars | 18.11 | 0.42 | 17.69 |
| Humins/Proteins | 42.02 | 42.02 | 0.00 |
| Ash | 4.91 | 4.91 | 0.00 |

### Example 24

### Lignin Wash

The cake adhered to the screen produced in Example 23 contains both lignin and ionic liquid. The cake is washed 3 times with a volume of water about equal to the volume of the cake. The water passes through the screen carrying ionic liquid and dissolved components. During each wash, both the remaining cake and washed liquid is weighed. The washed liquid is measured in a calibrated UV/Vis spectrophotometer to determine the concentration of ionic liquid. After the first wash, about 90% of the ionic liquid is washed and recovered from lignin. The second and third washes also recover about 90% of remaining ionic liquid, each. The concentration of ionic liquid in lignin is reduced from 39.8% to 0.1% after the three consecutive washes.

TABLE 10
Lignin Washes.
LigninWash 1.

| | WET CAKE | WASH H2O | WET CAKE | FILTRATE |
|---|---|---|---|---|
| WASH 1 | IN | IN | OUT | OUT |
| Water | 1.45 | 249.69 | 224.87 | 26.27 |
| IL/HCI | 247.83 | 0 | 24.78 | 223.04 |
| Liginins | 327.61 | 0 | 327.61 | 0.00 |
| Sugars | 0.42 | 0 | 0.04 | 0.38 |
| Humins/Proteins | 42.02 | 0 | 42.02 | 0.00 |
| Ash | 4.91 | 0 | 4.91 | 0.00 |

Lignin Wash 2.

| | WET CAKE | WASH H2O | WET CAKE | FILTRATE |
|---|---|---|---|---|
| WASH 2 | IN | IN | OUT | OUT |
| Water | 224.87 | 249.69 | 247.21 | 227.35 |
| IL/HCl | 24.78 | 0 | 2.48 | 22.33 |
| Lignins | 327.61 | 0 | 327.61 | 0.04 |
| Sugars | 0.04 | 0 | 0.00 | 0.04 |
| Hurnns/Proteins | 42.02 | 0 | 42.02 | 0.00 |
| Ash | 4.91 | 0 | 4.91 | 0.00 |

Lignin Wash 3.

| | WET CAKE | WASH H2O | WET CAKE | FILTRATE |
|---|---|---|---|---|
| WASH 3 | IN | IN | OUT | OUT |
| Water | 247.21 | 249.69 | 249.45 | 247.46 |
| IL/HCl | 2.48 | 0 | 0.25 | 2.23 |
| Lignins | 327.61 | 0 | 327.61 | 0.00 |
| Sugars | 0.00 | 0 | 0.00 | 0.00 |
| Hurrtns/Proteins | 42.02 | 0 | 42.02 | 0.00 |
| Ash | 4.91 | 0 | 4.91 | 0.00 |

### Example 25

### Hydrolyzate Filter and Wash

A hydrolysate mixture is formed by an ionic liquid hydrolysis reaction. The mixture is approximately 8% water, 82% [EMIM]Cl ionic liquid, 7% sugars, 2.5% lignin, <1% alcohols, acetate and tall oils, and residual cellulose, proteins, humins and ash also comprise <1%. Lignin, residual cellulose, protein and humin precipitate from solution. About 100 g of hydrolysate is loaded into a flask. Not all particulates settle to the bottom of the flask. The mixture equilibrates to room temperature. An Büchner flask, which has a suction port, is prepared. A Büchner funnel with a weighed moist filter paper (stainless steel mesh) is fitted to the flask with a rubber bung. A rubber hose is attached to the flask and turned on (about less than 100 Torr). The hydrolysate is poured on the funnel One minute is allowed for the mixture to filter through the filter paper and aerate. The filter paper is carefully removed with the cake and weighed again. The mass change is 4.8 g. A sample of the cake is analyzed in an NMR following a protocol detailed elsewhere. The amount of residual ionic liquid is determined at 5.0% of the dry mass of solids.

The cake is placed on the funnel and the vacuum line is re-opened. The cake is washed with an even spray of distilled and deionized water broken up into small droplets. The water is at room temperature. The amount of water used is 10-fold more than the volume of cake (a wash ratio of about 10). The filter paper is again carefully removed with the cake and weighed again. The mass change is also 4.8 g. A sample of the cake is analyzed in an NMR following the same protocol. The amount of residual ionic liquid has dropped to ˜1% of the dry mass of solids.

### Example 26

### High Pressure and Hot Hydrolyzate Filter and Wash

A hydrolysate mixture is formed by an ionic liquid hydrolysis reaction. The mixture composition and precipitate formation is the same as before. About 100 g of hydrolysate is loaded into a flask. Not all particulates settle to the bottom of the flask. The mixture equilibrates to room temperature. An Büchner flask, which has a suction port, is prepared. A Büchner funnel with a weighed moist filter paper (polypropylene) is fitted to the flask with a rubber bung. A rubber hose is attached to the flask. The whole system is encased in a glass transparent pressure vessel. The vessel is sealed and pressurized to 4 MPa with CO₂.

The hydrolysate is poured on the funnel at 50° C. as the vacuum line is turned on (about less than 100 Torr). One minute is allowed for the mixture to filter through the filter paper and aerate. After slow depressurization of the enclosing vessel, the filter paper is carefully removed with the cake and weighed again. The mass change is 4.1 g. A sample of the cake is analyzed by NMR as before. The amount of residual ionic liquid is determined at 2.3% of the dry mass of solids.

The cake is re-placed on the funnel and the process is repeated but without enclosing in a pressurized vessel. The cake is washed with an even spray of distilled and deionized water broken up into small droplets. The water is again at 50° C., but pressure is atmospheric. The amount of water used is 10-fold more than the volume of cake (a wash ratio of about 10). The filter paper is again carefully removed with the cake and weighed again. The mass change is up to 4.8 g. A sample of the cake is analyzed in an NMR following the same protocol. The amount of residual ionic liquid has dropped to 0.2% of the dry mass of solids.

### Example 27

### Extensive Hot Wash

The cake resulting from Example 25 and Example 26 are washed again. The extra washing is done as before, using 10-fold more water volume relative to the cake volume. Washing is done by an even spray of distilled and deionized water, as before, set to 60° C. After two additional identical washes, the residual IL has dropped to 0.13% and 0.08% of the dry solid mass, respectively.

### Example 28

### Characterization of Lignin

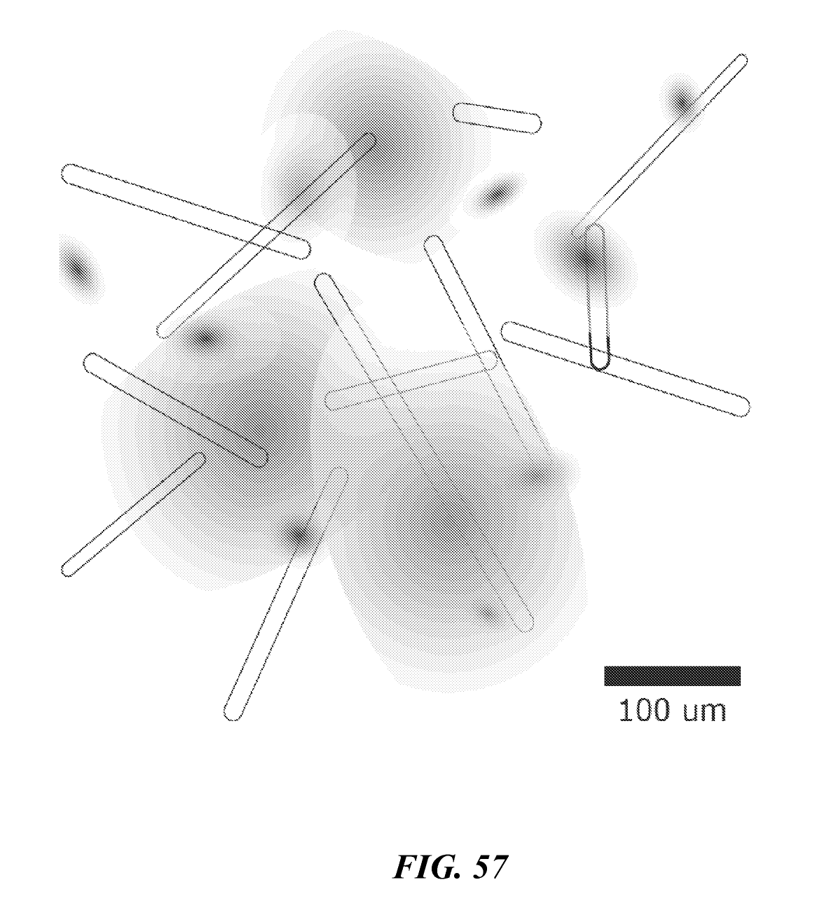

The composition and structure of washed lignins produced according to Example 24 is measured. The isolated solids are measured using a 2D NMR Heteronuclear Single Quantum Coherence (HSQC) spectroscopy technique adapted for biomass studies. The result shows many of the same structures in native lignin, indicating that it is well-preserved through the process. For instance, side-chain (δC/δH 50-90/2.5-5.8) and aromatic/unsaturated (δC/δH 90-155/5.5-8.0) regions in the 2D HSQC NMR spectrum of lignins produced in Example 24 appear largely intact. The labile β-aryl ethers are prominent, as are the phenylcoumarans, both prominent inter-unit linkage types in lignin. Aromatic guaiacyl units, syringyl units, as well as tricin ethers and p-coumarate esters are readily seen. In addition, the spectra reveals that saccharification is much more thorough than enzymatic hydrolysis by a typical cellulase cocktail. That is, signals corresponding to polysaccharides are much less prominent in lignins produced in Example 24 than by enzymatic hydrolysis. FIG. 57 shows a drawing of the isolated solids as viewed under a microscope.

### Example 29

### Lignin Depolymerization in Supercritical Water

Lignin produced in Example 24 and optionally characterized in Example 28 are depolymerized. Lignin is dissolved in acetone/water 10:2 (v/v) at 0.40 g/mL. A 100 mL reactor is sparged with carbon dioxide to remove oxygen and other contaminants. About 1 g of lignin in solution is pumped at about 15 mL/min by a HPLC pump, pre-heated to 400° C. and injected into the reactor. Hydrochloric acid is also introduced into the reactor. Carbon dioxide is pressurized into the reactor to about 120 bar, giving a supercritical phase of CO₂/acetone/water. The reaction lasts 200 minutes, after which pressure is released. The final reaction mixture is run through both a gas chromatograph and high performance liquid chromatograph with appropriate method and calibration. Phenolic oil and dried char is also measured gravimetrically.

### Example 30

### Characterization of Lignin Depolymerization Products

Monomeric and dimeric aromatic products are identified and quantified by gas chromatography equipped with mass analyzer and flame ionization detection, respectively. Identification of compounds is performed by comparison to data from the NIST library and internal standards. A total yield of phenolic oils can be about 10% based on starting lignin, which consists mostly of oligomeric fragments and monomeric aromatic compounds.

### Example 31

### Conversion of High Quality Lignin into Several Products

Benzene, Toluene and Xylene (BTX).

High quality lignin product can yield aromatic compounds such as benzene, toluene, xylene, or combinations thereof upon processing. One possible route for the manufacture of BTX is the bioconversion of lignin by industrial microbes. However, toxic compounds generally present in extracted lignin inhibit the growth and survival of microbes necessary for this route. One strategy is to use recombinant microbes with imparted resistance to those toxins (e.g., US Patent Publication No. 2011/049619). Another possibility is to start from high quality lignin product described in the present invention and use industrial microbes without this engineered trait.

Concrete.

High quality lignin product can yield high performance concrete aid, concrete grinding aid, and other concrete compositions with desirable properties. For example, high quality lignin product can reduce damage of building external walls caused by moisture and acid rain. It can also serve as a retarder for cement compositions. High quality lignin product can also improve compressive strength of cement pastes.

Antioxidant.

High quality lignin product may be used as free radical scavengers to reduce, retard or eliminate oxidation in styrene, butadiene, rubber, polypropylene, polycaprolactam, and other polymers. High quality lignin product natural antioxidant properties can be used in cosmetic and topical formulations.

Asphalt.

High quality lignin product may be used in compositions to fill asphalt cracks. In some embodiments, the composition comprises high quality lignin product and one of a quaternary ammonium salt, aliphatic amine, lignin amine, imidazoline, and amide.

Carbon Fiber and Related Fibers.

High quality lignin product may be used as a precursor to carbon fibers. In some embodiments, carbon nanotubes may be manufactured from high quality lignin product. In some embodiments, high quality lignin product may result in higher yields, lower cost and/or simpler processing to end-product than lignosulfonates, kraft lignin, organosolv lignin, or other lignin precursors.

High quality lignin product presents a practical and low-cost means to obtain carbon fibers compared to other lignins. For example, kraft lignin is present in black liquor and must be modified in order to form a melt that can be spun into a fiber. Others have suggested the catalytic acetylation of lignin (e.g., U.S. patent application Ser. No. 11/767,608), which results in a complex and costly process. Starting from high quality lignin product of the present invention bypasses these problems.

Board Binders.

High quality lignin product may be used in compositions of fiberboards, strawboards, particleboards, oriented strand boards, wood fiber insulated boards, and the like. High quality lignin product may yield low cost composite materials that has a reasonable wet strength. In some embodiments the composition may include a binder system such as phenol formaldehyde, urea formaldehyde, melamine formaldehyde, resorcinol formaldehyde, and/or tannin formaldehyde resins. The high quality lignin product modifier may be used for panel boards such as plywood, hardboard, medium density fiberboard or particleboards.

Foams, Plastics and Other Polymers.

High quality lignin product may be used in compositions with polyurethane for manufacturing flame retardants. In some embodiments, high quality lignin product may be converted into an acid anhydride to be used as an epoxy curing agent. In some embodiments, high quality lignin product may be used in compositions for automotive breaks. In some embodiments, high quality lignin product may be used in compositions blended with polyphenylene oxide-based polymers for reduced cost and/or improved performance. In some embodiments, high quality lignin product may be used in compositions as a water absorption inhibitor. In some embodiments, high quality lignin product may be used in polymer compositions as a fluidization agent for processing by injection molding, blow molding, extrusion, or blow extrusion to fabricate articles. In some embodiments, high quality lignin product may be used in compositions for improving thermal stability and mechanical properties.

Dust Control.

High quality lignin product may be used to reduce airborne concentration of dust in coal mines, coal transportation, stock yards, and the like. In some embodiments, high quality lignin product may be used in compositions that stabilize contamination following a nuclear accident.

Paper.

High quality lignin product may be used as a paper sizing agent. In some embodiments, high quality lignin product may be used to produce phenolic resin for wet curtain paper. In some embodiments, high quality lignin product may be used in compositions for packaging laminate comprising a barrier layer.

Chemicals.

High quality lignin product may be reacted with hydrogen to produce phenols. High quality lignin product may be depolymerized to produce cresols, catechols, resorsinols, quinones, vanillin, guaiacols, and the like. Vanillin may be produced in larger yields and/or reduced cost starting from high quality lignin product.

Batteries.

High quality lignin product may be used in compositions that enhance performance of energy storage devices. In some embodiments, high quality lignin product may be used in compositions that decreases overvoltage, increases energy efficiency, increases lifespan, and confers other advantages to the performance of batteries.

Fuels.

Lignin product may be used to manufacture a wide range of fuels. These include drop-in and non-drop-in fuels. Gasoline replacements, diesel replacements, blendstocks, and the like. In some embodiments, high quality lignin product may be used in diesel compositions. In some embodiments, high quality lignin product may undergo hydroliquefaction to produce useful fuels including hydrocarbons. In some embodiments, high quality lignin product may be converted to hydrocarbons by use of one or several catalysts. In some embodiments, high quality lignin product may be converted to gasoline, diesel, and the like by pyrolysis, thermal cracking, hydrocracking, catalytic cracking, hydrotreatment, or combinations thereof. In some embodiments, high quality lignin product may be converted to gasoline, diesel, and the like by catalytic hydrogen reduction of carbon-oxygen bonds, catalytic disproportionation of carbon-oxygen and/or carbon-carbon bonds.

Heat.

High quality lignin product may be used to generate heat. In some embodiments, high quality lignin product may be combusted to generate heat/gasses. In some embodiments, combusted lignin may be used to generate power. In some embodiments, high quality lignin product may be used in compositions for artificial fire logs with improved flame properties.

Grease.

High quality lignin product may be used to thicken base grease to form lubrication grease or greases with other improved properties. In some embodiments, high quality lignin product may be used in compositions of grease with improved corrosion protection properties. In some embodiments, high quality lignin product may be used in compositions of grease with improved wear resistance. In some embodiments, high quality lignin product may be used in compositions of grease with improved anti-friction properties providing longer lubrication life.

Dispersants.

High quality lignin product may be used to manufacture dispersants. In some embodiments, high quality lignin product may be used in compositions of dye dispersants with improved dispersion, heat-resistant stability, high temperature dispersion, fiber staining, azo dye reducing property, and the like. In some embodiments, high quality lignin product may be used in compositions for dispersing agents, complexing agents, flocculants, thickeners, coating agents, paint agents, adhesive agents, and the like. In some embodiments, high quality lignin product may be used in compositions for oil well drilling muds. In some embodiments, high quality lignin product may be used in compositions for coal-water slurry dispersants. In some embodiments, high quality lignin product may be used in compositions for soil dispersants. In some embodiments, high quality lignin product may be used in compositions for cleaning and/or laundry detergent compounds. In some embodiments, high quality lignin product may be used in compositions for aluminum cleaning. In some embodiments, high quality lignin product may be used in compositions for emulsifiers or dispersants for emulsion. In some embodiments, high quality lignin product may be used in compositions for dispersion polymerization. In some embodiments, high quality lignin product may be used in compositions for jet printing ink.

Agriculture.

High quality lignin product may be used in compositions of slow-release urea and/or fertilizer. In some embodiments, high quality lignin product may be used in compositions for fertilizer binders. In some embodiments, high quality lignin product may be used in compositions for binders. In some embodiments, high quality lignin product may be used in compositions for dispersant agents for pesticides and/or herbicides. In some embodiments, high quality lignin product may be used in compositions for emulsifiers. In some embodiments, high quality lignin product may be used in compositions for heavy metal sequestrates. In some embodiments, high quality lignin product may be used as an additive for restoring vegetation or road slope and bare mountain. In some embodiments, high quality lignin product may be used in compositions for soil water retention agents.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A method for recovering biomass components from an ionic liquid, the method comprising: forming a first phase and a second phase from a hydrolyzed biomass composition comprising an ionic liquid, water and one or more biomass components, wherein the first phase comprises the ionic liquid and the second phase comprises water and one or more biomass components.

2. The method of any of the preceding claims, wherein the hydrolyzed biomass composition is obtained by hydrolyzing the biomass and/or biomass component in the ionic liquid.

3. The method of any of the preceding claims wherein the biomass component is a sugar.

4. The method of any of the preceding claims, wherein the sugar comprises glucose.

5. The method of any of the preceding claims, wherein the sugar at least partially stabilizes the second phase.

6. The method of any of the preceding claims, wherein the composition is pressurized to form the first phase and the second phase.

7. The method of any of the preceding claims, wherein the temperature of the composition is reduced to form the first phase and the second phase.

8. The method of any of the preceding claims, wherein the composition is contacted with pressurized carbon dioxide to form the first phase and the second phase.

9. A method for recovering biomass components from an ionic liquid comprising:
(a) contacting a composition comprising ionic liquid, water and a hydrogen bonding solute with a fluid to form a first phase comprising ionic liquid and a second phase comprising water and the hydrogen bonding solute;
(b) recovering or concentrating at least some of the hydrogen bonding solute from the second phase; and
(c) returning at least some of the hydrogen bonding solute from (b) to the mixture.

10. The method of any of the preceding claims, wherein the hydrogen bonding solute is recovered or concentrated by reverse osmosis.

11. A method for recovering biomass components from an ionic liquid comprising:
(a) contacting a composition comprising ionic liquid, water and a hydrogen bonding solute with a fluid to form a first phase comprising ionic liquid and a second phase comprising ionic liquid, water and the hydrogen bonding solute; and
(b) recovering or concentrating at least some of the ionic liquid from the second phase.

12. The method of any of the preceding claims, wherein the ionic liquid is recovered by electrodialysis.

13. The method of any of the preceding claims, wherein an aqueous biphasic system (ABS) is produced.

14. The method of any of the preceding claims further comprising partitioning the second phase from the first phase.

15. The method of any of the preceding claims, wherein contacting the composition with the fluid forms a third phase comprising the fluid.

16. The method of any of the preceding claims, wherein the fluid comprises carbon dioxide.

17. The method of any of the preceding claims, wherein the composition is contacted with the fluid at a pressure greater than atmospheric pressure.

18. The method of any of the preceding claims, wherein contacting the composition with the fluid decreases the viscosity of the composition.

19. A method for recovering biomass components from an ionic liquid, the method comprising:
(a) forming a first phase and a second phase from a hydrolyzed biomass composition comprising ionic liquid, water and one or more biomass components, wherein the first phase comprises ionic liquid and the second phase comprises water and one or more biomass components;
(b) recovering or concentrating at least some of the biomass components from the second phase; and
(c) returning at least some of the biomass components from (b) to the hydrolyzed biomass composition.

20. The method of any of the preceding claims, wherein the biomass components are recovered or concentrated by reverse osmosis.

21. A method for recovering biomass components from an ionic liquid, the method comprising:
(a) forming a first phase and a second phase from a hydrolyzed biomass composition comprising ionic liquid, water and one or more biomass components, wherein the first phase comprises ionic liquid and the second phase comprises ionic liquid, water and one or more biomass components; and
(b) recovering or concentrating at least some of the ionic liquid from the second phase.

22. The method of any of the preceding claims, wherein the ionic liquid is recovered by electrodialysis.

23. The method of any of the preceding claims, wherein an aqueous biphasic system (ABS) is produced.

24. The method of any of the preceding claims, wherein the hydrolyzed biomass composition is obtained by hydrolyzing the biomass and/or biomass component in the ionic liquid.

25. The method of any of the preceding claims wherein the biomass component is a sugar.

26. The method of any of the preceding claims, wherein the sugar at least partially stabilizes the second phase.

27. The method of any of the preceding claims, wherein the composition is pressurized to form the first phase and the second phase.

28. The method of any of the preceding claims, wherein the composition is contacted with pressurized carbon dioxide to form the first phase and the second phase.

29. The method of any of the preceding claims, further comprising lowering the temperature of the composition, first phase and/or second phase.

30. An extractor capable of performing the method of any of the preceding claims.

31. An extractor comprising an inlet, a first outlet and a second outlet, wherein the inlet is configured to feed a composition comprising ionic liquid, water and a solute into the extractor, the extractor is capable of forming a first phase comprising ionic liquid and a second phase comprising water and the solute; and at least one of:
(a) the first outlet is in fluid communication with a first unit capable of recovering or concentrating the solute in the second phase; and
(b) the second outlet is in fluid communication with a second unit capable of recovering gases dissolved in the first phase.

32. The extractor of any of the preceding claims, wherein the first unit is a reverse osmosis unit.

33. The extractor of any of the preceding claims, wherein the second unit is a flash tank or a heated tank.

34. A method for recovering biomass components from an ionic liquid, the method comprising: pressurizing a hydrolyzed biomass composition comprising ionic liquid, water and one or more biomass components to form a first phase and a second phase, wherein the first phase comprises ionic liquid and the second phase comprises water and one or more biomass components.

35. The method of any of the preceding claims, wherein the composition is pressurized to greater than atmospheric pressure.

36. The method of any of the preceding claims, wherein the gas is carbon dioxide.

37. A method for recovering biomass components from an ionic liquid comprising:
contacting a composition comprising ionic liquid and water with a pressurized gas to form a first phase comprising ionic liquid and a second phase comprising water.

38. The method of any of the preceding claims, wherein the composition further comprises a hydrogen bonding solute.

39. The method of any of the preceding claims, wherein the gas is carbon dioxide.

40. A method for dissolving biomass and components thereof, the method comprising contacting the biomass or component thereof with an ionic liquid at a pressure greater than atmospheric pressure.

41. The method of any of the preceding claims, wherein the pressure is increased to more than 2 atmospheres, more than 5 atmospheres, or more than 20 atmospheres.

42. The method of any of the preceding claims, wherein the applied pressure is stationary or non-stationary.

43. The method of any of the preceding claims, wherein the non-stationary pressure takes the form of vibration, acoustic waves, ultrasound, agitation, and the like.

44. The method of any of the preceding claims, wherein the pressure is oscillated.

45. The method of any of the preceding claims, wherein the increased pressure increases the rate at which the biomass or components thereof dissolve in the ionic liquid by at least 1% relative to the rate at which the biomass dissolves in the ionic liquid at atmospheric pressure.

46. The method of any of the preceding claims, wherein the increased pressure increases the solubility of the biomass or components thereof in the ionic liquid by at least 1% relative to the solubility of the biomass in the ionic liquid at atmospheric pressure.

47. The method of any of the preceding claims, wherein the ionic liquid is contacted with a pressurized gas.

48. The method of any of the preceding claims, wherein contacting the ionic liquid with the pressurized gas reduces the viscosity of the ionic liquid by at least 5%.

49. The method of any of the preceding claims, wherein the rate at which the biomass dissolves in the ionic liquid is at least 20% of the maximum rate at pressures between atmospheric pressure and 100 atm.

50. The method of any of the preceding claims, wherein the solubility of the biomass in the ionic liquid is at least 20% of the maximum solubility at pressures between atmospheric pressure and 100 atm.

51. A method for dissolving biomass comprising contacting biomass with ionic liquid, wherein the ionic liquid is in contact with a pressurized gas.

52. The method of any of the preceding claims, wherein the rate at which the biomass dissolves in the ionic liquid is at least 5% greater than the rate at which the biomass dissolves in the ionic liquid when the ionic liquid is not in contact with the pressurized gas.

53. The method of any of the preceding claims, wherein the solubility of the biomass in the ionic liquid is at least 1% greater than the solubility of the biomass in the ionic liquid when the ionic liquid is not in contact with the pressurized gas.

54. The method of any of the preceding claims, wherein contacting the ionic liquid with the pressurized gas reduces the viscosity of the ionic liquid by at least 5%.

55. The method of any of the preceding claims, wherein the rate at which the biomass dissolves in the ionic liquid is at least 20% of the maximum rate at pressures between atmospheric pressure and 100 atm.

56. The method of any of the preceding claims, wherein the solubility of the biomass in the ionic liquid is at least 20% of the maximum solubility at pressures between atmospheric pressure and 100 atm.

57. The method of any of the preceding claims, wherein the gas comprises carbon dioxide.

58. A method for hydrolyzing biomass, the method comprising adding water to a reaction mixture comprising ionic liquid and biomass, wherein the water is added at a rate such that the ratio (by mass) of side-products (HMF, furan) to sugar is less than the ratio (by mass) of side-products (HMF, furan, etc) to sugar obtained by the identical reaction comprising a fixed amount of water added.

59. A method for hydrolyzing biomass, the method comprising adding water to a reaction mixture comprising ionic liquid and biomass, wherein the water is added at a rate such that biomass solubilization is not substantially inhibited and hydrolysis is not substantially inhibited.

60. A method for hydrolyzing biomass, the method comprising adding water to a reaction mixture comprising ionic liquid and biomass, wherein the water is added at a rate that is approximately equal to the rate at which water is consumed in the reaction.

61. A method for hydrolyzing biomass, the method comprising adding water to a reaction mixture comprising ionic liquid and biomass, wherein the water is added at a rate that maintains the water concentration in the reaction mixture below about 5% (w/w).

62. The method of any of the preceding claims, wherein the reaction mixture further comprises a pressurized gas.

63. The method of any of the preceding claims, wherein the reaction mixture further comprises an acid.

64. The method of any of the preceding claims, wherein the reaction mixture further comprises carbon dioxide and at least some of the carbon dioxide is present as carbonic acid.

65. A method for hydrolyzing biomass comprising adding water to a mixture, wherein the mixture comprises biomass, water and ionic liquid, and wherein the water is added at a rate that maintains the ratio of the concentration of water to the concentration of the biomass in the mixture.

66. The method of any of the preceding claims, wherein the ratio is maintained until the biomass is at least 75% hydrolyzed.

67. The method of any of the preceding claims, further comprising, when the biomass is at least 90% hydrolyzed, adding water to the mixture to increase the ratio to at least 200% of the ratio before water is added.

68. A method for hydrolyzing biomass comprising adding water to a mixture, wherein the mixture comprises biomass, water and ionic liquid, and wherein the water is added at a rate that maintains the ratio of the concentration of water to the concentration of the ionic liquid in the mixture.

69. The method of any of the preceding claims, wherein the ratio is maintained between 50% and 150% of the ratio before water is added.

70. The method of any of the preceding claims, wherein the mixture further comprises an acid.

71. A method for hydrolyzing biomass, comprising adding water to a mixture, wherein the mixture comprises ionic liquid, water and biomass having glycosidic bonds, and wherein the water is added to the mixture at a rate that maintains a greater than stoichiometric concentration of water relative to the glycosidic bonds.

72. The method of any of the preceding claims, wherein the ratio of the concentration of water to the concentration of glycosidic bonds is about 2.

73. A method for hydrolyzing biomass, wherein water is added to a mixture, wherein the mixture comprises ionic liquid, water and biomass, and wherein the water is added at a rate that reduces the electrical conductivity of the reaction mixture over time.

74. The method of any of the preceding claims, wherein the electrical conductivity is reduced in proportion to the extent to which the biomass is hydrolyzed.

75. A method for hydrolyzing biomass, wherein a volume of water is added to a mixture comprising biomass and ionic liquid at least 2 times during the time period in which the biomass is being hydrolyzed.

76. A method for hydrolyzing biomass, the method comprising:
a. dissolving at least some biomass in an ionic liquid;
b. hydrolyzing at least some of the biomass; and
c. adding water to the biomass and ionic liquid.

77. The method of any of the preceding claims, wherein the water is added after at least some of the biomass is converted to water-soluble carbohydrates.

78. A method for hydrolyzing biomass, the method comprising:
a. contacting biomass with ionic liquid; and
b. adding water to the biomass and ionic liquid at least 5 minutes after the contacting.

79. The method of any of the preceding claims, wherein the water is added at least 20 minutes after the contacting.

80. The method of any of the preceding claims, wherein the biomass and ionic liquid are contacted in the presence of an acid.

81. A method for hydrolyzing biomass, the method comprising (a) adding biomass comprising cellulose to ionic liquid such that the cellulose dissolves in the ionic liquid over a period of time; and (b) adding water to the ionic liquid when the degree of polymerization of the dissolved cellulose is less than 99% of the degree of polymerization of the cellulose in the biomass before being dissolved in the ionic liquid.

82. A method for hydrolyzing biomass, the method comprising (a) adding biomass comprising hemicellulose to ionic liquid such that the hemicellulose dissolves in the ionic liquid over a period of time; and (b) adding water to the ionic liquid when the degree of polymerization of the dissolved hemicellulose is less than 99% of the degree of polymerization of the hemicellulose in the biomass before being dissolved in the ionic liquid.

83. A method for hydrolyzing biomass comprising adding water to a mixture of biomass and ionic liquid after the degree of polymerization of cellulose or hemicellulose dissolved in the ionic liquid has been reduced by at least 1% compared with the degree of polymerization of the cellulose or hemicellulose in the biomass before being dissolved in the ionic liquid.

84. The method of any of the preceding claims, wherein the concentration of water increases over time.

85. The method of any of the preceding claims, wherein water is added at a faster rate than it is consumed in the hydrolysis reaction.

86. A method for hydrolyzing biomass, comprising adding water to a mixture of biomass and ionic liquid at a rate such that the concentration of furanic compounds in the mixture is less than 1% (w/w).

87. A method for hydrolyzing a biomass polysaccharide substrate comprising hydrolyzing a reaction mixture comprising the biomass polysaccharide substrate and an ionic liquid in which the biomass polysaccharide substrate is soluble and adding water to the reaction mixture, wherein water is added at a rate such that the polysaccharide of the biomass polysaccharide substrate is not precipitated from the reaction mixture and hydrolysis is not substantially inhibited, and following hydrolysis, lowering the temperature of the reaction mixture from the temperature at which hydrolysis is performed.

88. The method of any of the preceding claims, wherein the biomass polysaccharide substrate is lignocellulosic biomass.

89. The method of any of the preceding claims, wherein the reaction mixture is heated to a temperature of about 70 to 140° C. during hydrolysis.

90. The method of any of the preceding claims, wherein the reaction mixture is cooled to a temperature of about 20 to 100° C. following hydrolysis.

91. The method of any of the preceding claims, wherein the temperature is lowered such that the yield of 5-hydroxymethylfurfural in the hydrolysis product is 10% or less.

92. The method of any of the preceding claims, wherein a co-solvent is added to the reaction mixture in an amount ranging from 1 to 25 weight % of the reaction mixture.

93. A hydrolysis product prepared by the method of any of the preceding claims.

94. A method for making a monosaccharide feedstock which comprises preparing a hydrolysis product as in any of the preceding claims and separating the hydrolysis product from ionic liquid.

95. A method for generating ethanol by fermentation which comprises employing the hydrolysis product of the method of any of the preceding claims as a monosaccharide feedstock for fermentation by an ethanologenic microorganism.

96. A method for hydrolyzing a biomass polysaccharide substrate comprising hydrolyzing a reaction mixture comprising the biomass polysaccharide substrate and an ionic liquid in which the biomass polysaccharide substrate is soluble and adding water to the reaction mixture, wherein water is added at a rate such that the polysaccharide of the biomass polysaccharide substrate is not precipitated from the reaction mixture and hydrolysis is not substantially inhibited, wherein the pressure at which hydrolysis is performed is not atmospheric pressure.

97. The method of any of the preceding claims, wherein the pressure is greater than atmospheric pressure.

98. The method of any of the preceding claims, wherein the pressure is increased as the hydrolysis reaction proceeds.

99. The method of any of the preceding claims, wherein the reaction mixture further comprises acid.

100. The method of any of the preceding claims, wherein the reaction mixture is heated to a temperature of about 70 to 140° C. during hydrolysis.

101. The method of any of the preceding claims, wherein the reaction mixture is cooled to a temperature of about 20 to 100° C. following hydrolysis.

102. The method of any of the preceding claims, wherein the pressure is such that the yield of 5-hydroxymethylfurfural in the hydrolysis product is 10% or less.

103. The method of any of the preceding claims, wherein a co-solvent is added to the reaction mixture in an amount ranging from 1 to 25 weight % of the reaction mixture.

104. A hydrolysis product prepared by the method of any of the preceding claims.

105. A method for making a monosaccharide feedstock which comprises preparing a hydrolysis product as in any of the preceding claims and separating the hydrolysis product from ionic liquid.

106. A method for hydrolyzing biomass, the method comprising applying a variable pressure to a mixture comprising biomass, water and ionic liquid.

107. The method of any of the preceding claims, wherein the pressure is varied such that the solubility of the biomass in the ionic liquid is not substantially decreased and the rate of hydrolysis is not substantially decreased.

108. A method for hydrolyzing biomass, the method comprising contacting biomass with ionic liquid at a pressure greater than atmospheric pressure, wherein the biomass is hydrolyzed in the ionic liquid.

109. The method of any of the preceding claims, wherein the pressure is greater than 5 atm.

110. The method of any of the preceding claims, wherein the rate of hydrolysis is at least 20% greater than the rate of hydrolysis at atmospheric pressure.

111. The method of any of the preceding claims, wherein water is added to the biomass and ionic liquid at a rate that is approximately equal to the rate at which water is consumed in the hydrolysis reaction.

112. A method for hydrolyzing biomass comprising contacting biomass with ionic liquid, wherein the ionic liquid is in contact with a pressurized gas and the biomass is hydrolyzed in the ionic liquid.

113. The method of any of the preceding claims, wherein the rate of hydrolysis is at least 5% greater than the rate of hydrolysis when the ionic liquid is not in contact with the pressurized gas.

114. The method of any of the preceding claims, wherein the ionic liquid comprises carbonic acid.

115. The method of any of the preceding claims, wherein the biomass is contacted with the ionic liquid at a temperature, the ionic liquid is a liquid at the temperature when in contact with the pressurized gas, and the ionic liquid is a solid at the temperature when not in contact with the pressurized gas.

116. The method of any of the preceding claims, wherein contacting the ionic liquid with the pressurized gas reduces the viscosity of the hydrolysis reaction by at least 5%.

117. A method comprising:
a) adding biomass to a vessel comprising ionic liquid; and
b) adding a pressurized gas to the vessel,
wherein the biomass is dissolved and hydrolyzed to sugar in the ionic liquid and at least one of (i) lignin is not dissolved in the ionic liquid, (ii) lignin is precipitated from the ionic liquid, (iii) the sugar is extracted in an aqueous phase, (iv) the sugar is extracted in the pressurized gas, (v) oils are removed by phase separation, and (vi) oils are extracted in the pressurized gas.

118. The method of any of the preceding claims, wherein the vessel maintains a pressure gradient.

119. The method of any of the preceding claims, wherein the ionic liquid comprises acid.

120. A method comprising (a) contacting biomass with a mixture comprising ionic liquid and gas, and (b) applying a varying pressure, wherein the contacting and varying pressure results in a first phase comprising ionic liquid and a second phase comprising sugar.

121. The method of any of the preceding claims, wherein the second phase comprises water.

122. The method of any of the preceding claims, wherein the gas is carbon dioxide.

123. The method of any of the preceding claims, further comprising recovering lignin and/or oils from the ionic liquid.

124. A method comprising hydrolyzing biomass in ionic liquid in a vessel and separating the hydrolysate from the ionic liquid in the vessel.

125. The method of any of the preceding claims, wherein the vessel is a column.

126. The method of any of the preceding claims, wherein the vessel comprises pressurized gas.

127. The method of any of the preceding claims, wherein the water soluble sugars of the hydrolysate are separated from the ionic liquid in a water phase.

128. The method of any of the preceding claims, wherein the solids of the hydrolysate are separated from the ionic liquid with a filter.

129. The method of any of the preceding claims, wherein the solids comprise lignin, ash, or any combination thereof.

130. A method for removing a biomass component from ionic liquid, the method comprising contacting a fluid with ionic liquid having a dissolved biomass component, wherein the biomass component precipitates from the ionic liquid.

131. The method of any of the preceding claims, wherein the fluid is miscible in the ionic liquid.

132. The method of any of the preceding claims, wherein the fluid comprises carbon dioxide.

133. The method of any of the preceding claims, wherein the fluid is a gas pressurized above atmospheric pressure.

134. The method of any of the preceding claims, wherein the biomass component is derived from lignocellulose.

135. The method of any of the preceding claims, wherein the biomass component is lignin, cellulose, hemicellulose, ash, protein, starch, or any combination thereof.

136. The method of any of the preceding claims, further comprising adding a co-solvent to the ionic liquid.

137. The method of any of the preceding claims, wherein the co-solvent is water, ethanol, a ketone, or any combination thereof.

138. The method of any of the preceding claims, further comprising partitioning the precipitated biomass component from the ionic liquid, optionally washing the partitioned biomass component, and optionally drying the washed biomass component.

139. The method of any of the preceding claims, wherein the biomass component is partitioned by filtration or centrifugation.

140. The method of any of the preceding claims, wherein the biomass component is washed with water, ethanol, or any combination thereof.

141. The method of any of the preceding claims, wherein ionic liquid is recovered from the wash.

142. The method of any of the preceding claims, wherein the partitioned, washed and/or dried biomass component comprises less than 1% (w/w) ionic liquid.

143. The method of any of the preceding claims, wherein the partitioned, washed and/or dried biomass component comprises less than 0.1% (w/w) ionic liquid.

144. A method for removing solids from an ionic liquid, the method comprising contacting a pressurized gas with ionic liquid having dissolved solids, wherein the solids precipitate from the ionic liquid.

145. The method of any of the preceding claims, wherein the solids comprise lignin, cellulose, hemicellulose, ash, or any combination thereof.

146. The method of any of the preceding claims, wherein the gas comprises carbon dioxide.

147. The method of any of the preceding claims, wherein the gas is pressurized to greater than atmospheric pressure.

148. A method comprising:
a. providing a composition comprising ionic liquid and dissolved solids;
b. providing an extractant above the boiling point temperature of the extractant; and
c. contacting said composition with said extractant, wherein said contacting precipitates the solids.

149. The method of any of the preceding claims, further comprising recovering the precipitated solids from the ionic liquid.

150. The method of any of the preceding claims, wherein the solids are recovered by filtration.

151. A method comprising providing a composition comprising ionic liquid and solids dissolved therein and recovering the solids from the ionic liquid, wherein recovering the solids results in a loss of less than 1% (w/w) of the ionic liquid.

152. The method of any of the preceding claims, wherein the method results in a loss of less than 0.5 (w/w) of the ionic liquid.

153. The method of any of the preceding claims, wherein the solids comprise lignin, cellulose, hemicellulose, ash, or any combination thereof.

154. A method comprising providing a composition comprising ionic liquid and solids dissolved therein and recovering the solids from the ionic liquid, wherein the recovered solids comprise less than 1% (w/w) ionic liquid.

155. The method of any of the preceding claims, wherein the recovered solids comprise less than 0.1% (w/w) ionic liquid.

156. The method of any of the preceding claims, wherein the solids comprise lignin, cellulose, hemicellulose, ash, or any combination thereof.

157. The solids produced by any of the preceding claims.

158. The lignin produced by any of the preceding claims.

159. An aromatic compound, concrete additive, antioxidant, asphalt additive, carbon fiber or other fiber, board binder, foam, plastic or other polymer, dust control product, paper product, chemical product, battery, fuel, heat, grease, dispersant, or fertilizer produced from the lignin of any of the previous claims.
